(19) United States
(12) Patent Application Publication
(10) Pub. No.: US 2026/0098253 A1
(43) Pub. Date: Apr. 9, 2026

(54) LYSIS VESSEL FOR ON-BOARD LYSIS

(71) Applicant: Gen-Probe Incorporated, San Diego, CA (US)

(72) Inventors: Byron J. KNIGHT, San Diego, CA (US); Norbert D. HAGEN, Carlsbad, CA (US); David OPALSKY, San Diego, CA (US); Daniel J. SOLIS, Escondido, CA (US)

(73) Assignee: Gen-Probe Incorporated, San Diego, CA (US)

(21) Appl. No.: 19/416,385

(22) Filed: Dec. 11, 2025

Related U.S. Application Data
(63) Continuation of application No. PCT/US2025/042626, filed on Aug. 19, 2025.
(60) Provisional application No. 63/685,122, filed on Aug. 20, 2024.
(60) Provisional application No. 63/752,023, filed on Jan. 31, 2025.

(51) Int. Cl. C12N 15/10; B01L 3/00; G01N 1/28; G01N 1/34

(52) U.S. Cl. CPC C12N 15/1013; B01L 3/502761; G01N 1/286; G01N 1/34; B01L 2200/04; B01L 2200/16; B01L 2300/0681

(57) ABSTRACT

A lysis vessel for performing cell lysis includes a laterally extending member and a sleeve having a bottom end defining an open bottom end of the vessel. A first porous membrane is affixed to a top or bottom surface of the laterally extending member and covers a vent in the member. A second porous membrane is affixed to the bottom end of the sleeve and covers the open bottom end. The bottom surface of the laterally extending member, an inner surface of the sleeve, and the first and second porous membranes define a lysis chamber. A plurality of non-magnetic beads are contained within the lysis chamber, and at least one magnetic element is contained within the lysis chamber. The first and the second porous membranes are sized to retain the plurality of non-magnetic beads and the at least one magnetic element within the lysis chamber.

### CROSS REFERENCE TO RELATED APPLICATIONS

This application is a continuation application of International Application No. PCT/US2025/042626 designating the United States and having an international filing date of Aug. 19, 2025, and which claims the benefit of the filing date of U.S. Provisional Application No. 63/685,122, filed Aug. 20, 2024, and U.S. Provisional Application No. 63/752,023, filed Jan. 31, 2025, the disclosures of which are incorporated by reference herein for all purposes.

### FIELD OF THE DISCLOSURE

This disclosure relates to systems and methods for performing mechanical lysis of a sample within a sample chamber of a test platform, such as a fluidic cartridge, by providing the sample to a lysis chamber containing a magnetic element and a plurality of non-magnetic beads and agitating the magnetic element with a magnetic field, whereby the agitated magnetic element contacting the non-magnetic beads agitates the non-magnetic beads to lyse cells contained in the sample. This disclosure additionally relates to a fluidic cartridge with an expansion well and a chamber expander that may be hermetically sealed to a body of the cartridge to expand the volumetric capacity of the expansion well. This disclosure further relates to means and methods for providing an internal control to a sample chamber prior to sample addition. The internal control may be provided in a dried (non-liquid), soluble form, or it may be contained within a capsule or pellet that is disintegrated during mechanical lysis, thereby releasing the internal control into the sample chamber.

### BACKGROUND

Molecular assay procedures performed in test platforms, such as fluidic cartridges, often require that cells contained in a sample be lysed to release nucleic acids therefrom. Lysis may be by, for example, chemical, acoustic, mechanical (physical disruption), and/or enzymatic methods. The cells may be lysed prior to introducing the sample into the fluidic cartridge, thereby requiring extra sample handling and processing prior to introducing the sample into the fluidic cartridge if, for example, acoustic or mechanical lysis methods are employed. Lysing the cells, at least in part, on-board the fluidic cartridge could eliminate the need for such sample handling and processing prior to introducing the sample into the fluidic cartridge.

In addition, fluidic cartridges for performing molecular assay procedures or other tests include multiple wells, or chambers, that are interconnected by channels, often with valves controlling flow through the channels. The volumetric capacity of each chamber is determined by the width and height of the interior space of the chamber. As such fluidic cartridges are often manufactured of molded plastic, limitations in molding techniques may limit the variability in volume metric capacity that can be implemented in the cartridge. For example, limitations in molding techniques may render it impractical to mold a cartridge with multiple wells where one of the wells is significantly taller than the remaining wells. Thus, if one of the wells requires a significantly larger volumetric capacity than the remaining wells, the only way to achieve such larger capacity may be to make the well much wider than the other wells of the cartridge. This will make the width of the overall cartridge larger, or, if the permissible width of the cartridge is constrained, for example, by the size of the instrument in which the cartridge is to be processed, the other wells of the cartridge will need to be made smaller.

Accordingly, a need exists for increasing the volumetric capacity of at least one chamber of a fluidic cartridge.

Where a molecular assay is being performed on fluidic cartridge, it may be desirable for a reaction mixture to include an internal control. An internal control, such as, for example, a plasmid, nucleic acid transcript or a nucleic acid extracted from a whole organism, such as yeast, will be exposed to the same assay conditions as the sample, such as lysis (in the case of a whole organism containing the internal control), sample purification, combination with amplification reagents and detection probes, thermal cycling, etc., so that if the amplification and detection procedures are performed correctly, i.e., all steps of the molecular assay process have been properly conducted with viable reagents used in the assay, detection of a signal indicating the presence of the internal control (i.e., a positive result for the internal control nucleic acid) can be expected. On the other hand, failure to detect a signal indicating the presence of the internal control (i.e., a negative result for the internal control nucleic acid), or detecting less of the internal control than anticipated, may indicate an error or malfunction in one or more steps of the sample preparation (e.g., lysis or analyte purification), the material transport, the amplification, and/or the detection steps and/or that a reagent did not perform as expected. Such errors or malfunctions may be system-based—e.g., the instrument or a module within the instrument has malfunctioned—and/or material-based—e.g., one or more reagents has degraded or become unstable.

An internal control could be provided to the reaction mixture by simply dispensing an amount of a reagent containing the internal control (“internal control reagent” or “ICR”) into a sample chamber along with the sample, or the internal control could be provided to the sample before it is dispensed into the sample chamber. However, these approaches introduce additional steps to the sample preparation process, which can reduce throughput and could lead to errors, spills, or contamination.

### SUMMARY

The following presents a simplified summary in order to provide a basic understanding of some aspects described herein. This summary is not an extensive overview of the claimed subject matter. It is intended to neither identify key or critical elements of the claimed subject matter nor delineate the scope thereof. Its sole purpose is to present some concepts in a simplified form as a prelude to the more detailed description that is presented later.

Implementations of the disclosure can be described in view of the following embodiments, the features of which can be combined in any reasonable manner.

Some embodiments encompass:

A1. A lysis capsule for performing a cell lysis procedure on a fluid sample, wherein the lysis capsule comprises: a hollow body having an open first end and an open second end; a first porous membrane affixed to the body, the first porous membrane covering the open first end; a second porous membrane affixed to the body, the second porous membrane covering the open second end, wherein the hollow body defines a lysis chamber between the first and second porous membranes; a plurality of non-magnetic beads disposed within the lysis chamber; and at least one magnetic element disposed within the lysis chamber, wherein the pores of the first and the second porous membranes are sized to retain the plurality of non-magnetic beads and the at least one magnetic element within the lysis chamber.

A2. The lysis capsule of embodiment A1, wherein the first porous membrane comprises a mesh.

A3. The lysis capsule of embodiment A1 or A2, wherein the first porous membrane is hydrophilic.

A4. The lysis capsule of any one of embodiments A1 to A3, wherein the second porous membrane comprises a mesh.

A5. The lysis capsule of any one of embodiments A1 to A3, wherein the second porous membrane is a filter matrix.

A6. The lysis capsule of any one of embodiments A1 to A5, wherein the first and second porous membranes comprise different porosities.

A7. The lysis capsule of embodiment A6, wherein the first porous membrane has a porosity or range of porosities that is greater than a porosity or range of porosities of the second porous membrane.

A8. The lysis capsule of embodiment A7, wherein the first porous membrane has a porosity of 70 μm to 500 μm, and the second porous membrane has a porosity of 30 μm to 100 μm.

A9. The lysis capsule of any one of embodiments A1 to A8, wherein the first porous membrane is affixed to a top rim of the body defining the open first end, and wherein the second porous membrane is affixed to a bottom rim of the body defining the open second end.

A10. The lysis capsule of any one of embodiments A1 to A9, wherein each of the plurality of non-magnetic beads is comprised of a ceramic or a glass.

A11. The lysis capsule of any one of embodiments A1 to A10, wherein each of the plurality of non-magnetic beads has a spherical shape.

A12. The lysis capsule of embodiment A11, wherein each of the plurality of non-magnetic beads has diameter of 100 μm to 2000 μm.

A13. The lysis capsule of any one of embodiments A1 to A12, wherein the at least one magnetic element is plated or encapsulated with a non-magnetic material.

A14. The lysis capsule of embodiment A13, wherein the non-magnetic material is a metal or a plastic.

A15. The lysis capsule of any one of embodiments A1 to A14, wherein the at least one magnetic element occupies a greater volume than any of the plurality of non-magnetic beads.

A16. The lysis capsule of any one of embodiments A1 to A15, wherein the at least one magnetic element comprises multiple edges.

A17. The lysis capsule of embodiment A16, wherein each of the multiple edges is rounded.

A18. The lysis capsule of embodiment A16 or A17, wherein the at least one magnetic element has the shape of a cube.

A19. The lysis capsule of embodiment A18, wherein the width of each face of the cube is 2.0 millimeters to 4.3 millimeters.

A20. The lysis capsule of any one of embodiments A1 to A19, wherein the at least one magnetic element is comprised of neodymium.

A21. The lysis capsule of embodiment A20, wherein the neodymium is N52 grade or N42 grade.

A22. The lysis capsule of any one of embodiments A1 to A21, wherein the at least one magnetic element and each of the plurality of non-magnetic beads are inert.

A23. The lysis capsule of any one of embodiments A1 to A22, wherein the plurality of non-magnetic beads occupies a volume of 50% to 75% of the volume of the lysis chamber.

A24. The lysis capsule of embodiment A23, wherein the at least one magnetic element occupies a volume of 4.5% to 11% of the volume of the lysis chamber.

A25. The lysis capsule of any one of embodiments A1 to A24, further comprising an internal control contained within the lysis chamber, wherein the internal control is provided to validate an assay result and/or to validate the effectiveness of the cell lysis procedure.

A26. The lysis capsule of embodiment A25, wherein at least a portion of the internal control is contained in an internal control reagent disposed on at least one of the first porous membrane and the second porous membrane, and wherein the internal control reagent is adapted to dissolve when contacted by the fluid sample.

A27. The lysis capsule of embodiment A25, wherein at least a portion of the internal control is contained in an internal control reagent disposed on at least a portion of the plurality of non-magnetic beads and/or the at least one magnetic element, and wherein the internal control reagent is adapted to dissolve when contacted by the fluid sample.

A28. The lysis capsule of embodiment A25, wherein at least a portion of the internal control is contained in an internal control reagent disposed on an internal wall of the hollow body, and wherein the internal control reagent is adapted to dissolve when contacted by the fluid sample.

A29. The lysis capsule of embodiment A25, wherein at least a portion of the internal control is embedded in or contained within an internal control pellet contained within the lysis chamber, and wherein the internal control pellet is adapted to dissolve when contacted by the fluid sample and/or to disintegrate when the plurality of magnetic beads is agitated.

A30. The lysis capsule of embodiment A29, wherein the internal control pellet comprises: a core including an excipient within which the internal control is embedded; and a coating surrounding the core and adapted to be disrupted by mechanical lysing shearing forces imparted by movement of the plurality of non-magnetic beads within the lysis chamber, wherein the excipient is adapted to at least partially dissolve when exposed to fluid after the coating is disrupted.

A31. The lysis capsule of embodiment A30, wherein the excipient comprises at least one of microcrystalline cellulose and hydroxypropylcellulose, and wherein the coating comprises a cellulose derivative.

A32. The lysis capsule of any one of embodiments A25 to A31, wherein the internal control is a whole organism, a plasmid, or a nucleic acid transcript.

Some embodiments encompass:

B1. A fluidic cartridge, comprising: a cartridge body comprising a sample chamber, the sample chamber having an open top end; and the lysis capsule of any one of embodiments A1 to A24 disposed within the sample chamber.

B2. The fluidic cartridge of embodiment B1, further comprising a syringe barrel in communication with the sample chamber, the syringe barrel being adapted to receive a syringe stopper connected to a syringe plunger for actuating fluids within the fluidic cartridge.

B3. The fluidic cartridge of embodiment B1 or B2, further comprising a dead space within the sample chamber situated below the lysis capsule.

B4. The fluidic cartridge of any one of embodiments B1 to B3, wherein the sample chamber is covered by a removable seal.

B5. The fluidic cartridge of any one of embodiments B1 to B3, further comprising a cap adapted to be inserted into the open top end of the sample chamber.

B6. The fluidic cartridge of embodiment B5, wherein the cap comprises a sleeve, and wherein an outer surface of the sleeve is in sealing engagement with an inner surface of the sample chamber when the cap is inserted into the open top end of the sample chamber.

B7. The fluidic cartridge of embodiment B5 or B6, wherein a bottom end of the cap is disposed adjacent a top end of the lysis capsule when the cap is inserted into the open top end of the sample chamber.

B8. The fluidic cartridge of embodiment B7, wherein a gap exists between the top end of the lysis capsule and the bottom end of the cap when the cap is inserted into the open top end of the sample chamber.

B9. The fluidic cartridge of any one of embodiments B6 to B8, wherein the cap comprises a laterally extending member, wherein a peripheral region of the laterally extending member is seated on a top surface of the cartridge body, and wherein the sleeve depends from the laterally extending member such that an inner surface of the sleeve and a bottom surface of the laterally extending member define a recess extending upward from the bottom end of the cap.

B10. The fluidic cartridge of embodiment B9, wherein the laterally extending member has a vent extending therethrough, the vent enabling air to escape from the sample chamber when the cap is inserted into the sample chamber.

B11. The fluidic cartridge of embodiment B10, wherein the cap comprises a porous vent membrane affixed to a top or bottom surface of the laterally extending member and covering the vent.

B12. The fluidic cartridge of embodiment B11, wherein the porous vent membrane is permeable to air but impermeable to liquids.

B13. The fluidic cartridge of embodiment B11 or B12, wherein the porous vent membrane has a porosity of 0.2 μm to 0.4 μm.

B14. The fluidic cartridge of any one of embodiments B9 to B13, wherein the cap comprises a peripheral wall extending upward from a periphery of the laterally extending member.

B15. The fluidic cartridge of embodiment B14, wherein the wall is adapted for manual gripping.

B16. The fluidic cartridge of any one of embodiments B1 to B15, further comprising an internal control contained within the lysis chamber, wherein the internal control is provided to validate an assay result and/or to validate the effectiveness of the cell lysis procedure.

B17. The fluidic cartridge of embodiment B16, wherein at least a portion of the internal control is contained in an internal control reagent disposed on at least one of the first porous membrane and the second porous membrane, and wherein the internal control reagent is adapted to dissolve when contacted by a fluid sample.

B18. The fluidic cartridge of embodiment B16 or B17, wherein at least a portion of the internal control is contained in an internal control reagent disposed on at least a portion of the plurality of non-magnetic beads and/or the at least one magnetic element within the lysis chamber, and wherein the internal control reagent is adapted to dissolve when contacted by a fluid sample.

B19. The fluidic cartridge of embodiment B16, wherein at least a portion of the internal control is embedded in or contained within an internal control pellet contained within the lysis chamber, and wherein the internal control pellet is adapted to dissolve when contacted by a fluid sample and/or to disintegrate when the plurality of non-magnetic beads is agitated.

B20. The fluidic cartridge of embodiment B19, wherein the internal control pellet comprises: a core including an excipient within which the internal control is embedded; and a coating surrounding the core and adapted to be disrupted by mechanical lysing shearing forces imparted by movement of the plurality of non-magnetic beads within the receptacle, wherein the excipient is adapted to at least partially dissolve when exposed to fluid after the coating is disrupted.

B21. The fluidic cartridge of embodiment B20, wherein the excipient comprises at least one of microcrystalline cellulose and hydroxypropylcellulose, and wherein the coating comprises a cellulose derivative.

B22. The fluidic cartridge of any one of embodiments B16 to B21, wherein the internal control is a whole organism, a plasmid, or a nucleic acid transcript.

Some embodiments encompass:

C1. A method for processing cells contained in a fluid sample, comprising: (a) dispensing the fluid sample into the sample chamber of the fluidic cartridge of any one of embodiments B1 to B3 to at least partially fill the lysis chamber of the lysis capsule with the fluid sample; (b) after (a), covering the open top end of the sample chamber with a cap; and (c) after (b), subjecting the at least one magnetic element to a magnetic field, thereby causing movement of the at least one magnetic element within the lysis chamber of the lysis capsule, the movement of the at least one magnetic element within the lysis chamber of the lysis capsule causing movement of the plurality of non-magnetic beads within the lysis chamber of the lysis capsule, and the movement of the plurality of non-magnetic beads within the lysis chamber of the lysis capsule causing cells contained within the fluid sample within the lysis chamber of the lysis capsule to lyse and release nucleic acids.

C2. The method of embodiment C1, wherein the fluid sample dispensed in (a) occupies 39% to 45% of a volume of the lysis chamber of the lysis capsule.

C3. The method of embodiment C1 or C2, wherein (b) comprises inserting the cap into the sample chamber, such that the cap is in sealing engagement with the sample chamber.

C4. The method of embodiment C3, wherein a gap separates a top end of the lysis capsule from a bottom end of the cap following insertion of the cap into the sample chamber.

C5. The method of any one of embodiments C1 to C4, wherein (b) comprises passing air from the sample chamber to an external environment through a vent in the cap.

C6. The method of embodiment C5, wherein (b) further comprises passing the air through a porous vent membrane affixed to the cap and covering the vent, the porous vent membrane being impermeable to liquids.

C7. The method of any one of embodiments C1 to C6, wherein the magnetic field is created by an electromagnet during (c).

C8. The method of embodiment C7, wherein (c) comprises alternating a current to the electromagnet to alternate a polarity of the electromagnet.

C9. The method of embodiment C8, wherein (c) comprises alternating the current to alternate the polarity of the electromagnet at a frequency of 20 Hertz to 200 Hertz.

C10. The method of embodiment C9, wherein (c) comprises pulsing the current to alternate the polarity of the electromagnet at two or more different frequencies.

C11. The method of any one of embodiments C1 to C10, further comprising: (d) after (c), transporting at least a portion of the fluid sample from the sample chamber to a processing chamber of the fluidic cartridge.

C12. The method of embodiment C11, further comprising: (e) during (d), retaining lysed cellular material from (c) within the lysis chamber while allowing the released nucleic acids to pass through the second porous membrane.

C13. The method of embodiment C12, further comprising: (f) during (d) subjecting the at least one magnetic element to the magnetic field, thereby causing movement of the at least one magnetic element within the lysis chamber of the lysis capsule, the movement of the at least one magnetic element within the lysis chamber causing movement of the plurality of non-magnetic beads within the lysis chamber, and the movement of the plurality of non-magnetic beads within the lysis chamber causing at least a portion of the lysed cellular material within the lysis chamber to remain in suspension at least until the fluid sample has been removed from the lysis chamber.

C14. The method of any one of embodiments C11 to C13, further comprising: (g) in the processing chamber, immobilizing at least a portion of the released nucleic acids on a solid support and removing non-immobilized components of the fluid sample to a waste chamber of the fluidic cartridge.

C15. The method of embodiment C14, further comprising: (h) after (g), eluting the immobilized nucleic acids from the solid support and transporting the eluted nucleic acids to a reaction chamber of the fluidic cartridge.

C16. The method of embodiment C15, further comprising: (i) after (h), subjecting the eluted nucleic acids to conditions of a first reaction, the first reaction providing an indication of the presence or amount of an analyte of interest.

C17. The method of embodiment C16, further comprising: (j) during or after (a), releasing an internal control into the fluid sample, the internal control being contained within the lysis chamber prior to (a); (k) immobilizing nucleic acids associated with the internal control (“IC nucleic acids”) on the solid support during (g); (l) after (g), eluting the IC nucleic acids from the solid support and transporting the eluted IC nucleic acids to the reaction chamber; and (m) after (l), subjecting the IC nucleic acids to conditions of a second reaction, a result of (m) being used to validate a result of (i) and/or to validate the effectiveness of the lysis in (c).

C18. The method of embodiment C17, wherein at least a portion of the internal control is contained in an internal control reagent disposed on at least one of the first porous membrane and the second porous membrane when (a) is initiated, and wherein the internal control reagent dissolves in the fluid sample during any of (a) to (c).

C19. The method of embodiment C17, wherein at least a portion of the internal control is contained in an internal control reagent disposed on at least a portion of the plurality of non-magnetic beads and/or the at least one magnetic element when (a) is initiated, and wherein the internal control reagent disposed on the at least a portion of the plurality of non-magnetic beads and/or the at least one magnetic element dissolves in the fluid sample during any of (a) to (c).

C20. The method of embodiment C17, wherein at least a portion of the internal control is contained in an internal control reagent disposed on an internal wall of the hollow body when (a) is initiated, and wherein the internal control reagent dissolves in the fluid sample during any of (a) to (c).

C21. The method of embodiment C17, wherein the internal control is embedded in or contained within an internal control pellet, and wherein the internal control pellet dissolves in the presence of the fluid sample and/or is disintegrated by the movement of the plurality of non-magnetic beads during (c), thereby releasing the internal control into the fluid sample.

C22. The method of any one of embodiments C17 to C21, wherein the conditions of the first reaction and the conditions of the second reaction are the same conditions.

C23. The method of any one of embodiments C17 to C22, wherein each of the first and second reactions is a nucleic acid amplification reaction.

C24. The method of embodiment C23, wherein the nucleic acid amplification reaction is a polymerase chain reaction (“PCR”).

Some embodiments encompass:

D1. A lysis vessel for performing cell lysis, comprising: a laterally extending member having a vent extending therethrough; a sleeve depending from the laterally extending member, wherein a bottom end of the sleeve defines an open bottom end of the vessel; a first porous membrane affixed to a top or bottom surface of the laterally extending member, the first porous membrane covering the vent, a second porous membrane affixed to the bottom end of the sleeve, the second porous membrane covering the open bottom end, wherein the bottom surface of the laterally extending member, an inner surface of the sleeve and the first and second porous membranes define a lysis chamber; a plurality of non-magnetic beads contained within the lysis chamber; and at least one magnetic element contained within the lysis chamber, wherein the first and the second porous membranes are sized to retain the plurality of non-magnetic beads and the at least one magnetic element within the lysis chamber.

D2. The lysis vessel of embodiment D1, further comprising a peripheral wall extending upward from the periphery of the laterally extending member.

D3. The lysis vessel of embodiment D2, wherein the peripheral wall is adapted for manual gripping.

D4. The lysis vessel of any one of embodiments D1 to D3, wherein the second porous membrane comprises a mesh, the mesh being liquid permeable.

D5. The lysis vessel of embodiment D4, wherein the second porous membrane is hydrophilic.

D6. The lysis vessel of embodiment D4 or D5, wherein the first porous membrane is gas permeable but not liquid permeable.

D7. The lysis vessel of embodiment D6, wherein the first porous membrane has a porosity of 0.2 μm to 0.4 μm, and the second porous membrane has a porosity of 30 μm to 100 μm.

D8. The lysis vessel of any one of embodiments D1 to D7, wherein each of the plurality of non-magnetic beads is comprised of a ceramic or a glass.

D9. The lysis vessel of any one of embodiments D1 to D8, wherein each of the plurality of non-magnetic beads has a spherical shape.

D10. The lysis vessel of embodiment D9, wherein each of the plurality of non-magnetic beads has diameter of 100 μm to 2000 μm.

D11. The lysis vessel of any one of embodiments D1 to D10, wherein the at least one magnetic element is plated or encapsulated with a non-magnetic material.

D12. The lysis vessel of embodiment D11, wherein the non-magnetic material is a metal or a plastic.

D13. The lysis vessel of any one of embodiments D1 to D12, wherein the at least one magnetic element occupies a greater volume than any of the plurality of non-magnetic beads.

D14. The lysis vessel of any one of embodiments D1 to D13, wherein the at least one magnetic element comprises multiple edges.

D15. The lysis vessel of embodiment D14, wherein each of the multiple edges is rounded.

D16. The lysis vessel of embodiment D14 or D15, wherein the at least one magnetic element has the shape of a cube.

D17. The lysis vessel of embodiment D16, wherein the width of each face of the cube is 2.0 millimeters to 4.3 millimeters.

D18. The lysis vessel of any one of embodiments D1 to D17, wherein the at least one magnetic element is comprised of neodymium.

D19. The lysis vessel of embodiment D18, wherein the neodymium is N52 grade or N42 grade.

D20. The lysis vessel of any one of embodiments D1 to D19, wherein the at least one magnetic element and each of the plurality of non-magnetic beads are inert.

D21. The lysis vessel of any one of embodiments D1 to D20, wherein the plurality of non-magnetic beads occupies a volume of 50% to 75% of the volume of the lysis chamber.

D22. The lysis vessel of embodiment D21, wherein the at least one magnetic element occupies a volume of 4.5% to 11% of the volume of the lysis chamber.

D23. The lysis vessel of any one of embodiments D1 to D22, further comprising an internal control contained within the lysis chamber, wherein the internal control is provided to validate an assay result and/or to validate the effectiveness of the cell lysis procedure.

D24. The lysis vessel of embodiment D23, wherein at least a portion of the internal control is contained in an internal control reagent disposed on the second porous membrane, and wherein the internal control reagent is adapted to dissolve when contacted by a fluid sample.

D25. The lysis vessel of embodiment D23, wherein at least a portion of the internal control is contained in an internal control reagent disposed on at least a portion of the plurality of non-magnetic beads, and wherein the internal control reagent is adapted to dissolve when contacted by a fluid sample.

D26. The lysis vessel of embodiment D23, wherein at least a portion of the internal control is contained in an internal control reagent disposed on at least one of the bottom surface of the laterally extending member and the inner surface of the sleeve, and wherein the internal control reagent is adapted to dissolve when contacted by a fluid sample.

D27. The lysis vessel of embodiment D23, wherein at least a portion of the internal control is embedded in or contained within an internal control pellet adapted to dissolve when contacted by a fluid sample and/or to disintegrate when the plurality of non-magnetic beads is agitated, the internal control pellet being contained within the lysis chamber.

Some embodiments encompass:

E1. A fluidic cartridge, comprising: a cartridge body comprising a sample chamber, the sample chamber having an open top end; and the lysis vessel of any one of embodiments D1 to D28 disposed within the sample chamber.

E2. The fluidic cartridge of embodiment E1, further comprising a syringe barrel in communication with the sample chamber, the syringe barrel being adapted to receive a syringe stopper connected or connectable to a syringe plunger for actuating the syringe stopper within the syringe barrel to actuate fluids within the fluidic cartridge.

E3. The fluidic cartridge of embodiment E1 or E2, further comprising a dead space within the sample chamber situated below the lysis vessel.

E4. The fluidic cartridge of any one of embodiments E1 to E3, wherein an outer surface of the sleeve is in sealing engagement with an inner surface of the sample chamber.

E5. The fluidic cartridge of any one of embodiments E1 to E4, wherein a peripheral region of the laterally extending member is seated on a top surface of the cartridge body.

Some embodiments encompass:

F1. A method for processing cells contained in a fluid sample, comprising: (a) dispensing the fluid sample into a sample chamber of a fluidic cartridge, the sample chamber having an open top end; (b) after (a), inserting the sleeve of the lysis vessel of any one of embodiments D1 to D22 into the open top end of the sample chamber; (c) after (b), subjecting the at least one magnetic element to a magnetic field, thereby causing movement of the at least one magnetic element within the lysis chamber of the lysis vessel, the movement of the at least one magnetic element within the lysis chamber of the lysis vessel causing movement of the plurality of non-magnetic beads within the lysis chamber of the lysis vessel, and the movement of the plurality of non-magnetic beads within the lysis chamber of the lysis vessel causing cells contained within the fluid sample within the lysis chamber of the lysis vessel to lyse and release nucleic acids.

F2. The method of embodiment F1, wherein the fluidic cartridge further comprises a syringe barrel in communication with the sample chamber, the syringe barrel being adapted to receive a syringe stopper connected or connectable to a syringe plunger for actuating the syringe stopper within the syringe barrel to actuate fluids within the fluidic cartridge.

F3. The method of embodiment F1 or F2, wherein the fluidic cartridge further comprises a dead space within the sample chamber situated below the lysis vessel.

F4. The method of any one of embodiments F1 to F3, wherein an outer surface of the sleeve is in sealing engagement with an inner surface of the sample chamber after (b).

F5. The method of any one of embodiments F1 to F4, wherein a peripheral region of the laterally extending member is seated on a top surface of the cartridge body.

F6. The method of any one of embodiments F1 to F5, wherein the fluid sample dispensed in (a) occupies a volume of 39% to 45% of the volume of the lysis chamber of the lysis vessel.

F7. The method of any one of embodiments F1 to F6, wherein (b) further comprises passing air from the sample chamber to an external environment through the vent in the lysis vessel and the first porous membrane.

F8. The method of any one of embodiments F1 to F7, wherein the magnetic field is created by an electromagnet during (c).

F9. The method of embodiment F8, wherein (c) comprises alternating a current to the electromagnet to alternate a polarity of the electromagnet.

F10. The method of embodiment F9, wherein (c) comprises alternating the current at a frequency of 20 Hertz to 200 Hertz.

F11. The method of any one of embodiments F1 to F10, further comprising: (d) after (c), transporting at least a portion of the fluid sample from the sample chamber to a processing chamber of the fluidic cartridge.

F12. The method of embodiment F11, further comprising: (e) during (d), retaining lysed cellular material from (c) within the lysis chamber while allowing the released nucleic acids to pass through the second porous membrane.

F13. The method of embodiment F12, further comprising: (f) during (d) subjecting the at least one magnetic element to the magnetic field, thereby causing movement of the at least one magnetic element within the lysis chamber of the lysis vessel, the movement of the at least one magnetic element within the lysis chamber causing movement of the plurality of non-magnetic beads within the lysis chamber, and the movement of the plurality of non-magnetic beads within the lysis chamber causing at least a portion of the lysed cellular material within the lysis chamber to remain in suspension at least until the fluid sample has been removed from the lysis chamber.

F14. The method of any one of embodiment F11 to F13, further comprising: (g) in the processing chamber, immobilizing at least a portion of the released nucleic acids on a solid support and removing non-immobilized components of the fluid sample to a waste chamber of the fluidic cartridge.

F15. The method of embodiment F14, further comprising: (h) after (g), eluting the immobilized nucleic acids from the solid support and transporting the eluted nucleic acids to a reaction chamber of the fluidic cartridge.

F16. The method of embodiment F15, further comprising: (i) after (h), subjecting the eluted nucleic acids to conditions of a first reaction, the first reaction providing an indication of the presence or amount of an analyte of interest.

F17. The method of embodiment F16, further comprising: (j) during or after (a), releasing an internal control into the fluid sample within the lysis chamber prior to (a); (k) immobilizing nucleic acids associated with the internal control (“IC nucleic acids”) on the solid support during (g); (l) after (g), eluting the IC nucleic acids from the solid support and transporting the eluted IC nucleic acids to the reaction chamber; and (m) after (l), subjecting the IC nucleic acids to conditions of a second reaction, a result of (m) being used to validate a result of (i) and/or to validate the effectiveness of the lysis in (c).

F18. The method of embodiment F17, wherein at least a portion of the internal control is contained in an internal control reagent disposed on the second porous membrane when (a) is initiated, and wherein the internal control reagent dissolves in the fluid sample after (b).

F19. The method of embodiment F17, wherein at least a portion of the internal control is contained in an internal control reagent disposed on at least a portion of the plurality of non-magnetic beads and/or the at least one magnetic element when (b) is initiated, and wherein the internal control reagent disposed on the at least a portion of the plurality of non-magnetic beads and/or the at least one magnetic element dissolves in the fluid sample during (b) and/or (c).

F20. The method of embodiment F17, wherein at least a portion of the internal control is embedded in or contained within an internal control pellet, and wherein the internal control pellet dissolves in the presence of the fluid sample and/or is disintegrated by the movement of the plurality of non-magnetic beads during (c), thereby releasing the internal control into the fluid sample.

F21. The method of any one of embodiments F17 to F19, wherein, prior to (a), the internal control reagent is a dried reagent prior to contact with the fluid sample.

F22. The method of any one of embodiments F17 to F21, wherein the conditions of the first reaction and the conditions of the second reaction are the same conditions.

F23. The method of any one of embodiments F17 to F22, wherein each of the first and second reactions is a nucleic acid amplification reaction.

F24. The method of embodiment F23, wherein the nucleic acid amplification reaction is a polymerase chain reaction (“PCR”).

Some embodiments encompass:

G1. A method of manufacturing a fluidic cartridge containing a lysis capsule, comprising: assembling the lysis capsule by: (a) providing a hollow body having open first and second ends; (b) providing first and second porous membranes and affixing the second porous membrane to the hollow body, the second porous membrane covering the open second end of the hollow body; (c) introducing a plurality of non-magnetic beads into the hollow body through the open first end of the hollow body; (d) introducing at least one magnetic element into the hollow body through the open first end of the hollow body; (e) after (c) and (d), affixing the first porous membrane to the hollow body, the first porous membrane covering the open first end of the hollow body, wherein the hollow body and the affixed first and second porous membranes define a lysis chamber, and wherein the pores of the first and second porous membranes are sized to retain the plurality of non-magnetic beads and the at least one magnetic element within the lysis chamber; and (f) after (e), securing the lysis capsule within a sample chamber of a fluidic cartridge having a plurality of chambers in fluid communication with the sample chamber.

G2. The method of embodiment G1, wherein the first porous membrane comprises a mesh.

G3. The method of embodiment G1 or G2, wherein the second porous membrane comprises a mesh.

G4. The method of any one of embodiments G1 to G3, wherein the first porous membrane is hydrophilic.

G5. The method of any one of embodiments G1 to G4, wherein the first and second porous membranes comprise different porosities.

G6. The method of embodiment G5, wherein the first porous membrane has a porosity or range of porosities that is greater than a porosity or range of porosities of the second porous membrane.

G7. The method of embodiment G6, wherein the first porous membrane has a porosity of 70 μm to 500 μm, and the second porous membrane has a porosity of 30 μm to 100 μm.

G8. The method of any one of embodiments G1 to G7, wherein the first porous membrane is affixed to a top rim of the hollow body defining the open first end, and wherein the second porous membrane is affixed to a bottom rim of the hollow body defining the open second end.

G9. The method of any one of embodiments G1 to G8, wherein each of the plurality of non-magnetic beads is comprised of a ceramic or a glass.

G10. The method of any one of embodiments G1 to G9, wherein each of the plurality of non-magnetic beads has a spherical shape.

G11. The method of embodiment G10, wherein each of the plurality of non-magnetic beads has a diameter of 100 μm to 2000 μm.

G12. The method of any one of embodiments G1 to G11, wherein the at least one magnetic element is plated or encapsulated with a non-magnetic material.

G13. The method of embodiment G12, wherein the non-magnetic material is a metal or a plastic.

G14. The method of any one of embodiments G1 to G13, wherein the at least one magnetic element occupies a greater volume than any of the plurality of non-magnetic beads.

G15. The method of any one of embodiments G1 to G14, wherein the at least one magnetic element comprises multiple edges.

G16. The method of embodiment G15, wherein each of the multiple edges is rounded.

G17. The method of embodiment G15 or G16, wherein the at least one magnetic element has the shape of a cube.

G18. The method of embodiment G17, wherein the width of each face of the cube is 2.0 millimeters to 4.3 millimeters.

G19. The method of any one of embodiments G1 to G18, wherein the at least one magnetic element is comprised of neodymium.

G20. The method of embodiment G19, wherein the neodymium is N52 grade or N42 grade.

G21. The method of any one of embodiments G1 to G20, wherein the at least one magnetic element and each of the plurality of non-magnetic beads is inert.

G22. The method of any one of embodiments G1 to G21, wherein the plurality of non-magnetic beads occupies a volume of 50% to 75% of the volume of the lysis chamber.

G23. The method of embodiment G22, wherein the at least one magnetic element occupies a volume of 4.5% to 11% of the volume of the lysis chamber.

G24. The method of any one of embodiments G1 to G23, further comprising disposing an internal control reagent onto a component of the lysis capsule, the internal control reagent containing an internal control provided to validate an assay and/or to validate the effectiveness of a cell lysis procedure performed with the plurality of non-magnetic beads and the at least one magnetic element.

G25. The method of embodiment G24, wherein at least a portion of the internal control reagent is disposed onto at least a portion of the plurality of non-magnetic beads and/or the at least one magnetic element prior to (c).

G26. The method of embodiment G24, wherein at least a portion of the internal control reagent is disposed onto at least a portion of the plurality of non-magnetic beads and/or the at least one magnetic element after (c).

G27. The method of embodiment G24, wherein the internal control reagent is disposed onto at least one of (i) the first porous membrane, (ii) the second porous membrane, (iii) an inner surface of the hollow body, (iv) at least a portion of the plurality of non-magnetic beads, and (v) the at least one magnetic element before (e).

G28. The method of any one of embodiments G24 to G27, wherein the internal control reagent is disposed in a liquid form, and wherein the method further comprises drying the internal control reagent after it has been disposed onto the lysis capsule.

G29. The method of any one of embodiments G1 to G23, wherein an internal control is embedded in or contained within an internal control pellet adapted to dissolve when contacted by a fluid sample and/or to disintegrate when the plurality of magnetic beads is agitated in the lysis chamber, and wherein the internal control pellet is contained within the hollow body prior to (e), and wherein internal control provided to validate an assay and/or to validate the effectiveness of a cell lysis procedure performed with the plurality of non-magnetic beads and the at least one magnetic element.

G30. The method of any one of embodiments G24 to G29, wherein the internal control is a whole organism, a plasmid or a nucleic acid transcript.

G31. The method of any one of embodiments G1 to G30, wherein each of the first and second porous membranes is affixed to the hollow body by adhesive, heat sealing, or ultrasonic welding.

G32. The method of any one of embodiments G1 to G31, wherein the lysis capsule is press-fitted within the sample chamber.

G33. The method of any one of embodiments G1 to G31, wherein an outer surface of the hollow body is threadedly mated with an inner surface of the sample chamber.

G34. The method of any one of embodiments G1 to G33, further comprising, affixing a removable protective cover to a top surface of the fluidic cartridge, thereby covering the sample chamber.

Some embodiments encompass:

H1. A method for lysing cells contained in a fluid sample, comprising: (a) dispensing the fluid sample into a sample chamber of a fluidic cartridge, the sample chamber containing a plurality of non-magnetic beads and at least one magnetic element, wherein at least a portion of the plurality of non-magnetic beads and/or the at least one magnetic element have an internal control reagent deposited thereon, and wherein an internal control contained in the internal control reagent is provided to validate an assay result and/or to validate the effectiveness of a cell lysis procedure; and (b) after (a), subjecting the fluid sample to the cell lysis procedure, the cell lysis procedure comprising exposing the at least one magnetic element to a magnetic field, thereby causing movement of the at least one magnetic element contained within the sample chamber, the movement of the at least one magnetic element causing movement of the plurality of non-magnetic beads contained within the sample chamber, and the movement of the plurality of non-magnetic beads within the sample chamber causing cells contained within the fluid sample to lyse and release nucleic acids, wherein the internal control reagent dissolves in the presence of the fluid sample, thereby releasing the internal control into the fluid sample, and wherein the movement of the at least one magnetic element and the plurality of non-magnetic beads causes the internal control contained within the dissolved internal control reagent to be distributed within the fluid sample.

H2. A method for lysing cells contained in a fluid sample, comprising: (a) dispensing the fluid sample into a sample chamber of the fluidic cartridge, the sample chamber containing a (i) plurality of non-magnetic beads, (ii) at least one magnetic element, and (iii) an internal control reagent contained within an internal control pellet, wherein an internal control contained in the internal control reagent is provided to validate an assay result and/or to validate the effectiveness of a cell lysis procedure; and (b) after (a), subjecting fluid sample to the cell lysis procedure, the cell lysis procedure comprising exposing the at least one magnetic element to a magnetic field, thereby causing movement of the at least one magnetic element contained within the sample chamber, the movement of the at least one magnetic element causing movement of the plurality of non-magnetic beads contained within the sample chamber, and the movement of the plurality of non-magnetic beads within the sample chamber causes the cells contained within the fluid sample to lyse and release nucleic acids, wherein the internal control pellet dissolves when contacted by the fluid sample and/or the movement of the plurality of non-magnetic beads within the sample chamber causes the internal control pellet to disintegrate, thereby releasing the internal control into the fluid sample, and wherein the movement of the at least one magnetic element and the plurality of non-magnetic beads causes the internal control contained within the dissolved internal control reagent to be distributed within the fluid sample.

H3. The method of embodiment H1 or H2, wherein the plurality of non-magnetic beads and the at least one magnetic element are contained within a hollow body defining a lysis chamber disposed within the sample chamber during (a) and (b), and wherein the receptacle is liquid permeable.

H4. The method of any one of embodiments H1 to H3, wherein each of the plurality of non-magnetic beads has a spherical shape.

H5. The method of embodiment H4, wherein each of the plurality of non-magnetic beads has diameter of 100 μm to 2000 μm.

H6. The method of any one of embodiments H1 to H5, wherein the at least one magnetic element is plated or encapsulated with a non-magnetic material.

H7. The method of embodiment H6, wherein the non-magnetic material is a metal or a plastic.

H8. The method of any one of embodiments H1 to H7, wherein the at least one magnetic element occupies a greater volume than any of the plurality of non-magnetic beads.

H9. The method of any one of embodiments H1 to H8, wherein the at least one magnetic element comprises multiple edges.

H10. The method of embodiment H9, wherein each of the multiple edges is rounded.

H11. The method of embodiment H9 or H10, wherein the at least one magnetic element has the shape of a cube.

H12. The method of embodiment H11, wherein the width of each face of the cube is 2.0 millimeters to 4.3 millimeters.

H13. The method of any one of embodiments H1 to H12, wherein the at least one magnetic element is comprised of neodymium.

H14. The method of embodiment H13, wherein the neodymium is N52 grade or N42 grade.

H15. The method of any one of embodiments H1 to H14, wherein the at least one magnetic element and each of the plurality of non-magnetic beads are inert.

H16. The method of any one of embodiments H1 to H15, wherein the plurality of non-magnetic beads occupies a volume of 50% to 70% of the volume of the sample chamber.

H17. The method of embodiment H16, wherein the at least one magnetic element occupies a volume of 4.5% to 11% of the volume of the sample chamber.

H18. The method of any one of embodiments H1 to H17, wherein the magnetic field is created by an electromagnet during (b).

H19. The method of embodiment H18, wherein (b) comprises alternating a current to the electromagnet to alternate a polarity of the electromagnet.

H20. The method of embodiment H19, wherein (b) comprises alternating the current at a frequency of 20 Hertz to 200 Hertz.

H21. The method of any one of embodiments H1 to H20, further comprising: (c) after (b), transporting at least a portion of the fluid sample from the sample chamber to a processing chamber of the fluidic cartridge.

H22. The method of embodiment H21, further comprising: (d) during (c), retaining lysed cellular material from (c) within the sample chamber while the released nucleic acids is transported to the processing chamber.

H23. The method of embodiment H21 or H22, further comprising: (e) in the processing chamber, immobilizing at least a portion of the released nucleic acids on a solid support and removing non-immobilized components of the fluid sample to a waste chamber of the fluidic cartridge.

H24. The method of embodiment H23, further comprising: (f) after (e), eluting the immobilized nucleic acids from the solid support and transporting the eluted nucleic acids to a reaction chamber of the fluidic cartridge.

H25. The method of embodiment H24, further comprising: (g) after (f), subjecting the eluted nucleic acids to conditions of a first reaction, the first reaction providing an indication of the presence or amount of an analyte of interest.

H26. The method of embodiment H25, further comprising (h) immobilizing nucleic acids associated with the internal control (“IC nucleic acids”) on the solid support during (e); (i) after (h), eluting the IC nucleic acids from the solid support and transporting the eluted IC nucleic acids to the reaction chamber; and (j) after (i), subjecting the IC nucleic acids to conditions of a second reaction, the second reaction providing an indication of the extent of lysis in (b).

H27. The method of embodiment H26, wherein the conditions of the first reaction and the conditions of the second reaction are the same conditions.

H28. The method of embodiment H26 or H27, wherein each of the first and second reactions is a nucleic acid amplification reaction.

H29. The method of embodiment H28, wherein the nucleic acid amplification reaction is a polymerase chain reaction (“PCR”).

Some embodiments encompass:

I1. A fluidic cartridge including a cartridge body defining two or more wells that are fluidly connected or connectable, wherein at least one well of the two or more wells comprises an expansion well, and wherein the cartridge body includes a first coupling structure at least partially surrounding the expansion well, and wherein the cartridge further comprises a chamber expander attached to the cartridge body and comprising: a base with a second coupling structure located on a bottom side of the base and configured to be operatively coupled to the first coupling structure to form a hermetic seal between the chamber expander and the cartridge body; an expansion chamber extending from the base, wherein the expansion chamber expands a volumetric capacity of the expansion well by at least a volumetric capacity of the expansion chamber; a mouth defining an opening into an interior space of the expansion chamber; and a cap configured to be coupled to the mouth to close the opening.

I2. The fluidic cartridge of embodiment I1, wherein the first coupling structure comprises a first peripheral wall formed in the cartridge body and at least partially surrounding the expansion well and the second coupling structure comprises a second peripheral wall extending below the bottom side of the base and configured to conform to an inner surface or an outer surface of the first peripheral wall.

I3. The fluidic cartridge of embodiment I2, wherein the second coupling structure comprises a third peripheral wall extending below the bottom side of the base and spaced apart from the second peripheral wall to form a peripheral groove on the bottom side of the base, and wherein the peripheral groove is configured to receive the first peripheral wall between the second peripheral wall and the third peripheral wall.

I4. The fluidic cartridge of any one of embodiments I1 to I3, wherein the expansion chamber and the mouth have different shapes.

I5. The fluidic cartridge of embodiment I4, wherein the expansion chamber has three straight sides and the mouth has a circular shape.

I6. The fluidic cartridge of any one of embodiments I1 to I5, further comprising a perimeter chamfer formed in an inner surface of the mouth about the opening.

I7. The fluidic cartridge of embodiment I3, wherein the first peripheral wall and the peripheral groove each have three sides.

I8. The fluidic cartridge of embodiment I7, wherein the first peripheral wall has three straight sides connected by rounded corners, and wherein the peripheral groove has three straight sides connected by rounded corners.

I9. The fluidic cartridge of embodiment I2, wherein the first peripheral wall is affixed to the second peripheral wall by an adhesive or by welding.

I10. The fluidic cartridge of embodiment I3, wherein the first peripheral wall is affixed to at least one of the second peripheral wall and the third peripheral wall by an adhesive or by welding.

I11. The fluidic cartridge of any one of embodiments I1 to I10, wherein the cap comprises: an insert sleeve configured to be inserted into the opening defined by the mouth; and a shroud that is wider than the insert sleeve and includes a top wall that substantially closes one end of the insert sleeve.

I12. The fluidic cartridge of embodiment I11, wherein the insert sleeve is hollow, and the shroud includes a vent hole which extends through the top wall of the shroud and is open to an interior space of the insert sleeve, and wherein the cap further comprises a venting membrane disposed over the vent hole, wherein the venting membrane is configured to permit the passage of a gas but prevent the passage of liquid.

I13. The fluidic cartridge of embodiment I12, wherein the venting membrane has a pore size of about 0.2 μm.

I14. The fluidic cartridge of embodiment I12 or I13, wherein the vent hole comprises an inner vent hole portion formed on an inner surface of the top wall of the shroud, and an outer vent hole portion formed in an outer surface of the top wall of the shroud, wherein a width of the inner vent hole is different from a width of the outer vent hole.

I15. The fluidic cartridge of embodiment I14, wherein the width of the inner vent hole portion is less than the width of the outer vent hole portion.

I16. The fluidic cartridge of any one of embodiments I11 to I15, wherein the cap further comprises radial ribs extending between an inner surface of the shroud and an outer surface of the insert sleeve, wherein, when the cap is coupled to the mouth to close the opening, the insert sleeve is inserted into the opening until the radial ribs contact an edge of the mouth surrounding the opening.

I17. The fluidic cartridge of any one of embodiments I11 to I16, wherein the insert sleeve is secured within the opening defined by the mouth by a friction fit.

I18. The fluidic cartridge of any one of embodiments I11 to I17, wherein the cap includes a circumferential rib extending around an outer surface of the insert sleeve.

I19. The fluidic cartridge of any one of embodiments I12 to I18, further comprising at least one groove formed in a top surface of the top wall of the shroud, wherein the at least one groove extends through the vent hole.

I20. The fluidic cartridge of embodiment I19, further comprising at least two grooves formed in the top surface of the shroud, wherein the at least two grooves cross each other through the vent hole.

I21. The fluidic cartridge of any one of embodiments I1 to I20, wherein the chamber expander further comprises a stanchion extending from the base, and wherein the cap is hingedly connected to the stanchion.

I22. The fluidic cartridge of embodiment I21, further comprising a tab extending from the cap, and wherein the cap is connected to the stanchion by a living hinge connecting a free end of the stanchion to a free end of the tab.

I23. The fluidic cartridge of any one of embodiments I1 to I22, wherein the expansion well includes a sloped surface surrounding an opening thereof.

I24. The fluidic cartridge of any one of embodiments I1 to I23, further comprising a lysis capsule for performing cell lysis disposed within the expansion well, wherein the lysis capsule comprises: a hollow body having an open first end and an open second end; a first porous membrane affixed to the body, the first porous membrane covering the open first end; a second porous membrane affixed to the body, the second porous membrane covering the open second end, wherein the hollow body defines a lysis chamber between the first and second porous membranes; a plurality of non-magnetic beads contained within the lysis chamber; and at least one magnetic element contained within the lysis chamber, wherein the pores of the first and the second porous membranes are sized to retain the plurality of non-magnetic beads and the at least one magnetic element within the lysis chamber.

I25. The fluidic cartridge of embodiment I24, wherein the expansion well and the hollow body of the lysis capsule have conforming three-sided shapes.

I26. The fluidic cartridge of any one of embodiments I1 to I25, wherein the chamber expander is comprised of a transparent or translucent material.

I27. The fluidic cartridge of any one of embodiments I1 to I26, wherein the chamber expander is comprised of a polypropylene.

I28. The fluidic cartridge of any one of embodiments I1 to I27, wherein the cartridge body is comprised of an opaque material.

I29. The fluidic cartridge of any one of embodiments I1 to I28, wherein the cartridge body is comprised of a thermoplastic polymer material.

I30. The fluidic cartridge of any one of embodiments I1 to I29, wherein the cartridge body is comprised of a cyclic olefin copolymer (COC) or a cyclic olefin polymer (COP).

I31. The fluidic cartridge of any one of embodiments I1 to I30, wherein the cartridge body is comprised of a material selected from the group consisting of polycarbonate, polyacrylamide, polyethylene, polymethyl-methacrylate (PMMA), polydimethylsiloxane (PDMS), and polyvinyl chloride (PVC), and polypropylene (PP).

Some embodiments encompass:

J1. In a fluidic cartridge including a cartridge body defining two or more wells that are fluidly connected or connectable, a method for expanding a volumetric capacity of an expansion well of the two or more wells, the method comprising: securing a chamber expander to the cartridge body by coupling a first coupling structure at least partially surrounding the expansion well to a second coupling structure of the chamber expander and forming a hermetic seal between the chamber expander and the cartridge body, wherein the chamber expander comprises: a base, wherein the second coupling structure is located on a bottom side of the base; an expansion chamber extending from the base, wherein the expansion chamber expands a volumetric capacity of the expansion well by at least a volumetric capacity of the expansion chamber; a mouth defining an opening into an interior space of the expansion chamber; and a cap configured to be coupled to the mouth to close the opening.

J2. The method of embodiment J1, wherein the first coupling structure comprises a first peripheral wall formed in the cartridge body and surrounding the expansion well and the second coupling structure comprises a second peripheral wall extending below the bottom side of the base and configured to conform to an inner surface or to an outer surface of the first peripheral wall, and wherein coupling the first coupling structure to the second coupling structure comprises affixing the second peripheral wall to the inner surface or to the outer surface of the first peripheral wall.

J3. The method of embodiment J2, wherein affixing the second peripheral wall to the inner surface or to the outer surface of the first peripheral wall comprises securing the second peripheral wall to the inner surface or to the outer surface of the first peripheral wall by an adhesive or by welding.

J4. The method of embodiment J1, wherein the first coupling structure comprises a first peripheral wall formed in the cartridge body and surrounding the expansion well and the second coupling structure comprises a second peripheral wall extending below the bottom side of the base and a third peripheral wall extending below the bottom side of the base and spaced apart from the second peripheral wall to form a peripheral groove on the bottom side of the base, and wherein coupling the first coupling structure to the second coupling structure comprises inserting the first peripheral wall into the peripheral groove between the second peripheral wall and the third peripheral wall and affixing the first peripheral wall to at least one of the second peripheral wall and the third peripheral wall.

J5. The method of embodiment J4, wherein affixing the first peripheral wall to at least one of the second peripheral wall and the third peripheral wall comprises securing the first peripheral wall to at least one of the second peripheral wall and the third peripheral wall by an adhesive or by welding.

J6. The method of any one of embodiments J1 to J5, wherein the cap comprises an insert sleeve and a shroud that is wider than the insert sleeve, and wherein the method comprises coupling the cap to the mouth by inserting the insert sleeve into the opening defined by the mouth.

J7. The method of any one of embodiments J1 to J6, further comprising: dispensing an amount of liquid substance into the opening defined by the mouth, wherein the amount of liquid substance completely fills the expansion well and at least partially fills the interior space of the expansion chamber; and coupling the cap to the mouth to close the opening.

J8. The method of any one of embodiments J1 to J7, wherein the expansion chamber and the mouth have different shapes.

J9. The method of embodiment J8, wherein the expansion chamber has three straight sides and the mouth has a circular shape.

J10. The method of any one of embodiments J1 to J9, wherein the chamber expander further comprises a perimeter chamfer formed in an inner surface of the mouth about the opening.

J11. The method of embodiment J4, wherein the first peripheral wall and the peripheral groove each have three sides.

J12. The method of embodiment J11, wherein the first peripheral wall has three straight sides connected by rounded corners, and wherein the peripheral groove has three straight sides connected by rounded corners.

J13. The method of any one of embodiments J1 to J4, wherein the cap comprises: an insert sleeve configured to be inserted into the opening defined by the mouth; and a shroud that is wider than the insert sleeve and includes a top wall that substantially closes one end of the insert sleeve.

J14. The method of embodiment J13, wherein the insert sleeve is hollow, and the shroud includes a vent hole which extends through the top wall of the shroud and is open to an interior space of the insert sleeve, and wherein the cap further comprises a venting membrane disposed over the vent hole, wherein the venting membrane is configured to permit the passage of a gas but prevent the passage of liquid.

J15. The method of embodiment J13 or J14, wherein the cap further comprises radial ribs extending between an inner surface of the shroud and an outer surface of the insert sleeve, wherein coupling the cap to the mouth to close the opening comprises inserting the insert sleeve into the opening until the radial ribs contact an edge of the mouth surrounding the opening.

J16. The method of embodiment J15, wherein the insert sleeve is secured within the opening defined by the mouth by a friction fit.

J17. The method of embodiment J16, wherein the cap includes a circumferential rib extending around an outer surface of the insert sleeve.

J18. The method of any one of embodiments J14 to J17, further comprising at least one groove formed in a top surface of the top wall of the shroud, wherein the at least one groove extends through the vent hole.

J19. The method of embodiment J18, further comprising at least two grooves formed in the top surface of the shroud, wherein the at least two grooves cross each other through the vent hole.

J20. The method of any one of embodiments J1 to J19, wherein the chamber expander further comprises a stanchion extending from the base, and wherein the cap is hingedly connected to the stanchion.

J21. The method of embodiment J20, further comprising a tab extending from the cap, and wherein the cap is connected to the stanchion by a living hinge connecting a free end of the stanchion to a free end of the tab.

J22. The method of any one of embodiments J1 to J21, wherein the expansion well includes a sloped surface surrounding an opening thereof.

J23. The method of any one of embodiments J1 to J22, further comprising, before securing the chamber expander to the cartridge body, inserting a lysis capsule into a well opening of the expansion well, the lysis capsule comprising a hollow body having an open first end and an open second end, a first porous membrane affixed to the hollow body and covering the open first end, a second porous membrane affixed to the hollow body and covering the open second end, wherein the hollow body defines a lysis chamber between the first and second porous membranes, and a plurality of beads contained within the lysis chamber.

J24. The method of embodiment J23, wherein the plurality of beads comprises a plurality of non-magnetic beads and at least one magnetic element.

Some embodiments encompass:

K1. A method of manufacturing a fluidic cartridge comprising: (a) providing a cartridge body comprising two or more wells that are that are fluidly connected or connectable; (b) inserting a lysis capsule into a well opening of a first well of the two or more wells, the lysis capsule comprising a lysis chamber and a plurality of lysis beads contained within the lysis chamber; and (c) securing a chamber expander to the cartridge body over the well opening of the first well, wherein the chamber expander comprises an expansion chamber which expands a volumetric capacity of the first well by at least a volumetric capacity of the expansion chamber, a mouth defining an expansion chamber opening into an interior space of the expansion chamber, and a cap configured to be coupled to the mouth to close the expansion chamber opening.

K2. The method of embodiment K1, wherein (c) comprises securing the chamber expander to the cartridge body by coupling a first coupling structure of the cartridge body at least partially surrounding the first well to a second coupling structure of the chamber expander and forming a hermetic seal between the chamber expander and the cartridge body.

K3. The method of embodiment K2, wherein the first coupling structure comprises a first peripheral wall formed on the cartridge body and surrounding the first well, and the second coupling structure comprises a second peripheral wall extending below a bottom side of a base of the chamber expander and configured to conform to an inner surface or to an outer surface of the first peripheral wall, and wherein coupling the first coupling structure to the second coupling structure comprises affixing the second peripheral wall to the inner surface or to the outer surface of the first peripheral wall.

K4. The method of embodiment K3, wherein affixing the second peripheral wall to the inner surface or to the outer surface of the first peripheral wall comprises securing the second peripheral wall to the inner surface or to the outer surface of the first peripheral wall by an adhesive or by welding.

K5. The method of embodiment K2, wherein the first coupling structure comprises a first peripheral wall formed on the cartridge body and surrounding the first well, and the second coupling structure comprises a second peripheral wall extending below a bottom side of a base of the chamber expander and a third peripheral wall extending below the bottom side of the base and spaced apart from the second peripheral wall to form a peripheral groove on the bottom side of the base, and wherein coupling the first coupling structure to the second coupling structure comprises inserting the first peripheral wall into the peripheral groove between the second peripheral wall and the third peripheral wall and affixing the first peripheral wall to at least one of the second peripheral wall and the third peripheral wall.

K6. The method of embodiment K5, wherein affixing the first peripheral wall to at least one of the second peripheral wall and the third peripheral wall comprises securing the first peripheral wall to at least one of the second peripheral wall and the third peripheral wall by an adhesive or by welding.

K7. The method of any one of embodiments K1 to K6, further comprising dispensing a reagent into each of one or more of the two or more wells, other than the first well, and sealing each well into which a reagent has been dispensed.

K8. The method of embodiment K7, wherein at least one reagent is a non-liquid reagent.

K9. The method of any one of embodiments K1 to K8, wherein the lysis capsule comprises a hollow body having an open first end and an open second end, a first porous membrane affixed to the hollow body and covering the open first end, a second porous membrane affixed to the hollow body and covering the open second end, wherein the hollow body defines the lysis chamber between the first and second porous membranes.

K10. The method of any one of embodiments K1 to K9, wherein the lysis beads comprise a plurality of non-magnetic beads contained within the lysis chamber; and wherein the lysis capsule comprises at least one magnetic element contained within the lysis chamber.

K11. The method of any one of embodiments K1 to K10, further comprising covering a well opening of at least one well of the two or more wells, other than the first well, to form an at least partially enclosed chamber in each covered well.

K12. The method of any one of embodiments K1 to K11, wherein the cap comprises an insert sleeve and a shroud that is wider than the insert sleeve and wherein the method comprises coupling the cap to the mouth by inserting the insert sleeve into the expansion chamber opening.

K13. The method of any one of embodiments K1 to K11, further comprising: dispensing an amount of liquid substance into the expansion chamber opening, wherein the amount of liquid substance completely fills the first well and at least partially fills the interior space of the expansion chamber; and coupling the cap to the mouth to close the opening.

K14. The method of any one of embodiments K1 to K13, wherein the expansion chamber and the mouth have different shapes.

K15. The method of embodiment K14, wherein the expansion chamber has three straight sides and the mouth has a circular shape.

K16. The method of any one of embodiments K1 to K15, wherein the chamber expander further comprises a perimeter chamfer formed in an inner surface of the mouth and extending about the expansion chamber opening.

K17. The method of embodiment K5, wherein the first peripheral wall and the peripheral groove each have three sides.

K18. The method of embodiment K17, wherein the first peripheral wall has three straight sides connected by rounded corners, and wherein the peripheral groove has three straight sides connected by rounded corners.

K19. The method of embodiment K12, wherein the insert sleeve is hollow, and the shroud includes a vent hole which extends through a top wall of the shroud and is open to an interior space of the insert sleeve, wherein the cap further comprises a venting membrane disposed over the vent hole, and wherein the venting membrane is configured to permit the passage of a gas but prevent the passage of liquid.

K20. The method of embodiment K19, wherein the vent hole comprises an inner vent hole portion formed on an inner surface of the top wall of the shroud, and an outer vent hole portion formed in an outer surface of the top wall of the shroud, and wherein a width of the inner vent hole portion is different from a width of the outer vent hole portion.

K21. The method of embodiment K20, wherein the width of the inner vent hole portion is less than the width of the outer vent hole portion.

K22. The method of any one of embodiments K12 and K19 to K21, wherein the cap further comprises radial ribs extending between an inner surface of the shroud and an outer surface of the insert sleeve, and wherein coupling the cap to the mouth to close the opening comprises inserting the insert sleeve into the opening until the radial ribs contact an edge of the mouth surrounding the expansion chamber opening.

K23. The method of embodiment K22, wherein the insert sleeve is secured within the expansion chamber opening by a friction fit.

K24. The method of embodiment K23, wherein the cap includes a circumferential rib extending around an outer surface of the insert sleeve.

K25. The method of any one of embodiments K19 to K24, further comprising at least one groove formed in a top surface of the top wall of the shroud, wherein the at least one groove extends through the vent hole.

K26. The method of embodiment K25, further comprising at least two grooves formed in the top surface of the shroud, wherein the at least two grooves cross each other through the vent hole.

K27. The method of any one of embodiments K1 to K26, wherein the chamber expander further comprises a base and a stanchion extending from the base, and wherein the cap is hingedly connected to the stanchion.

K28. The method of embodiment K27, further comprising a tab extending from the cap, wherein the cap is connected to the stanchion by a living hinge connecting a free end of the stanchion to a free end of the tab.

K29. The method of any one of embodiments K1 to K28, wherein the first well includes a sloped surface surrounding an opening thereof.

Some embodiments encompass:

L1. A method for processing a fluid sample in a fluidic cartridge comprising two or more wells of uniform height and a chamber expander secured with respect to one of the two or more wells to expand a volumetric capacity of the one well by the volumetric capacity of the chamber expander, wherein the two or more wells are fluidly connected or connectable, and wherein the method comprises: (a) dispensing an amount of the fluid sample into an opening defined by a mouth of the chamber expander, wherein the amount of the fluid sample completely fills the one well and at least partially fills an interior space of the chamber expander; and (b) coupling a cap to the mouth of the chamber expander to close the opening.

L2. The method of embodiment L1, further comprising (c) passing air from the interior space of the chamber expander to an external environment through a vent hole in the cap.

L3. The method of embodiment L2, wherein (c) comprises passing the air through a porous vent membrane affixed to the cap and covering the vent hole, the porous vent membrane being impermeable to liquids.

L4. The method of any one of embodiments L1 to L3, wherein the chamber expander is secured by coupling a first coupling structure at least partially surrounding the one well to a second coupling structure of the chamber expander and forming a hermetic seal between the chamber expander and the one well.

L5. The method of any one of embodiments L1 to L4, wherein at least one of the two or more wells, other than the one well into which the fluid sample is dispensed, contains a reagent for processing the fluid sample.

L6. The method of embodiment L5, wherein the reagent is a non-liquid reagent.

L7. The method of any one of embodiments L1 to L6, wherein the cap comprises an insert sleeve and a shroud that is wider than the insert sleeve, and wherein (b) comprises inserting the insert sleeve into the opening.

L8. The method of embodiment L7, wherein the insert sleeve is secured within the opening by a friction fit.

L9. The method of any one of embodiments L1 to L8, wherein the one well comprises a lysis chamber comprising a first porous membrane and a second porous membrane defining a lysis chamber therebetween and a plurality of beads contained within the lysis chamber.

L10. The method of embodiment L9, further comprising (d) agitating the plurality of beads within the lysis chamber to lyse cells contained within the fluid sample, thereby releasing nucleic acids from the lysed cells.

L11. The method of embodiment L10, wherein the plurality of beads comprise a plurality of non-magnetic beads and at least one magnetic element, and wherein (d) comprises subjecting the at least one magnetic element to a magnetic field, thereby causing movement of the at least one magnetic element within the lysis chamber, the movement of the at least one magnetic element within the lysis chamber causing movement of the plurality of non-magnetic beads within the lysis chamber.

L12. The method of embodiment L11, wherein the magnetic field is created by an electromagnet.

L13. The method of claim L12, comprising alternating a current to the electromagnet to alternate a polarity of the electromagnet.

L14. The method of embodiment L13, comprising alternating the current to alternate the polarity of the electromagnet at a frequency of 20 Hertz to 200 Hertz.

L15. The method of embodiment L14, comprising pulsing the current to alternate the polarity of the electromagnet at two or more different frequencies.

L16. The method of any one of embodiments L10 to L15, further comprising (e) transporting the released nucleic acids from the one well to a processing chamber of the cartridge.

L17. The method of embodiment L16, further comprising (f) during (e), retaining lysed cellular material within the lysis chamber while allowing the released nucleic acids to pass through the second porous membrane.

L18. The method of embodiment L17, further comprising (g) during (e) agitating the plurality of beads within the lysis chamber, thereby causing at least a portion of the lysed cellular material within the lysis chamber to remain in suspension at least until the fluid sample has been removed from the lysis chamber.

L19. The method of embodiment L17 or L18, further comprising (h) immobilizing at least a portion of the released nucleic acids in the processing chamber on a solid support and removing non-immobilized components of the fluid sample to a waste chamber of the cartridge.

L20. The method of embodiment L19, further comprising (i) eluting the immobilized nucleic acids from the solid support and transporting the eluted nucleic acids to a reaction chamber of the cartridge.

L21. The method of embodiment L20, further comprising (j) subjecting the eluted nucleic acids to conditions of a first reaction, the first reaction providing an indication of the presence or amount of an analyte of interest.

L22. The method of embodiment L21, further comprising: (k) releasing an internal control into the fluid sample, the internal control being contained within the lysis chamber; (l) immobilizing nucleic acids associated with the internal control (“IC nucleic acids”) on the solid support; eluting the IC nucleic acids from the solid support; (m) transporting the eluted IC nucleic acids to the reaction chamber; and (n) subjecting the IC nucleic acids to conditions of a second reaction, the second reaction providing an indication of an extent of lysis, wherein the internal control is provided to validate an assay and/or to validate the effectiveness of cell lysis during (d).

L23. The method of embodiment L22, wherein at least a portion of the internal control is contained in an internal control reagent disposed on the first porous membrane, and wherein the internal control reagent dissolves in the fluid sample.

L24. The method of embodiment L22 or L23, wherein the internal control is contained in an internal control reagent disposed on at least a portion of the plurality of beads, and wherein the internal control reagent disposed on the at least a portion of the plurality of beads dissolves in the fluid sample.

L25. The method of embodiment L22, wherein the internal control is embedded in or contained within an internal control pellet contained within the lysis chamber, and wherein the internal control pellet is adapted to dissolve when contacted by the fluid sample and/or disintegrate during (d).

L26. The method of any one of embodiments L22 to L25, wherein the conditions of the first reaction and the conditions of the second reaction are the same conditions.

L27. The method of any one of embodiments L22 to L26, wherein each of the first and second reactions is a nucleic acid amplification reaction.

L28. The method of embodiment L27, wherein the nucleic acid amplification reaction is a polymerase chain reaction (“PCR”).

Some embodiments encompass:

M1. A bead delivery cap for dispensing lytic agents into a sample well of a fluidic cartridge, the cap comprising: a cap body comprising a deformable wall defining a chamber; a lower sleeve situated beneath the deformable wall and defining a recess that is open to the chamber; a frangible membrane affixed to an open bottom end of the lower sleeve and enclosing the recess and the chamber; and lytic agents comprising a plurality of non-magnetic beads and at least one magnetic element, wherein the lytic agents are contained within the chamber and the recess in a quantity such that deformation of the deformable wall causes the lytic agents to rupture the frangible membrane, thereby releasing the lytic agents from the chamber and the recess.

M2. The bead delivery cap of embodiment M1, wherein the lower sleeve includes at least one sealing rib extending about an outer surface of the lower sleeve.

M3. The bead delivery cap of embodiment M1 or M2, wherein the lower sleeve has a cylindrical shape.

M4. The bead delivery cap of any one of embodiments M1 to M3, wherein the cap body comprises a laterally extending member to which an upper end of the lower sleeve is connected, and wherein the deformable wall extends upward from the laterally extending member.

M5. The bead delivery cap of any one of embodiments M1 to M4, wherein the deformable wall is dome shaped when not in a deformed state.

M6. The bead delivery cap of embodiment M4 or M5, further comprising an upper peripheral wall spaced apart from the deformable wall and projecting upwardly from an outer perimeter of the laterally extending member.

M7. The bead delivery cap of embodiment M6, wherein a top end of the upper peripheral wall is situated above the deformable wall.

M8. The bead delivery cap of any one of embodiments M1 to M7, wherein the cap body is unitary structure composed of a polymeric material.

M9. The bead delivery cap of embodiment M8, wherein the polymeric material is a thermoplastic elastomer.

M10. The bead delivery cap of any one of embodiments M1 to M9, wherein the frangible membrane comprises a porous film.

M11. The bead delivery cap of any one of embodiments M1 to M10, further comprising a peelable cover film covering an outer surface of the frangible membrane.

M12. The bead delivery cap of any one of embodiments M1 to M11, wherein the frangible membrane comprises one or more rupture lines configured to make the frangible membrane more susceptible to rupturing.

M13. The bead delivery cap of embodiment M12, wherein the one or more rupture lines consist of a single line having a C-shape or U-shape.

M14. The bead delivery cap of any one of embodiments M1 to M13, wherein the at least one magnetic element is disposed adjacent an inner surface of the deformable wall.

M15. The bead delivery cap of any one of embodiments M1 to M14, wherein the deformable wall includes a vent hole formed in the deformable wall and in fluid communication with the chamber.

M16. The bead delivery cap of embodiment M15, further comprising a porous membrane covering the vent hole, the porous membrane being affixed to a top or bottom surface of the deformable wall.

M17. The bead delivery cap of any one of embodiments M1 to M16, wherein each of the plurality of non-magnetic beads is comprised of a ceramic or a glass.

M18. The bead delivery cap of any one of embodiments M1 to M17, wherein each of the plurality of non-magnetic beads has a spherical shape.

M19. The bead delivery cap of embodiment M18, wherein each of the plurality of non-magnetic beads has diameter of 100 μm to 2000 μm.

M20. The bead delivery cap of any one of embodiments M1 to M19, wherein the at least one magnetic element is plated or encapsulated with a non-magnetic material.

M21. The bead delivery cap of embodiment M20, wherein the non-magnetic material is a metal or a plastic.

M22. The bead delivery cap of any one of embodiments M1 to M21, wherein the at least one magnetic element occupies a greater volume than any of the plurality of non-magnetic beads.

M23. The bead delivery cap of any one of embodiments M1 to M22, wherein the at least one magnetic element has the shape of a cube.

M24. The bead delivery cap of embodiment M23, wherein the width of each face of the cube is 2.0 millimeters to 4.3 millimeters.

M25. The bead delivery cap of any one of embodiments M1 to M24, wherein the at least one magnetic element is comprised of neodymium.

M26. The bead delivery cap of embodiment M25, wherein the neodymium is N52 grade or N42 grade.

M27. The bead delivery cap of any one of embodiments M1 to M26, wherein the at least one magnetic element and each of the plurality of non-magnetic beads are inert.

M28. The bead delivery cap of any one of embodiments M1 to M27, wherein a force of 1.0 to 5.0 pounds applied to the deformable wall is required to rupture the frangible membrane.

M29. The bead delivery cap of any one of embodiments M1 to M28 further comprising an internal control contained within the cap body, wherein the internal control is provided to validate an assay result and/or to validate the effectiveness of a cell lysis procedure performed with lytic agents.

M30. The bead delivery cap of embodiment M29, wherein the internal control is contained in an internal control reagent, wherein at least a portion of the internal control reagent is disposed on at least a portion of the plurality of non-magnetic beads and/or the at least one magnetic element, and wherein the internal control reagent is adapted to dissolve when contacted by a fluid sample.

M31. The bead delivery cap of embodiment M29, wherein the internal control is embedded in or contained within an internal control pellet adapted to dissolve when contacted by a fluid sample and/or to disintegrate when the plurality of magnetic beads is agitated, the internal control pellet being contained within the cap body.

Some embodiments encompass:

N1. A fluidic cartridge, comprising: a cartridge body comprising a sample chamber, the sample chamber having an open top end; and the bead delivery cap of any one of embodiments M1 to M32 inserted into the open top end of the sample chamber.

N2. The fluidic cartridge of embodiment N1, further comprising a syringe barrel in communication with the sample chamber, the syringe barrel being adapted to receive a syringe stopper connected to a syringe plunger for actuating fluids within the fluidic cartridge.

Some embodiments encompass:

O1. A method for lysing cells contained in a sample, comprising: (a) providing the sample to a sample chamber of a fluidic cartridge; (b) inserting the bead delivery cap of any one of embodiments M1 to M32 into the sample well, such that (i) an outer surface of the lower sleeve is in sealing engagement with an inner surface of a sidewall of the sample chamber, and (ii) the frangible membrane is situated above the sample in the sample chamber; (c) applying a force to the deformable wall, thereby deforming the deformable wall to an extent that the lytic agents rupture the frangible membrane and are released from the chamber and the recess into the sample chamber; and (d) subjecting the sample and the lytic agents to a magnetic field, the magnetic field causing the at least one magnetic element to agitate the plurality of non-magnetic beads to lyse cells contained within the sample.

O2. The method of embodiment O1, wherein (c) comprises moving a bead delivery cap actuator to apply the force to the deformable wall.

O3. The method of embodiment O1, wherein (c) comprises manually applying the force to the deformable wall.

O4. The method of any one of embodiments O1 to O3, wherein the lytic agents occupy at least 90% of the volume of the chamber and the recess of the cap.

O5. The method of any one of embodiments O1 to O4, wherein (a) comprises providing sample to the sample chamber with a pipettor.

O6. The method of any one of embodiments O1 to O5, wherein (c) comprises applying a force of 1.0 to 5.0 pounds to the deformable wall.

Some embodiments encompass:

P1. A method for lysing cells contained in a fluid sample, comprising (a) providing the fluid sample to a sample chamber of a fluidic cartridge; (b) securing a cap to an open end of the sample chamber, the cap including a deformable wall defining a chamber containing lytic agents and enclosed by a frangible membrane, the lytic agents comprising a plurality of non-magnetic beads and at least one magnetic element, and the size of the at least one magnetic element being greater than the size of any of the plurality of non-magnetic beads; (c) applying a force to a top end of the deformable wall, thereby collapsing the chamber to an extent that the lytic agents contained within the chamber rupture the frangible membrane, thereby releasing the lytic agents from the chamber into the sample chamber; and (d) subjecting the fluid sample and the lytic agents to a magnetic field, the magnetic field causing the at least one magnetic element to agitate the plurality of non-magnetic beads to lyse cells contained within the fluid sample.

P2. The method of embodiment P1, wherein the deformable wall is dome shaped when not in a deformed state.

P3. The method of embodiment P1 or P2, wherein the cap is unitary structure composed of a polymeric material.

P4. The method of embodiment P3, wherein the polymeric material is a thermoplastic elastomer.

P5. The method of any one of embodiments P1 to P4, wherein the frangible membrane comprises a porous film.

P6. The method of any one of embodiments P1 to P5, further comprising removing a peelable cover film from an outer surface of the frangible membrane prior to (b).

P7. The method of any one of embodiments P1 to P6, wherein the frangible membrane comprises one or more rupture lines configured to make the frangible membrane more susceptible to rupturing.

P8. The method of embodiment P7, wherein the one or more rupture lines consist of a single line having a C-shape or U-shape.

P9. The method of any one of embodiments P1 to P8, wherein the at least one magnetic element is disposed adjacent an inner surface of the deformable wall.

P10. The method of any one of embodiments P1 to P9, wherein the deformable wall includes a vent hole formed in the deformable wall and in fluid communication with the chamber.

P11. The method of embodiment P10, further comprising a porous membrane covering the vent hole, the porous membrane being affixed to a top or bottom surface of the deformable wall.

P12. The method of any one of embodiments P1 to P11, wherein each of the plurality of non-magnetic beads is comprised of a ceramic or a glass.

P13. The method of any one of embodiments P1 to P12, wherein each of the plurality of non-magnetic beads has a spherical shape.

P14. The method of embodiment P13, wherein each of the plurality of non-magnetic beads has diameter of 100 μm to 2000 μm.

P15. The method of any one of embodiments P1 to P14, wherein the at least one magnetic element is plated or encapsulated with a non-magnetic material.

P16. The method of embodiment P15, wherein the non-magnetic material is a metal or a plastic.

P17. The method of any one of embodiments P1 to P16, wherein the at least one magnetic element occupies a greater volume than any of the plurality of non-magnetic beads.

P18. The method of any one of embodiments P1 to P17, wherein the at least one magnetic element has the shape of a cube.

P19. The method of embodiment P18, wherein the width of each face of the cube is 2.0 millimeters to 4.3 millimeters.

P20. The method of any one of embodiments P1 to P19, wherein the at least one magnetic element is comprised of neodymium.

P21. The method of embodiment P20, wherein the neodymium is N52 grade or N42 grade.

P22. The method of any one of embodiments P1 to P21, wherein the at least one magnetic element and each of the plurality of non-magnetic beads are inert.

P23. The method of any one of embodiments P1 to P22, wherein (c) comprises applying a force of 1.0 to 5.0 pounds to the deformable wall.

P24. The method of any one of embodiments P1 to P23 further comprising disposing an internal control within the chamber, wherein the internal control is provided to validate an assay performed on the fluid sample after (d) and/or to validate the effectiveness of (d) to lyse the cells.

P25. The method of embodiment P24, wherein at least a portion of the internal control is contained in an internal control reagent disposed on at least a portion of the plurality of non-magnetic beads and/or the at least one magnetic element, wherein the internal control reagent is adapted to dissolve when contacted by the fluid sample.

P26. The method of embodiment P24, wherein at least a portion of the internal control is embedded in or contained within an internal control pellet adapted to dissolve when contacted by the fluid sample and/or to disintegrate when the plurality of magnetic beads is agitated, the internal control pellet being contained within the chamber.

Some embodiments encompass:

Q1. A method for lysing cells contained in a fluid sample, comprising: (a) positioning an electromagnet within a predetermined distance from a sample chamber of a microfluidic device, the sample well containing the fluid sample and lytic agents comprising a plurality of non-magnetic beads and at least one magnetic element; (b) generating, by the electromagnet, a magnetic field targeted at the sample and the lytic agents contained with the sample chamber; and (c) reversing a polarity of the magnetic field, causing the at least one magnetic element to move in a random manner within the sample chamber to agitate the plurality of non-magnetic beads to lyse cells contained within the fluid sample such that nucleic acids are released from the lysed cells, wherein the electromagnet is spatially separated from the sample chamber and held stationary while generating the magnetic field targeted at the fluid sample and the lytic agents contained within the sample chamber.

Q2. The method of embodiment Q1, wherein (c) comprises reversing the polarity of the magnetic field at a predetermined frequency of 60 Hertz to 200 Hertz.

Q3. The method of embodiment Q1, where (b) comprises driving the electromagnet with a predetermined voltage of 10 volts to 50 volts.

Q4. The method of embodiment Q1, wherein (c) comprises charging the electromagnet with a switching amplifier to reverse the polarity of the magnetic field.

Q5. The method of any one of embodiments Q1 to Q4, further comprising, prior to (a), (d) manually or robotically dispensing the fluid sample into the sample chamber, optionally wherein (d) is performed with a pipettor, and wherein the sample chamber contains the lytic agents prior to (d).

Q6. The method of embodiment Q5, further comprising (e) covering an open top end of the sample chamber with a cap after (d) and prior to (b) and (c).

Q7. The method of any one of embodiments Q1 to Q6, further comprising: (f) after (c), transporting at least a portion of the fluid sample from the sample chamber to a processing chamber of the fluidic cartridge.

Q8. The method of embodiment Q7, further comprising: (g) during (e), retaining lysed cellular material from (c) within the sample chamber while allowing released nucleic acids from lysed cells to pass through a porous membrane.

Q9. The method of embodiment Q8, further comprising: (h) during (g) reversing the polarity of the magnetic field, thereby causing movement of the at least one magnetic element within the sample chamber, the movement of the at least one magnetic element within the sample chamber causing movement of the plurality of non-magnetic beads within the sample chamber, and the movement of the plurality of non-magnetic beads within the sample chamber causing at least a portion of the lysed cellular material within the sample chamber to remain in suspension at least until the fluid sample has been removed from the sample chamber.

Q10. The method of any one of embodiments Q8 to Q9, further comprising: (i) in the processing chamber, immobilizing at least a portion of the released nucleic acids on a solid support and removing non-immobilized components of the fluid sample to a waste chamber of the microfluidic device.

Q11. The method of embodiment Q10, further comprising: (j) after (i), eluting the immobilized nucleic acids from the solid support and transporting the eluted nucleic acids to a reaction chamber of the microfluidic device.

Q12. The method of embodiment Q11, further comprising: (k) after (j), subjecting the eluted nucleic acids to conditions of a first reaction, the first reaction providing an indication of the presence or amount of an analyte of interest.

Q13. The method of embodiment Q12, further comprising: (l) during or after (a), releasing an internal control into the fluid sample, the internal control being contained within the sample chamber prior to (a); (m) immobilizing nucleic acids associated with the internal control (“IC nucleic acids”) on the solid support during (i); (n) after (i), eluting the IC nucleic acids from the solid support and transporting the eluted IC nucleic acids to the reaction chamber; and (o) after (n), subjecting the IC nucleic acids to conditions of a second reaction, a result of (o) being used to validate a result of (k) and/or to validate the effectiveness of the lysis in (c).

Q14. The method of embodiment Q13, wherein at least a portion of the internal control is contained in an internal control reagent disposed on at least portion of the sample chamber when (a) is initiated, and wherein the internal control reagent dissolves in the fluid sample during any of (a) to (c).

Q15. The method of embodiment Q13, wherein at least a portion of the internal control is contained in an internal control reagent disposed on at least a portion of the plurality of non-magnetic beads and/or the at least one magnetic element when (a) is initiated, and wherein the internal control reagent disposed on the at least a portion of the plurality of non-magnetic beads and/or the at least one magnetic element dissolves in the fluid sample during any of (a) to (c).

Q16. The method of embodiment Q13, wherein at least a portion of the internal control is contained in an internal control reagent disposed on an internal wall of the sample chamber when (a) is initiated, and wherein the internal control reagent dissolves in the fluid sample during any of (a) to (c).

Q17. The method of embodiment Q13, wherein the internal control is embedded in or contained within an internal control pellet, and wherein the internal control pellet dissolves in the presence of the fluid sample and/or is disintegrated by the movement of the plurality of non-magnetic beads during (c), thereby releasing the internal control into the fluid sample.

Q18. The method of any one of embodiments Q13 to Q17, wherein the conditions of the first reaction and the conditions of the second reaction are the same conditions.

Q19. The method of any one of embodiments Q13 to Q18, wherein each of the first and second reactions is a nucleic acid amplification reaction.

Q20. The method of embodiment Q19, wherein the nucleic acid amplification reaction is a polymerase chain reaction (“PCR”).

Some embodiments encompass:

R1. A fluidic cartridge, comprising: a sample chamber, the sample chamber having an open top end and a sample exit port; and a lysis chamber within the sample chamber, wherein the lysis chamber is defined by an inner wall of the sample chamber, a first porous membrane affixed within the sample chamber, the first porous membrane overlapping the open top end; and a second porous membrane spaced apart from the first porous membrane affixed within the sample chamber, the second porous membrane overlapping the sample exit port; a plurality of non-magnetic beads contained within the lysis chamber; and at least one magnetic element contained within the lysis chamber, wherein the pores of the first and the second porous membranes are sized to retain the plurality of non-magnetic beads and the at least one magnetic element within the lysis chamber.

R2. The fluidic cartridge of embodiment R1, wherein at least one of the first porous membrane and the second porous membrane comprises a mesh.

R3. The fluidic cartridge of embodiment R1, wherein at least one of the first porous membrane and the second porous membrane comprises a filter matrix.

R4. The fluidic cartridge of any one of embodiments R1 to R3, wherein the first porous membrane has a porosity or range of porosities that is greater than a porosity or range of porosities of the second porous membrane.

R5. The fluidic cartridge of embodiment R4, wherein the first porous membrane has a porosity of 70 μm to 500 μm, and wherein the second porous membrane has a porosity of 30 μm to 100 μm.

R6. The fluidic cartridge of any one of embodiments R1 to R5, wherein each of the plurality of non-magnetic beads is comprised of a ceramic or a glass.

R7. The fluidic cartridge of any one of embodiments R1 to R6, wherein each of the plurality of non-magnetic beads has a spherical shape, and wherein each of the plurality of non-magnetic beads optionally has diameter of 100 μm to 2000 μm.

R8. The fluidic cartridge of any one of embodiments R1 to R7, wherein the at least one magnetic element is plated or encapsulated with a non-magnetic material.

R9. The fluidic cartridge of any one of embodiments R1 to R8, wherein the at least one magnetic element occupies a greater volume than any of the plurality of non-magnetic beads.

R10. The fluidic cartridge of any one of embodiments R1 to R9, wherein the at least one magnetic element has the shape of a cube, and wherein each face of the cube optionally has a width of 2.0 millimeters to 4.3 millimeters.

R11. The fluidic cartridge of any one of embodiments R1 to R10, wherein the at least one magnetic element is comprised of neodymium, and wherein the neodymium is optionally N52 grade or N42 grade.

R12. The fluidic cartridge of any one of embodiments R1 to R11, further comprising an internal control contained within the lysis chamber, wherein the internal control is provided to validate an assay result and/or to validate the effectiveness of a cell lysis procedure performed with the plurality of non-magnetic beads and the at least one magnetic element.

R13. The fluidic cartridge of embodiment R12, wherein at least a portion of the internal control is contained in an internal control reagent disposed on at least one of the first porous membrane and the second porous membrane, and wherein the internal control reagent is adapted to dissolve when contacted by a fluid sample.

R14. The fluidic cartridge of embodiment R12, wherein at least a portion of the internal control is contained in an internal control reagent disposed on at least a portion of the plurality of non-magnetic beads and/or the at least one magnetic element, and wherein the internal control reagent is adapted to dissolve when contacted by a fluid sample.

R15. The fluidic cartridge of embodiment R12, wherein at least a portion of the internal control is contained in an internal control reagent disposed on an internal wall of the sample chamber, and wherein the internal control reagent is adapted to dissolve when contacted by a fluid sample.

R16. The fluidic cartridge of embodiment R12, wherein at least a portion of the internal control is embedded in or contained within an internal control pellet contained within the lysis chamber, and wherein the internal control pellet is adapted to dissolve when contacted by a fluid sample and/or to disintegrate when the plurality of magnetic beads is agitated.

R17. The fluidic cartridge of any one of embodiments R1 to R16, further comprising a syringe barrel in communication with the sample chamber, the syringe barrel being adapted to receive a syringe stopper connected to a syringe plunger for actuating fluids within the fluidic cartridge.

R18. The fluidic cartridge of any one of embodiments R1 to R17, wherein the sample chamber is covered by a removable seal.

R19. The fluidic cartridge of any one of embodiments R1 to R18, further comprising a cap adapted to be inserted into the open top end of the sample chamber.

R20. The fluidic cartridge of any one of embodiments R1 to R19, wherein the first porous membrane is press fit within a portion of the sample chamber.

R21. The fluidic cartridge of any one of embodiments R1 to R19, wherein the first porous membrane is heat sealed within a portion of the sample chamber, and optionally wherein the sample chamber includes a first lateral ledge with energy directors on which the first porous membrane is heat sealed.

R22. The fluidic cartridge of any one of embodiments R1 to R21, wherein the second porous membrane is press fit within a portion of the sample chamber.

R23. The fluidic cartridge of any one of embodiments R1 to R21, wherein the second porous membrane is heat sealed within a portion of the sample chamber, and optionally wherein the sample chamber includes a second lateral ledge with energy directors on which the second porous membrane is heat sealed.

Some embodiments encompass:

S1. A method for lysing cells contained in a fluid sample, comprising: (a) dispensing the fluid sample into the sample chamber of the fluidic cartridge of embodiment R1 to at least partially fill the lysis chamber with the fluid sample; (b) after (a), covering the open top end of the sample chamber with a cap; and (c) after (b), subjecting the at least one magnetic element to a magnetic field, thereby causing movement of the at least one magnetic element within the lysis chamber, the movement of the at least one magnetic element within the lysis chamber causing movement of the plurality of non-magnetic beads within the lysis chamber, and the movement of the non-magnetic beads within the lysis chamber causing cells contained within the fluid sample within the lysis chamber to lyse and release nucleic acids.

S2. The method of embodiment S1, further comprising (d) prior to (c), contacting the fluid sample with an internal control disposed within the sample chamber, and optionally disposed within the lysis chamber, wherein the internal control is provided to validate an assay result and/or to validate the effectiveness of (c) in causing cells contained within the fluid sample within the lysis chamber to lyse and release nucleic acids.

S3. The method of embodiment S2, wherein at least a portion of the internal control is contained within an internal control reagent disposed within the sample chamber, and optionally disposed within the lysis chamber.

S4. The method of embodiment S3, wherein at least a portion of the internal control reagent is disposed on at least one of the first porous membrane and the second porous membrane, and wherein the internal control reagent is adapted to dissolve when contacted by the fluid sample.

S5. The method of embodiment S3, wherein at least a portion of the internal control reagent is disposed on at least a portion of the plurality of non-magnetic beads and/or the at least one magnetic element, and wherein the internal control reagent is adapted to dissolve when contacted by the fluid sample.

S6. The method of embodiment S3, wherein at least a portion of the internal control reagent is disposed on an internal wall of the sample chamber, and wherein the internal control reagent is adapted to dissolve when contacted by the fluid sample.

S7. The method of embodiment S2, wherein at least a portion of the internal control is embedded in or contained within an internal control pellet adapted to dissolve when contacted by the fluid sample and/or to disintegrate during (c).

S8. The method of any one of embodiments S1 to S7, wherein the fluidic cartridge comprises a syringe barrel in communication with the sample chamber, the syringe barrel being adapted to receive a syringe stopper connected to a syringe plunger for actuating fluids within the fluidic cartridge.

S11. The method of any one of embodiments S1 to S8, further comprising covering the sample chamber with a removable seal.

S10. The method of any one of embodiments S1 to S9, further comprising inserting a cap into the open top end of the sample chamber.

S11. The method of any one of embodiments S1 to S10, comprising press fitting the first porous membrane within a portion of the sample chamber.

S12. The method of any one of embodiments S1 to S10, comprising heat sealing the first porous membrane within a portion of the sample chamber, and optionally wherein the sample chamber includes a first lateral ledge with energy directors on which the first porous membrane is heat sealed.

S13. The method of any one of embodiments S1 to S12, comprising press fitting the second porous membrane within a portion of the sample chamber.

S14. The method of any one of embodiments S1 to S12, comprising heat sealing the second porous membrane within a portion of the sample chamber, and optionally wherein the sample chamber includes a second lateral ledge with energy directors on which the second porous membrane is heat sealed.

S15. The method of any one of embodiments S1 to S14, wherein the magnetic field is created by an electromagnet during (c).

S16. The method of embodiment S15, wherein (c) comprises alternating a current to the electromagnet to alternate a polarity of the electromagnet.

S17. The method of embodiment S16, wherein (c) comprises alternating the current to alternate the polarity of the electromagnet at a frequency of 20 Hertz to 200 Hertz.

S18. The method of embodiment S17, wherein (c) comprises pulsing the current to alternate the field polarity of the electromagnet at two or more different frequencies.

S19. The method of any one of embodiments S1 to S18, further comprising: (e) after (c), transporting at least a portion of the fluid sample from the sample chamber to a processing chamber of the fluidic cartridge.

S20. The method of embodiment S19, further comprising: (f) during (e), retaining lysed cellular material from (c) within the lysis chamber while allowing the released nucleic acids to pass through the second porous membrane.

S21. The method of embodiment S20, further comprising: (g) during (e) subjecting the at least one magnetic element to the magnetic field, thereby causing movement of the at least one magnetic element within the lysis chamber, the movement of the at least one magnetic element within the lysis chamber causing movement of the plurality of non-magnetic beads within the lysis chamber, and the movement of the non-magnetic beads within the lysis chamber causing at least a portion of the lysed cellular material within the lysis chamber to remain in suspension until the fluid sample is removed from the lysis chamber.

S22. The method of any one of embodiments S19 to S21, further comprising: (h) in the processing chamber, immobilizing at least a portion of the released nucleic acids on a solid support and removing non-immobilized components of the fluid sample to a waste chamber of the fluidic cartridge.

S23. The method of embodiment S22, further comprising: (i) after (h), eluting the immobilized nucleic acids from the solid support and transporting the eluted nucleic acids to a reaction chamber of the fluidic cartridge.

S24. The method of embodiment S23, further comprising: (j) after (i), subjecting the eluted nucleic acids to conditions of a reaction, the reaction providing an indication of the presence or amount of an analyte of interest.

S25. The method of embodiment S24, wherein the reaction is a nucleic acid amplification reaction, and optionally wherein the nucleic acid amplification reaction is a polymerase chain reaction (“PCR”).

Some embodiments encompass:

T1. A method of manufacturing a fluidic cartridge comprising: (a) providing a cartridge body comprising a sample chamber with an open first end and a sample exit port at a second end of the sample chamber and one or more chambers that are that are fluidly connected or connectable, with the sample chamber; (b) providing first and second porous membranes and affixing the second porous membrane within the sample chamber, the second porous membrane covering the sample exit port; (c) introducing a plurality of non-magnetic beads into the sample chamber through the open first end of the sample chamber; (d) introducing at least one magnetic element into the sample chamber through the open first end of the sample chamber; and (e) after (c) and (d), affixing the first porous membrane within the sample chamber, the first porous membrane overlapping the open first end of the sample chamber, wherein an internal wall of the sample chamber and the affixed first and second porous membranes define a lysis chamber, and wherein the pores of the first and second porous membranes are sized to retain the plurality of non-magnetic beads and the at least one magnetic element within the lysis chamber.

T2. The method of embodiment T1, wherein the first porous membrane comprises a mesh or a filter matrix.

T3. The method of embodiment T1 or T2, wherein the second porous membrane comprises a mesh or a filter matrix.

T4. The method of any one of embodiments T1 to T3, wherein the first porous membrane is hydrophilic.

T5. The method of any one of embodiments T1 to T4, wherein the first porous membrane has a porosity or range of porosities that is greater than a porosity or range of porosities of the second porous membrane.

T6. The method of embodiment T5, wherein the first porous membrane has a porosity of 70 μm to 500 μm, and the second porous membrane has a porosity of 30 μm to 100 μm.

T7. The method of any one of embodiments T1 to T6, wherein the first porous membrane is press fit into the sample chamber.

T8. The method of any one of embodiments T1 to T6, wherein the first porous membrane is heat sealed within the sample chamber.

T9. The method of any one of embodiments T1 to T8, wherein the second porous membrane is press fit into the sample chamber.

T10. The method of any one of embodiments T1 to T8, wherein the second porous membrane is heat sealed within the sample chamber.

T11. The method of any one of embodiments T1 to T10, wherein each of the plurality of non-magnetic beads is comprised of a ceramic or a glass.

T12. The method of any one of embodiments T1 to T11, wherein each of the plurality of non-magnetic beads has a spherical shape, and wherein each of the plurality of non-magnetic beads optionally has a diameter of 100 μm to 2000 μm.

T13. The method of any one of embodiments T1 to T12, wherein the at least one magnetic element is plated or encapsulated with a non-magnetic material.

T14. The method of any one of embodiments T1 to T13, wherein the at least one magnetic element occupies a greater volume than any of the plurality of non-magnetic beads.

T15. The method of any one of embodiments T1 to T14, wherein the at least one magnetic element has the shape of a cube, and wherein the width of each face of the cube is optionally 2.0 millimeters to 4.3 millimeters.

T16. The method of any one of embodiments T1 to T15, wherein the at least one magnetic element is comprised of neodymium, and wherein the neodymium is optionally N52 grade or N42 grade.

T17. The method of any one of embodiments T1 to T16, wherein the at least one magnetic element and each of the plurality of non-magnetic beads is inert.

T18. The method of any one of embodiments T1 to T18, further comprising disposing an internal control onto a component of the lysis capsule, wherein the internal control is provided to validate an assay result and/or to validate the effectiveness of a cell lysis procedure performed with the plurality of non-magnetic beads and the at least one magnetic element.

T19. The method of embodiment T18, comprising disposing at least a portion of the internal control onto at least a portion of the plurality of non-magnetic beads and/or the magnetic element prior to (c) or after (c), wherein the internal control is contained in an internal control reagent adapted to dissolve when contacted by a fluid sample.

T20. The method of embodiment T18, comprising disposing the internal control onto at least one of (i) the first porous membrane, (ii) the second porous membrane, (iii) the internal wall of the sample chamber, (iv) at least a portion of the plurality of non-magnetic beads, and (v) the at least one magnetic element before (e), wherein the internal control is contained in an internal control reagent adapted to dissolve when contacted by a fluid sample.

T21. The method of any one of embodiments T18 to T20, wherein disposing the internal control comprises disposing the internal control reagent in a liquid form, and drying the internal control reagent after it has been disposed.

T22. The method of any one of embodiments T1 to T17, wherein an internal control is embedded in or contained within an internal control pellet adapted to dissolve when contacted by a fluid sample and/or to disintegrate when the plurality of magnetic beads is agitated in the lysis chamber, and wherein the internal control pellet is disposed within the lysis chamber prior to (e).

T23. The method of any one of embodiments T18 to T22, wherein the internal control is a whole organism, a plasmid, or a nucleic acid transcript.

Some embodiments encompass:

U1. A method for performing cell lysis on a fluid sample, the method comprising: (a) dispensing the fluid sample into a lysis chamber, wherein the lysis chamber comprises a side wall, a first porous membrane, a second porous membrane spaced apart from the first porous membrane, a plurality of non-magnetic beads contained within a space between the first porous membrane and the second porous membrane, and at least one magnetic element contained within the space, wherein pores of the first porous membrane and the second porous membrane are sized to retain the plurality of non-magnetic beads and the at least one magnetic element within the space, and wherein the fluid sample is dispensed into the lysis chamber through the first porous membrane; and (b) subjecting the at least one magnetic element to a varying magnetic field of varying polarity to cause movement of the magnetic element as the magnetic element seeks to realign with the varying polarity of the varying magnetic field, wherein the movement of the magnetic element imparts motion to the plurality of non-magnetic beads to effect mechanical lysis of cells (“cell lysis”) present in the fluid sample contained within the lysis chamber.

U2. The method of embodiment U1, wherein the varying magnetic field is created by an electromagnet.

U3. The method of embodiment U2, wherein (b) comprises alternating a current to the electromagnet to alternate a polarity of the electromagnet.

U4. The method of embodiment U3, wherein (b) comprises alternating the current to alternate the polarity of the electromagnet at a frequency of 20 Hertz to 200 Hertz.

U5. The method of embodiment U4, wherein (b) comprises pulsing the current to alternate the polarity of the electromagnet at two or more different frequencies.

U6. The method of any one of embodiments U1 to U5, further comprising: (c) after (b), transporting at least a portion of the fluid sample from the lysis chamber to a processing chamber fluidly connected to or connectable with the lysis chamber, wherein the portion of the fluid sample transported from the lysis chamber passes through the second porous membrane.

U7. The method of embodiment U6, further comprising: (d) during (c), retaining lysed cellular material within the lysis chamber while allowing released nucleic acids to pass through the second porous membrane.

U8. The method of embodiment U7, further comprising: (e) during (d) subjecting the at least one magnetic element to the varying magnetic field of varying polarity to cause movement of the magnetic element within the lysis chamber, the movement of the at least one magnetic element within the lysis chamber imparting movement of the plurality of non-magnetic beads, and the movement of the plurality of non-magnetic beads within the lysis chamber of the lysis capsule causing at least a portion of the lysed cellular material within the lysis chamber to remain in suspension at least until the fluid sample has been removed from the lysis chamber.

U9. The method of embodiment U7 or U8, further comprising: (f) in the processing chamber, immobilizing at least a portion of the released nucleic acids on a solid support and removing non-immobilized components of the fluid sample from the processing chamber.

U10. The method of embodiment U9, further comprising: (g) after (h), eluting the immobilized nucleic acids from the solid support and transporting the eluted nucleic acids to a reaction chamber of the fluidic cartridge.

U12. The method of embodiment U11, further comprising: (i) during or after (a), releasing an internal control into the fluid sample; (j) immobilizing nucleic acids associated with the internal control (“IC nucleic acids”) on the solid support during (f); (k) after (f), eluting the IC nucleic acids from the solid support and transporting the eluted IC nucleic acids to the reaction chamber; and (l) after (k), subjecting the IC nucleic acids to conditions of a second reaction, a result of (l) being used to validate a result of (h) and/or to validate the effectiveness of the cell lysis in (b).

U13. The method of embodiment U12, wherein at least a portion of the internal control is contained in an internal control reagent disposed on at least one of the first porous membrane and the second porous membrane when (a) is initiated, and wherein the internal control reagent dissolves in the fluid sample during any of (a) and (b).

U14. The method of embodiment U12, wherein at least a portion of the internal control is contained in an internal control reagent disposed on at least a portion of the plurality of non-magnetic beads and/or the at least one magnetic element when (a) is initiated, and wherein the internal control reagent disposed on the at least a portion of the plurality of non-magnetic beads and/or the at least one magnetic element dissolves in the fluid sample during any of (a) and (b).

U15. The method of embodiment U12, wherein at least a portion of the internal control is contained in an internal control reagent disposed on the side wall of the lysis chamber when (a) is initiated, and wherein the internal control reagent dissolves in the fluid sample during any of (a) and (b).

U16. The method of embodiment U12, wherein at least a portion of the internal control is embedded in or contained within an internal control pellet, and wherein the internal control pellet dissolves in the presence of the fluid sample and/or is disintegrated by the movement of the plurality of non-magnetic beads during (c), thereby releasing the internal control into the fluid sample.

U17. The method of any one of embodiments U12 to U16, wherein the conditions of the first reaction and the conditions of the second reaction are the same conditions.

U18. The method of any one of embodiments U12 to U17, wherein each of the first and second reactions is a nucleic acid amplification reaction.

U19. The method of embodiment U18, wherein the nucleic acid amplification reaction is a polymerase chain reaction (“PCR”).

Some embodiments encompass:

V1. A pellet containing an internal control for use in a nucleic acid amplification reaction, wherein the internal control is provided to validate an assay result and/or to validate the effectiveness of a cell lysis procedure, and wherein the pellet is adapted to be disrupted when subjected to collisional forces required for cell disruption to thereby release the internal control.

V2. The pellet of embodiment V1, wherein the internal control pellet comprises: a core including an excipient within which the internal control is embedded; and a coating surrounding the core and adapted to be disrupted by mechanical lysing shearing forces imparted by movement of the plurality of non-magnetic beads within the receptacle, wherein the excipient is adapted to at least partially dissolve when exposed to fluid after the coating is disrupted.

V3. The pellet of embodiment V2, wherein the excipient comprises at least one of microcrystalline cellulose and hydroxypropylcellulose, and wherein the coating comprises a cellulose derivative.

V4. The pellet of any one of embodiments V1 to V3, wherein the internal control is a whole organism, a plasmid, or a nucleic acid transcript.

Some embodiments encompass:

W1. A device with which to perform cell lysis, the device comprising: a fluid receptacle; a plurality of non-magnetic beads and at least one magnetic element within the receptacle; and an internal control disposed within a pellet located within the receptacle, wherein the pellet is adapted to be disrupted when subjected to forces imparted by movement of the plurality of non-magnetic beads within the receptacle to thereby release the internal control from the pellet, and wherein the internal control is provided to validate an assay result and/or to validate the effectiveness of a cell lysis procedure within the receptacle.

W2. The device of claim W1, comprising at least one porous membrane disposed in a flow path through the receptacle.

W3. The device of embodiment W1 or W2, wherein the internal control pellet comprises: a core including an excipient within which the internal control is embedded; and a coating surrounding the core and adapted to be disrupted by mechanical lysing shearing forces imparted by movement of the plurality of non-magnetic beads within the receptacle, wherein the excipient is adapted to at least partially dissolve when exposed to fluid after the coating is disrupted.

W4. The device of embodiment W3, wherein the excipient comprises at least one of microcrystalline cellulose and hydroxypropylcellulose, and wherein the coating comprises a cellulose derivative.

W5. The device of any one of embodiments W1 to W4, wherein the internal control is a whole organism, a plasmid, or a nucleic acid transcript.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, where like reference numerals designate corresponding parts in the various figures.

### BRIEF DESCRIPTION OF THE DRAWINGS

The accompanying drawings, which are incorporated herein and form part of the specification, illustrate various embodiments of the subject matter of this disclosure. In the drawings, like reference numbers indicate identical or functionally similar elements.

FIG. 1 is a rear perspective view of an instrument for processing a fluidic cartridge as described herein.

FIG. 2 is a front perspective view of the instrument.

FIG. 3 is an exploded top perspective view of an example of a fluidic cartridge that is processed in the instrument described herein.

FIG. 4 is a top plan view of a cartridge body of the fluidic cartridge.

FIG. 5 is a bottom plan view of the cartridge body.

FIG. 6 is a top perspective view of the cartridge body.

FIG. 7 is a bottom perspective view of the cartridge body.

FIG. 8 is a schematic transverse cross-section of the fluidic cartridge through reaction/detection chambers of the fluidic cartridge.

FIG. 9 is a bottom perspective view of a protective venting cover of the fluidic cartridge.

FIG. 10 and FIG. 10 “Detail A” show a cross-section of the protective venting cover along the line A-A in FIG. 9.

FIG. 11 is an exploded, top perspective view of a blocker, a blocker ring, and a syringe stopper of the fluidic cartridge.

FIG. 12 is a top perspective view of the blocker.

FIG. 13 is a top view of the blocker.

FIG. 14 is a bottom view of the blocker.

FIG. 15 is a top perspective view of a sample chamber cap of the fluidic cartridge.

FIG. 16 is a side view of the sample chamber cap.

FIG. 17 is a transverse cross-section of the sample chamber cap along the line A-A in FIG. 15.

FIG. 18 is a cross-section of a portion of the fluidic cartridge along the line B-B in FIG. 4 showing the cartridge body.

FIG. 19 is a cross-section of a portion of the fluidic cartridge along the line A-A in FIG. 4 showing the cartridge body.

FIG. 20 is a perspective view of a syringe driver of the instrument.

FIG. 21 is a longitudinal cross-section of a portion of the fluidic cartridge through a sample chamber and a syringe barrel of the cartridge body and through a portion of the instrument and showing a syringe plunger, a plunger of a contact detector, a pressure plate, and an upper block of the instrument in raised positions with respect to the cartridge.

FIG. 22 is a longitudinal cross-section of a portion of the fluidic cartridge through the sample chamber and the syringe barrel of the cartridge body and through a portion of the instrument and showing the syringe plunger, the plunger of the contact detector, the pressure plate, and the upper block of the instrument in lowered positions engaged with the cartridge.

FIG. 23 is a plot of motor current demand versus stopper travel for four different fluidic cartridges.

FIG. 24 is a flow diagram illustrating a method for using the demand of a motor of the syringe drive module and the output of an encoder coupled to the motor to control the position of the syringe stopper and thus the volume of fluid drawn into the syringe barrel of the fluidic cartridge.

FIG. 25 is a partial, top perspective view showing a cartridge support frame of the instrument.

FIG. 26 is a partial, top perspective view showing the cartridge support frame supporting a fluidic cartridge.

FIG. 27 is a schematic cross-section through first and second thermal modules of the instrument and through reaction/detection chambers of the cartridge and with the first thermal module in a raised position with respect to the second thermal module and the cartridge.

FIG. 28 is a schematic cross-section through the first and second thermal modules of the instrument and through the reaction/detection chambers of the cartridge and with the first thermal module in a lowered position with respect to the second thermal module and the cartridge.

FIG. 29 is a top perspective view of an upper chassis of the instrument.

FIG. 30 is a side view of the upper chassis.

FIG. 31 is a bottom perspective view of the upper chassis.

FIG. 32 is a top, partial perspective view of the instrument showing the first (top) and second (bottom) thermal modules.

FIG. 33 is a top, partial perspective view of a first (top) thermal module and second (bottom) thermal module.

FIG. 34 is a bottom, partial perspective view of the first (top) thermal module and the second (bottom) thermal module.

FIG. 35 is a top perspective view of the first (top) thermal module.

FIG. 36 is a bottom perspective view of the first (top) thermal module.

FIG. 37 is a cross-sectional view of the first (top) thermal module through the line A-A in FIG. 35.

FIG. 38 is a perspective view of the first thermal module with a first thermal assembly of the first thermal module shown in an exploded view.

FIG. 39 is an exploded, perspective view of a second thermal assembly of the second thermal module.

FIG. 40 is a front view of the first and second thermal assemblies of the second thermal module.

FIG. 41 is a left-side view of the second thermal assembly of the second thermal module.

FIG. 42 is a right-side view of the first thermal assembly of the second thermal module.

FIG. 43 is a top perspective view of the second thermal assembly of the second thermal module.

FIG. 44 is a partial, top, left-side perspective view of a contact detector of the instrument when the first thermal module is in the raised position with respect to the cartridge.

FIG. 45 is a partial, top, left-side perspective view of the contact detector of the instrument when the first thermal module is in the lowered position with respect to the cartridge and the contact detector is in contact with the cartridge.

FIG. 46 is a partial, top, front perspective view of the contact detector of the instrument.

FIG. 47 shows a flow diagram illustrating an embodiment of a method for performing a molecular assay using the instrument and fluidic cartridge described herein.

FIG. 48 is a plot of a temperature profile of a thermal cycler as described herein.

FIG. 49 is a perspective view of a first embodiment of a lysis capsule for use in a fluidic cartridge.

FIG. 50 is a cross-section of the lysis capsule along the line A-A in FIG. 49.

FIG. 51 is a transverse cross-section of a second embodiment of a lysis capsule situated within a sample chamber of a fluidic cartridge with the sample chamber closed by a cap.

FIG. 52 is a transverse cross-section of a third embodiment of a lysis capsule situated within a sample chamber of a fluidic cartridge with the sample chamber closed by a cap.

FIG. 53 is a perspective view of an embodiment of a lysis vessel.

FIG. 54 is a cross-section of the lysis vessel along the line A-A in FIG. 53.

FIG. 55 is a transverse cross-section of the lysis vessel situated within a sample chamber of a fluidic cartridge.

FIG. 56 is a perspective view of a fluidic cartridge supported in the cartridge holder with a magnet housing within which a variable magnet—e.g., an electromagnet—is housed.

FIG. 57 is a schematic view showing an electromagnet within the magnet housing and connected to an oscillating circuit configured to cause polarity of the electromagnet to repeatedly change.

FIG. 58 is an exploded perspective view of an electromagnet assembly.

FIG. 59 is a side view of the electromagnet assembly.

FIG. 60 is a cross-section of the electromagnet assembly along the line A-A in FIG. 59.

FIG. 61 is a flow diagram of a method for performing a cell lysis and a reaction using an instrument and a fluidic cartridge including a lysis capsule.

FIG. 62 is a flow chart of a method for using an internal control in a reaction using an instrument and a fluidic cartridge.

FIG. 63 is a flow chart of an alternate method for introducing a fluid sample into a lysis chamber of a lysis capsule of a fluidic cartridge.

FIG. 64 is a flow chart of a method of manufacturing a fluidic cartridge containing a lysis capsule.

FIG. 65 is a partial top perspective view of the fluidic cartridge with a chamber expander and with the cap of the chamber expander in an open position.

FIG. 66 is a partial cross-sectional view of the fluidic cartridge and chamber expander along the line X-X in FIG. 65.

FIG. 67 is an exploded cross-sectional view of the fluidic cartridge, including a lysis capsule and a chamber expander, along the line X-X in FIG. 65.

FIG. 68 is a cross-sectional view of the fluidic cartridge, lysis capsule, and chamber expander along the line Y-Y in FIG. 65.

FIG. 69 is a top perspective view of a chamber expander attachable to the fluidic cartridge with the cap of the chamber expander in an open position.

FIG. 70 is a bottom perspective view of the chamber expander with the cap of the chamber expander in an open position.

FIG. 71 is a top plan view of the chamber expander with the cap of the chamber expander in an open position.

FIG. 72 is a bottom plan view of the chamber expander with the cap of the chamber expander in an open position.

FIG. 73 is a top perspective, partial cross-sectional view of the cap of the chamber expander along the line Z-Z in FIG. 69.

FIG. 74 is a top perspective view of a hollow body of a lysis capsule for use in the fluidic cartridge of FIG. 65.

FIG. 75 is top perspective view of a bead delivery cap having a rupturable chamber for containing lytic agents (i.e., a magnetic element and non-magnetic lysis beads).

FIG. 76 is a bottom view of the bead delivery cap.

FIG. 77 is a cross-sectional view of the bead delivery cap along the line A-A in FIG. 75 with an intact bead-containing chamber.

FIG. 78 is a cross-sectional view of the bead delivery cap along the line A-A in FIG. 75 with the bead-containing chamber ruptured to release the lytic agents.

FIG. 79 is a partial cross-section through the sample chamber of the cartridge body showing the bead delivery cap inserted into the sample chamber.

FIG. 80 is a partial cross-section of the fluidic cartridge, a pressure plate of the instrument, and a contact detector having a bead delivery cap actuator, and where the pressure plate and the contact detector are in a raised position with respect to the fluidic cartridge so that the bead delivery cap actuator is not engaged with the bead delivery cap.

FIG. 81 is a partial cross-section of the fluidic cartridge, the pressure plate, and the contact detector with the bead delivery cap actuator, and where the pressure plate and the contact detector are in a lowered position with respect to the fluidic cartridge so that the bead delivery cap actuator is engaged with the bead delivery cap.

FIG. 82 is a partial cross-section of a fluidic cartridge through a mechanical lysis sample chamber that provides a lysis chamber and lytic agents for performing mechanical lysis within the sample chamber.

FIG. 83 is a partial cross-section of a fluidic cartridge through an alternate mechanical lysis sample chamber.

FIG. 84 is a partial cross-section of a fluidic cartridge through an alternate mechanical lysis sample chamber.

FIG. 85 is a schematic, cross-sectional view of a coated micropellet containing an internal control (“IC micropellet”).

FIG. 86 is a flow chart of a method of manufacturing a lysis chamber within a sample chamber of a fluidic cartridge.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

### Definitions

Unless defined otherwise, all terms of art, notations and other technical terms or terminology used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

Unless otherwise indicated or the context suggests otherwise, as used herein, “a” or “an” means “at least one” or “one or more.”

References in the specification to “one embodiment,” “an embodiment,” a “further embodiment,” “an example,” “some aspects,” “a further aspect,” “aspects,” etc., indicate that the embodiment, example, or aspect described may include a particular feature, structure, or characteristic, but every embodiment encompassed by this disclosure may not necessarily include the particular feature, structure, or characteristic. Moreover, such phrases are not necessarily referring to the same embodiment, example, or aspect. Further, when a particular feature, structure, or characteristic is described in connection with an embodiment, such feature, structure, or characteristic is also a description in connection with other embodiments, examples, or aspects, whether or not explicitly described.

This description may use various terms describing relative spatial arrangements and/or orientations or directions in describing the position and/or orientation of a component, apparatus, location, feature, or a portion thereof or direction of movement, force, or other dynamic action. Unless specifically stated, or otherwise dictated by the context of the description, such terms, including, without limitation, top, bottom, above, below, under, on top of, upper, lower, left, right, in front of, behind, beneath, next to, adjacent, between, horizontal, vertical, diagonal, longitudinal, transverse, radial, axial, clockwise, counter-clockwise, etc., are used for convenience in referring to such component, apparatus, location, feature, or a portion thereof or movement, force, or other dynamic action represented in the drawings and are not intended to be limiting.

Unless otherwise indicated, or the context suggests otherwise, terms used herein to describe a physical and/or spatial relationship between a first component, structure, or portion thereof and a second component, structure, or portion thereof, such as, attached, connected, fixed, joined, linked, coupled, or similar terms or variations of such terms, shall encompass both a direct relationship in which the first component, structure, or portion thereof is in direct contact with the second component, structure, or portion thereof or there are one or more intervening components, structures, or portions thereof between the first component, structure, or portion thereof and the second component, structure, or portion thereof.

Unless otherwise stated, any specific dimensions mentioned in this description are merely representative of an example of an implementation of a device embodying aspects of the disclosure and are not intended to be limiting.

To the extent used herein, the terms “about” or “approximately” apply to all numeric values and terms indicating specific physical orientations or relationships such as horizontal, vertical, parallel, perpendicular, concentric, or similar terms, specified herein, whether or not explicitly indicated. This term generally refers to a range of numbers, orientations, and relationships that one of ordinary skill in the art would consider as a reasonable amount of deviation to the recited numeric values, orientations, and relationships (i.e., having the equivalent function or result) in the context of the present disclosure. For example, and not intended to be limiting, this term can be construed as including a deviation of ±10 percent of the given numeric value, orientation, or relationship, provided such a deviation does not alter the end function or result of the stated value, orientation, or relationship. Therefore, under some circumstances as would be appreciated by one of ordinary skill in the art a value of about or approximately 1% can be construed to be a range from 0.9% to 1.1%.

To the extent used herein, the term “adjacent” refers to being near (spatial proximity) or adjoining. Adjacent objects or portions thereof can be spaced apart from one another or can be in actual or direct contact with one another. In some instances, adjacent objects or portions thereof can be coupled to one another or can be formed integrally with one another.

To the extent used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as stated as well as instances in which the event, circumstance, characteristic, or property occurs to a close approximation, such as accounting for typical tolerance levels or variability of the embodiments described herein.

To the extent used herein, the terms “optional” and “optionally” or the term “may” (e.g., as in the phrase “may include,” “may comprise,” “may produce,” “may provide,” or similar phrases) mean that the subsequently described component, structure, element, event, circumstance, characteristic, property, etc. may or may not be included or occur and that the description includes instances where the component, structure, element, event, circumstance, characteristic, property, etc. is included or occurs and instances in which it is not or does not.

To the extent used herein, the terms “first,” “second,” and similar terms preceding the name of an element (e.g., a component, apparatus, location, feature, or a portion thereof or a direction of movement, force, or other dynamic action) are used for identification purposes to distinguish between similar elements, and are not intended to necessarily imply order or rank, nor are the terms “first” and “second” intended to preclude the inclusion of additional similar elements. Furthermore, unless the context indicates otherwise, use of the term “first” preceding the name of an element (e.g., a component, apparatus, location, feature, or a portion thereof or a direction of movement, force, or other dynamic action) does not necessarily imply or require that there be additional, e.g., “second,” “third,” etc., such element(s).

To the extent used herein, the terms or phrases “configured to,” “adapted to,” “operable to,” “constructed and arranged to,” and similar terms mean that the subject of the term or phrase includes, constitutes, or otherwise encompasses the requisite structure(s), mechanism(s), arrangement(s), component(s), material(s), algorithm(s), circuit(s), programming, etc. to perform a specified task or tasks or achieve a specified output or characteristic, either automatically or perpetually or selectively when called upon to do so.

To the extent used herein, the term “amplification reaction” or “nucleic acid amplification reaction” means a procedure used to produce multiple copies of a specific segment of nucleic acid. Amplification reactions may be isothermal or require repetitive cycling between different temperatures, such as is required with a Polymerase Chain Reaction (PCR).

The term “amplification reagent” means a material containing one or more components needed for an amplification reaction. In a nucleic acid amplification, such components may include primers, nucleoside triphosphates, and/or cofactors needed for amplification of a target nucleic acid (e.g., divalent cations such as Mg++).

To the extent used herein, the term “analyte” refers to a molecule or substance that is detected or subjected to analysis in an assay. Examples of analytes include nucleic acids, proteins (e.g., antibodies, polypeptides, and prions), and antigens.

To the extent used herein, the term “assay” refers to a procedure for detecting and/or quantifying an analyte in a sample. A sample containing or suspected of containing the analyte is contacted with one or more reagents and subjected to conditions permissive for generating a detectable signal informative of whether the analyte is present or an amount (e.g., mass or concentration) of the analyte in the sample.

To the extent used herein, the term “analyzer” refers to an automated instrument that is capable of performing one or more steps of an assay, including the step of determining the presence or absence of one or more analytes suspected of being present in a fluid sample.

To the extent used herein, the term “molecular assay” refers to a procedure for specifically detecting and/or quantifying a target molecule, such as a particular nucleic acid. A sample comprising or suspected of comprising the target molecule is contacted with one or more reagents, including at least one reagent specific for the target molecule, and subjected to conditions permissive for generating a detectable signal informative of whether the target molecule is present. For example, where the molecular assay includes an amplification reaction, such as a polymerase chain reaction (PCR), the reagents include primers that may be specific for a target nucleic acid, and the generation of a detectable signal can be accomplished, at least in part, by providing a labeled probe (e.g., fluorescently labeled probe) that hybridizes in a target-specific manner to the amplicon produced by the primers in the presence of the target. Alternatively, the reagents can include an intercalating dye (e.g., SYBR® Green) for detecting the formation of double-stranded nucleic acids.

To the extent used herein, the term “point-of-care testing” (POCT), sometimes referred to as near-patient testing, is testing conducted close to the site of patient care or treatment. This may be in the context of a hospital, doctor's office, or field testing. Unlike high-throughput systems, POCT systems are generally small and may be easily portable. Most POCT systems are capable of running an assay on a single or limited number of samples simultaneously.

To the extent used herein, the term “reagent” refers to any substance or mixture that participates in an assay, other than sample material and products of the assay. Examples of reagents for use in a molecular assay may include nucleotides, enzymes, primers, probes, and salts.

To the extent used herein, the term “receptacle” or “fluid receptacle” refers to any type of fluid container, including, for example, a tube, a vial, a cuvette, a well or cartridge or other article having one or more wells or chambers formed therein or attached thereto, a microtiter plate, etc., that is configured to contain a sample or another fluid (collectively referred to herein as fluid). Tubes may be cylindrical (i.e., circular in cross-section) or non-cylindrical and may have flat or rounded closed ends. A non-limiting example of such a receptacle is the Aptima® Multitest Swab Collection Kit (Hologic, Inc.; Marlborough, MA).

To the extent used herein, the term “sample” refers to any substance suspected of containing at least one analyte of interest. The analyte of interest may be, for example, a nucleic acid, a protein, a chemical, or the like. The substance may be derived from any source, including an animal, an industrial process, the environment, a water source, a food product, or a solid surface (e.g., surface in a medical facility). Substances obtained from animals may include, for example, blood or blood products, urine, mucus, sputum, saliva, semen, tears, pus, stool, nasopharyngeal or genitourinary specimen obtained with a swab or other collection device, and other bodily fluids or materials. The term “sample” will be understood to mean a specimen in its native form or any stage of processing.

To the extent used herein, the term “thermal contact” or “thermal communication” means the ability to allow thermal energy transfer between two systems or bodies at different temperatures. The two systems or bodies may be in direct physical contact such that the thermal energy transfer occurs directly from one system or body to the other system or body, or an intervening material, including air, may be disposed between the two systems or bodies such that thermal energy transfer occurs from one system or body to the other system or body through the intervening material.

To the extent used herein, the term “unit dose form” means an amount that is sufficient for performing a single assay. That is, as opposed to a bulk reagent, which is provided in amount that can be used to perform multiple assays, a “unit dose” or “unitized” reagent is an amount of a reagent that can be used for a single assay (the single assay may be designed to determine the presence of one or more analytes).

A “fluidic cartridge” is a device including a fluidic network of two or more chambers, or wells, for containing fluid which are fluidly interconnected, or interconnectable, by one or more fluid channels. The device is configured to interface with a processing instrument or analyzer for processing the fluidic cartridge. A fluidic cartridge may include one or more of a sample chamber for receiving a fluid sample and by which the fluid sample is introduced to the fluidic cartridge, storage chamber(s) within which one or more materials, such as reagent, buffers, or probes, processing chamber(s) within which one or more processes are performed on fluid materials, such as combining/mixing, filtering, purifying, etc., waste chamber(s) within which one or more waste materials are stored, and reaction chamber(s) within which a chemical or biochemical reaction takes place.

To the extent used herein, “processing” a fluidic cartridge means effecting one or more processes on fluids or other materials contained in the cartridge, including, for example, one or more of applying positive or negative pressure to the cartridge, applying physical pressure to at least one chamber of the cartridge to at least partially collapse the chamber, or actuating a pump mechanism operatively coupled to the cartridge to effect fluid movement between chambers within the fluidic network of the cartridge, actuating or otherwise altering flow control mechanisms, such as valves, to alter the flow control mechanism between an open state permitting fluid flow past the flow control mechanism and a closed state blocking fluid flow past the flow control mechanism, combining two or more materials within a chamber of the cartridge, filtering or otherwise purifying fluid sample within the cartridge, heating and/or cooling the fluid within one or more chambers of the cartridge, and detecting and recording signals based on optical emissions from fluids contained in one or more chambers of the cartridge.

To the extent used herein, an “internal control” refers to a molecule detected in order to validate an assay result, such as a negative assay result in which no analyte was detected. An amplification reaction (e.g., PCR), can be affected by, for example, the presence of inhibitors in a sample (e.g., hemoglobin), errors in a sample extraction process, or a thermal cycler malfunction. In the case of amplification reactions, the internal control is used to demonstrate that the reagents and conditions are such that a target analyte, if present in the sample, should be successfully amplified and detected during the assay. In amplification reactions in which the target analyte is a nucleic acid, an internal control typically has a sequence different from the target analyte, at least in part, but can have properties that result in similar amplification and detection characteristics (e.g., similar GC content). A nucleic acid internal control can be amplified with dedicated amplification primers or with the same amplification primers as a target analyte. An internal control nucleic acid can lack the sequence targeted by a probe for the target analyte and contain a sequence targeted by a probe specific for the internal control nucleic acid. The nucleic acid internal control may be in the form of a nucleic acid transcript or it may be a nucleic acid contained within a plasmid or cell, such as yeast, in which case the cell may, in addition to harboring the nucleic acid internal control, serve as an “extraction control” for monitoring the effectiveness of a lysis procedure in releasing nucleic acids from targeted microorganisms, as well as other extraction procedures, such as filtering, target capture, purification, etc.

To the extent used herein, the term “porous membrane” refers to a selective barrier that controls the passage of substances-allowing some substances to pass through the barrier while preventing other substances from passing through the barrier depending on the size of the substances. A porous membrane may be, for example, a woven mesh, a thin porous substrate, or a filter matrix (e.g., spun or sintered), but, unless specifically indicated for a particular example or application, the term porous membrane is not intended to connote, and should not be interpreted to imply, an particular composition, configuration, form factor, or thickness.

### DETAILED DESCRIPTION OF DRAWINGS

### Instrument Overview

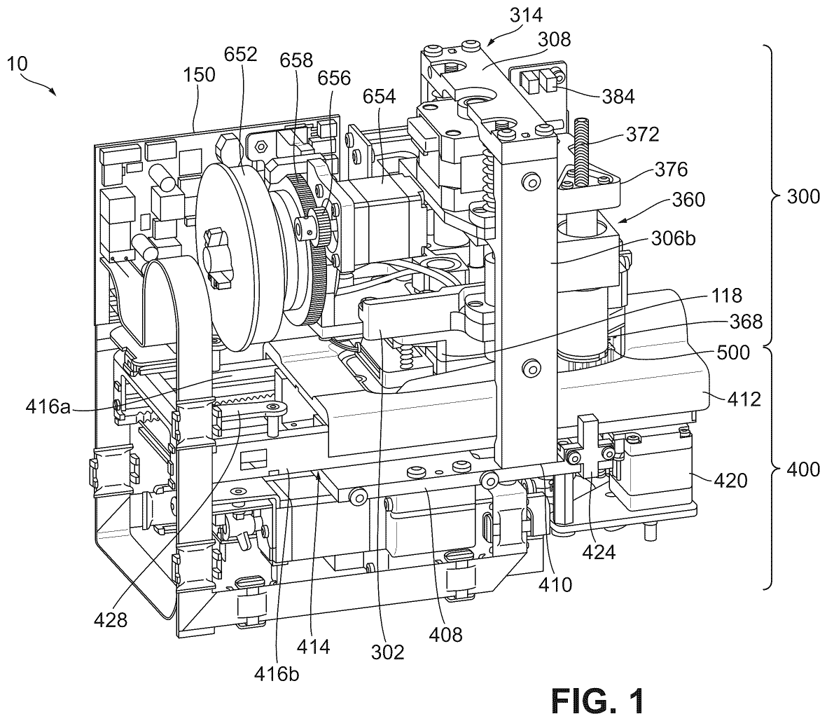

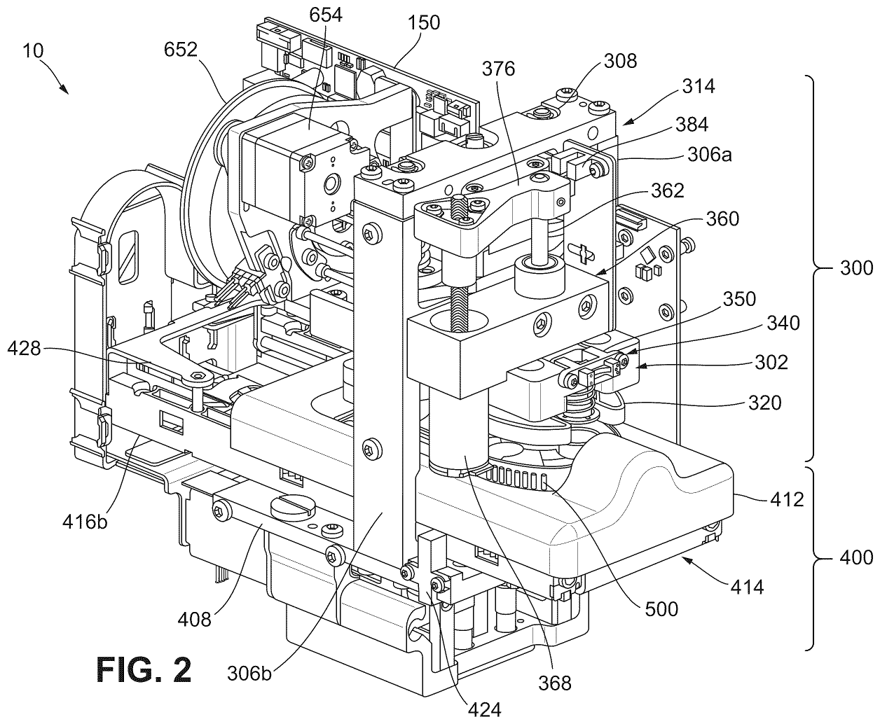

FIGS. 1 and 2 show the internal components of an instrument 10 as described herein for receiving and operating on a test platform, such as a fluidic cartridge (i.e., a device configured to be placed into and interface with a processing instrument and which includes reagent and sample storage and fluid handling components, such as fluid flow channels and flow control valves), to process a sample (e.g., perform an assay, such as a molecular assay, and collect data regarding the results of the assay) on or within the test platform. Instrument 10 includes components for applying thermal energy to one or more reaction/detection regions of the test platform, components for transmitting optical signals to and/or from the reaction/detection region(s), and a component for actuating a syringe pump within the test platform. FIG. 1 is a rear perspective view of the instrument 10, and FIG. 2 is a front perspective view of the instrument 10. Instrument 10 may be a point-of-care testing system for providing sample-to-result testing employing disposable fluidic cartridges comprising interconnected chambers (or wells) and reaction chambers that can be prepackaged in unit dose form with all of the reagents needed to perform the desired testing. The fluidic cartridges may be closed systems that minimize opportunities for contamination.

Typically, such an instrument would include a housing within which the internal components would be enclosed, but such a housing is omitted from FIG. 1 so that the internal components can be seen.

As shown in FIGS. 1 and 2, a test platform, e.g., a fluidic cartridge 500, is situated within the instrument 10, and the internal components of the instrument can be generally grouped into a first chassis, or upper chassis, 300, referring to those internal components situated above the cartridge 500, and a second chassis, or lower chassis, 400, referring to those internal components situated below the fluidic cartridge 500 and on which the fluidic cartridge 500 is supported. Fluidic cartridge 500 may be a microfluidic cartridge, meaning that at least a portion of any fluid passages, channels, chambers, wells, reaction chambers, etc. within which fluid flows and/or is retained is geometrically constrained to a small scale (for example, sub-millimeter) at which surface forces acting on the fluids meet or exceed volumetric forces. Upper chassis 300 may include a syringe driver 360 configured to actuate a syringe plunger coupled to a syringe stopper within the cartridge 500, as will be described herein.

### Fluidic Cartridge

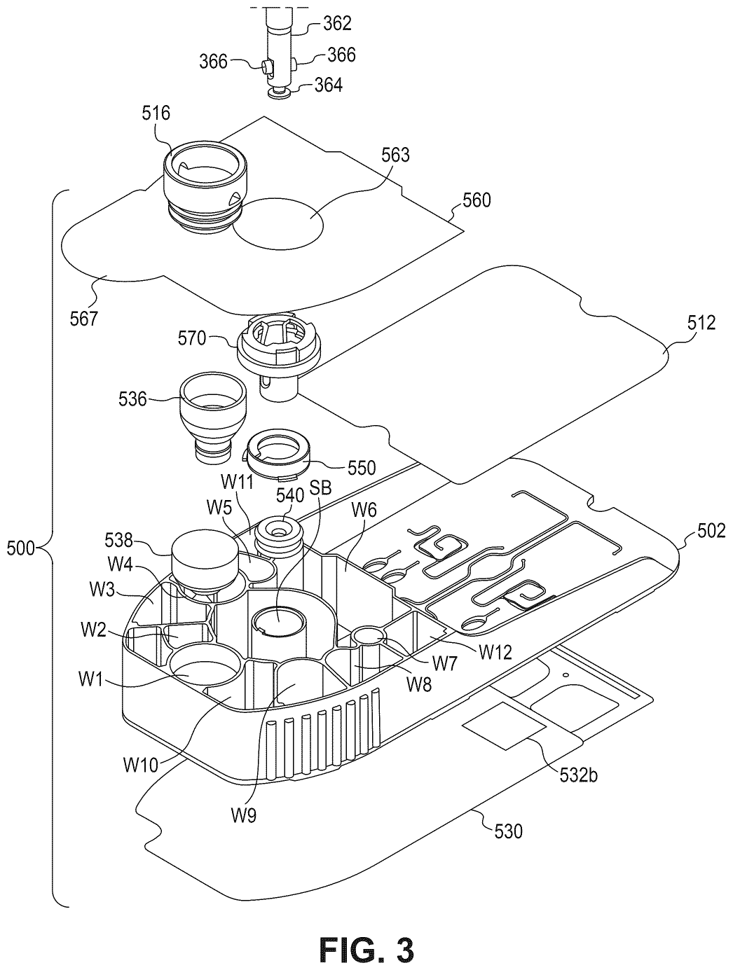

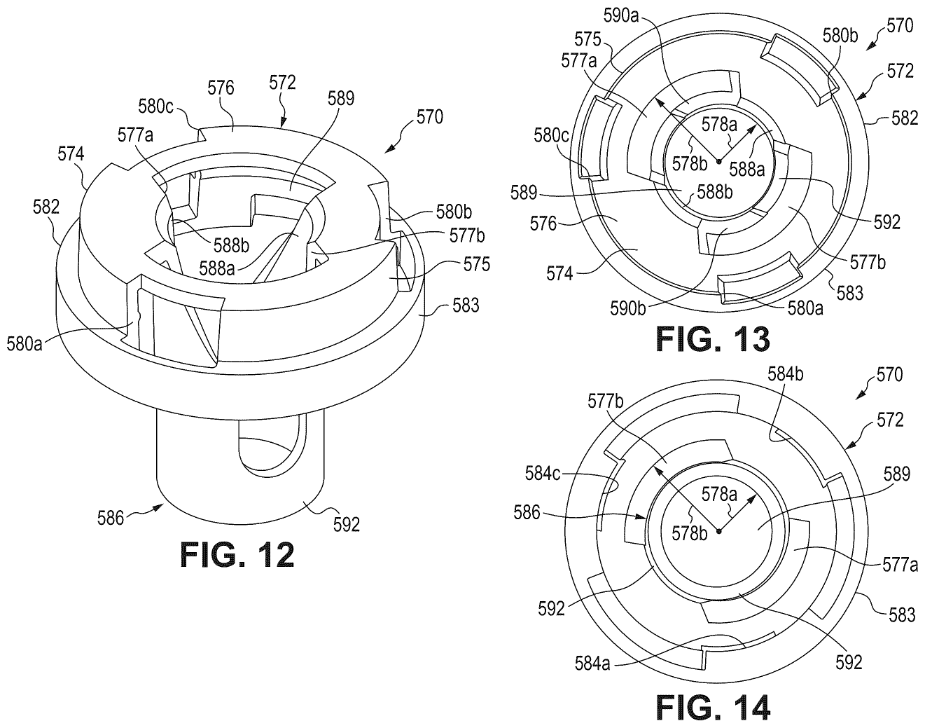

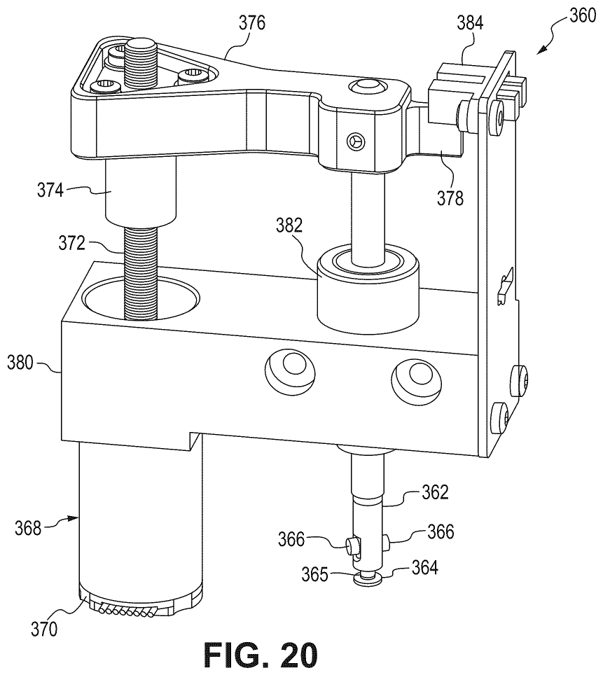

An embodiment of a fluidic cartridge 500 and components thereof are shown in FIGS. 3 to 19. FIG. 3 shows an exploded, top perspective view of cartridge 500. Fluidic cartridge 500 includes a cartridge body 502, a first (e.g., top) film 512, a second (e.g., bottom) film 530, an elastomeric syringe stopper 540, a blocker ring 550, a blocker 570, a filter 538, a purification column insert 536 that positions and holds a purification column (e.g., a silica column, Grade GF51 Hahnemühle Life Science, Dassel, Germany (Item No. GF51RL01550)), which may be in the form of a disc, a cap 516, and a protective venting cover 560. For convenience and consistent with the examples shown in the drawings, film 512 will be referred to herein as the top film and film 530 will be referred to herein as the bottom film. A plunger 362 coupled to syringe driver 360 of the instrument 10 (see FIG. 20, described below) includes a plunger head 364 that is received within a recess formed in the stopper 540 and plunger ribs 366 that engage the blocker 570 as described below. Cartridge body 502 of the fluidic cartridge 500 includes a plurality of chambers, or wells, W1 to W12 and SB, (i) including the sample chamber and storage chambers containing or configured to receive materials (e.g., sample material, reagents, buffers, etc.) used in performing an assay (e.g., a molecular assay), within the cartridge, (ii) chambers, or wells, within which two or more materials may be combined and mixed, (iii) chambers, or wells, for receiving and holding waste material, and (iv) reaction/detection chambers 510a1, 510a2, 510b1, 510b2 (i.e., detection regions) within which reactions may take place and/or from which detectable signals emitted by a reaction within the chamber are detected. In the context of the present disclosure, although the terms “well” and “chamber” may be used interchangeably in some descriptions, in general, the term “well” refers, but is not limited to, an open-ended reservoir or depression formed in the cartridge body 502, such as wells W1 to W12 and SB, and the term “chamber” refers, but is not limited, to a well of the cartridge body 502 that is at least partially enclosed, e.g., by first film 512, second film 530, and/or venting cover 560, to form an at least partially enclosed compartment or space. More than one of the functions of containing, combining, reacting, and detecting may occur within one or more functional chambers of the cartridge 500. As described below, functional chambers within the cartridge may be fluidly interconnected by fluid channels, or conduits, and the cartridge includes one or more fluid flow control valves, which may be selectively acted upon, e.g., by valve actuators (not shown) of instrument 10, to controllably permit or prevent fluid flow within a fluid channel with which the valve is operatively associated. The illustrated example has four reaction/detection chambers 510a1, 510a2, 510b1, 510b2, arranged in two pairs (or sets or groups) 510a1, 510a2 and 510b1, 510b2. In other examples, the cartridge has fewer than or more than four reaction/detection chambers. For example, a cartridge may have one or more groups or sets of three clustered reaction/detection chambers.

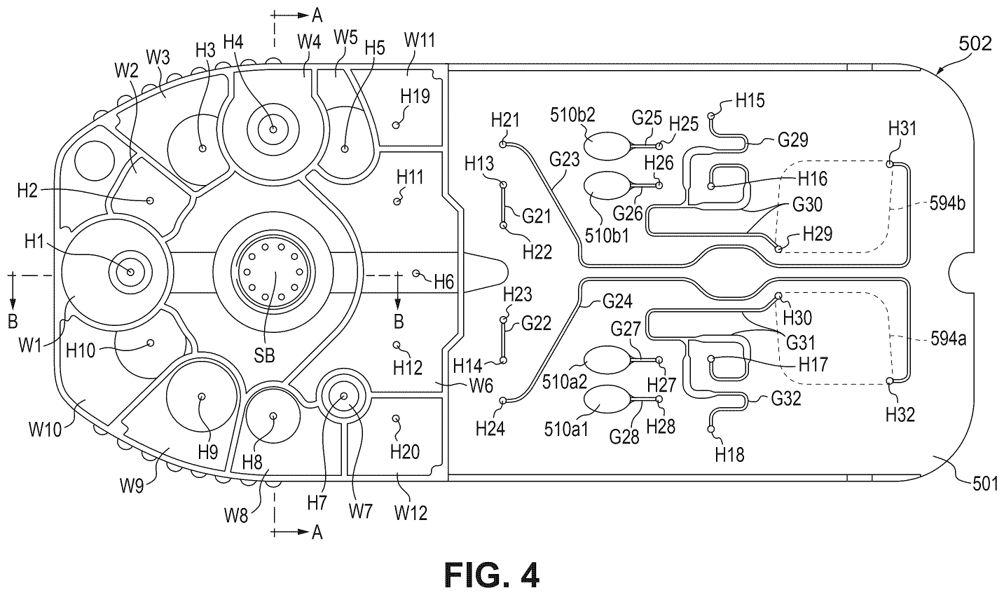

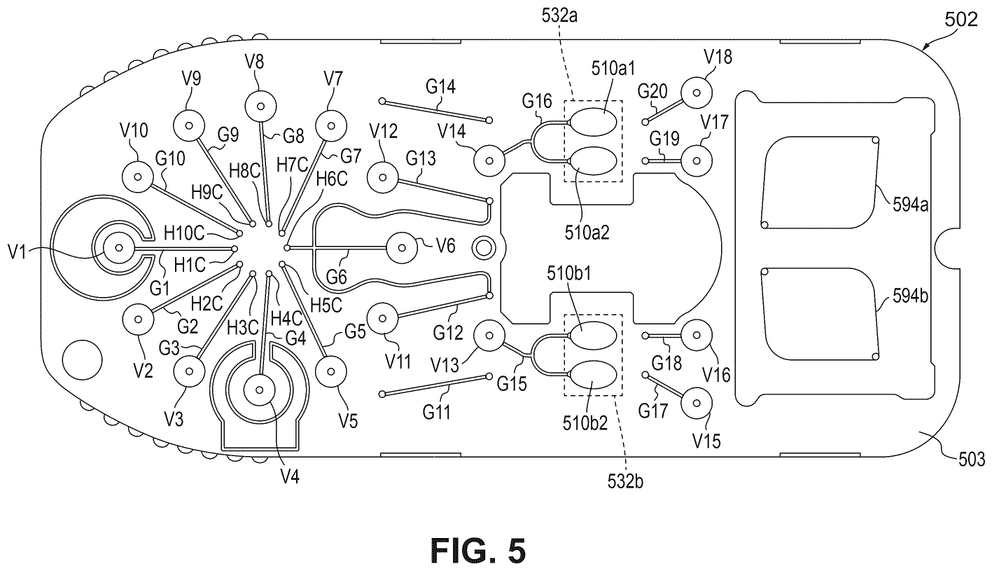

Cartridge body 502 has a first (e.g., top) face 501 (FIG. 4) and a second (e.g., bottom) face 503 (FIG. 5). For convenience and consistent with the examples shown in the drawings, face 501 will be referred to herein as the top face and face 503 will be referred to herein as the bottom face. Cartridge body 502 may be made by injection molding of a thermoplastic polymer material, such as, cyclic olefin copolymers (COC) or cyclic olefin polymers (COP), including polycarbonate, polyacrylamide, polyethylene, polymethyl-methacrylate (PMMA), polydimethylsiloxane (PDMS), and polyvinyl chloride (PVC) and is preferably made of polypropylene (PP). In some embodiments, the cartridge body 502 is made by stereolithography or by sintering. Cartridge body 502 may be made from an opaque material.

As shown in FIG. 4—a top plan view of cartridge body 502—and FIG. 5—a bottom plan view of cartridge body 502—cartridge body 502 includes a plurality of through-holes H1 to H32 extending between the top face 501 and the bottom face 503 to fluidically connect elements from either face to the other. To avoid cluttering the figures, through holes H1 to H32 are only labeled in FIG. 4. Cartridge body 502 includes a plurality of bottom grooves G1 to G20 formed in the bottom face 503 and a plurality of top grooves G21 to G32 formed in the top face 501. Each of grooves G1 to G32 may have a depth of between 0.01 mm and 0.5 mm, preferably between 0.2 mm and 0.4 mm, most preferably about 0.3 mm, and may have a width of about 0.5 mm. Each of through-holes H1 to H18 is associated with a corresponding valve V1 to V18, comprising a cylindrical recess formed in the bottom face 503 of the cartridge body 502 and which is generally coaxially arranged with respect to the associated through-hole and has a diameter that is larger than the associated through-hole. In one non-limiting example, the recess associated with each of valves V1 to V18 may have a diameter of between 1 mm and 10 mm, preferably between 2 mm and 8 mm, preferably about 4 mm, and a depth of between 0.02 mm and 0.4 mm, preferably between 0.05 mm and 0.15 mm, and most preferably about 0.1 mm. One or two of the grooves G1 to G32 terminates at an associated valve V1 to V18. Through-holes H1 to H10 are also associated with chambers W1 to W10, through-holes H11 and H12 are associated with chamber W6, through-hole H19 is associated with chamber W11, and through-hole H20 is associated with chamber W12. Through-holes H21 to H32 are not directly associated with either a valve or a chamber and provide connections between a groove or other feature on the top face 501 and a groove or other feature on the bottom face 503. Cartridge body 502 also includes central through-holes H1c to H10c arranged in a circle within well SB (syringe barrel).

The through-holes H1 to H32 and H1c to H10c, valves V1 to V18 and associated recesses, and the bottom grooves G1 to G21 and top grooves G22 to G32 formed in the cartridge body 502 form a fluidic network of channels and the fluid control valves in these channels. For that purpose, it is necessary to close the through-holes, recesses, and grooves that are open to the top face 501 or the bottom face 503 of the cartridge body 502. Bottom film 530 is secured to the bottom face 503 of the cartridge body 502 to cover bottom grooves G1 to G20 to form corresponding channels (which may be microfluidic channels), the recesses of valves V1 to V18 to form the corresponding valves, central through-holes H1c to H10c, and through-holes H19 to H32 flush with the bottom face 503. Bottom film 530 may comprise a material similar to the cartridge body 502 including, for example, polypropylene (PP). Bottom film 530 may comprise a thermoplastic film with a thickness between 0.1 mm and 0.2 mm (100 μm-200 μm), which is bonded or welded to the surface of the bottom face 503 by a thermal welding technique (e.g., by laser-welding), bonding, adhesive, or chemical linking methods.

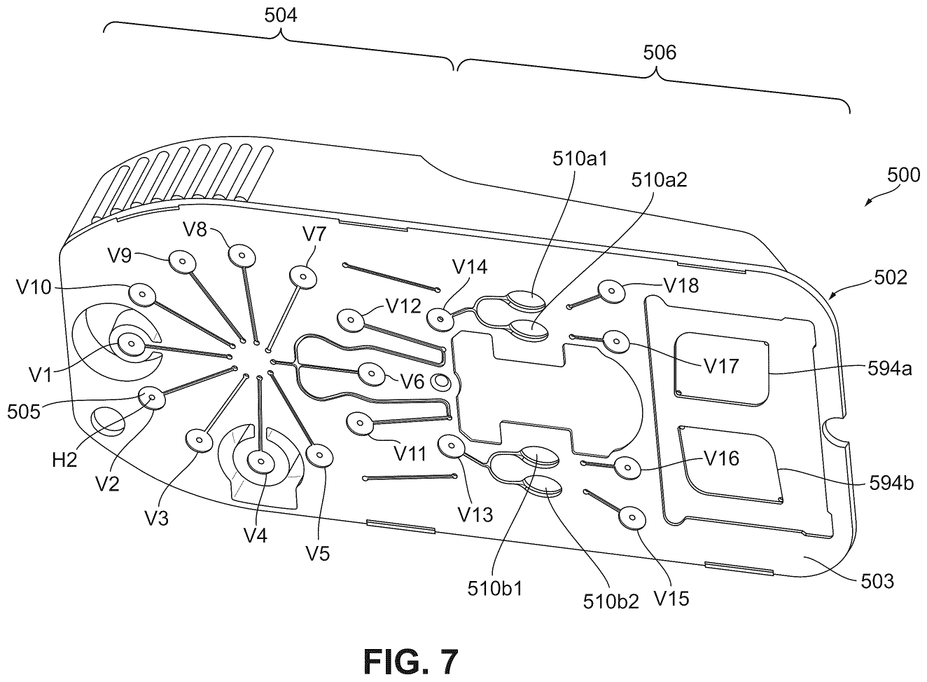

Valves V1 to V18 are formed by the bottom film 530, which may be deformable, extending across each recess opposite an annular valve seat defined between the recess of each valve V1 to V18, and the associated through-hole H1 to H18, respectively, of the valve. A single valve seat 505 between the recess of valve V2 and associated through hole H2 is labeled in FIG. 7. In one non-limiting example, the surface of the deformable bottom film 530, positioned opposite the recesses of valves V1 to V18 is, when un-deformed, approximately planar and parallel to the bottom face 503 of the cartridge body 502 and spaced apart from the valve seat between the recess and the through-hole. Bottom film 530 is capable of being deformed by an external actuator (not shown) having a width that is greater than the width (diameter) of the through hole, but not larger than the width (diameter) of the recess forming the valve seat and locally pushing the film into the recess. The deformation of the bottom film 530 into contact with each valve seat of valves V1 to V18 blocks the associated through-holes H1 to H18, whose diameter is smaller than that of each associated recess so that the film contacts the valve seat and seals the associated through-hole.

Top film 512 may be secured to top face 501 of the cartridge body 502, e.g., by thermo-welding, adhesive, or chemical linking methods, to close the top grooves G21 to G32 flush with the top face 501 to form corresponding channels (which may be microfluidic channels) in the same way bottom film 530 closes bottom grooves G1 to G20 to form corresponding channels. Top film 512 may be made of a material similar to the cartridge body 502, e.g., polypropylene, and may have a thickness of about 0.1 mm.

Fluidic cartridge 500 may include auxiliary detection regions 594a, 594b (see FIGS. 4, 5, 7). In one non-limiting example, each of auxiliary detection regions 594a, 594b comprises a micro-array slide (or biochip) bonded on the bottom face 503 of the cartridge body 502 within a recessed cavity that, when covered, e.g., by bottom film 530, forms a detection chamber for nucleic acid analysis. Instrument 10 may include means for optical excitation of the micro-array slide (not shown) and means for optical detection of a micro-array image (not shown) that is representative of an analyte of interest (e.g., a nucleic acid) of the sample being analyzed in the cartridge. See, e.g., U.S. Pat. No. 10,654,039 for further descriptions of a micro-array slide.

Referring to FIGS. 4 and 5, bottom grooves G1 to G10 extend between central through-holes H1c to H10c, respectively, and a recess associated with each of valves V1 to V10, respectively, each of the valves V1 to V10 being associated with a through-hole H1 to H10, respectively. Each of through-holes H1 to H10, associated with valves V1 to V10, respectively, connects chambers W1 to W10, respectively, to bottom grooves G1 to G10, respectively. In this context, reference to connections to or by the top or bottom grooves means connections to or by the corresponding channels formed by each groove when covered, such as by top film 512 or bottom film 530. Through-hole H11, associated with valve V11, connects chamber W6 to bottom groove G12. Through-hole H12, associated with valve V12, connects chamber W6 to bottom groove G13. Through-hole H13, associated with valve 13, connects bottom groove G15, which is connected to reaction/detection chambers 510b1 and 510b2, to top groove G21. Through-hole H14, associated with valve V14, connects bottom groove G16, which is connected to reaction/detection chambers 510a1 and 510a2, to top groove G22. Through-hole H15, associated with valve V15, connects bottom groove G17 to top groove G29, which merges with top groove G30. Through-hole H16, associated with valve V16, connects bottom groove G18 to top groove G30. Through-hole H17, associated with valve V17, connects bottom groove G19 to top groove G31. Through-hole H18, associated with valve V18, connects bottom groove G20 to top groove G32, which merges with top groove G31. Through-hole H19 connects bottom groove G11 to chamber W11. Through-hole H20 connects bottom groove G14 to chamber W12. Through-hole H21 connects bottom groove G11 to top groove G23. Through-hole H22 connects bottom groove G12 to top groove G21. Through-hole H23 connects bottom groove G13 to top groove G22. Through-hole H24 connects bottom groove G14 to top groove G24. Through-hole H25 connects bottom groove G17 to top groove G25, which is connected to chamber 510b2. Through-hole H26 connects bottom groove G18 to top groove G26, which is connected to chamber 510b1. Through-hole H27 connects bottom groove G19 to top groove G27, which is connected to chamber 510a2. Through-hole H28 connects bottom groove G20 to top groove G28, which is connected to chamber 510a1. Through-hole H29 connects top groove G30 to auxiliary detection region 594b of the cartridge. Through-hole H31 connects auxiliary detection region 594b to top groove G23, which is connected, via through-hole H21, to bottom groove G11, which is connected, via through-hole H19, to chamber W11 (e.g., a waste chamber). Through-hole H30 connects top groove G31 to auxiliary detection region 594a of the cartridge. Through-hole H32 connects auxiliary detection region 594a to top groove G24, which is connected, via through-hole H24, to bottom groove G14, which is connected, via through-hole H20, to chamber W12 (e.g., a waste chamber). Thus, when valve V8 is open, reaction chamber 510a1 is connected, via grooves G28, G20, G32, and G 31, to auxiliary detection region 594a. When valve V17 is open reaction chamber 510a2 is connected, via grooves G27, G19, and G31, to auxiliary detection region 594a. When valve V15 is open, reaction chamber 510b2 is connected, via channels G25, G17, G29, and G30, to auxiliary detection region 594b. When valve V16 is open, reaction chamber 510b1 is connected, via channels G26, G18, and G30, to auxiliary detection region 594b.

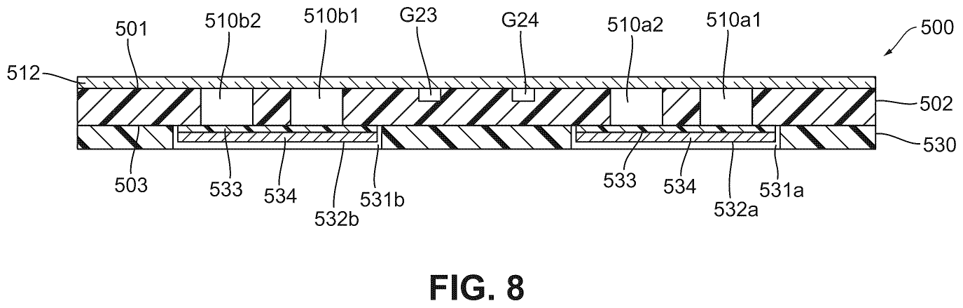

As shown in FIG. 8, which is a schematic, transverse cross-section of the cartridge 500, chambers 510a1, 510a2, 510b1, and 510b2 are defined by openings formed in the cartridge body 502 which extend between the top face 501 and bottom face 503 and which are enclosed by the bottom film 530 and the top film 512. Reaction/detection chambers 510a1, 510a2, 510b1, 510b2 receive reaction mixtures prepared from the contents of one or more of chambers W1 to W10, the reaction mixtures are exposed to heat (e.g., isothermal or thermocyclic profiles) within the chambers 510a1, 510a2, 510b1, 510b2 by contacting a top portion of the fluidic cartridge 500 in the vicinity of chambers 510a1, 510a2, 510b1, 510b2 with a top heater and contacting a bottom portion of the fluidic cartridge 500 in the vicinity of chambers 510a1, 510a2, 510b1, 510b2 with a bottom heater, and a reaction (e.g., an amplification reaction) occurs within the chambers 510a1, 510a2, 510b1, 510b2. The reaction mixtures within chambers 510a1, 510a2, 510b1, 510b2 may include detectable probes that, upon hybridization to a molecule of interest, emit detectable optical signals during a reaction, e.g., a fluorescent signal of a certain emission wavelength when exited by light of a certain excitation wavelength, for which purpose at least one wall of the chambers 510a1, 510a2, 510b1, 510b2 may be transparent or translucent. For example, where the cartridge body 502 is made from an opaque material, top film 512 may be transparent or translucent, or at least a portion of top film 512 covering chambers 510a1, 510a2, 510b1, 510b2 may be transparent or translucent, to permit an excitation signal to be delivered to the chambers from above the chambers and to permit an emission signal to be detected from above the chambers.

To promote even heat distribution over the chambers 510a1, 510a2, 510b1, 510b2, bottom film 530 may comprise a layer of thermally-conductive material, such as metallic foil (e.g., aluminum), disposed over the bottom face 503 of the cartridge body 502, at least in the vicinity of the chambers 510a1, 510a2 and in the vicinity of chambers 510b1, 510b2. As shown in FIG. 8, lower film 530 may have cutouts 531a, 531b over chambers 510a1, 510a2 and chambers 510b1, 510b2, respectively. A thermally-conductive laminate seal 532a is disposed within cutout 531a and affixed to bottom face 503 of cartridge body 502 over chambers 510a1, 510a2, and a thermally-conductive laminate seal 532b is disposed within cutout 531b and affixed to bottom face 503 of cartridge body 502 over chambers 510b1, 510b2. Locations of the thermally-conductive laminates 532a, 532b are shown in dashed lines in FIG. 5 The cutout 531a, 531b and associated thermally-conductive laminate seal 532a, 532b may be rectangular, as shown in FIGS. 3 and 5, circular, oval-shaped, or any desired shape. Where the reaction/detection chambers are arranged as spatially separated groups of chambers (wherein a “group” may include one or more chambers), a discrete thermally-conductive laminate seal may be provided to cover each group. For example, as the chambers 510a1, 510a2 and 510b1, 510b2 of fluidic cartridge 500 are arranged as spatially-separated groups (e.g., pairs), two separate thermally-conductive laminates are provided: laminate seal 532a for covering the group 510a1, 510a2 and laminate seal 532b for covering group 510b1, 510b2.

In one non-limiting example, each thermally-conductive laminate seal 532a, 532b comprises a plastic layer 533 (e.g., polypropylene) to which a conductive foil layer 534 is laminated. Suitable, commercially-available products include Thermo-Fisher AB 3599, available from Thermo-Fisher Scientific of Waltham, Massachusetts. Conductive foil layer 534 may also be optically reflective (e.g., aluminum or metallized PET film). The plastic layer 533 and conductive foil layer 534 may be secured together by a suitable adhesive or other means suitable for securing plastic to foil. In one non-limiting example, the conductive foil layer 534 has a thickness of 60 μm to 80 μm, and the plastic layer 533 has a thickness of 10 μm to 20 μm for a total thickness of each thermally-conductive laminate seal 532a, 532b of 70 μm to 100 μm. As noted above, the bottom film 530 film may have a thickness of about 0.1-0.2 mm (100 μm-200 μm). In another example, each thermally-conductive laminate seal 532a, 532b includes a second plastic layer (not shown) affixed to an opposite side of the conductive foil layer 534.

Each thermally-conductive laminate seal 532a, 532b is affixed to the cartridge body 502 by heat sealing, ultrasonic welding, adhesive, or other suitable method for bonding the plastic layer 533 of each thermally-conductive laminate seal 532a, 532b to the cartridge body 502 to prevent fluid leakage from the chambers 510a1, 510a2, 510b1, 510b2. In this regard, for heat sealing or ultrasonic welding, cartridge body 502 may include energy directors to facilitate the heat sealing or ultrasonic welding process. Energy directors are components or features in heat sealing applications that help focus and control the flow of energy (heat or vibrations) to the area where the seal is being created. Examples of energy directors include raised features (e.g., a rib) adjacent to or surrounding each of the chambers 510a1, 510a2, 510b1, 510b2 to form a narrow edge (e.g., a dome-shaped cross-section or a knife-edge (triangular) cross-section) that will focus energy at the edge and facilitate localized material melting at the edge to promote sealing to the laminate seals 532a, 532b. The conductive laminate seals 532a, 532b are heat sealed by melting and fusing the energy directors around the chambers 510a1, 510a2, 510b1, 510b2 with the plastic layer 533 of each of the laminate seals 532a, 532b. An example of a fluidic cartridge employing energy directors for facilitating heat sealing or ultrasonic welding of a seal to a cartridge body is described in International Application No. PCT/US2025/026844, entitled “Fluidic Cartridge and Apparatuses for Processing Fluidic Cartridges,” filed Apr. 29, 2025.

The conductive foil layer 534 of each thermally-conductive laminate seal 532a, 532b, being an effective thermal conductor, combined with a relatively thin plastic layer such as polypropylene, which acts as an insulator, facilitates rapid conductive thermal transfer from a heater disposed beneath the chambers 510a1, 510a2, 510b1, 510b2, thereby rapidly heating the chambers by the heater disposed beneath the chambers, and promotes even heat distribution to minimize thermal gradients across the chambers 510a1, 510a2, 510b1, 510b2.

In some examples, conductive foil layer 534 may improve the strength and accuracy of optical emission signal detection from the chambers 510a1, 510a2, 510b1, 510b2. The conductive foil layer 534 of each thermally-conductive laminate seal 532a, 532b may provide a reflective surface that increases optical emission signal strength. An optical excitation signal introduced from above each of the chambers 510a1, 510a2, 510b1, 510b2 passes through reaction mixtures within the chambers and excites probe-associated labels. Then, as the optical excitation signal is reflected off the conductive foil layer 534 at the bottom of each chamber, the reflected excitation signal again passes through reaction mixtures within the chambers, once again exciting probe-associated labels. Moreover, optical emission signal collected from above the chambers 510a1, 510a2, 510b1, 510b2 will be strengthened as both optical signal emitted directly toward the top of each chamber as well as optical signal emitted toward the bottom of each chamber and reflected toward the top of the chamber by the conductive foil layer 534 at the bottom of the chamber can be collected.

Furthermore, the laminate seals may increase the accuracy of emission signals collected from the chambers 510a1, 510a2, 510b1, 510b2. A relatively thick layer of transparent or translucent film (e.g., such as the thickness 100 μm to 200 μm of the bottom film 530) directly covering the chambers 510a1, 510a2, 510b1, 510b2 may act as an optical transmitter (i.e., a light pipe) that can transmit optical signals laterally from one chamber to an adjacent chamber (e.g., between chamber 510a1 and chamber 510a2 and between chamber 510b1 and chamber 510b2). Such inter-chamber optical transmissions are reduced or eliminated by thermally-conductive laminate seals 532a, 532b having a plastic layer 533 that may be as thin as 10 μm to 20 μm directly covering the chambers 510a1, 510a2, 510b1, 510b2. In addition, a metallic foil such as aluminum foil is impermeable to water, thereby preventing vapor transmissions to or from the chambers 510a1, 510a2, 510b1, 510b2 to enhance the stability of dry (dehydrated or lyophilized) reagents stored in the chambers.

Reagent(s) required for performing specified reactions within the reaction chambers 510a1, 510a2, 510b1, 510b2 may be pre-applied in a wet form and then dried to a surface of the laminate seal 532a, 532b facing the interior of the chambers, i.e., on an outer surface of the plastic layer 533 of the laminate seal 532a, 532b, as described in International Application No. PCT/US2025/026844, entitled “Fluidic Cartridge and Apparatuses for Processing Fluidic Cartridges,” filed Apr. 29, 2025.

The laminate seals 532a, 532b are separate from the bottom film 530—i.e., the laminate seals 532a, 532b are structurally and functionally isolated from the bottom film 530. Accordingly, different formulations and configurations of the bottom film 530 can be adopted, depending on specific operational, functional, and/or structural requirements for the bottom film, such as defining channels, without requiring a change in the laminate seals. In other examples, the bottom film covers a portion of a face of the cartridge that is spatially separated, or isolated, from the one or more reaction/detection chambers covered by one or more laminate seals, in which case cutouts formed in the bottom film are not necessary.

Functional chambers W1 to W12 and SB of the cartridge body 502 contain or are configured to receive during the use of the fluidic cartridge 500 at least one of a fluid sample, different reagent products, and a purification column, as well as fluids or solids intended for the preparation, amplification, and analysis of the sample. Other wells may serve as mixing chambers to temporarily hold two or more different materials combined therein or may serve as waste chambers. Examples of the contents contained within and/or the functions of wells W1 to W12 and CW are set forth in Table 1 below:

TABLE 1

| Chamber | Content/Function |
|---|---|
| W1 | Sample Chamber |
| W2 | Wash Buffer |
| W3 | Wash Buffer |
| W4 | Purification Column |
| W5 | PCR Mix 1 |
| W6 | Metering |
| W7 | PCR Mix 2 |
| W8 | Hybridization Buffer |
| W9 | Binding Buffer |
| W10 | Elution Buffer |
| W11 | Waste 1 |
| W12 | Waste 2 |
| SB | Syringe Barrel |

As explained above, chambers W1 to W5 and W7 to W10 include through-holes H1 to H5 and H7 to H10, respectively, formed through a bottom wall of the respective chamber, and functional chamber W6 includes three through-holes H6, H11, H12 formed through a bottom wall of the chamber. Through hole H1 forms a sample exit port from the sample chamber W1. Syringe barrel SB includes central through-holes H1c, H2c, H3c, H4c, H5c, H6c, H7c, H8c, H9c, and H10c formed through a bottom wall of the barrel. Each of chambers W1-W10 is independently in fluidic communication with the central chamber SB via channels formed by grooves G1, G2, G3, G4, G5, G6, G7, G8, G9, and G10, respectively, controlled by the valves V1, V2, V3, V4, V5, V6, V7, V8, V9, and V10, respectively, and fluids can flow, in one direction or the other between these different functional chambers (i.e., from each one of the chambers W1 to W10 to the syringe barrel SB or vice versa).

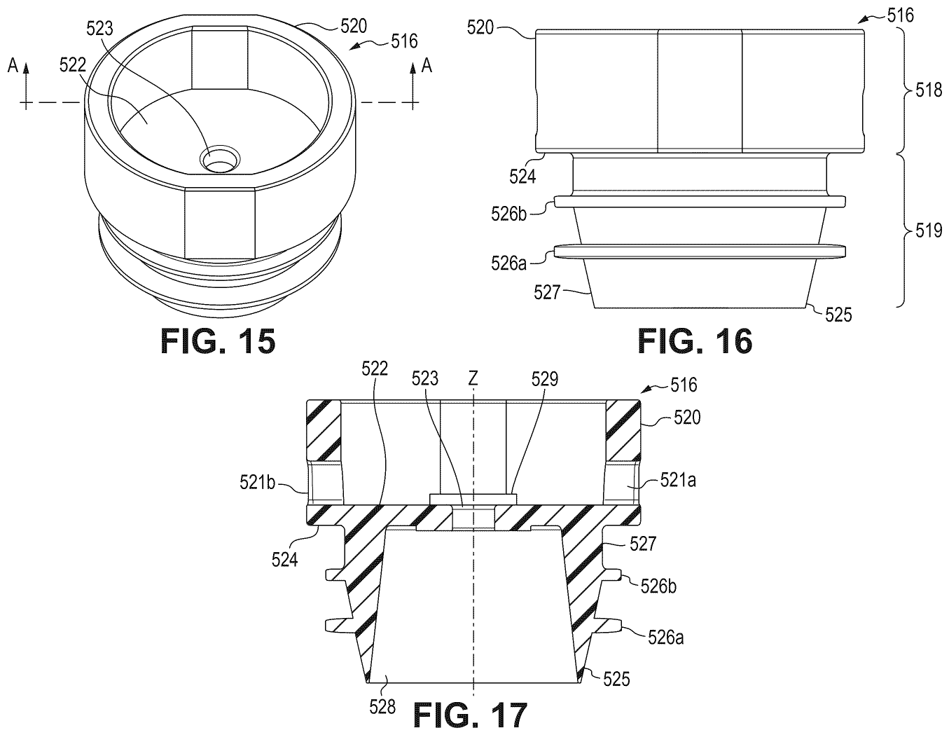

Details of an example of cap 516 are shown in FIGS. 15-17, and FIG. 18 is a partial cross-section of the cartridge body 502 showing cap 516 inserted into sample chamber W1. Desirable properties of cap 516 include that it effectively seal sample chamber W1, that it be easily pressed into the sample chamber by a user and not fall out, that it be vented to prevent pressurizing during insertion, and that it include material covering the vent hole(s) to prevent liquid escape and have sufficiently small openings (pores) (e.g., 0.2 μm) to prevent viral particles or aerosols from escaping the vent hole(s). Cap 516 may be rotationally symmetric about an axis Z (see FIG. 17) and includes an upper portion 518 having a laterally extending member, which, in the illustrated example, is in the form of a radial wall 522 oriented radially with respect to axis Z with a peripheral wall 520 surrounding the radial wall 522 and extending in an axial direction with respect to axis Z. Peripheral wall 520 may include a flattened grip portion 517 or may otherwise be adapted for improved manual gripping.

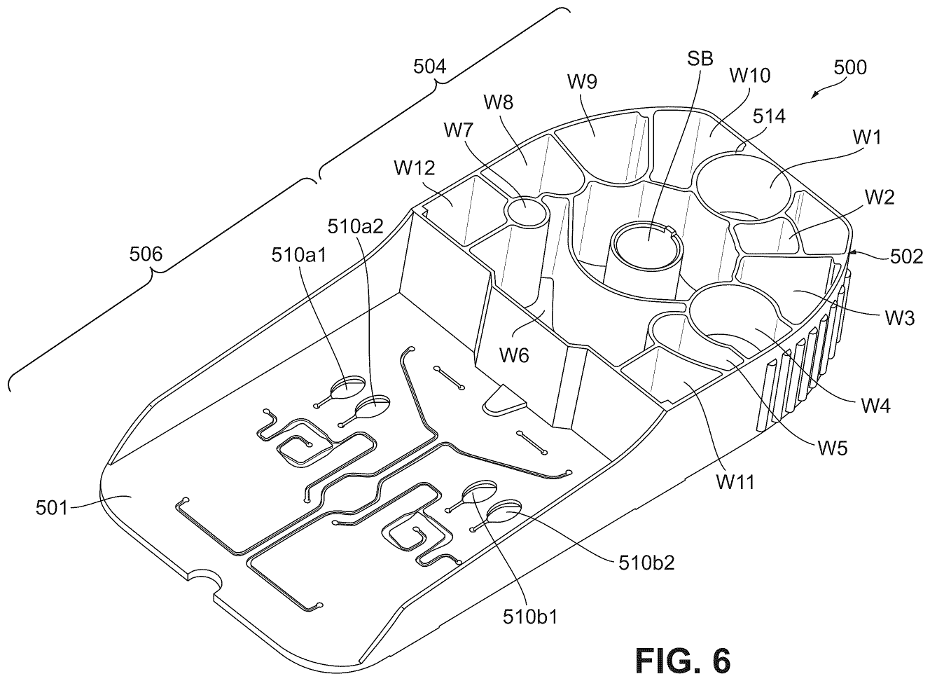

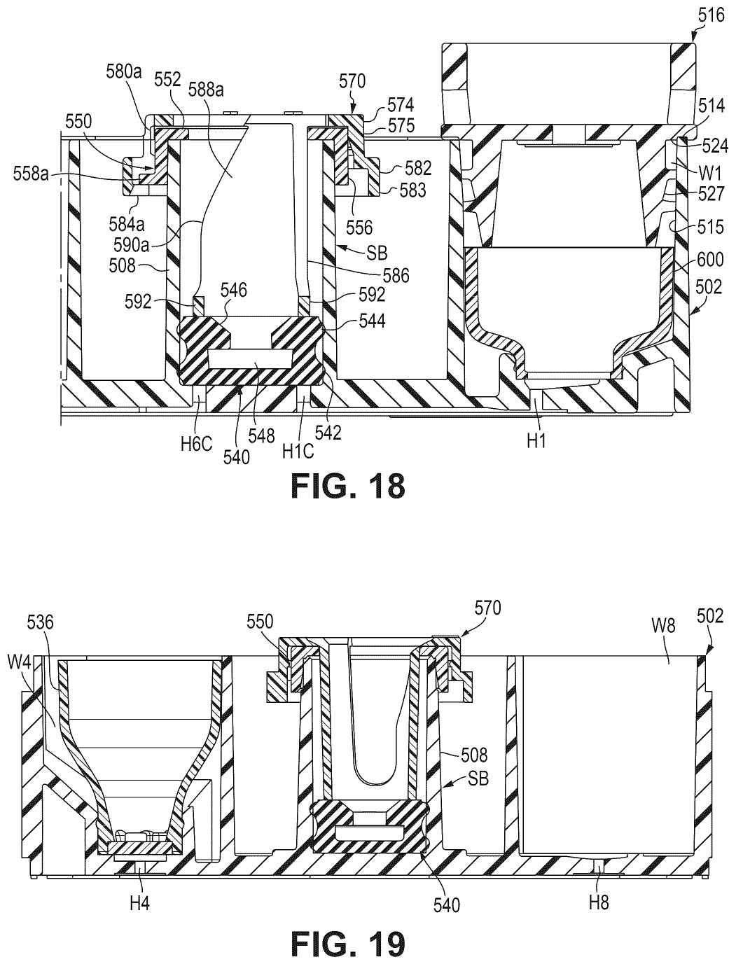

Cap 516 also includes a lower portion 519 defined by a sleeve, or peripheral wall, 525 depending from (e.g., extending below) the radial wall 522 and extending in an axial direction with respect to axis Z. An inner surface of the sleeve, or peripheral wall, 525 and a bottom surface of the laterally extending member, or radial wall, 522 define a recess, or cavity, 528 extending upward from the bottom end of the cap 516. The upper portion 518 of the cap 516 is wider than the lower portion 519, thereby defining a radial, annular shoulder 524 at a peripheral region of a lower surface of the radial wall 522. Peripheral wall 525 is inserted into the sample chamber W1, for which purpose the wall 525 may be tapered, and the radial shoulder 524 contacts a top edge surface 514 (see FIGS. 6 and 18) of the wall of the sample chamber W1. An outer surface 527 of the peripheral wall 525 is in sealing engagement with an inner surface 515 of the sample chamber W1 (see FIG. 18), and lower portion 519 may also include radially-extending annular ribs 526a, 526b projecting from the outer surface 527 of the peripheral wall 525 to contact inner surface 515 of the sample chamber W1 (see FIGS. 16-18) to enhance sealing between the peripheral wall 525 of cap 516 and the sample chamber W1. The outer surface 527 of the peripheral wall 525 may have a frictional fit with inner surface 515 of the wall of the sample chamber W1, or the peripheral wall 525 may be coupled to the wall of the sample chamber W1 by mated threads (not shown) on the peripheral wall 525 and the wall of the sample chamber W1.

A vent hole 523 is formed in the radial wall 522, and side vent holes 521a, 521b are formed in the peripheral wall 520. Vent hole 523 may have a width (e.g., diameter) of about 2 mm and is preferably covered by a porous vent membrane 529 (shown in FIG. 17 only) affixed to a top surface of radial wall 522 or a bottom surface of radial wall 522. Suitable material for membrane 529 includes Traketch® Pet/Pet 0.2 Vent R300 part no. 063390, SABEU GmbH & Co. KG, of Northeim, Germany, which includes membrane material PET 23 μm thick, with a backing of non-woven PET 60 g/m², a pore size of 0.2±0.4 μm, a pore density of 320±50×10⁶/cm², and an overall thickness of 140±50 μm.

Cap 516 may be formed (e.g., injection molded) from a thermoplastic elastomer, such as TPE Thermolast® M TM6MHD, KRAIBURG TPE GmbH & Co. KG, of Waldkraiburg, Germany.

Fluidic cartridge 500 may comprise two functional sections. As shown in FIGS. 6 and 7, sample preparation section 504 of the fluidic cartridge 500 includes a number of chambers (e.g., chambers W1 to W12) that contain, or may receive during operations on the cartridge by instrument 10, various materials (which may include liquids or other fluids) used in preparing a sample for the performance of an assay or other procedure on the sample within the cartridge. Sample preparation section 504 is configured to receive a sample specimen in a sample chamber (e.g., chamber W1) (which may comprise or be connected to a fluid inlet port at which fluid sample is introduced to the sample chamber) and to process the sample using materials contained in one or more other chambers within the sample preparation section 504, for example, to isolate target molecules (e.g., lysis and purification of nucleic acids using silica based purification) from other components of the sample and to combine the isolated molecules with materials used in the performance of an assay, such as amplification reagents and/or detection probes, to form a reaction mixture. Amplification reagents and/or detection probes may be provided in one or more of the chambers W2 to W10 of the sample preparation section 504 in a dry (e.g., lyophilized or spotted) form and reconstitution fluids for combining with and reconstituting the reagents and/or probes may be contained within one or more of chambers W2 to W10 of the sample preparation section 504. Valves V1-V10, controlling fluid flow to and from chambers W1-W10, respectively, and valves V11 and V12 controlling fluid flow to and from chamber W6, may be referred to as sample preparation (or process) valves, as they are located within and control fluid flow for chambers W1-W10 within the sample preparation section 504 of fluidic cartridge 500.

Referring to FIGS. 6 and 7, reaction/detection section 506 of the fluidic cartridge 500 is configured to receive the processed sample (reaction mixture) from the sample preparation section 504 and to provide a platform at which one or more reactions take place, for example to amplify and detect target molecules (e.g., real-time PCR). Reaction/detection section 506 includes one or more reaction chamber(s) (e.g., reaction/detection chambers 510a1, 510a2, 510b1, 510b2), each of which defines an enclosure capable of containing a fluid substance and within which reactions may take place and from which detectable signals emitted during a reaction may be detected. The detectable signal may be an optical signal, such as fluorescence, and detection of the detectable signal may indicate the presence and/or amount of target molecules in a sample. Valves V13-V18, controlling fluid flow to and from reaction chambers 510a1, 510a2, 510b1, 510b2, may be referred to as reaction valves, as they are located within and control fluid flow for reaction chambers 510a1, 510a2, 510b1, 510b2 within the reaction/detection section 506 of cartridge 500.

### Onboard Mechanical Lysis

Fluidic cartridge 500 may be configured to facilitate the performance of onboard mechanical lysis to break open cells (e.g., pathogenic microorganisms including bacteria, viruses, parasites, etc.) contained in the sample material dispensed into the sample chamber W1 to release nucleic acids (DNA or RNA) from the cells of the sample material for downstream molecular assays. In such an embodiment, a lysis chamber is provided in sample chamber W1 (see FIG. 3) which contains components (including lytic agents) with which mechanical lysis of sample material may be performed. The lysis chamber may comprise a variety of form factors, examples of which are described herein.

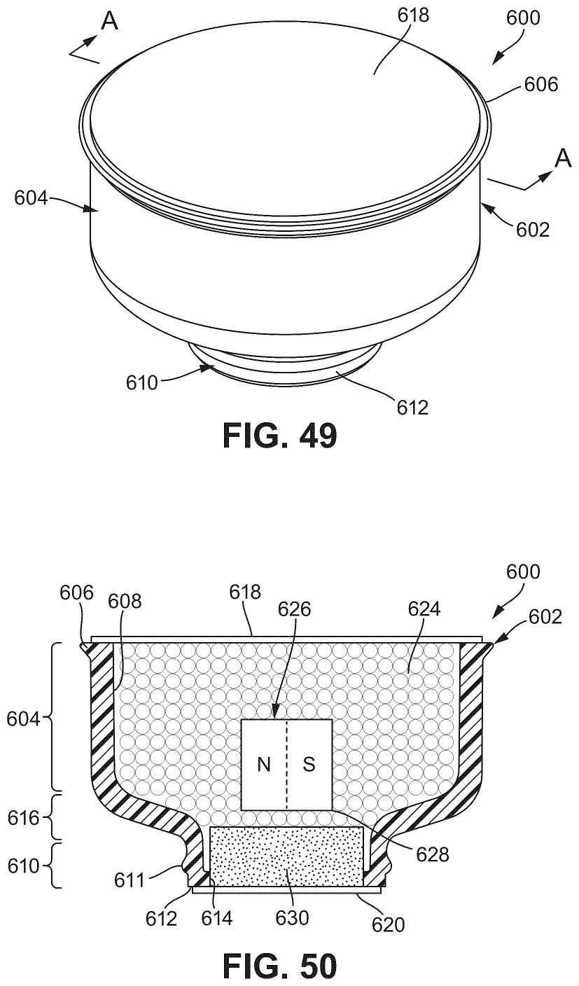

FIG. 49 is a perspective view of a first embodiment of a lysis capsule 600, and FIG. 50 is a cross-section of the lysis capsule 600 along the line A-A in FIG. 49. Capsule 600 is placed in the sample chamber W1 of a fluidic cartridge 500, which is closed by cap 516. Capsule 600 may be press-fitted into the sample chamber W1, or capsule 600 may be threadedly mated with an inner surface of the sample chamber W1.

Referring to FIGS. 49 and 50, capsule 600 comprises a hollow body 602 including, in the example shown, a first (e.g., upper) portion 604, which may be cylindrical or generally cylindrical, a second (e.g., lower) portion 610, which may be cylindrical or generally cylindrical, which is centered—or coaxial—with respect to the first portion 604, and which has a smaller width (diameter) than the first portion 604, and a tapered transition portion 616 between the first portion 604 and the narrower second portion 610. In another example, a transition between first portion 604 and narrower second portion 610 is not tapered. Section 610 may include a raised sealing rib 611 surrounding section 610 for providing a sealing interface between the capsule 600 and the sample chamber W1. The hollow body 602 may comprise an integral component molded from a plastic material (e.g., injection molded), such as, polypropylene, polyethylene, acrylonitrile butadiene styrene (“ABS”), or polyethylene terephthalate (“PET”).

A first rim 606 surrounds an open first end 608 at one end of the hollow body 602 (the top end in the illustrated embodiment), and a second rim 612 surrounds an open second end 614 at an opposite end of the hollow body 602 (the bottom end in the illustrated embodiment).

A first porous membrane, or barrier, 618 is affixed to the first rim 606, for example, by an adhesive, by heat sealing, or by ultrasonic welding. A second porous membrane 620 is affixed to the second rim 612, for example, by an adhesive, by heat sealing, or by ultrasonic welding. The first membrane 618 covering the open first end 608 and the second membrane, or barrier, 620 covering the open second end 614 define a lysis chamber 622 within the hollow body 602 between the membranes 618 and 620 and containing lytic elements, such as beads, described below. The first and second membranes may be filters, with the first membrane 618 filtering out larger sample components (e.g., undigested food particles and mucus found in stool or other gastrointestinal samples), thereby inhibiting such larger components from entering the lysis chamber 622, and the second membrane 620 filtering out cellular debris following lysis, thereby inhibiting such cellular debris from exiting the lysis chamber 622, while allowing the target of interest (e.g., DNA and RNA) to pass out of the lysis chamber.

First porous membrane 618 may be a mesh and is preferably hydrophilic (either naturally hydrophilic or treated so as to be hydrophilic) to facilitate passage of fluid sample material through the first membrane 618. Suitable materials include a polyamide, polypropylene, polyethylene terephthalate (PETP), ethylene tetrafluoroethylene (ETFE), or polyether ether ketone (PEEK). The porosity (pore size) of the first porous membrane 618 may, for example, be 70 μm to 500 μm, e.g., about 300 μm, the maximum size being limited by the size of lytic beads to be retained within lysis chamber 622. A suitable mesh for the first porous membrane 618 is available from Sefar, Inc. Buffalo, NY part no. 03-300/51 HPL having a pore size of 300 μm.

Vent membrane 529 covering the vent hole 523 of cap 516 (see FIG. 17) is preferably finer than the first porous membrane 618 to allow air to be vented from the sample chamber W1 through the vent hole 523 without permitting liquid passage.

Second porous membrane 620 may be a mesh, or a filter matrix (e.g., a sintered or spun filter), and is preferably hydrophilic (either naturally hydrophilic or treated so as to be hydrophilic). Suitable materials include a polyamide, polypropylene, polyethylene terephthalate (PETP), ethylene tetrafluoroethylene (ETFE), or polyether ether ketone (PEEK). The porosity (pore size) of the second porous membrane 620 is preferably smaller than that of the first porous membrane 618, as the first porous membrane is intended to permit sample fluid to pass through into the lysis chamber 622, and the second porous membrane 620 is intended to capture post-lysis cellular material. The porosity (pore size) of the second porous membrane 620 may, for example, be 30 μm to 100 μm, e.g., about 70 μm. The pore size of the second porous membrane 620 should be small enough to capture post-lysis cellular material but not too small so as to be vulnerable to clogging. A suitable mesh for the second porous membrane 620 is available from Sefar, Inc. Buffalo, NY, part no. 03-70/33 HPL having a pore size of 70 μm.

Protective mesh and supports (not shown) may be added on either side of the first porous membrane 618 and/or second porous membrane 620 to help maintain the membrane's integrity during lysis. As membrane materials (e.g., polyethersulfone (PES)) may be fragile, they are susceptible to rupture during lysing by the beads and the magnet. Supportive mesh (such as woven nylon or polyester mesh) or structure (such as injection molded or 3D printed mesh) that is mechanically strong can be layered on the top and/or bottom of the membrane for support and protection. The supportive mesh may have a porosity of up to about 300-350 μm, the maximum size being limited by the size of the lytic beads within the lysis chamber 622.

Optionally, a filter element 630, e.g., a sintered filter, may be provided within the lysis chamber 622 with or without second porous membrane 620, for example, at a position covering the open second end 614, as shown in FIG. 50. In some applications, such a filter element will trap additional post lysis cellular debris that is not trapped by the second porous membrane 620. The porosity (pore size) of the filter element 630 may be the same as second porous membrane 620 (e.g., a range of 30 μm to 100 μm or about 70 μm). The porosity of the filter element 630 may vary through its thickness, e.g., having a pore size that progressively decreases from a larger pore size (e.g., 100 μm) at a top surface of the filter element 630 to a smaller pore size (e.g., 30 μm) at a bottom surface of the filter element 630. In one non-limiting example, if a filter element 630 is included within the lysis chamber 622, the pore size of the second porous membrane 620 may be much larger than stated above, e.g., as large as 300 μm, since the additional filter element may be employed to capture post-lysis cellular material. In another example, the second porous membrane 620 may be omitted, in which case the filter element 630 may be retained within the open second end 614 by an interference, frictional fit, or by a ledge or shelf extending inwardly from the open second end 614 below the filter element 630. In another example, a filter element may be located within the sample chamber W1 outside the lysis chamber 622 between the second membrane 620 and through hole H1. In another embodiment, a filter element is omitted because, while the filter element may be effective at trapping cellular debris, it may also trap lysis-released nucleic acids that are intended to pass out of the sample chamber.

Lysis chamber 622 contains a plurality of non-magnetic beads 624 filling a portion of the volume of the lysis chamber 622 and a magnetic element 626. The non-magnetic beads 624 and the magnetic element 626 may be collectively referred to as “lysis beads” or “lytic agents.” Fluid sample is provided to the lysis chamber 622, and the magnetic element 626 is agitated, as described below, to impart motion to the non-magnetic beads 624 to effect mechanical lysis of cells present the sample contained within the lysis chamber 622 (known as bead beating). The pore sizes of the first porous member 618 and the second porous member 620 are sufficiently small to retain the non-magnetic beads 624 and the magnetic element 626 within the lysis chamber 622. In one non-limiting example, the volume of the lysis chamber 622 is about 600 μl, the volume of the non-magnetic beads 624 is about 300 μl (i.e., about 50% of lysis chamber volume), and the volume of the magnetic element 626 is about 27-64 μl (i.e., about 4.5% to about 11% of lysis chamber volume), leaving space for about 236-273 μl (39-45% of a 600 μl lysis chamber) of fluid sample material in the lysis chamber 622. In an example, the non-magnetic beads 624 occupy a volume of the lysis chamber 622 of 50% to 75% of an available volume of the lysis chamber 622 (i.e., the total volume of the lysis chamber 622 less the volume occupied by the magnetic element 626). Factors that influence the amount of non-magnetic beads 624 to provide relative to the total volume of the lysis chamber 622 include (1) providing sufficient beads to efficiently and effectively grind (lyse) sample molecules within the lysis chamber 622 and (2) not providing too much non-magnetic beads 624 such that movement of the magnetic element 626 is unduly restricted. In another implementation, an open volume is available within the sample chamber above the capsule 600—i.e., above the first membrane 618—and the volume of fluid sample material added to the sample chamber W1 exceeds the volume available within the lysis chamber 622.

Suitable non-magnetic beads 624 include beads made from ceramic, glass, silica, or zirconium and may be spherical or approximately spherical in shape with a size (e.g., diameter) ranging from 100 μm to 2000 μm, e.g., about 500 μm (0.5 mm), depending on the intended application (i.e., the intended lysing target). In one non-limiting example, the non-magnetic beads are inert with respect to the sample material (i.e., the beads will not react with the cellular material or bind with released nucleic acids). Suitable beads include those available from Final Advanced Materials SARL, of Didenheim, France, Item No. 055-0120. ZrO2 beads, Cerium stabilized. ZrO2: 83%-CeO2: 17%, Ø 0.40-0.70:3.75+/−0.05 kg/L.

Magnetic element 626 is a permanent magnet made from a magnetic material, such as, N52 or N42 grade neodymium (NdFeB) and is preferably axially magnetized (i.e., north and south (“N” and “S”) poles are located at two points 180° from each other). As will be described below, the magnetic element 626 will be exposed to a varying magnetic field, thereby causing the magnetic element 626 to move within the lysis chamber 622, imparting motion to the non-magnetic beads 624 to mechanically lyse or disrupt cells contained within a sample provided to the lysis chamber 622, thereby releasing their internal components (e.g., DNA, RNA, proteins and organelles). Magnetic element 626 may be any shape, including a cube, sphere, rod, disc, etc. A magnetic element 626 with edges 628 (e.g., cube or other parallelepiped) exhibits better performance as being more effective to impart the desired motion to the non-magnetic beads 624. Edges 628 may be rounded. In one non-limiting example, magnetic element 626 is cubic in shape with side faces having a width of about 2-3 mm (e.g., up to about ⅛ inch). One factor to be considered in sizing the magnetic element 626 is that, while a larger, stronger magnet may be preferable in some applications, the size of the magnet will be limited by the available volume within the lysis chamber 622 to permit adequate movement of magnetic element 626.

Magnetic element 626 may be coated or encapsulated, e.g., over-molded, with a non-magnetic material that is non-reactive with the sample solution to prevent reaction between the magnetic element 626 and the sample solution and/or to protect the magnetic element 626 from abrasion from the non-magnetic beads 624. Coating materials may include Teflon® (polytetrafluoroethylene), polypropylene, epoxy, urethane, nickel, or gold. Coating thickness may increase the width of the faces of magnetic element 626—e.g., up to a total thickness of about 4.3 mm.

In one non-limiting example, the magnetic element 626, and/or its coating, is inert with respect to the sample material (i.e., the element will not react with the cellular material or bind with released nucleic acids).

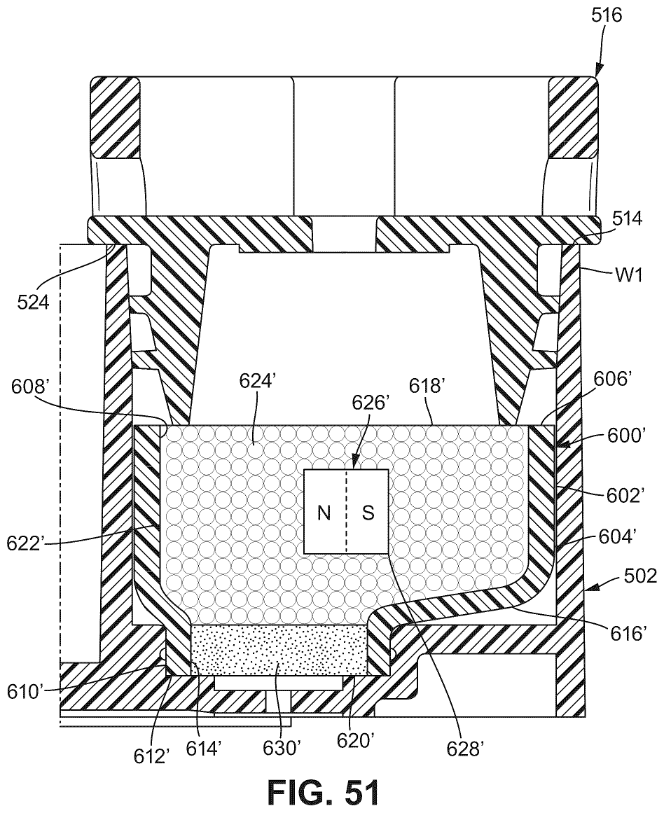

FIG. 51 is a transverse cross-section of the sample chamber W1 of the cartridge body 502 with a cap 516 and an alternate embodiment of a capsule 600′ positioned within the sample chamber W1 beneath the cap 516. Capsule 600′ may be press-fitted into the sample chamber W1, or capsule 600′ may be threadedly mated with an inner surface of the sample chamber W1.

Capsule 600′ has a hollow body 602′ with a first rim 606′ surrounding an open first end 608′ of the hollow body 602′ (top end in the illustrated embodiment) and a second rim 612′ surrounding an open second end 614′ (bottom end in the illustrated embodiment) of the hollow body 602′. A first porous membrane 618′, which may be identical to first porous membrane 618 described above, is affixed to the first rim 606′, for example, by an adhesive, heat sealing, or ultrasonic welding. A second porous membrane 620′, which may be identical to second porous membrane 620 described above, is affixed to the second rim 612′, for example, by an adhesive, by heat sealing, or by ultrasonic welding.

The first membrane 618′ and the second membrane 620′ defines a lysis chamber 622′ within the hollow body 602′ between the membranes 618′ and 620′. Protective mesh and supports (not shown) may be added on either side of the first porous membrane 618′ and/or second porous membrane 620′ to help maintain the membrane's integrity during lysis.

Like hollow body 602 of capsule 600 described above, hollow body 602′ includes a first portion 604′, which may be cylindrical or generally cylindrical, a second portion 610′, which may be cylindrical or generally cylindrical and which has a smaller width (diameter) than the first portion 604′, and a transition portion 616′ between the first portion 604′ and the narrower second portion 610′ and which may be tapered, as shown, or not tapered. Section 610′ may include a raised sealing rib surrounding section 610′ for providing a sealing interface between the capsule 600′ and the sample chamber W1 (not labeled in FIG. 51, see sealing rib 611 in FIG. 50). Hollow body 602′ differs from hollow body 602 described above in that the second portion 610′ is not centered—or coaxial—with respect to the first portion 604′. The hollow body 602′ may comprise an integral component molded from a plastic material (e.g., injection molded), such as, polypropylene, polyethylene, acrylonitrile butadiene styrene (“ABS”), or polyethylene terephthalate (“PET”).

Lysis chamber 622′ of capsule 600′ contains a plurality of non-magnetic beads 624′, which may be identical to non-magnetic beads 624 described above, filling a portion of the volume of the lysis chamber 622′ and a magnetic element 626′, which may be identical to magnetic element 626 described above and which may include edges 628′. The non-magnetic beads 624′ and the magnetic element 626′ may be collectively referred to as “lysis beads” or “lytic agents.” Fluid sample is provided to the lysis chamber 622′, and the magnetic element 626′ is agitated, as described below, to impart motion to the non-magnetic beads 624′ to effect mechanical lysis of cells present in the sample contained within the lysis chamber 622′. The relative volumes of the lysis chamber 622′, non-magnetic beads 624′, and magnetic element 626′ may be as described above for the lysis chamber 622, non-magnetic beads 624, and magnetic element 626.

Lysis capsule 600′ may include an optional filter element 630′, which may be identical to filter element 630 described above, within the lysis chamber 622′. Alternatively, filter element 630′ may be disposed within the sample chamber W1 outside the hollow body 602′, or filter element 630′ may be omitted.

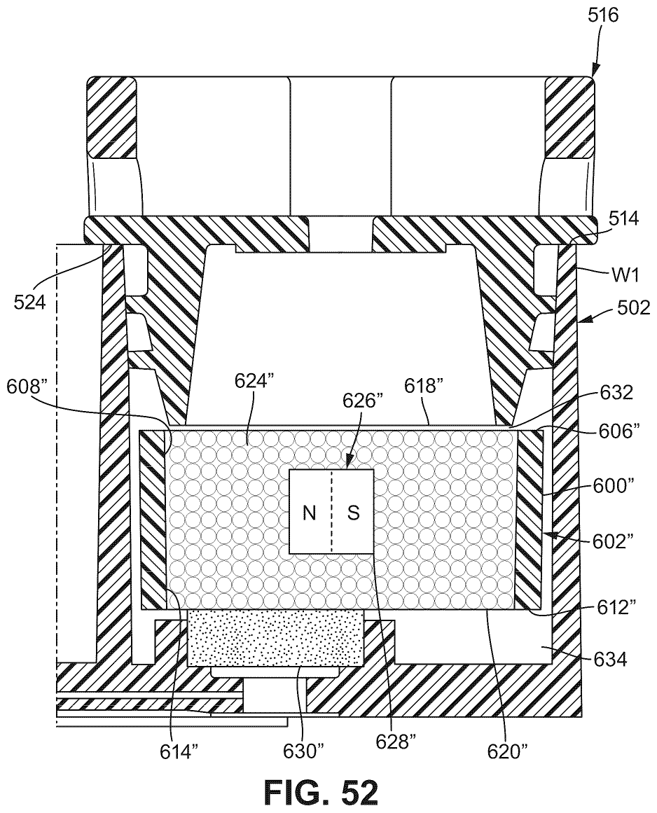

FIG. 52 is a transverse cross-section of the sample chamber W1 of the cartridge body 502 with a cap 516 and an alternate embodiment of a capsule 600″ positioned within the sample chamber W1 beneath the cap 516. A gap 632 may be provided between a bottom end of cap 516 and a top end of capsule 600″. A similar gap may be provided between cap 516 and capsule 600 described herein and/or between cap 516 and capsule 600′ described herein. Capsule 600″ may be press-fitted into the sample chamber W1, or capsule 600″ may be threadedly mated with an inner surface of the sample chamber W1.

Capsule 600″ has a hollow body 602″ with a first rim 606″ surrounding an open first end 608″ (top end of the illustrated embodiment) of the hollow body 602″ and a second rim 612″ surrounding an open second end 614″ (bottom end of the illustrated embodiment) of the hollow body 602″. The hollow body 602″ may comprise an integral component molded from a plastic material (e.g., injection molded), such as, polypropylene, polyethylene, acrylonitrile butadiene styrene (“ABS”), or polyethylene terephthalate (“PET”). Hollow body 602″ comprises a sleeve, which may be cylindrical or generally cylindrical, and which has a constant width (e.g., diameter) between the first rim 606″ and the second rim 612″. A first porous membrane 618″, which may be identical to first porous membrane 618 described above, is affixed to the first rim 606″, for example, by an adhesive, heat sealing, or ultrasonic welding. A second porous membrane 620″, which may be identical to second porous membrane 620 described above, is affixed to the second rim 612″, for example, by an adhesive, heat sealing, or ultrasonic welding.

The first membrane 618″ and the second membrane 620″ define a lysis chamber 622″ within the hollow body 602″ between the membranes 618″ and 620″. Protective mesh and supports (not shown) may be added on either side of the first porous membrane 618″ and/or second porous membrane 620″ to help maintain the membrane's integrity during lysis.

Lysis chamber 622″ of capsule 600″ contains a plurality of non-magnetic beads 624″, which may be identical to non-magnetic beads 624 described above, filling a portion of the volume of the lysis chamber 622″ and a magnetic element 626″, which may be identical to magnetic element 626 described above and which may include edges 628″. The non-magnetic beads 624″ and the magnetic element 626″ may be collectively referred to as “lysis beads” or “lytic agents.” Fluid sample is provided to the lysis chamber 622″, and the magnetic element 626″ is agitated, as described below, to impart motion to the non-magnetic beads 624″ to effect mechanical lysis of cells present in the sample contained within the lysis chamber 622″. The relative volumes of the lysis chamber 622″, non-magnetic beads 624″, and magnetic element 626″ may be as described above for the lysis chamber 622, non-magnetic beads 624, and magnetic element 626.

An optional filter element 630″, which may be identical to filter element 630 described above, may be provided within the sample chamber W1 beneath the lysis capsule 600″. Alternatively, filter element 630″ may be omitted.

A dead space 634 may be provided within sample chamber W1 between a bottom wall of the sample chamber W1 and the lysis capsule 600″ for collecting post lysis cellular material that is able to pass through second membrane 620″ but not through additional filter element 630″.

Lysis capsules 600, 600′, or 600″ may include a multi-stage filtration system. A pre-filter (not shown), is integrated into the lysis capsule 600, 600′, or 600″ to replace or complement first porous membrane 618, 618′, or 618″ (e.g., where the first porous membrane comprises a mesh) and is configured to remove large gastrointestinal sample, e.g., stool, particles, such as undigested food and mucus (generally larger than 50 μm). The pre-filter may have a pore size that is large enough to allow target cells of interest to pass through. For example, if the target of interest is a parasite that is about 40 μm, the pore size of the pre-filter should be larger than 40 μm to allow the target to enter the lysis capsule. The pre-filter is preferably hydrophilic and can be a woven mesh filter, such as nylon or polyester or similar, or a membrane filter. The filter may be supported and protected by a protective layer (e.g., by a woven nylon or polyester mesh as described above) to prevent it from being damaged during mechanical lysis.

In another example, a pre-filter (not shown) may be integrated into a sample cap assembly, for example, across the open bottom end of the sleeve, or peripheral wall, 525 of lower portion 519 of cap 516 (see FIGS. 16, 17).

The pre-filter of the multi-stage filtration system may be followed by one or more enhanced post-filters (not shown) to complement or replace second porous membrane 620, 620′, or 620″ (e.g., where the second porous membrane comprises a mesh) (below the lysis chamber 622, 622′, or 622″). The post-filter(s) capture finer particles in the micrometer-size range and allow the target of interest (DNA and RNA molecules) to pass through for downstream processes. During lysis, microorganisms and other components in the sample are ground mechanically into smaller particles (sub-micrometer). The purpose of the finer post-filter(s) is to selectively remove smaller particles without impacting the transfer of target molecules and sample volume from the lysis chamber 622, 622′, or 622″. Where there are more than one post-filters, the filters may have progressively smaller porosities, e.g., a first post-filter nylon mesh with a porosity of about 30 μm, and a second post-filter nylon mesh with a porosity of about 6 μm.

The filter pore size (from 0.22 μm to 500 μm) and the type of filter can be selected based on the sample type and filtration requirements.

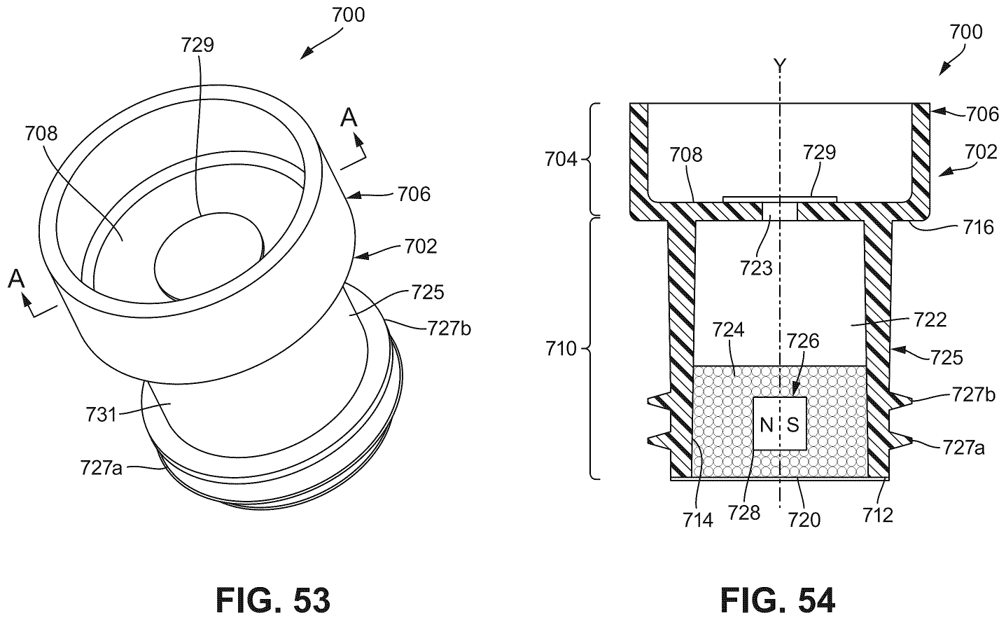

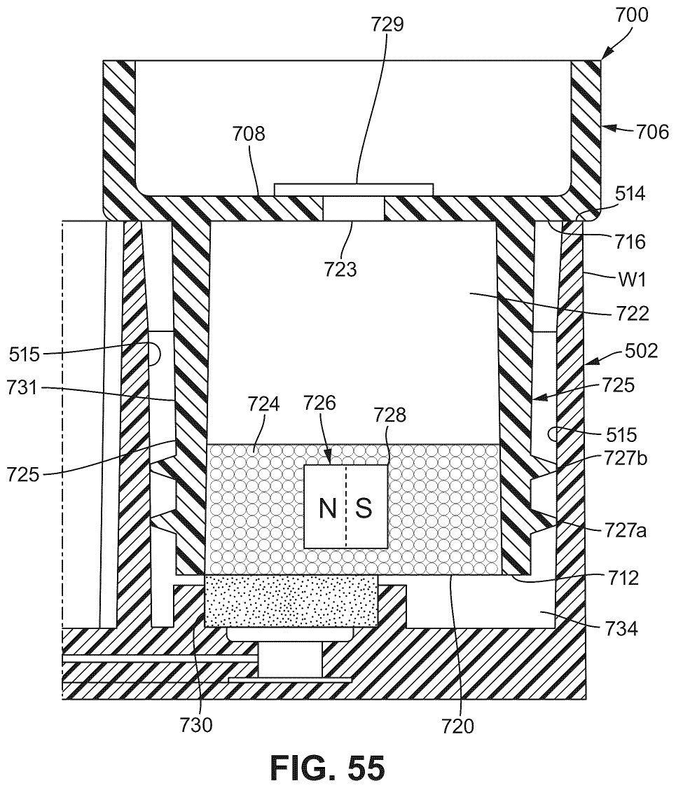

FIG. 53 is top perspective view of an alternate lysis vessel 700 comprising an integrated cap for closing the sample chamber W1 and a lysis capsule for containing the materials for performing lysis. FIG. 54 is a transverse cross-section of the lysis vessel 700 along the line A-A of FIG. 53, and FIG. 55 is a transverse cross-section of the sample chamber W1 of the cartridge body 502 with the lysis vessel 700 positioned within the sample chamber W1.

Details of the lysis vessel 700 are identified in FIGS. 53-55. As with the cap 516 described above, desirable properties of lysis vessel 700 combining a cap for closing the sample chamber W1 and a lysis capsule include that it effectively seal the sample chamber W1, that it be easily pressed into the sample chamber by a user and not inadvertently fall out, that it be vented to prevent pressurizing during insertion, and that it include material covering the vent hole(s) to prevent liquid escape and have sufficiently small openings (pores) to prevent viral particles or aerosols from escaping the vent hole(s).

Lysis vessel 700 includes a hollow body 702 which may be rotationally symmetric about an axis Y and include, in the example shown, a first (e.g., upper) portion 704, which may be cylindrical or generally cylindrical and a second (e.g., lower) portion 710, which may be cylindrical or generally cylindrical, which is centered—or coaxial—with respect to the first portion 704, and which has a smaller width (diameter) than the first portion 704. First portion 704 has a laterally extending member in the form of a radial wall 708 oriented radially with respect to axis Y with a peripheral wall 706 surrounding the radial wall 708 and extending in an axial direction with respect to axis Y. Lower portion 710 is defined by a sleeve 725 extending below radial wall 708, which may be cylindrical or generally cylindrical extending in an axial direction with respect to axis Y and which has a constant width (e.g., diameter) between the radial wall 708 and a bottom rim 712 surrounding a lower open end 714. The difference in width between first portion 704 and second portion 710 defines an annular shoulder 716 at a peripheral region of a lower surface of the radial wall 708. Second portion 710 may also include radially-extending annular ribs 727a, 727b projecting from the outer surface of the sleeve 725.

The hollow body 702 may comprise an integral component molded from a plastic material (e.g., injection molded), such as, polypropylene, polyethylene, acrylonitrile butadiene styrene (“ABS”), or polyethylene terephthalate (“PET”).

A vent hole 723 is formed in the radial wall 708, and side vent holes (not shown) may be formed in the peripheral wall 706. Vent hole 723 may have a width (e.g., diameter) of about 2 mm and may be covered by a membrane 729, which may be porous, such as Traketch® Pet/Pet 0.2 Vent R300, part no. 063390, SABEU GmbH & Co. KG, of Northeim, Germany, which includes membrane material PET 23 μm thick, with a backing of non-woven PET 60 g/m², a pore size of 0.2±0.4 μm, a pore density of 320±50×106/cm², and an overall thickness of 140±50 μm.

Hollow body 702 may be formed (e.g., injection molded) from a thermoplastic elastomer, such as TPE Thermolast® M TM6MHD KRAIBURG TPE GmbH & Co. KG, of Waldkraiburg, Germany.

A porous membrane 720 is affixed to the second rim 712, for example, by an adhesive, heat sealing, or ultrasonic welding. Protective mesh and support (not shown) may be added on either side of the porous membrane 720 to help maintain the membrane's integrity during lysis.

The radial wall 708 and membrane 729 and the porous membrane 720 define a lysis chamber 722 within the hollow body 702 between the radial wall 708/membrane 729 and porous membrane 720.

Lysis chamber 722 of lysis vessel 700 contains a plurality of non-magnetic beads 724, which may be identical to non-magnetic beads 624 described above, filling a portion of the volume of the lysis chamber 722 and a magnetic element 726, which may be identical to magnetic element 626 described above and which may include edges 728. The non-magnetic beads 724 and the magnetic element 726 may be collectively referred to as “lysis beads” or “lytic agents.” Fluid sample is placed within the lysis chamber 722, and the magnetic element 726 is agitated, as described below, to impart motion to the non-magnetic beads 724 to effect mechanical lysis of cells present in the sample contained within the lysis chamber 722.

In one non-limiting example, the volume of the lysis chamber 722 is about 870 μl, the volume of the non-magnetic beads 724 is about 300 μl, and the volume of the magnetic element 726 is about 27-64 μl, leaving space for about 506-543 μl of sample in the lysis chamber 622 if the sample is filled to the radial wall 708.

FIG. 55 is a cross-section through the sample chamber W1 showing the lysis vessel 700 inserted into the sample chamber W1. Sleeve 725 is inserted into the sample chamber W1, for which purpose the sleeve 725 may be tapered, and the radial shoulder 716 contacts top edge surface 514 of the wall of the sample chamber W1. An outer surface 731 of the sleeve 725 is in sealing engagement with inner surface 515 of the sample chamber W1. Ribs 727a, 727b contact inner surface 515 to enhance sealing between the sleeve 725 of vessel 700 and the sample chamber W1. The outer surface of the sleeve 725 may have a frictional fit with an inner surface of the inner wall of the sample chamber W1, or the sleeve 725 may be coupled to the inner wall of the sample chamber W1 by mated threads (not shown) on the sleeve 725 and the inner wall of the sample chamber W1.

Lysis vessel 700 is inserted into sample chamber W1 after sample is dispensed into chamber W1. Accordingly, the porosity of porous membrane 720 should be large enough to permit un-lysed sample to pass through the membrane 720 and may be larger than the porosity of second porous membrane 620 described above. Accordingly, an optional filter element 730, which may be identical to filter element 630 described above, may be provided within the sample chamber W1 beneath the lysis vessel 700 to capture post-lysis cellular material that will pass through porous membrane 720.

A dead space 734 may be provided within sample chamber W1 between a bottom wall of the sample chamber W1 and the lysis vessel 700 for collecting post-lysis cellular material.

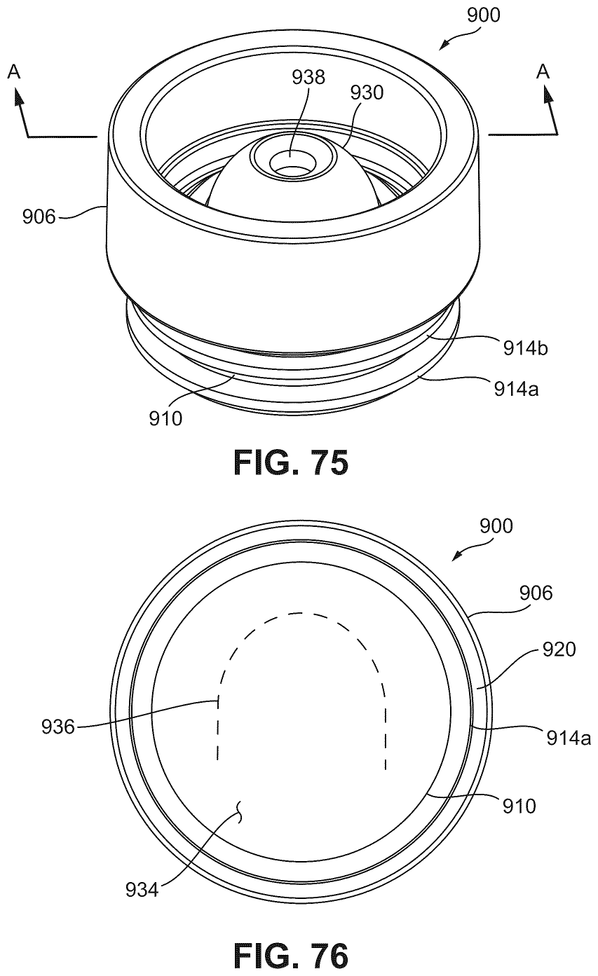

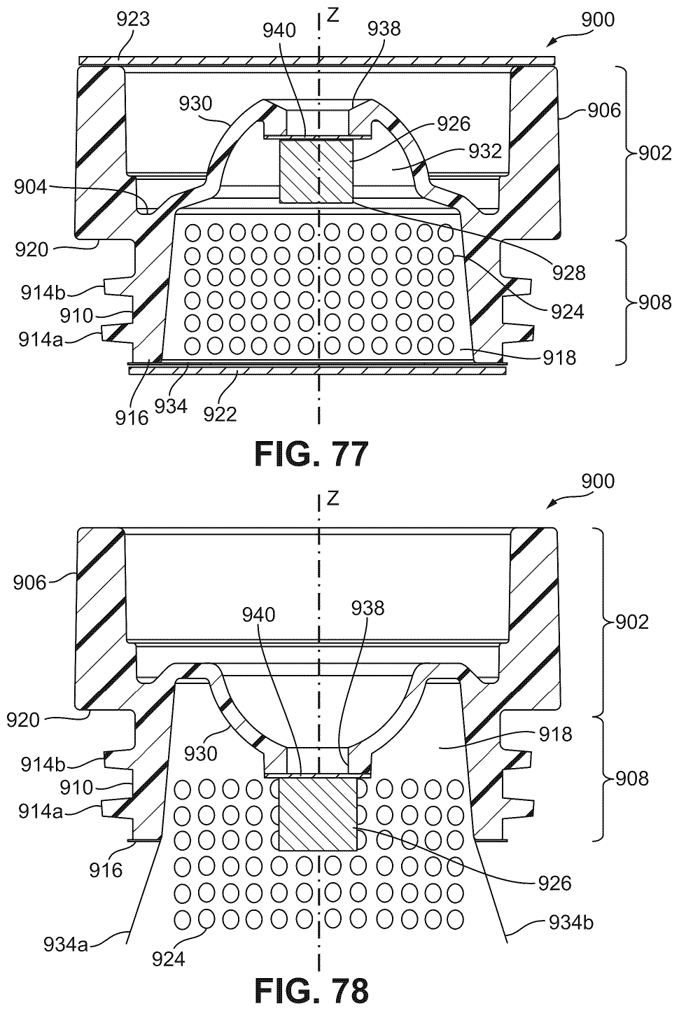

In an alternate embodiment, lysis beads are delivered to a sample contained in a sample chamber from a cap having a rupturable compartment, or chamber, for containing the beads and from which the beads can be released into the sample chamber. FIG. 75 is top perspective view and FIG. 76 is a bottom view of a bead delivery cap 900 having a rupturable chamber for containing magnetic and non-magnetic lysis beads. FIG. 77 is a cross-sectional view of the bead delivery cap 900 with a bead-containing chamber in tact, and FIG. 78 is a cross-sectional view of the bead delivery cap 900 with the bead-containing chamber ruptured to release the lysis beads.

Bead delivery cap 900 may be rotationally symmetric about an axis Z (see FIGS. 77, 78) and includes a cap body comprising an upper portion 902 and a lower portion 908. Upper portion 902 of cap body includes a laterally extending member, which, in the illustrated example, is in the form of a radial wall 904 oriented radially with respect to axis Z, and an upper peripheral wall 906 surrounding the radial wall 904 and extending in an axial direction with respect to axis Z. Lower portion 908 is defined by a lower sleeve, or lower peripheral wall, 910 depending from (e.g., extending below) the radial wall 904 and extending in an axial direction with respect to axis Z. Lower sleeve 910 surrounds a open space 918 extending upward from a bottom end 916 of the cap body. The upper portion 902 of the cap body is wider than the lower portion 908, thereby defining a radial, annular shoulder 920 at a peripheral region of a lower surface of the radial wall 904.

The cap body of bead delivery cap 900 includes a collapsible chamber defined by a deformable wall 930 that is initially outwardly convex and extending above the radial wall 904 to define an inner chamber 932 that is contiguous with (open to) the open space 918. At least one magnetic element 926 and a plurality of non-magnetic beads 924 are disposed in the open space 918 and the inner chamber 932 and are retained by a frangible membrane 934 affixed to bottom end 916 of the cap body, for example, by an adhesive, by heat sealing, or by ultrasonic welding, to enclose open space 918. The chamber 932 and open space 918 together define a lysis bead compartment that is at least partially collapsible and is enclosed by the frangible membrane 934. Non-magnetic beads 924 may be identical to non-magnetic beads 624 described above, and magnetic element 926 may be identical to magnetic element 626 described above and which may include edges 928. The non-magnetic beads 924 and the magnetic element 926 may be collectively referred to as “lysis beads” or “lytic agents.” As shown in FIG. 77, the lysis beads may be arranged within the bead delivery cap 900 with the magnetic element 926 disposed within the chamber 932 defined by the deformable wall 930 and the non-magnetic beads 924 disposed within the open space 918 defined by sleeve 910 and frangible membrane 934. In another configuration a portion of the plurality of non-magnetic beads 924 may be contained within the chamber 932 along with the magnetic element 926, or the magnetic element 926 may be contained within open space 918.

As shown in FIG. 77, deformable wall 930 may include a vent hole 938, which may be covered by a porous vent membrane 940 affixed to a portion of a lower surface deformable wall 930 surrounding vent hole 938. Vent membrane 940 may secured to a lower (inner) surface of the deformable wall 930 as shown in FIG. 77, or vent membrane 940 may secured to a upper (outer) surface of the deformable wall 930. Suitable material for membrane 940 includes Traketch® Pet/Pet 0.2 Vent R300 part no. 063390, SABEU GmbH & Co. KG, of Northeim, Germany, which includes membrane material PET 23 μm thick, with a backing of non-woven PET 60 g/m², a pore size of 0.2±0.4 μm, a pore density of 320±50×106/cm², and an overall thickness of 140±50 μm. Vent membrane 940 covering the vent hole 938 of bead delivery cap 900 preferably allows air to be vented from the sample chamber W1 through the vent hole 938 without permitting liquid passage.

The cap body of bead delivery cap 900 may be a unitary structure (i.e., a single piece) composed of a pliable polymeric material. For example, the polymeric material of bead delivery cap 900 may be formed from a thermoplastic elastomeric material.

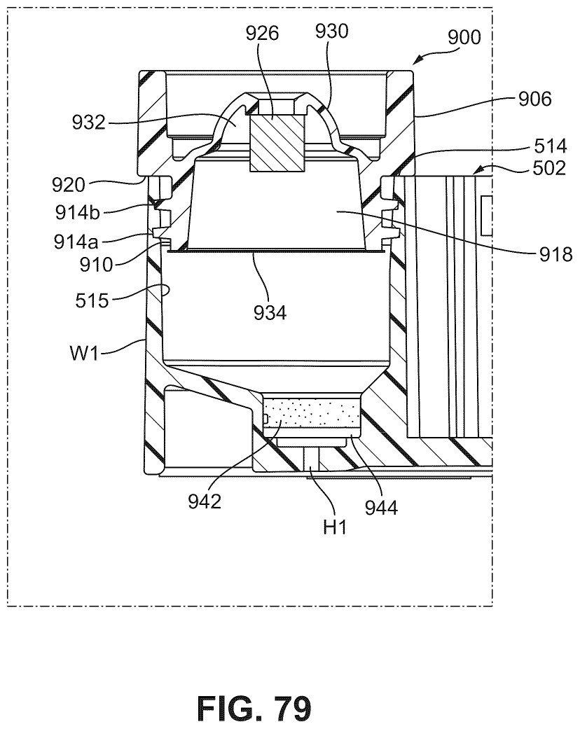

FIG. 79 is a cross-section through the sample chamber W1 of cartridge body 502 showing the bead delivery cap 900 inserted into the sample chamber W1. Bead delivery cap 900 is inserted into sample chamber W1 after sample is dispensed into chamber W1. Sleeve 910 is inserted into the sample chamber W1, for which purpose the sleeve 910 may be tapered, and the radial shoulder 920 contacts top edge surface 514 of the wall of the sample chamber W1. Sleeve 910 is in sealing engagement with inner surface 515 of the sample chamber W1 wall. Sealing ribs 914a, 914b contact inner surface 515 to enhance sealing between the sleeve 910 of bead delivery cap 900 and the sample chamber W1 wall. Sleeve 910 may have a frictional fit with inner surface 515 of the sample chamber W1 wall, or the sleeve 910 may be coupled to the sample chamber W1 wall by mated threads (not shown) on the sleeve 910 and the sample chamber W1 wall.

A filter element 942 and/or a porous membrane 944 may be provided in the bottom of the sample chamber W1 above exit port H1. Porous membrane 944 may be affixed to the bottom of the sample chamber W1, for example, by an adhesive, heat sealing, or ultrasonic welding. Porous membrane 944 may be a mesh, or a filter matrix, and is preferably hydrophilic (either naturally hydrophilic or treated so as to be hydrophilic). Suitable materials include a polyamide, polypropylene, polyethylene terephthalate (PETP), ethylene tetrafluoroethylene (ETFE), or polyether ether ketone (PEEK). The porosity (pore size) of the porous membrane 944 may, for example, be 30 μm to 100 μm, e.g., about 70 μm. The pore size of the porous membrane 944 should be small enough to capture post-lysis cellular material but not too small so as to be vulnerable to clogging. A suitable mesh for the second porous membrane 620 is available from Sefar, Inc. Buffalo, NY, part no. 03-70/33 HPL having a pore size of 70 μm. Filter element 942 may comprise a sintered filter having porosity (pore size) that is the same as porous membrane 944 (e.g., a range of 30 μm to 100 μm or about 70 μm). The porosity of the filter element 942 may vary through its thickness, e.g., having a pore size that progressively decreases from a larger pore size (e.g., 100 μm) at a top surface of the filter element 942 to a smaller pore size (e.g., 30 μm) at a bottom surface of the filter element 942.

Deformable wall 930 is configured to be collapsible from an undeformed state (shown in FIG. 77) to a deformed state (shown in FIG. 78) when a required amount of force is applied against the deformable wall 930. In the undeformed state, as shown in FIG. 77, deformable wall 930 is situated above open space 918 of sleeve 910. In the deformed state, as shown in FIG. 78, deformable wall 930 is outwardly concave and projects into open space 918 of sleeve 910. As deformable wall 930 deforms from the undeformed state to the deformed state, sleeve 910 maintains its shape such that sleeve 910 remains sealing engaged against inner surface 515 of the sample chamber W1. Although vent hole 938 permits gas flow from chamber 932 as deformable wall 930 is being deformed to prevent pressure from building up within the chamber 932, thereby allowing deformable wall 930 to be deformed more effectively, the collapsed wall 930 pushes on the magnetic element 926 (and any non-magnetic beads 924 that may be contained in the chamber 932) into the non-magnetic beads 924 contained within the open space 918. Collapsing the deformable wall 930 into open space 918 eliminates chamber 932 and reduces the volume of the open space 918. As the combined volume of the magnetic element 926 and non-magnetic beads 924 (i.e., the lysis beads) exceeds the remaining volume of the recess 918, the frangible membrane 934 ruptures, as represented by fragments 934a, 934b of the frangible membrane shown in FIG. 78, thereby releasing the lysis beads into the sample chamber W1. The lysis beads are retained within the sample chamber W1 by filter element 942 and/or porous membrane 944 (see FIG. 79), and sample well W1 functions as a lysis chamber.

To facilitate controlled rupturing of the frangible membrane 934, the film may be configured to rupture in response to the application of a certain amount of force to frangible membrane 934. Frangible membrane 934 may be a porous film and/or may be composed of a material susceptible to rupturing, such as aluminum, a polymeric material, or a combination thereof. In some embodiments, frangible membrane 934 may include a rupture line, as represented by dashed line 936 in FIG. 76, to make membrane 934 susceptible (or more susceptible) to rupturing at application of a required force. The rupture line 936 may be a score line or a series of perforations or other partial cuts, indentations, etc. formed in the membrane 934. The arrangement of the rupture line 936 may be tuned to alter the required amount of force required to rupture frangible membrane 934. For example, the rupture line 936 may be a single line as shown in FIG. 76 having, e.g., a “U” shape or a “C” shape, or the rupture line may comprise multiple lines that may cross each other in an “X,” cross hair “+” shape, or star “*” shape.

A top end of peripheral wall 906 may be higher than a top end of deformable wall 930 to prevent a user or an instrument from inadvertently deforming deformable wall 930.

Deformable wall 930 may have a thickness that is less than the thickness of sleeve 910 so that deformable wall 930 is more susceptible to being deformed upon application of an external force. For example, deformable wall 930 may have a thickness of 0.5 to 1.0 mm, and sleeve 910 may have a thickness of 1.0-2.0 mm. Deformable wall 930 may have any shape suitable for allowing deformable wall 930 to be deformable. For example, in the illustrated embodiment, deformable wall 930 may be rounded, such as a generally hemispherical or dome shape. In other embodiments, deformable wall 930 may have other shapes suitable for being deformed, such as, for example, cylindrical.

In some embodiments, as shown in FIG. 77, bead delivery cap 900 may include a peelable bottom cover film 922 covering an outer surface of frangible membrane 934. For illustration purposes, bottom cover film 922 is shown in FIG. 77 with an exaggerated thickness and a slight gap between the bottom cover film 922 and the frangible membrane 934. In practice the bottom cover film would be in adhered in surface-to-surface contact with the frangible membrane 934. Bottom cover film 922 is configured to be peeled off frangible membrane 934 without affecting frangible membrane 934 or compromising the sealed connection between bottom end 916 of sleeve 910 and frangible membrane 934. In some embodiments, bottom cover film 922 may be composed of a material possessing sufficient flexibility for peeling, such as aluminum. Bottom film 922 would be removed from the bead delivery cap 900 by a user before the bead delivery cap is inserted into a sample chamber.

In some embodiments, as shown in FIG. 77, bead delivery cap 900 may include a peelable top cover film 923 adhered to a top edge of peripheral wall 906 and covering deformable wall 930. Top cover film 923 is configured to be peeled off peripheral wall 906. In some embodiments, top cover film 923 may be composed of a materials possessing sufficient flexibility for peeling, such as aluminum. Top film 923 would be removed from the bead delivery cap 900 by a user before the bead delivery cap is inserted into a sample chamber.

In some embodiments, a sample chamber cap may include a sachet (not shown), rather than frangible membrane 934 or contained within frangible membrane 934, to encapsulate the lysis beads (at least one magnetic element 926 and plurality of non-magnetic beads 924) within open space 918. A sachet may be comprised of a liquid dissolvable film, such as, for example, a PVA film or a PVOh film. The sachet may be secured to interior surface of sleeve 910 and configured to be displaced from the open space 918 when deformable wall 930 is collapsed to the deformed state and to be dissolved within the sample well W1 before a lysis operation is performed.

In a process for loading and containing at least one magnetic element 926 and plurality of non-magnetic beads 924 in the cap body of the bead delivery cap 900, bottom end 916 of lower sleeve 910 is initially open to allow the lytic agents (or sachet) to be loaded into chamber 932 and open space 918. The cap body is inverted so that the open bottom end 916 is upwardly facing, and in some embodiments, magnetic element 926 is loaded first into chamber 932, such that magnetic element 926 is disposed adjacent an interior surface of deformable wall 930. Locating the larger magnetic element 926 adjacent to deformable wall 930 allows deformable wall 930 to deflect the smaller non-magnetic beads 924 more effectively against frangible membrane 934.

The plurality of non-magnetic beads 924 are then loaded into open space 918, and partially into chamber 932, of the cap body such that the magnetic element 926 and the plurality of non-magnetic beads 924 substantially fill chamber 932 and open space 918. The quantity (e.g., volume) of lysis beads (magnetic element 926 and/or non-magnetic beads 924) loaded into chamber 932 and open space 918 is tuned to ensure that the deformation of deformable wall 930 causes the non-magnetic beads 924 to press against and apply a sufficient amount of force to rupture frangible membrane 934. In one non-limiting example, the collective volume of the chamber 932 and open space 918 may be 700 μl to 800 μl (e.g., about 733.5 μl), and the volume of the sample chamber W1 beneath the bead delivery cap 900 may be 700 μl to 800 μl (e.g., about 718.10 μl). In one non-limiting example, the lysis beads 926, 924 are densely packed in the chamber 932 and open space 918, for example, occupying at least 90% of the volume of the chamber 932 and open space 918.

After filling the cap body with the lysis beads, frangible membrane 934 is affixed to bottom end 916 of sleeve 910 to enclose open space 918 and retain magnetic element 926 and the plurality of non-magnetic beads 924 in chamber 932 and open space 918. Frangible membrane 934 may be affixed to bottom end 916 any suitable means, such as adhesive, heat sealing, or ultrasonic welding. As discussed above, frangible membrane 934 is configured to rupture when deformable wall 930 is deformed from the undeformed state to the deformed state. When deforming deformable wall 930 to displace the lysis beads in chamber 932 and open space 918, a sufficient amount of force is applied to rupture frangible membrane 934. The amount of force required to rupture frangible membrane 934 is more than what would be applied to frangible membrane 934 by incidental contact by a user or a machine. In some embodiments, the required amount of force applied to deformable wall 930 to rupture frangible membrane 934 is about 1.0 pounds to about 5.0 pounds. The amount of force required to rupture frangible membrane 934 may be adjusted by tuning: (1) the size and shape of deformable wall 930; (2) the volume of chamber 932 and/or open space 918; (3) the volume of lysis beads loaded into chamber 932 and open space 918; and (4) the composition and structure of frangible membrane 934, including the arrangement of the one or more rupture lines 936 on frangible membrane 934. Deformable wall 930 may be manually collapsed or may be collapsed by bead delivery cap actuator of the instrument, as described below.

After frangible membrane 934 is affixed to bottom end 916 of lower sleeve 925, bottom cover film 922 may be adhered to frangible membrane 934, preferably by a releasable adhesive that does not leave a residue. Bottom cover film 922 may be peeled off immediately before inserting cap 900 into sample well W1 to keep frangible membrane 934 shielded and protected during handling before processing. By shielding frangible membrane 934, bottom cover film 922 helps prevent inadvertent rupture of frangible membrane 934, such as by being exposed to incidental contact. In some embodiments, bottom cover film 922 and frangible membrane 934 may be fixed together to bottom end 221 of sleeve 220 such that frangible membrane 934 and bottom cover film 922 are applied simultaneously. In some embodiments, bottom cover film 922 may be applied to frangible membrane 934 after fixing frangible membrane 934 to bottom end 916 of sleeve 910.

In each embodiment of a lysis capsule 600, 600′, 600″ or lysis vessel 700 described above, subjecting the magnetic element 626, 626′, 626″ or 726 to a magnetic field of varying polarity will cause movement of the magnetic element as the magnetic element constantly seeks to realign with the changing north and south poles of the varying magnetic field. This movement of the magnetic element 626, 626′, 626″ or 726 will cause corresponding movement of the non-magnetic beads 624, 624′, 624″ or 724 within the corresponding lysis chamber 622, 622′, 622″ or 722, and movement of the non-magnetic beads within the lysis chamber will effect mechanical lysis of sample material contained within the lysis chamber along with the magnetic element and the non-magnetic beads.

In an alternate embodiment, lytic agents may be pre-positioned in a sample chamber without the need for an independent containment capsule or vessel. Such an arrangement eliminates the need for a separate lysis capsule, e.g., one of lysis capsules 600, 600′, 600″, which needs to be inserted into the sample chamber, or a sample chamber cap containing lytic agents, such as bead delivery cap 900, which may further require a bead delivery cap actuator.

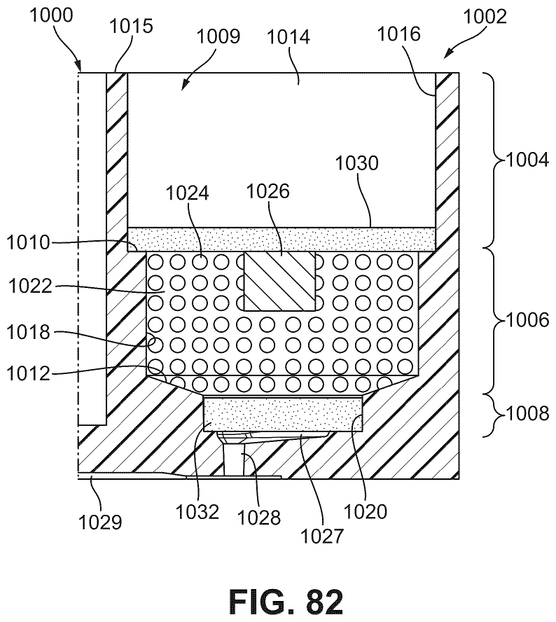

FIG. 82 is a partial cross-section of a fluidic cartridge 1000 through a mechanical lysis sample chamber 1002 that provides a lysis chamber and lytic agents for performing mechanical lysis directly within the sample chamber. Fluidic cartridge 1000 may be substantially identical to fluidic cartridge 500 described herein, with the exception of the inclusion of the mechanical lysis sample chamber 1002. Like fluidic cartridge 500, fluidic cartridge 1000 may comprise a cartridge body made by injection molding of a thermoplastic polymer material.

In general, mechanical lysis sample chamber 1002 may include a fluid sample chamber 1009 with an open first, e.g., upper, end 1014 and a sample exit port 1028 and a lysis chamber 1022 defined by an internal wall 1018 of the fluid sample chamber 1009, an upper, or first, porous membrane 1030 fixed within the sample chamber 1009, and a lower, or second, porous membrane 1032 spaced apart from the first porous membrane 1030 and fixed within the sample chamber 1009. A plurality of non-magnetic beads 1024 and at least one magnetic element 1026 is disposed within the lysis chamber 1022. Pores of the first porous membrane 1030 and the second porous membrane 1032 are sized to retain the plurality of non-magnetic beads 1024 and the at least one magnetic element 1026 within the lysis chamber 1022.

More specifically, in one non-limiting example, mechanical lysis sample chamber 1002 may include a fluid sample chamber 1009 comprised of lower section 1008, a middle section 1006 that is wider than lower section 1008, and an upper section 1004 that is wider than middle section 1006. The open upper end 1014 of the sample chamber 1002 is surrounded by an inner wall 1016 of upper section 1004. The sample chamber 1002 may include a sloped transition section 1012 between the middle section 1006 and the lower section 1008. The sample exit port 1028 extends from the lower section 1008 to a channel 1029 connecting the mechanical lysis sample chamber 1002 to a syringe barrel (such as syringe barrel SB, not shown in FIG. 82). A sloped transition surface 1027 may be provided between the lower section 1008 and the sample exit port 1028.

A transverse ledge 1010 between upper section 1004 and middle section 1006 supports first porous membrane 1030, which overlaps the open upper end 1014. First porous membrane 1030 may be press fit into the upper section 1002 with an interference fit between an outer periphery of the first porous membrane 1030 and the inner wall 1016 of the upper section 1002. For this purpose, first porous membrane 1030 may be a compressible material that is somewhat wider than the width of the upper section 1004 and having a thickness that is sufficient to permit press-fitting of the material into the upper section 1002.

A lower, or second, porous membrane 1032 overlaps the sample exit port 1028, as well as the sloped transition surface 1027 and may be press fit into the lower section 1008 with an interference fit between an outer periphery of the second porous membrane 1032 and an inner wall 1020 of the lower section 1008. For this purpose, second porous membrane 1032 may be a compressible material that is somewhat wider than the width of the lower section 1008 and has a thickness that is sufficient to permit press-fitting of the material into the lower section 1008.

The lysis chamber 1022 is defined by an inner wall 1018 of the middle section 1006 between the first porous membrane 1030 and the second porous membrane 1032. Lysis chamber 1022 contains a plurality of non-magnetic beads 1024, which may be identical to non-magnetic beads 624 described above, filling a portion of the volume of the lysis chamber 1022 and a magnetic element 1026, which may be identical to magnetic element 626 described above. Fluid sample is provided to the lysis chamber 1022, and the magnetic element 1026 is agitated, as described below, to impart motion to the non-magnetic beads 1024 to effect mechanical lysis of cells present in the sample contained within the lysis chamber 1022. The relative volumes of the lysis chamber 1022, non-magnetic beads 1024, and magnetic element 1026 may be as described above for the lysis chamber 622, non-magnetic beads 624, and magnetic element 626.

First porous membrane 1030 may be positioned at a distance below a top edge 1015 of the chamber 1002, as shown in FIG. 82. A portion of the upper section 1004 above the first porous membrane 1030 (headspace) may provide a sample loading reservoir to retain a volume of fluid sample as the fluid sample passes through the first porous membrane 1030. The volume of upper section 1004 lysis chamber 1022 will depend on the volume of sample desired to be processed in the mechanical lysis sample chamber 1002. In one non-limiting example, a volume of upper section 1004 is about 1000 μL and a volume of the lysis chamber 1022 may be about 800 μL. In one non-limiting example, a volume of the non-magnetic beads 1024 and the magnetic element 1026 may be about 200-300 μl (the volume of the magnetic element 1026 may be about 64 μl (4 mm×4 mm×4 mm)) leaving about 500-600 μl for fluid in the lysis chamber 1022. Chamber 1002 may be closed—after dispensing sample into the chamber—by inserting a cap, such as cap 516 described herein, into the open upper end 1014 of upper section 1004 or by securing a lid or other cover over the open upper end 1014. The distance between the first porous membrane 1030 and the top edge 1015 may be varied—by varying the position of the ledge 1010—depending on factors such as the desired volume of the lysis chamber 1022, the desired volume of a sample loading reservoir, and/or the form factor of the cover used to close the open upper end 1014.

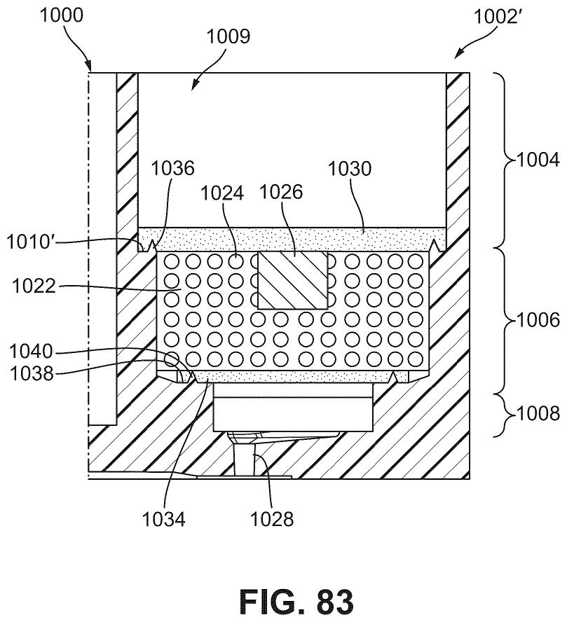

FIG. 83 is a partial cross-section of a fluidic cartridge 1000 through an alternate mechanical lysis sample chamber 1002′. Mechanical lysis sample chamber 1002′ is substantially identical to mechanical lysis sample chamber 1002 except that first porous membrane 1030 is heat sealed to ledge 1010′ using a heat sealer having a size and shape corresponding to first porous membrane 1030, and a second porous membrane 1034, which may be a post-filter, is heat sealed to a ledge 1038 disposed above the lower section 1008 using a heat sealer having a size and shape corresponding to second porous membrane 1034. To facilitate heat sealing, ledge 1010′ and ledge 1038 may include energy directors 1036, 1040, respectively. Energy directors are components or features in heat sealing applications that help focus and control the flow of energy to the area where the seal is being created. Examples of energy directors include raised features (e.g., a rib) projecting above ledges 1010′ and 1038 to form a narrow edge (e.g., a dome-shaped cross-section or a knife-edge (triangular) cross-section) that will focus energy at the edge and facilitate localized material melting at the edge to promote sealing. The heat sealing process melts the energy directors 1036, 1040, and the molten plastic penetrates the first porous membrane 1030 and the second porous membrane 1034, respectively, thereby forming a strong bond upon cooling and hardening. For this application, woven meshes may be well suited as the first porous membrane 1030 and the second porous membrane 1034, as an open mesh will allow penetration of the molten plastic.

The variations shown in FIGS. 82 and 83 may be combined. For example, the first porous membrane 1030 may be press fit and the second porous membrane 1034 may be heat sealed to ledge 1038, or the first porous membrane 1030 may be heat sealed and the second porous membrane 1032 may be press fit into the lower section 1008. Some variations may include both porous membrane 1034 heat sealed to, or otherwise supported on, ledge 1038 and porous membrane 1032 disposed within lower section 1008 of the sample chamber. For a variation having only one second porous membrane 1034 heat sealed to ledge 1038, lower section 1008 of the sample chamber may be omitted.

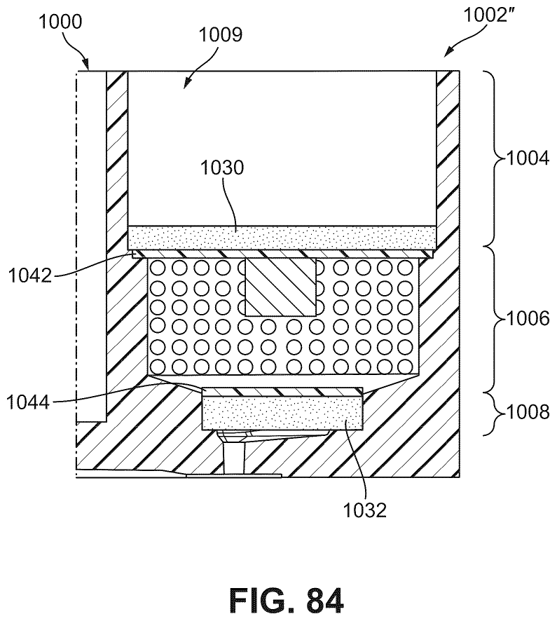

FIG. 84 is a partial cross-section of a fluidic cartridge 1000 through an alternate mechanical lysis sample chamber 1002″. Mechanical lysis sample chamber 1002″ is substantially identical to mechanical lysis sample chamber 1002 except that it includes a protective layer 1042 positioned below first porous membrane 1030 and a protective layer 1044 positioned above second porous membrane 1032. The first porous membrane 1030 and the second porous membrane 1032 must remain structurally intact throughout the mechanical lysis process. If either filter is susceptible to damage, protective layer 1042 or protective layer 1044 may added. Each of protective layers 1042 and 1044 may be a supportive mesh (such as woven nylon or polyester mesh) or structure (such as injection molded or 3D printed mesh) that is mechanically strong and may have a porosity that is at least as large as the porosity of the first and second porous membranes and up to about 300-350 μm, the maximum size being limited by the size of the lysis beads within the lysis chamber 1022.

Protective layer 1042 may be used in combination with a first porous membrane 1030 that is press fit into the upper section 1004 or with a first porous membrane 1030 that is heat sealed to ledge 1010′. For a heat sealing application, protective layer 1042 may be affixed to first porous membrane 1030, e.g., by an adhesive, so that molten plastic penetrating the pores of the protective layer 1042 will secure both the protective layer 1042 and the first porous membrane 1030 to the ledge 1010′. Similarly, protective layer 1044 may be used in combination with a second porous membrane 1032 that is press fit into the lower section 1008 or with a second porous membrane 1034 that is heat sealed to ledge 1038. For either application, protective layer 1044 may be placed on top of the second porous membrane 1032 or the second porous membrane 1034, where it is held in place by gravity. Alternatively, the protective layer 1044 may be affixed to the second porous membrane 1032 or the second porous membrane 1034, e.g., by an adhesive, before or after the second porous membrane 1032 is press fit into the lower section 1008 or before or after the second porous membrane 1034 is heat sealed to ledge 1038.

The pore size of the first porous membrane 1030 for each of mechanical lysis sample chamber 1002, 1002′, 1002″ should be larger than the pore size of the second porous membrane 1032 or 1034. First porous membrane 1030 should be sufficiently large to allow pathogens, such as 40 μm parasites, to pass through, and the second porous membrane 1032 or 1034 should provide sufficient filtration to prevent downstream clogging. The pore sizes of the first and second porous membranes should be small enough to retain the lysis beads within the lysis chamber 1022.

First porous membrane 1030 may be a mesh or a filter matrix and is preferably hydrophilic (either naturally hydrophilic or treated so as to be hydrophilic) to facilitate passage of fluid sample material through the first porous membrane 1030. Suitable materials include a polyamide, polypropylene, polyethylene terephthalate (PETP), ethylene tetrafluoroethylene (ETFE), or polyether ether ketone (PEEK). Other suitable materials include a woven mesh filter, such as nylon or polyester or similar, or a membrane filter. The porosity (pore size) of the first porous membrane 1030 may, for example, be 70 μm to 500 μm, e.g., about 300 μm, the maximum size being limited by the size of lytic beads to be retained within lysis chamber 622. A suitable mesh for the first porous membrane 1030 is available from Sefar, Inc. Buffalo, NY part no. 03-50/37 having a pore size of 50 μm and a suitable filter for first porous membrane 1030 is available from Porex Filtration Group, product number 4899 or 3677.

Second porous membrane 1032 or 1034 may be a mesh or a filter matrix and is preferably hydrophilic (either naturally hydrophilic or treated so as to be hydrophilic). Suitable materials include a polyamide, polypropylene, polyethylene terephthalate (PETP), ethylene tetrafluroethylene (ETFE), or polyether ether ketone (PEEK). The porosity (pore size) of the porous membrane 944 may, for example, be 30 μm to 100 μm, e.g., about 70 μm. The pore size of the porous membrane 944 should be small enough to capture post-lysis cellular material but not too small so as to be vulnerable to clogging. A suitable mesh for the second porous membrane 1032 or 1034 is available from Sefar, Inc. Buffalo, NY, part no. 03-1/1 having a pore size of 1 μm. Alternatively second porous membrane 1032 or 1034 may comprise a sintered filter having porosity (pore size) in a range of 30 μm to 100 μm or about 70 μm. The porosity of the second porous membrane 1032 or 1034 may vary through its thickness, e.g., having a pore size that progressively decreases from a larger pore size (e.g., 100 μm) at a top surface of the second porous membrane 1032 or 1034 to a smaller pore size (e.g., 30 μm) at a bottom surface of the second porous membrane 1032 or 1034. A suitable filter for second porous membrane 1032 or 1034 is available from Sterlitech PES 0.65-1.2 μm.

### Internal Control

In some applications, for example, where a molecular assay is being performed on a test platform, such as fluidic cartridge 500, 800 or 1000, it may be desirable to combine an internal control with a reaction mixture. An internal control, e.g., a nucleic acid (DNA and/or RNA), such as a nucleic acid transcript, plasmid, or nucleic acid extracted from a whole organism, such as yeast, will be exposed to the same assay conditions as the sample, such as, lysis (in the case of a whole organism containing the internal control or rupturable encapsulated pellets containing the internal control as described below), sample purification, combination with amplification reagents and/or detection probes, thermal cycling, emission signal detection, etc. The internal control nucleic acids may be responsive to the same amplification reagents as the sample target, but different detection probes will be provided to bind to the internal control nucleic acids and the sample target so as to distinguish the two. If the amplification and detection procedures are performed correctly, detection of a signal indicating the presence of the internal control (i.e., a positive result for the internal control nucleic acid) can be expected. On the other hand, failure to detect a signal indicating the presence of the internal control (i.e., a negative result for the internal control nucleic acid), or detecting less of the internal control than anticipated, may indicate an error or malfunction in one or more steps of the sample preparation (e.g., lysis or analyte purification), the material transport, the amplification, and/or the detection steps. Such errors or malfunctions may be system-based—e.g., the instrument or a module within the instrument has malfunctioned—and/or material-based—e.g., one or more reagents and/or probes has become unstable. Thus, the internal control is provided to validate an assay result and/or to validate the effectiveness of a cell lysis procedure, e.g., to confirm that all steps of the assay, including extraction, amplification and detection, should have performed as expected.

One way to introduce an internal control to the reaction mixture is simply to dispense an amount of a reagent containing the internal control (“internal control reagent” or “ICR”) into the sample chamber W1 along with the sample. Alternatively, the internal control reagent may be pre-positioned in the cartridge so that it will be combined with the sample after the sample is dispensed into the sample chamber, and without requiring a technician to add an internal control to the sample before or at the time of introducing the sample to the fluidic cartridge.

On-board mechanical lysis affords flexibility in the manner in which an internal control is added to a reaction mixture by providing other mechanisms for introducing the internal control. The internal control may be provided in a non-liquid form, where a dried reagent is an example of a non-liquid form. For example, an internal control reagent in a fluid form may be applied and dried onto a portion of the lysis capsule, lysis vessel, or sample chamber such that when that portion of the lysis capsule, lysis vessel, or sample chamber is contacted by a fluid sample, the internal control reagent will dissolve and combine with the fluid sample. For applications that do not involve on-board mechanical lysis, an internal control reagent in a fluid form may be applied and dried onto a portion of the sample chamber such that when that portion of the sample chamber is contacted by a fluid sample, the internal control reagent will dissolve and combine with the fluid sample.

More specifically, for example, for applications involving on-board mechanical lysis capsules 600, 600′, 600″, an internal control reagent in a fluid form may be applied and dried onto (sometimes referred to as spotting) the first porous membrane 618, 618′, 618″, respectively, so that when fluid sample is dispensed into the capsule through the first porous membrane, the dried internal control reagent dissolves and is “washed” from the first porous membrane and combined with the fluid sample that enters the lysis chamber 622, 622′, 622″. Alternatively, or additionally, an internal control reagent in a fluid form may be applied and dried onto the second porous membrane 620, 620′, or 620″ of lysis capsule 600, 600′, or 600″, respectively.

For lysis capsule 700, an internal control reagent in a fluid form may be applied and dried into a non-liquid form onto the porous membrane 720. When fluid sample is dispensed into sample chamber W1, and the capsule 700 is inserted into the sample chamber W1, sample will pass through the porous membrane 720, and the dried internal control reagent will dissolve and combine with the fluid sample that enters the lysis chamber 722.

For mechanical lysis processes performed directly within the sample chamber without a lysis capsule, e.g., mechanical lysis sample chamber 1002, 1002′, 1002″, an internal control reagent in a fluid form may be applied and dried onto the first porous membrane 1030, so that when fluid sample is dispensed into the sample chamber through the first porous membrane, the dried internal control reagent dissolves and is “washed” from the first porous membrane and combined with the fluid sample that enters the lysis chamber 1022. Alternatively, or additionally, an internal control reagent in a fluid form may be applied and dried onto the second porous membrane 1032 or 1034.

Alternatively, or additionally, an internal control reagent may be applied and dried on the non-magnetic beads 624, 624′, 624″ of lysis capsule 600, 600′, 600″, non-magnetic beads 724 of lysis capsule 700, non-magnetic beads 924 of bead delivery cap 900, or non-magnetic beads 1024 of mechanical lysis sample chamber 1002, 1002′, 1002″. When fluid sample is introduced into the lysis chamber 622, 622′, 622″ of lysis capsule 600, 600′, 600″, the lysis chamber 722 of lysis vessel 700, the lysis chamber 1022 of mechanical lysis sample chamber 1002, 1002′, 1002″, or when the non-magnetic beads 924 of bead delivery cap are released into the sample chamber W1, the dried internal control reagent dissolves and is “washed” from the non-magnetic beads (especially as the non-magnetic beads are moved throughout the lysis chamber by the magnetic element) and combines with the fluid sample.

Alternatively, or additionally, an internal control reagent may be applied and dried onto an internal wall of the hollow body 602, 602′, 602″, 702 of lysis capsules/vessel 600, 600′, 600″, 700, respectively, or on an internal wall of the lysis chamber 1022 of mechanical lysis sample chamber 1002, 1002′, 1002″, so that when fluid sample is introduced into the lysis chamber 622, 622′, 622″, 722, 1022, respectively, the dried internal control reagent dissolves and is “washed” from the internal wall and combines with the fluid sample within the lysis chamber 622, 622′, 622″, 722, 1022.

Alternatively, or additionally, an internal control may be embedded in or contained within an internal control pellet (micropellet), or capsule, adapted to dissolve in the presence of a fluid sample or disintegrate when subjected to mechanical lysis, e.g., by bead beating, to release the internal control nucleic acids into the sample. Where the internal control is released by disintegrating the internal control pellet by mechanical lysis, the internal control pellet is subject to the same shearing collision forces imparted by the lytic agents (e.g., the non-magnetic beads described above or other lytic agents), and the nucleic acids released from the pellet may function as both an internal control to validate assay results and as a process control to confirm that mechanical lysis has occurred, since the internal control nucleic acids will only be available for amplification and detection if released from a lysed internal control micropellet. In this regard, using an internal control pellet to validate a lysis process is not limited to the particular lysis systems or processes described herein whereby motion is imparted to a magnetic element surrounding by a plurality of non-magnetic elements by a varying magnetic field. An internal control pellet configured release nucleic acids when subjected to shearing collision forces imparted by moving lytic agents may be used to validate the lysis process in any system or process where cells are disrupted (i.e., lysed) by shearing collision forces imparted by moving lytic agents, regardless of what those lytic agents are or how motion is imparted to the lytic agents.

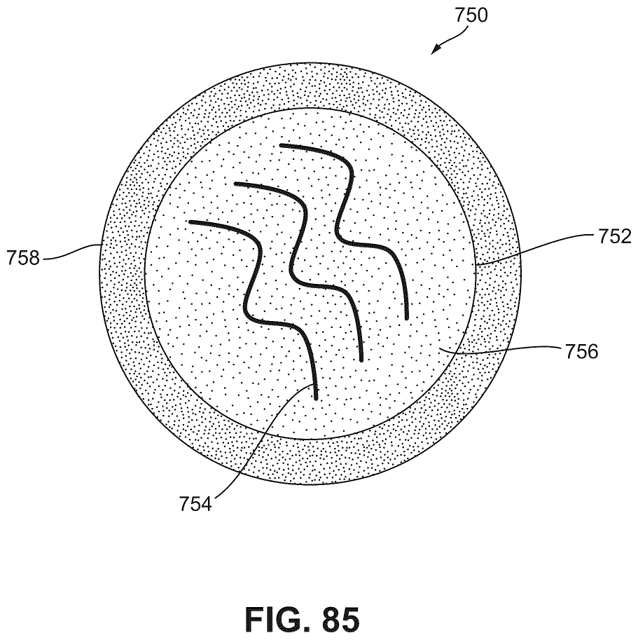

FIG. 85 is a schematic, cross-sectional view of a coated micropellet, or capsule, 750 containing an internal control (“IC micropellet”). IC micropellet 750 includes a core 752 including nucleic acids (e.g., DNA and/or RNA) 754 embedded in an excipient 756 and surrounded by a coating 758. Each IC micropellet may be generally spherical, with a diameter of about 0.5 to 1 mm, or rod shaped, with a length of about 1 mm. Desired performance parameters of an IC micropellet may include maintaining stability of the internal control nucleic acids for a certain specified period of shelf life under certain specified environmental conditions, such as temperature and relative humidity. For example, design parameters for the IC micropellet may be that it maintain stability of the internal control nucleic acids in an environment of 70-95% relative humidity at 30° C. for a shelf-life of up to 18 months and/or during exposure to uncontrolled product shipping—e.g., 90 hours at 55° C. Other desirable design parameters for the IC micropellet 750 may include that the micropellet, and particularly the protective coating 758, be able to withstand mechanical stresses during product shipping. It may also be desirable that the coating 758 is able to be mechanically lysed (i.e., at least partially disrupted or disintegrated to expose the core 752) within a short timeframe (e.g., less than or equal to five minutes) in an on-board lysing procedure and that the core 752 will dissolve when exposed to liquid sample to release the internal control nucleic acid(s) after the coating is lysed. In certain applications, it may be desirable that the coating 758 is able to be chemically lysed (i.e., at least partially disrupted or disintegrated to expose the core 752) when exposed to chemical lysis conditions, such as solutions having a pH of greater than 7, e.g., between 7.5 and 8.5, or other chemical lysis conditions. The excipient is adapted to at least partially dissolve when exposed to fluids after the coating is disrupted to thereby release the internal control. Other design parameters may include that the core 752 comprise material able to bind to the IC nucleic acids as a bulk/vehicle/excipient and that the micropellet minimize reaction with the sample target analyte while in turbulent conditions, such as during mechanical lysing.

In a non-limiting example, a DNA internal control 754 is a plasmid internal control, and the mass ratio of the DNA internal control is 6.10E-14 (mass/pellet). The internal control may be contained in an internal control reagent comprising nuclease-free, molecular grade water. An RNA internal control 754 is an in vitro transcript (IVT), and the mass ratio of the RNA internal control is 9.33E-15 (mass/pellet).

Suitable materials for a core excipient 756 include Avicel® PH101 (microcrystalline cellulose) & Klucel Fusion X™ (hydroxypropylcellulose) available from Ashland, Inc. of Wilmington, Delaware. Suitable materials for the coating 758 include Aquarius™ Protect Moisture Barrier VAA (cellulose derivative & natural wax blend based on polyvinyl alcohol), also available from Ashland, Inc.

Core 752 may be formed by wet granulation or extrusion from a “dough” formed by combining cellulose or carbohydrate powder with a liquid internal control formulation. Individual core pellets may be formed by spheronization (also referred to as Marumerization), which is a process where extrudates (the output from an extruder) are shaped into small rounded or spherical pellets. The extruded pellets are then dried, e.g., on a fluid bed, and coating 758 may be applied by a spray coating process, such as a top spray coating process, a bottom spray coating process (also known as a Wurster process or Wurster coating, or a tangential spray coating process (also known as rotor or HP coating).

In examples described herein, IC micropellets 750 may be combined with non-magnetic beads 624, 624′, 624″ within the lysis chambers 622, 622′, 622″ of lysis capsules 600, 600′, 600″, respectively, with non-magnetic beads 724 within the lysis chamber 722 of lysis vessel 700, with non-magnetic beads 1024 within the lysis chamber 1022 of mechanical lysis sample chamber 1002, 1002′, 1002″, or with non-magnetic beads 924 contained in bead delivery cap 900.

Pellets with embedded nucleic acids with protective coatings, such as enteric protective coatings, may be employed to provide a stable form of the nucleic acids for delivery of the nucleic acids (e.g., small interfering RNA or mRNA) in applications other than as an internal control for validating an assay result or for validating the effectiveness of a lysis procedure. For example, coated nucleic acid pellets may be employed for oral delivery of nucleic acid where the pellet coating is adapted to dissolve when exposed to a liquid environment or a liquid environment of a certain pH level, such as the more acidic environment of the stomach (low pH) or the less acidic environment of the small intestine (higher pH).

### Manufacturing a Fluidic Cartridge

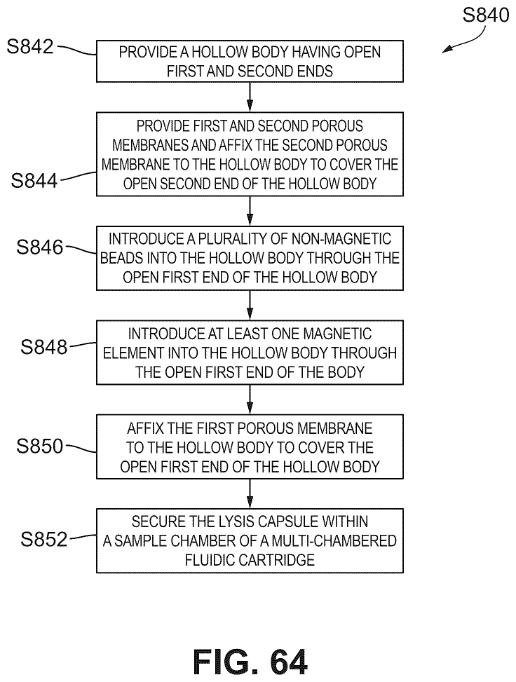

The following description presents an example of method of manufacturing a fluidic cartridge, such as fluidic cartridge 500, containing a lysis capsule, such as one of lysis capsules 600, 600′, 600″. FIG. 64 shows a flow diagram illustrating an embodiment of a method S840 for manufacturing a fluidic cartridge. In various embodiments, some of the method steps shown may be performed concurrently, in a different order than shown, or may be omitted. Additional method steps may also be performed as desired. The workflow begins at step S842.

The lysis capsule is assembled by, as step S842, providing a hollow body having open first and second ends, e.g., hollow body 602 with open ends 608, 614 (FIG. 50), hollow body 602′ with open ends 608′, 614′ (FIG. 51), or hollow body 602″ with open ends 608″, 614″ (FIG. 52).

Step S844 comprises providing first and second porous membranes, e.g., first and second membranes 618, 620, first and second membranes 618′, 620′, and first and second membranes 618″, 620″. The second membrane is affixed to the hollow body to cover the open second end of the hollow body. For example, second porous membrane 620 is affixed to the second rim 612 of hollow body 602 to cover open end 614 (FIG. 50), second porous membrane 620′ is affixed to the second rim 612′ of hollow body 602′ to cover open end 614′ (FIG. 51), or second porous membrane 620″ is affixed to the second rim 612″ of hollow body 602″ to cover open end 614″ (FIG. 52) by an adhesive, heat sealing, or ultrasonic welding.

Step S846 comprises introducing non-magnetic beads into the hollow body, retained by the second (lower) porous membrane. For example, non-magnetic beads 624 are introduced into hollow body 602 where they are retained by second porous membrane 620 covering the second end 614 (FIG. 50), non-magnetic beads 624′ are introduced into hollow body 602′ where they are retained by second porous membrane 620′ covering the second end 614′ (FIG. 51), or non-magnetic beads 624″ are introduced into hollow body 602″ where they are retained by second porous membrane 620″ covering the second end 614″ (FIG. 52).

Step S848 comprises introducing at least one magnetic element into the hollow body, retained by the second (lower) porous membrane. For example, magnetic element 626 is introduced into hollow body 602 where it is retained by second porous membrane 620 covering the second end 614 (FIG. 50), magnetic element 626′ is introduced into hollow body 602′ where it is retained by second porous membrane 620′ covering the second end 614′ (FIG. 51), or magnetic element 626″ is introduced into hollow body 602″ where it is retained by second porous membrane 620″ covering the second end 614″ (FIG. 52).

Step S850 comprises affixing first porous membrane to the hollow body to cover the open first end of the hollow body and form the lysis chamber. For example, first porous membrane 618 is affixed to the first rim 606 of hollow body 602 to cover open end 618 (FIG. 50), first porous membrane 618′ is affixed to the first rim 606′ of hollow body 602′ to cover open end 618′ (FIG. 51), or first porous membrane 618″ is affixed to the first rim 606″ of hollow body 602″ to cover open end 618″ (FIG. 52) by an adhesive, by heat sealing, or by ultrasonic welding.

Optionally, a non-liquid internal control reagent may be applied to the first porous membrane prior to step S850, to an internal surface of the hollow body prior to step S846, or to at least a portion of the non-magnetic beads prior to step S846. The internal control reagent may be applied in a liquid form to the first porous membrane, the internal surface of the hollow body, or the non-magnetic beads and dried thereafter. Optionally, an internal control capsule in which an internal control reagent is embedded or contained may be introduced into the hollow body after step S844.

In step S852, the lysis capsule constructed by steps S842 to S850 is secured within a sample chamber of the fluidic cartridge, for example, within sample chamber W1 of fluidic cartridge 500. In one non-limiting example, the lysis capsule, e.g., lysis capsule 600, 600′, 600″, may be press-fitted into the sample chamber W1, and, in another example, the lysis capsule may be threadedly mated with an inner surface of the sample chamber W1.

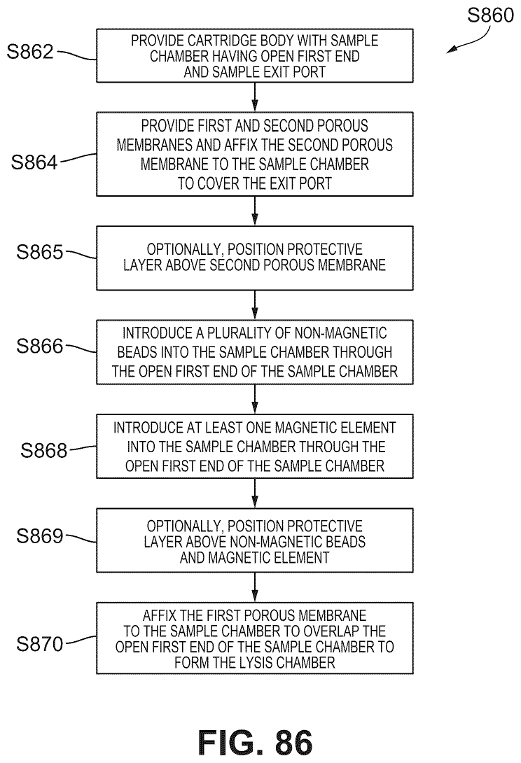

The following description presents an example of a method of manufacturing a fluidic cartridge, such as fluidic cartridge 1000, containing a mechanical lysis sample chamber, such as one of mechanical lysis sample chambers 1002, 1002′, 1002″. FIG. 86 shows a flow diagram illustrating an embodiment of a method S860 for manufacturing such a fluidic cartridge. In various examples, some of the method steps shown may be performed concurrently, in a different order than shown, or may be omitted. Additional method steps may also be performed as desired. The workflow begins at step S862.

In Step S862, a cartridge body is provided. The cartridge body includes a sample chamber having an open first end and a sample exit port at a second end of the sample chamber and, additionally, one or more chambers that are that are fluidly connected or connectable with the sample chamber. For example, the cartridge body may include sample chamber 1009 having open first end 1014 and sample exit port 1028 and which is connectible by channel 1029 with syringe barrel SB (FIG. 82—syringe barrel SB is not shown in FIG. 82). Sample chamber 1009 may include lower section 1008, middle section 1006, which is wider than lower section 1008, and upper section 1004, which is wider than middle section 1006. A transverse ledge 1010, 1010′ may be provided between upper section 1004 and middle section 1006 (FIGS. 82 and 83), and a ledge 1038 may disposed above the lower section 1008 (FIG. 83).

Step S864 comprises providing a first porous membrane and second porous membrane, e.g., first porous membrane 1030 and second porous membrane 1032 and/or second porous membrane 1034, and affixing one of the second membranes 1032, 1034 to the sample chamber to cover the sample exit port 1028. For example, second porous membrane 1032 may be press fit into the lower section 1008 of sample chamber 1009 with an interference fit between an outer periphery of the second porous membrane 1032 and an inner wall 1020 of the lower section 1008 to cover the sample exit port 1028 (FIG. 82). Alternatively, or in addition, second porous membrane 1034 may be heat sealed to ledge 1038, which may include melting the energy director(s) 1040 on ledge 1038 so that the molten plastic penetrates the second porous membrane 1034 (FIG. 83).

In optional Step S865, a protective layer may be positioned above the second porous membrane. For example, protective layer 1044 may be positioned above second porous membrane 1032 (FIG. 84) and/or second porous membrane 1034 (not shown). Protective layer 1044 may be placed on second membrane 1032 and/or second porous membrane 1034 after step S864, or the protective layer 1044 may be affixed, e.g., by an adhesive, to second porous membrane 1032 and/or the second porous membrane 1034 before or after step S864.

In Step S866, non-magnetic beads are introduced into the sample chamber through the open first end and are retained by the second (lower) porous membrane. For example, non-magnetic beads 1024 are introduced into sample chamber 1009 through open first end 1014, where they are retained by the second porous membrane 1032 (FIG. 82) or by the second porous membrane 1034 (FIG. 83).

In Step S868, at least one magnetic element is introduced into the sample chamber through the open first end and is retained by the second (lower) porous membrane. For example, magnetic element 1026 is introduced into the sample chamber 1009 through the open first end 1014, where it is retained by second porous membrane 1032 (FIG. 82) or by the second porous membrane 1034 (FIG. 83).

In Step S870, the first porous membrane is affixed to the sample chamber in a position overlapping the open first end of the sample chamber so that the first and second porous membranes and an internal wall of the sample chamber form the lysis chamber. For example, first porous membrane 1030 may be press fit into the upper section 1004 against ledge 1010 with an interference fit between an outer periphery of the first porous membrane 1030 and an inner wall 1016 of the upper section 1004 to overlap the open first end 1014 (FIG. 82). Alternatively, first porous membrane 1030 may be heat sealed to ledge 1010′, which may include melting the energy director(s) 1036 on ledge 1010′ so that the molten plastic penetrates the first porous membrane 1030 (FIG. 83).

In optional Step S869, a protective layer may be positioned above the non-magnetic beads and the magnetic element below the first porous membrane. Step S869 may precede step S870, for example, by positioning protective layer 1042 within upper section 1004 before the first porous membrane 1030 is press fit into the upper section 1004 (FIG. 84). Alternatively, Step S869 may precede step S870, for example, by affixing protective layer 1042 to the first porous membrane 1030 before the first porous membrane 1030 is heat sealed to ledge 1010′ (FIG. 83), which may include melting the energy director(s) 1036 on ledge 1010′ so that the molten plastic penetrates the protective layer 1042.

### Electromagnet

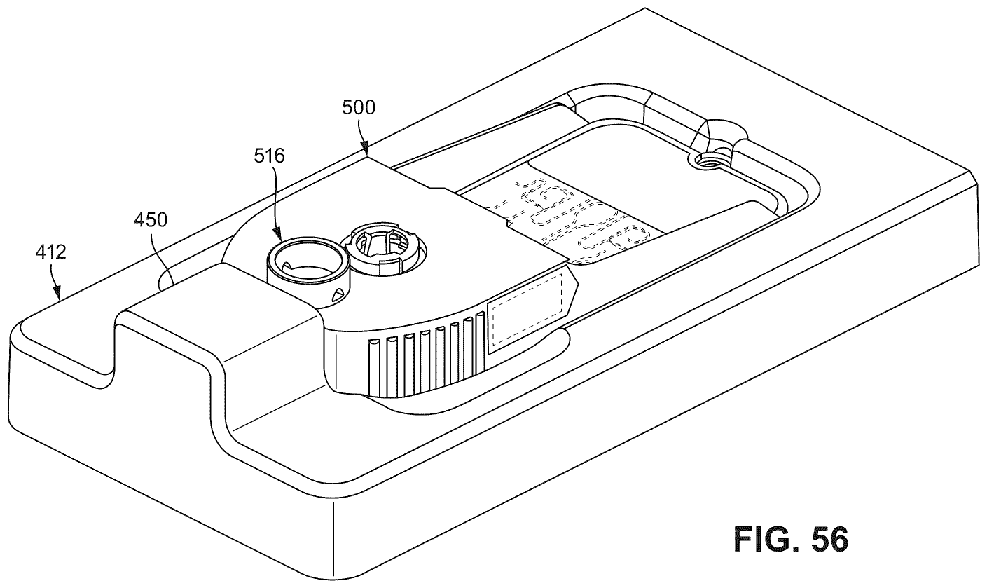

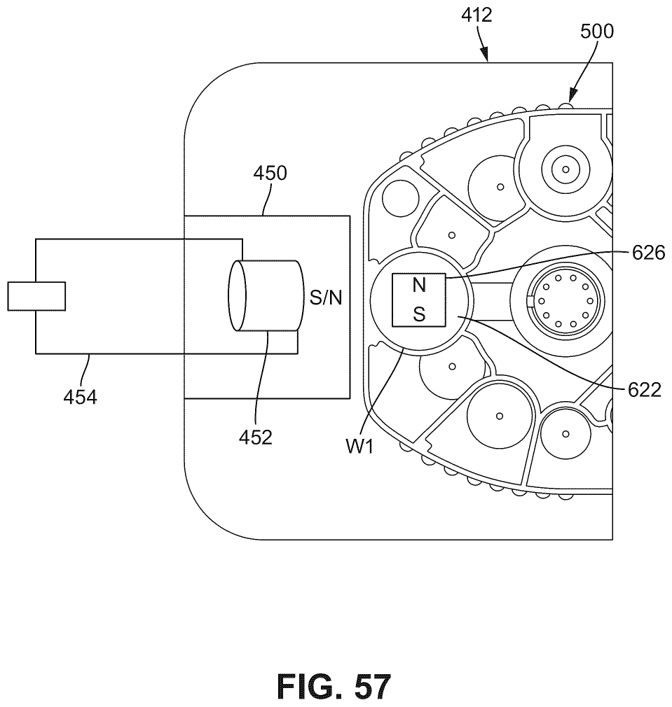

FIG. 56 is a perspective view of fluidic cartridge 500 supported in a cartridge holder 412 of the instrument 10, which will be described in more detail below. A variable magnet—e.g., an electromagnet—is housed within a magnet housing 450 of the cartridge holder 412 in close proximity to the sample chamber located beneath the cap 516. FIG. 57 is a schematic view showing an electromagnet 452 within the magnet housing 450 and connected to a circuit 454, e.g., an oscillating circuit, configured to alternate a current to the electromagnet 452 (e.g., in a sine wave) to cause the polarity of the electromagnet 452 to repeatedly change. The fluidic cartridge 500 is positioned in the cartridge holder such that the sample chamber W1, the lysis chamber 622, and the magnetic element 626 are as close as possible to the electromagnet 452 so that the magnetic element 626 will be affected by the magnetic field generated by the electromagnet 452.

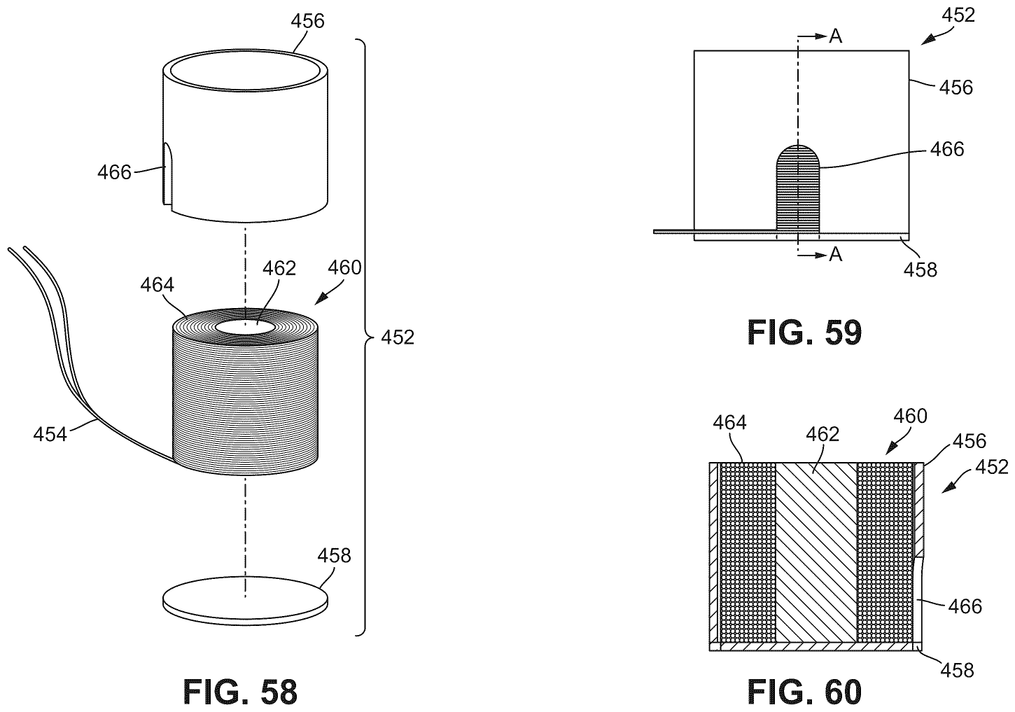

Features of electromagnet 452 are shown in FIGS. 58-60. Electromagnet, or electromagnet assembly, 452 comprises a coil 460 connected to circuit 454, a pot cylinder 456, and a pot base plate 458. Coil 460 comprises a core 462 surrounded by wire windings 464. Core 462 may be made from a material having high magnetic permeability and low electrical conductivity to permit current flow through core 462 while minimizing heat-inducing eddy currents. Suitable core materials include laminates typically used for electromagnet applications or a powdered metal, such as iron powder core 200C Series mix-70 available from Micrometals, Inc. (Anaheim, CA). Wire windings 464 may comprise conductive materials, such as copper, or 28 American Wire Gage “AWG” magnet wire, acrylic varnished. Pot cylinder 456 and pot base 458 may be formed from 410 stainless steel, annealed. An opening 466 may be formed in the pot cylinder 456 to receive a thermostat switch (not shown) for shutting off the electromagnet 452 if it overheats. Together the pot cylinder 456 and the pot base 458 form a housing that partially encapsulates the coil 460 leaving only one end of the coil 460 that faces the fluidic cartridge 500 exposed to contain and magnify the magnetic field emanating from the coil 460.

In one non-limiting example, a Hall effect sensor (not shown) may be positioned adjacent the fluidic cartridge 500—e.g., within the cartridge holder 412 beneath the sample chamber W1—to confirm that an oscillating magnetic field is being generated and that the magnet 426 is moving. In one non-limiting example, the Hall effect sensor will detect two magnetic fields: the oscillating electromagnetic field from the electromagnet 452, having a regular signal, such as a sine wave, and the magnetic field from the magnetic element 626, which may be a larger signal—if the magnetic element 626 within the sample chamber W1 is closer to the sensor than electromagnet 452—and a more chaotic signal due to the chaotic movement of the magnetic element 626. By detecting both magnetic fields, the Hall effect sensor confirms that the electromagnet 452 is working and that the magnetic element 626 is present and is moving.

The example shown in FIG. 57, references components corresponding to the embodiment of FIGS. 49 and 50—i.e., lysis capsule 600. It should be noted, however, that the following description of the interaction between the electromagnet and the magnetic element within the lysis chamber is applicable to any embodiment of a lysis capsule, lysis vessel, or bead delivery cap described herein.

In the context of this disclosure “close proximity” means that electromagnet and the magnetic element are sufficiently close together such that variations in the polarity of the electromagnet result in the desired movement of the magnetic element, wherein a proximity that is considered to be “close” can vary with the strength of the electromagnet, the strength of the magnetic element, the thickness of the sample chamber wall, the thickness of the wall of the magnet housing, and the materials of the magnet housing and the sample chamber. In one non-limiting example, the electromagnet 452 is spaced about 1.5 mm from the sample chamber W1 and about 7 mm from a surface of the magnetic element 626. The diameter of the sample chamber W1 is about 13 mm, and the wall of the sample chamber is about 0.5 mm thick.

Electromagnet 452 may comprise at least one individual electromagnet driven, e.g., by a switching amplifier. The frequency of the electromagnet 452 and the oscillating circuit 454 (i.e., the frequency with which the electromagnet reverses its polarity) may be in the range of 60 to 200 Hz or the range of 20 to 120 Hz, and a drive voltage of the oscillating circuit may be in the range of 10-50 V.

### Protective Venting Cover

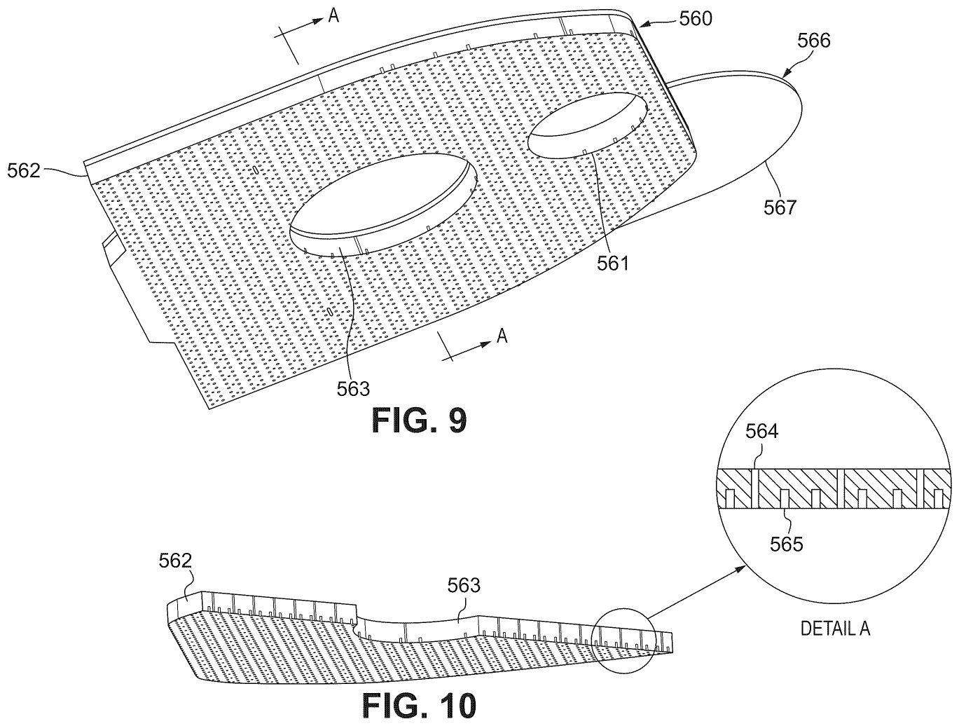

Referring to FIGS. 9 and 10, including Detail A, protective venting cover 560 may include at least two components: a venting membrane 562 that is hermetically sealed to the top of the cartridge body 502 to cover the chambers W1 to W12 of the sample preparation section 504 and a protective cover 566 heat laminated to a top surface of the venting membrane 562 and peelable from the venting membrane by a user prior to use of the cartridge. A plunger hole 563 formed in at least the venting membrane 562 (and optionally provided in the protective cover 566 as well) is positioned over and provides access to the syringe barrel SB by a syringe plunger. A sample chamber hole 561 is aligned with the sample chamber W1 and is covered by the protective cover 566 until the protective cover 566 is removed to permit access to the sample chamber W1. Protective cover 566 thus provides a removable seal covering the sample chamber W1 until the cartridge is to be used to perform a sample assay.

As shown in FIG. 10 and detail A, venting membrane 562 is a porous plastic membrane with two sets of pores: through pores 564 and blind pores 565. The through pores 564 extend completely through the thickness of the venting membrane, and the blind pores 565 extend from a bottom surface of the venting membrane (the surface in contact with the cartridge body 502) partially through the thickness of the membrane. The venting membrane allows gas/vapor circulation via the through pores and contains liquid within the chambers W1 to W12 when the protective cover 566 is removed. The blind pores 565 enhance adhesion of the membrane 562 to the cartridge body 502 as the plastic of the cartridge body melts into the blind pores 565 when the membrane 562 is attached, e.g., by heat sealing, to the cartridge body 565.

In one non-limiting example, protective cover 566 comprises a three-layer aluminum laminate: polyester (PET)/aluminum/polyethylene (PE), and is heat laminated to the top (exposed) surface of the venting membrane 562. The protective cover 566 may include a pull tab 567 extending beyond the venting membrane 562 to allow the user to grasp and peel the cover 566 from the membrane. The PE layer of the protective cover melts during a heat lamination process and partly flows into the venting membrane through pores 564 to limit or prevent evaporation of the liquids stored in one or more of the chambers W1 to W12 of the fluidic cartridge 500 while the protective cover 566 is in place during manufacturing, storage, and transportation of the cartridge. When the protective cover 566 is peeled from the venting membrane 562 prior to use of the cartridge 500, the through pores 564 of the venting membrane 562 are freed from that PE, and all PE “hairs” which were clogging the through pores 564 are removed and remain attached to the aluminum laminate of the protective cover 566. In one embodiment, protective venting cover 560 does not cover chamber W1 (the sample chamber) and may have an opening formed at the location of chamber W1 so as to permit access to the sample chamber when the protective venting cover is attached to the cartridge.

### Cartridge Syringe

Fluidic cartridge 500 includes a pump mechanism configured to be engaged by an actuating component of the instrument for moving fluids between the wells and chambers and through the grooves/channels and through-holes. In embodiment illustrated in FIG. 3, the pump mechanism comprises a syringe defined by the elastomeric syringe stopper 540 disposed within the syringe barrel SB and actuated by the syringe plunger 362 of the instrument 10, as described below. Raising the stopper 540 within the syringe barrel SB creates a vacuum within the syringe barrel SB that pulls fluid through the channels G1 to G10 and the holes H1c to H10c and into the syringe barrel SB. Valves V1 to V10 can be actuated to control which of channel(s) G1 to G10 is (are) open to the syringe barrel SB. Typically, all but one valve V1 to V10 would be closed so that fluid is drawn into the syringe barrel SB from one of the chambers W1 to W10 through one of the channels G1 to G10 and holes H1c to H10c.

Lowering the stopper 540 within the syringe barrel SB creates pressure within the syringe barrel SB that pushes fluid from the syringe barrel SB through the holes H1c to H10c and channels G1 to G10. Again, valves V1 to V10 can be actuated to control which channel(s) is (are) open to the syringe barrel SB. Typically, all but one valve V1 to V10 would be closed so that fluid is pushed from the syringe barrel SB through one of the holes H1c to H10c and associated channels G1 to G10.

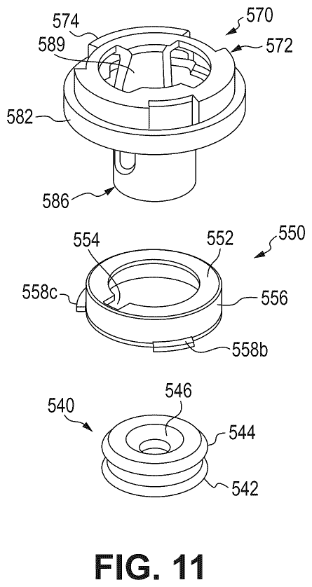

As seen in FIG. 11, stopper 540 is generally cylindrical and has a diameter that forms a sliding fit with a cylindrical wall 508 of the syringe barrel SB. Stopper 540 may include one or more peripheral rings (e.g., rings 542, 544) to promote a sealing contact between the stopper 540 and an inner surface of the cylindrical wall 508.

As shown in FIGS. 18 and 19, stopper 540 includes a plunger recess 546, for receiving plunger head 364 at the end of the syringe plunger 362 (see FIG. 20), and a plunger pocket 548 for releasably retaining the plunger head 364 of the syringe plunger 362, as will be described below. Plunger recess 546 may include a conical (chamfered) portion to help guide the plunger head 364 of the plunger into the plunger pocket 548.

During shipping and storage of the cartridge 500, and before the stopper 540 is engaged by a plunger 362, the stopper 540 is retained within the syringe barrel SB and pressed against a bottom wall of the syringe barrel SB by a blocker mechanism thereby blocking the holes H1c to H10c. As shown in FIG. 11, a blocker mechanism may comprise the blocker ring 550, secured to a top edge of the cylindrical wall 508 of the syringe barrel SB, and the blocker 570 is configured to be coupled to the blocker ring 550 and to be uncoupled from the blocker ring 550 when engaged by the syringe plunger 362 moving down through the blocker 570 and into engagement with the stopper 540, as will be described below.

Blocker ring 550 includes an annular rim 552 and an axial ring 556 circumscribing the outer periphery of the annular rim 552. A bottom side of the annular rim 552 contacts the top circular edge of the cylindrical wall 508 of the syringe barrel SB. An inner diameter of the axial ring 556 is preferably only slightly larger than an outer diameter of the cylindrical wall 508 so that there is little lateral play between the blocker ring 550 and the cylindrical wall 508. An inner diameter of the annular rim 552 is preferably smaller than an inner diameter of the cylindrical wall 508 (and smaller than the diameter of the stopper 540) so that the blocker ring 550 prevents the stopper 540 from being removed from the syringe barrel SB. A radial notch 554 is formed across the top of the annular wall 552 to mate with a (not shown) nub in the syringe barrel SB. This ensures proper clocking of the blocker ring 550 with respect to the cartridge body 502 such that subsequent assembly of the blocker mechanism is easily automated and also properly aligned with the syringe plunger in the instrument.

Blocker ring 550 includes three angularly-spaced, radially extending flanges, or tabs, 558a, 558b, 558c projecting outwardly from a bottom edge of the axial ring 556 (tab 558a is labeled in FIG. 18).

The blocker ring 550 is fixed to the top of the cylindrical wall 508, e.g., by an adhesive or thermal or ultrasonic welding, or the blocker ring 550 and the cylindrical wall 508 can be integrally formed as a single piece.

As shown in FIGS. 11-14, blocker 570 includes a cap portion 572 and a center tube 586. Cap portion 572 includes a top, first cap portion 574 and a lower, second cap portion 582 that is coaxial with and has a larger outer diameter than the first cap portion 574. First cap portion 574 is defined by a top, radially-oriented wall 576 and a side, axially-oriented wall 575. Side wall 575 has an inner diameter that is slightly larger than an outer diameter of the axial ring 556 of the blocker ring 550 so that the first cap portion 574 of blocker 570 fits over the blocker ring 550 and there is little lateral play between the first cap portion 574 of blocker 570 and the blocker ring 550. Second cap portion 582 is defined by a side, axial wall 583 having an inner diameter that is larger than an outer diameter of a circle circumscribing the outer edges of the flanges 558a, 558b, 558c of the blocker ring 550 so that the second cap portion 582 of the blocker 570 fits over and past the flanges 558a, 558b, 558c of the blocker ring 550.

Blocker 570 includes three angularly-spaced flanges 584a, 584b, 584c, projecting inwardly from a lower edge of the axial wall 583 of the second cap portion 582 of the cap portion 572. A distance between a top surface of each radial flange 584a, 584b, 584c and a bottom surface of the radial wall 576 of the first cap portion 574 is at least as great as the distance between a bottom surface of each flange 558a, 558b, 558c of the blocker ring 550 and a top surface of the annular rim 552 of the blocker ring 550. Accordingly, when the blocker 570 is placed on the blocker ring 550 with the top surface of the annular rim 552 of the blocker ring 550 contacting the bottom surface of the radial wall 576 of the blocker 570, the blocker 570 can be rotated with respect to the blocker ring 550 to place each of the flanges 584a, 584b, 584c of the blocker 570 beneath a corresponding one of the flanges 558a, 558b, 558c of the blocker ring 550, thereby releasably interlocking the blocker 570 and the blocker ring 550.

Center tube 586 extends below the top wall 576 of the first cap portion 574 of cap portion 572. The length of the center tube 586 is greater than a distance from the top of the stopper 540 to the top wall of the annular rim 552 of the blocker ring 550 when the stopper is in contact with the bottom wall of the syringe barrel SB. Accordingly, the center tube 586 must be pushed down to partially compress the stopper 540 to enable the bottom surface of the top wall 576 of the first cap portion 574 to contact the top of the annular rim 552 of the blocker ring 550. This compression of the stopper provides a seal blocking the through-holes H1c to H10c in the syringe barrel SB. Also, the resilience of the stopper 540 pushes up on the center tube 586, thereby causing the flanges 584a, 584b, 584c of the blocker 570 to push up on the flanges 558a, 558b, 558c of the blocker ring 550, thereby enhancing frictional force between the flanges 584a, 584b, 584c and the flanges 558a, 558b, 558c to retain the blocker 570 in a fixed position with respect to the blocker ring 550. The retained blocker 570 holds the stopper 540 in a compressed state against the bottom wall of the syringe barrel SB.

Top wall 576 of the first cap portion 574 includes a center opening 589. Center tube 586 extends down from the top wall 576 from a perimeter of the center opening 589. Center tube 586 comprises opposed cam walls 588a, 588b extending down from opposed sides of the center opening 589 formed in the top wall 576. Each cam wall 588a, 588b includes an associated cam edge 590a, 590b with a helical curve extending along one side of each cam wall 588a, 588b, respectively, from the top wall 576 to a terminal ring 592 extending continuously around the circumference of a lower end of the center tube 586 (see also FIG. 18).

Radial clearances 577a, 577b are formed on opposite sides of the center opening 589 of the top wall 576 and are disposed between the cam walls 588a, 588b. Thus, a radius 578a from the center of the opening 589 to each cam wall 588a, 588b (which is half the diameter between the opposed walls 588a, 588b and the inner diameter of terminal ring 592) is smaller than a radius 578b from the center of the opening 589 to an outer edge of each clearance 577a, 577b (which is half the diameter between the opposed clearances 577a, 577b.).

First cap portion 574 of the cap portion 572 of blocker 570 includes angularly-spaced cut outs 580a, 580b, 580c formed in the axially-oriented sidewall 575 to facilitate molding of internal features, such as the flanges 584a, 584b, 584c.

### Expandible Cartridge

In some applications, it may be desirable to expand a volumetric capacity of one or more chambers of a fluidic cartridge to accommodate a larger volume of fluid in the expanded chamber. For example, it may be desirable to expand the volumetric capacity of the sample chamber W1 of fluidic cartridge 500 so that a larger volume of sample material can be added to the cartridge to provide more material from which a target analyte may be extracted, thereby improving the sensitivity of a test for detecting the target analyte.

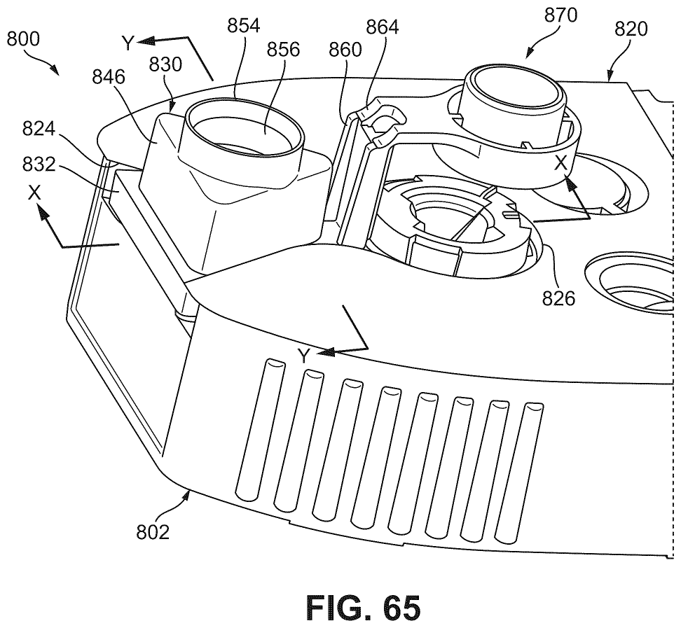

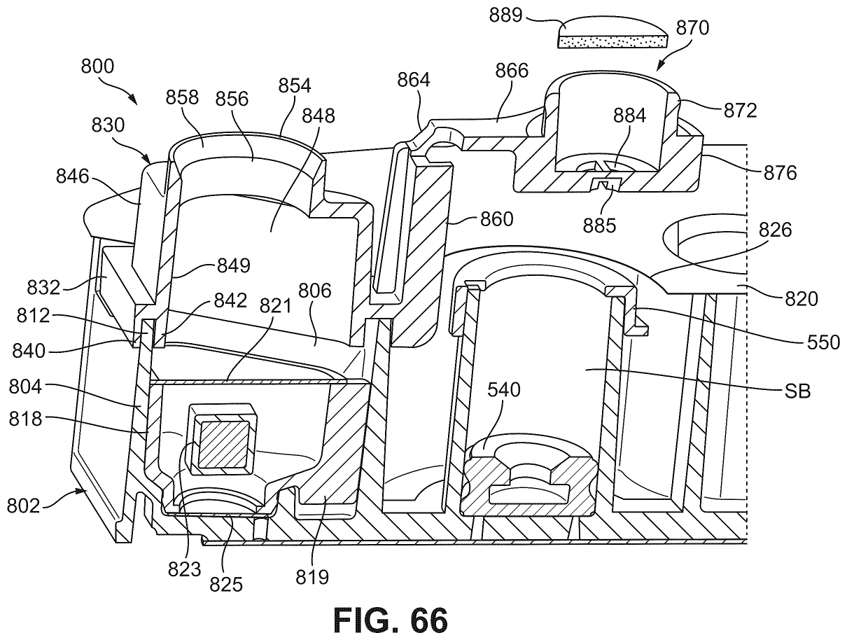

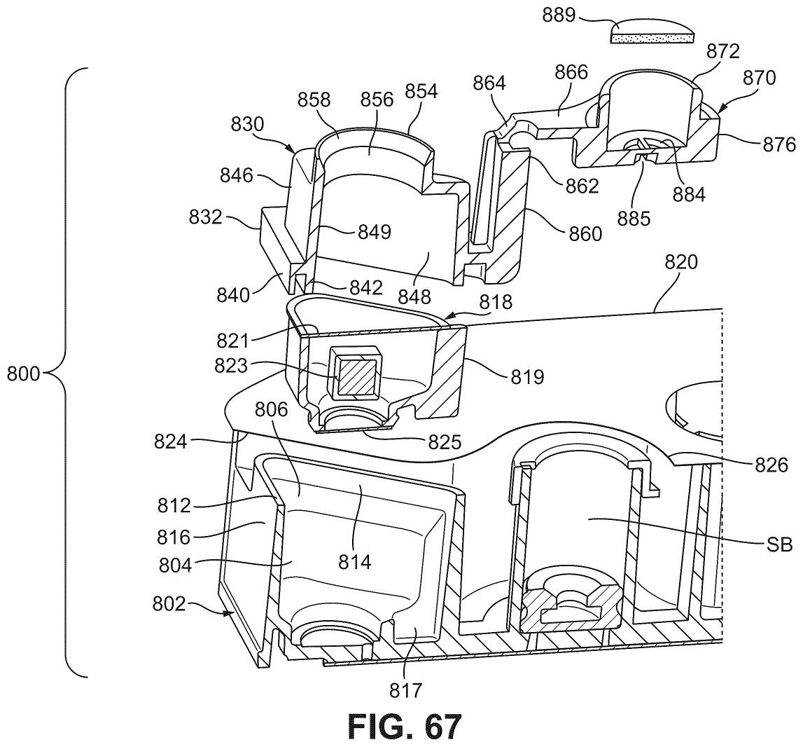

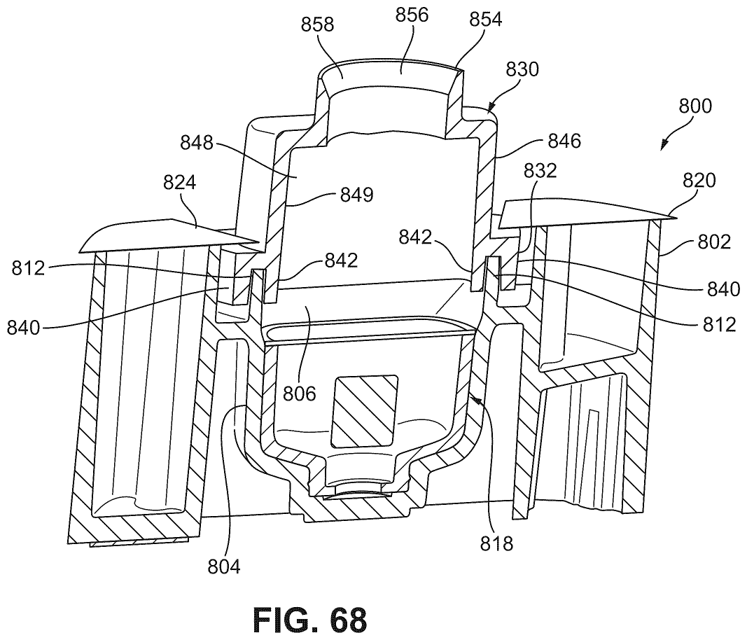

FIG. 65 is a partial top perspective view of an expandable fluidic cartridge 800 with a chamber expander 830, FIG. 66 is a partial cross-sectional view of the fluidic cartridge 800 and chamber expander 830 along the line X-X in FIG. 65, FIG. 67 is an exploded version of the cross-sectional view of FIG. 66, and FIG. 68 is a partial cross-sectional view of the fluidic cartridge 800 and chamber expander 830 along the line Y-Y in FIG. 65.

Cartridge 800 has a cartridge body 802 to which chamber expander 830 is hermetically sealed to expand the volumetric capacity of an expansion well, such as sample chamber 804. In this regard, an “expansion well” is a well—in this case the well of the sample chamber 804—that is configured for attaching a chamber expander 830. Chamber expander 830 is a separate piece from the cartridge body 802 for reasons of manufacturability as it would not be practicable to manufacture the cartridge with such an expanded chamber as a single integrated piece.

Cartridge 800 may include a lysis capsule 818 configured to conform to the sample chamber 804 as described in more detail herein (e.g., lysis capsule 600′ inserted into sample chamber W1 in FIG. 51). Lysis capsule 818 may otherwise be configured as a lysis capsule described herein, such as lysis capsule 600 shown in FIGS. 49-50, and include a hollow body with a first porous membrane 821 attached to a top opening of the hollow body and a second porous membrane 825 attached to a bottom opening of the hollow body and defining a lysis chamber between the membranes 821, 825 within which is contained non-magnetic beads (not shown) and a magnetic element 823.

Cartridge 800 may also include a venting cover 820 having an expander cut-out 824 sized and shaped to permit the chamber expander 830 to project through the cover 820. Cover 820 may otherwise be configured as venting cover 560 described herein and shown in FIGS. 9-10 with a venting membrane having through holes and blind pores, a protective cover sealed to the venting membrane, and a plunger hole 826 to permit access to and operation of a syringe of the cartridge 800 disposed in a syringe barrel SB.

Expansion well 804 (the sample chamber in the illustrated embodiment) may have a generally triangular shape with three straight sides and be configured to receive a lysis capsule 818 having a conforming triangular shape (see also FIG. 74). The triangular shape may be used to maximize the volume of the expansion well 804 and the chamber expander 830, but it otherwise is optional. In other examples, the sample chamber and lysis capsule may have a different shape, such as, circular, square, or rectangular. The lysis capsule is also optional. In other examples, the lysis capsule is omitted from the sample chamber.

Cartridge body 802 may include a first coupling structure configured to be operatively coupled to a second coupling structure (described herein) of the chamber expander 830 to hermetically seal the chamber expander 830 to the cartridge body 802. As shown in FIGS. 66-68, in the illustrated embodiment, the first coupling structure comprises a first peripheral wall 812 at least partially surrounding the expansion well 804 and having an inner surface 814 and an outer surface 816. First peripheral wall 812 may comprise a continuous, triangular wall with straight sides (and, optionally, rounded corners), which may be parallel to corresponding straight sides of the expansion well 804. As with the sides of the expansion well 804, the triangular shape of the first peripheral wall 812 is optional, and first peripheral wall 812 may have a circular, rectangular, or other shape.

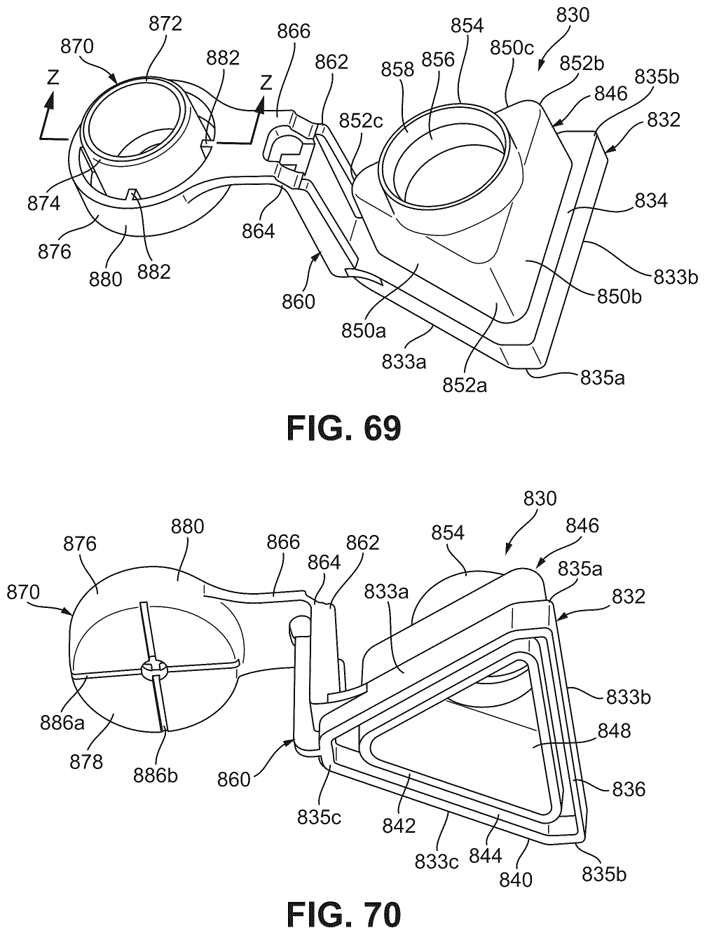

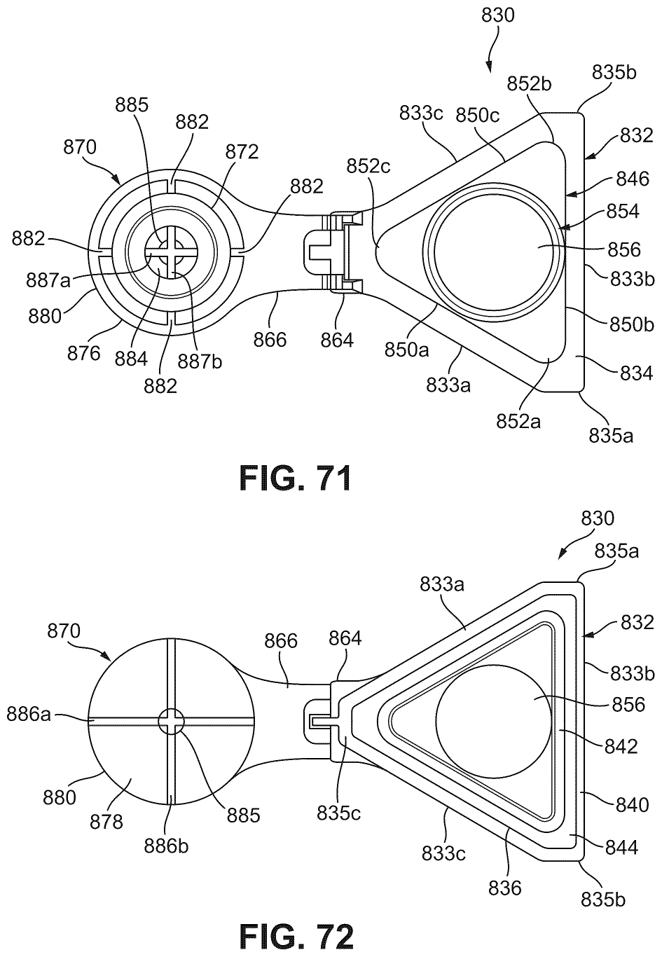

Referring to FIGS. 65-72, chamber expander 830 includes a base 832, an expansion chamber 846 extending above the base 832, a mouth 854 at the top of the expansion chamber 846 and defining an expansion chamber opening 856, and a cap 870 (not shown in FIG. 68) for closing the opening 856 attached to a top end (free end) 862 of a stanchion 860 extending up from the base 832 by a hinge 864, which may be a living hinge. Chamber expander 830 may be injection molded from a suitable plastic, such as polypropylene. Base 832 has a top side 834 (FIGS. 69 and 71), a bottom side 836 (FIGS. 70 and 72), and a triangular shape configured to overlap the triangular shape of first peripheral wall 812 surrounding the expansion well 804. The triangular shape of the base 832 may be defined by three straight sides 833a, 833b, 833c (to which the straight sides of first peripheral wall 812, the expansion well 804, and the lysis capsule 818 may be parallel) connected at corners 835a, 835b, 835c.

Chamber expander 830 includes second coupling structure on the bottom side 836 of the base 832 that is configured to be operatively coupled to first coupling structure surrounding expansion well 804. Referring to FIG. 70, in a first embodiment, the second coupling structure comprises a second peripheral wall 840 conforming to outer surface 816 of first peripheral wall 812. In a second embodiment, the second coupling structure comprises a third peripheral wall 842 conforming to inner surface 814 of first peripheral wall 812. In a third embodiment, the second coupling structure comprises both the second peripheral wall 840 (outer peripheral wall) and third peripheral wall 842 (inner peripheral wall) defining a peripheral groove 844 extending about the perimeter of the bottom side 836 of the base 832 and conforming in shape to the first peripheral wall 812.

For the first embodiment of the second coupling structure, the second coupling structure is operatively coupled to the first coupling structure by affixing second peripheral wall 840 to first peripheral wall 812. Second peripheral wall 840 may be affixed to first peripheral wall 812 by affixing (e.g., by adhesive or laser or ultrasonic welding) outer surface 816 of first peripheral wall 812 (FIGS. 66 and 72) to a facing surface (inner surface) of the second peripheral wall 840.

For the second embodiment of the second coupling structure, the second coupling structure is operatively coupled to the first coupling structure by affixing third peripheral wall 842 to first peripheral wall 812. Third peripheral wall 842 may be affixed to first peripheral wall 812 by affixing (e.g., by adhesive or laser or ultrasonic welding) inner surface 814 of first peripheral wall 812 (FIGS. 66 and 72) to a facing surface (outer surface) of the third peripheral wall 842.

For the third embodiment of the second coupling structure, the second coupling structure is operatively coupled to the first coupling structure by inserting first peripheral wall 812 into the peripheral groove 844 and affixing second peripheral wall 840 to outer surface 816 of first peripheral wall 812 (e.g., by adhesive or laser or ultrasonic welding) and/or by affixing third peripheral wall 842 to inner surface 814 of first peripheral wall 812 (e.g., by adhesive or laser or ultrasonic welding).

Referring to FIGS. 67-72, expansion chamber 846 of chamber expander 830 may have a triangular shape defined by three generally vertical, straight walls 850a, 850b, 850c (FIG. 69), which may be parallel to the straight sides 833a, 833b, 833c of the base 832 and which are connected at corners 852a, 852b, 852c. Expansion chamber 846 defines an interior space 848, and, as shown in FIGS. 66-68, internal surfaces 849 of the interior space 848 may be contiguous with inner surfaces of the third peripheral wall 842.

The width of the interior space 848 of the expansion chamber 846 may be greater than the width of the expansion well 804 (or greater than the width of the capsule 818 within the expansion well 804). Accordingly, as shown in FIGS. 66 and 67, the expansion well 804 may have sloped surfaces 806 surrounding an upper perimeter of the expansion well 804. Sloped surfaces 806 slope inwardly from a bottom edge of third peripheral wall 842 of the chamber expander 830 (also from a base of the first peripheral wall 812 surrounding expansion well 804).

As shown in FIG. 67, expansion well 804 may include a vertically oriented slot 817 that receives a capsule extension 819 extending from the capsule 818, as shown in FIGS. 66, 67, and 74. Extension 819 and slot 817 ensure that the lysis capsule 818 is always installed into the expansion well 804 in the correct orientation, as the capsule extension 819 must be aligned with the slot 817 to allow the lysis capsule 818 to be inserted into the expansion well 804.

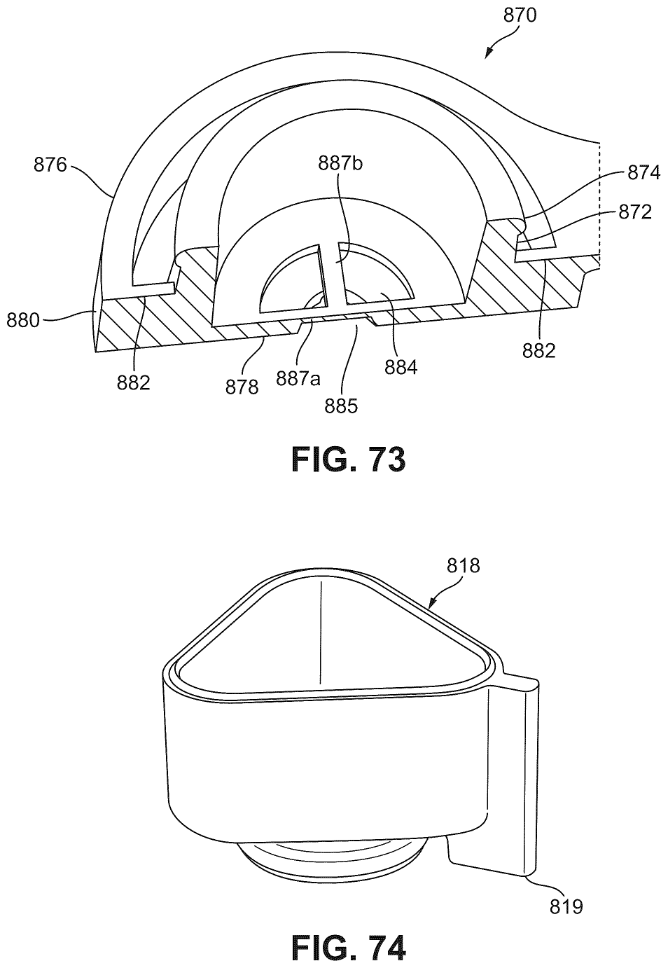

Referring to FIGS. 69, 71, and 73, cap 870 includes an insert sleeve 872 configured to be inserted into opening 856 defined by mouth 854. The insert sleeve 872 and opening 856 are sized so that insert sleeve 872 has a friction fit within the opening 856 of mouth 854. Insert sleeve 872 may include a circumferential rib 874 to enhance a seal between the sleeve 872 and the mouth 856. As shown in FIGS. 65-68, mouth 854 may include a peripheral chamfer (or bevel) 858 surrounding the opening 856 to facilitate insertion of the insert sleeve 872 into the opening 856.

As shown FIGS. 69-73, cap 870 may include an outer shroud 876 defined by a top wall 878 and an axial side wall 880 and may include a tab 866 between the axial wall 880 of the outer shroud 876 and the hinge 864.

The width (e.g., diameter) of the outer shroud 876 may be greater than the width (e.g., diameter) of the insert sleeve 872. Outer shroud 876 may include radial ribs 882 extending between the axial wall 880 of the outer shroud 876 and the insert sleeve 872.

As shown in FIG. 71, cap 870 may include a vent hole formed through the top wall 878. In one non-limiting example, the vent hole includes an inner vent hole portion 884, and, as shown in FIGS. 71 and 72, an outer vent hole portion 885. Cap 870 may include cross ribs 887a, 887b extending across the inner vent hole 884. A porous venting membrane 889 that is impervious to liquid due to intrinsic hydrophobic properties and pervious to gas may be positioned over the inner vent hole 884, within the insert sleeve 872. Membrane 889 may have a pore size of about 0.2 μm. A suitable membrane material is hydrophobic nonwoven polyester (PETE) available from Sterlitech. The membrane may be heat sealed or over molded to a bottom surface of top wall 878. Alternatively, a porous venting membrane be positioned within the outer vent hole portion 885. Cross ribs 887a, 887b prevent the venting membrane from collapsing into the inner vent hole 884. Inner vent hole portion 884 may be larger (e.g., in diameter) than outer vent hole portion 885. The larger inner vent hole 884 allows for more of the venting membrane to be exposed, and the smaller outer vent hole 885 reduces the likelihood of damage to the membrane by intrusion into the vent hole 885.

Cap 870 may include surface venting grooves 886a, 886b formed in the top wall 878 and crossing through the outer vent hole 885.

### Thermal/Detector Mechanism

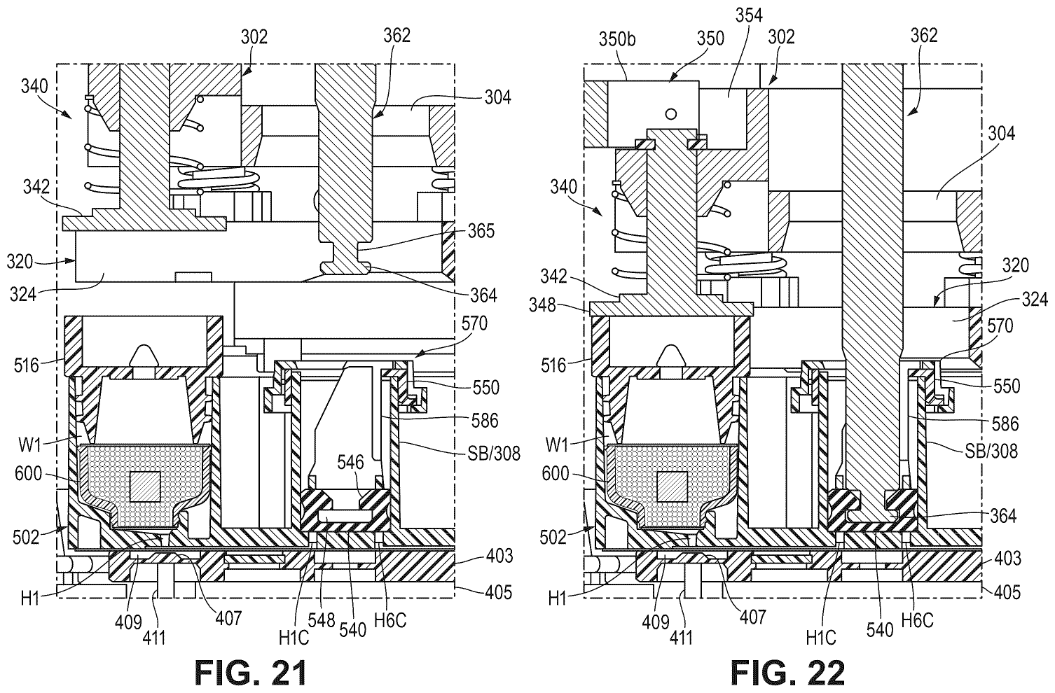

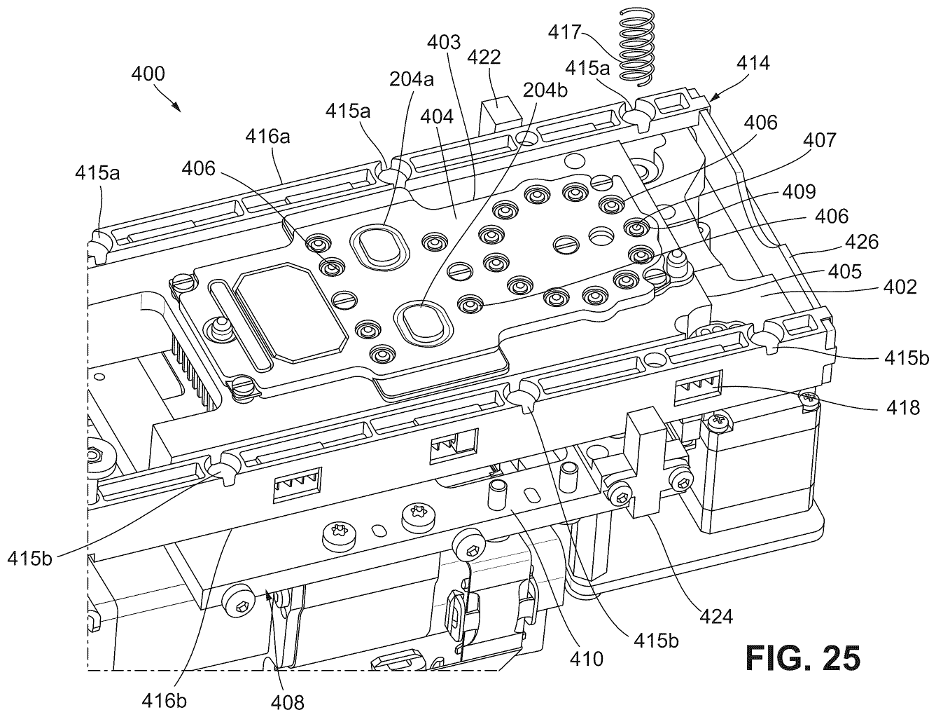

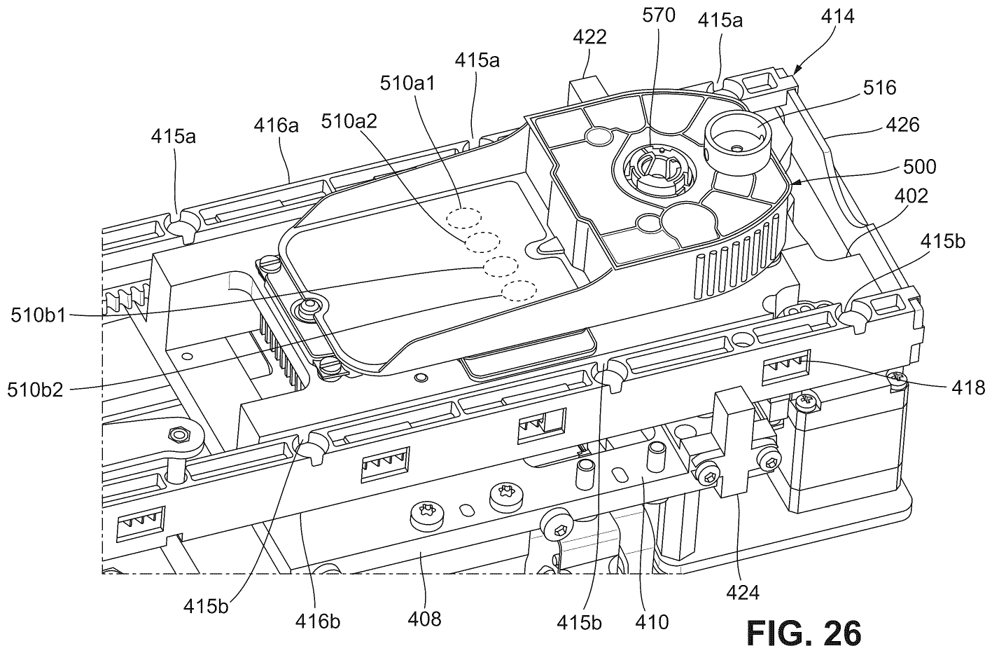

Instrument 10 includes a thermal/detector mechanism that may comprise a component or subsystem of instrument 10 and which operates to heat or cool the reaction/detection chambers 510a1, 510a2, 510b1, 510b2 and to detect optical signals emitted by reactions occurring within reaction/detection chambers 510a1, 510a2, 510b1, 510b2 when the fluidic cartridge 500 is within the instrument 10. FIGS. 25 and 26 are partial, top perspective views of the lower chassis 400 showing a cartridge support frame 402, respectively, with and without a cartridge 500. FIG. 25 shows the cartridge support frame 402 which includes a cartridge support cradle 404 on which a cartridge can be operatively supported, and FIG. 26 shows the cartridge support frame 402 supporting the cartridge 500. Cartridge support cradle 404 may include a gasket 403 made of a resilient material, such as rubber, secured to a platform 405 of the cartridge support frame 402. As shown in FIG. 25, instrument 10 may include a plurality of valve actuator heads 406. There are eighteen actuator heads 406 in the example shown (three of which are labeled in FIG. 25), each being associated with one of the valves V1 to V18 of the cartridge 500. Each actuator head 406 includes a cap 407 within a recess 409 formed in the gasket 403 and is associated with a rod 411 (see FIGS. 21, 22) extending into the corresponding cap 407 and which is actuated by an actuator (not shown) to move the cap 407 between a first position flush or recessed with respect to a top surface of the cartridge support cradle 404 (i.e., top surface of gasket 403) and a second position protruding above the top surface of the cartridge support cradle 404. When in the second, protruding position, a valve actuator head 406 associated with each valve V1 to V18 of fluidic cartridge 500 (see FIG. 7) selectively closes the associated valve by pressing the deformable bottom film 530 of the cartridge into contact with the valve seat of the valve. The number of valve actuators, the operation of the valve actuators, or the manner in which the valve actuators engage valves within the fluidic cartridge 500 are not critical to this disclosure and will not be described in detail herein. U.S. Pat. No. 10,654,039 describes examples of valve actuators that may be employed in the instrument 10 for moving rods 411 between their first and second positions. International Application No. PCT/US2025/026844, entitled “Fluidic Cartridge and Apparatuses for Processing Fluidic Cartridges,” filed Apr. 29, 2025 describes other valve actuators that may be employed in the instrument 10 for moving each of rods 411 between its first and second positions.

The cartridge support cradle 404 is supported on, attached to, or an integral part of cartridge support frame 402 of the lower chassis 400, and cartridge support frame 402 is supported on, attached to, or an integral part of a base plate 408.

Instrument 10 includes a movable holder that supports a test platform, such as a cartridge 500, and which may be selectively moved laterally with respect to the rest of the instrument between a position at which the holder is extended from the instrument 10 so that a cartridge 500, or other test platform, may be placed into or removed from the holder and a position retracted into the instrument to move a fluidic cartridge 500 supported on the holder to an operative position within the instrument in which the cartridge, or a portion thereof, is positioned between first and second heaters, as will be described below. As shown in FIG. 25, a movable frame 414 encompasses the cartridge support frame 402 and the cartridge support cradle 404. Frame 414 comprises rails 416a, 416b held together in a spaced-apart arrangement by a cross piece 426 extending between ends of the rails 416a, 416b. Opposite ends of the rails 416a, 416b, not visible in FIG. 25, are held together in a spaced-apart arrangement by another cross piece 428 (see FIGS. 1 and 2) so that the rails 416a, 416b are generally parallel to one another. The movable frame 414 is movable with respect to the cartridge support frame 402, cartridge support cradle 404, and the base plate 408 from the retracted position shown in FIG. 25 to an extended position to the right of the position shown in FIG. 25. Instrument 10 includes an actuator for effecting automated—e.g., motorized—movement of the frame 414 relative to the cartridge support frame 402 and cartridge support cradle 404. In one non-limiting example, rail 416b includes a rack 418, and a motor (not shown) includes a drive shaft and gear (not shown) engaged with the rack 418 to effect powered movement of the frame 414 between the extended and retracted positions as the motor rotates the drive shaft and gear in one direction or the other.

Referring to FIGS. 1 and 2, cartridge holder 412 is supported on the frame 414 and moves laterally with the frame 414 between the extended and retracted positions. Fluidic cartridge 500 is supported within cartridge holder 412 on short lateral side flanges that extend beneath the fluidic cartridge 500 along opposite sides of the cartridge and that will not overlap or otherwise interfere with the cartridge support cradle 404 when the cartridge holder 412 and the frame 414 are in the retracted position to hold the fluidic cartridge 500 above the cartridge support cradle 404. Cartridge holder 412 is supported with respect to the frame 414 by springs 417 (see FIG. 25, only one spring is shown) disposed within recesses 415a, 415b formed in the tops of rails 416a, 416b, respectively. The springs are positioned between the holder 412 and rails 416a, 416b to hold the holder 412 in a raised position above the rails 416a, 416b, so that a fluidic cartridge 500 carried on the cartridge holder 412 can move over the cartridge support cradle 404 without contacting the cartridge support cradle 404 when the frame 414 is moved between the extended and retracted positions. When the frame 414 and the cartridge support holder 412 are retracted to position a fluidic cartridge 500 carried on the holder 412 above the cartridge support cradle 404, and a downward force is applied to the top of the fluidic cartridge 500—as will be described below—the springs between the cartridge holder 412 and rails 416a, 416b will allow the fluidic cartridge 500 and holder 412 to deflect downwardly and place the fluidic cartridge 500 supported by the holder 412 in contact with the cartridge support cradle 404. When the downward force is removed, the spring will again lift the holder 412 and fluidic cartridge 500 above the frame 414 and the cartridge support cradle 404 so that the frame 414, holder 412, and fluidic cartridge 500 are free to move relative cartridge support cradle 404 without contacting the cartridge support cradle 404.

Instrument 10 may further include sensors 422, 424 for detecting when the holder 412 and frame 414 are in the extended or retracted position. In one non-limiting example, each sensor comprises an optical sensor with an optical emitter and an optical receiver. The emitter emits a light beam that is blocked from reaching the receiver by the rail 416a or 416b until the rail 416a or 416b is at a position at which a notch or opening formed in the corresponding rail allows the beam from the sensor emitter to be received by the sensor receiver. For example, as illustrated in FIG. 25, sensor 424 may be a holder extension sensor for which a beam from the sensor emitter is blocked by rail 416b until frame 414 is in the extended position and a notch formed in the rail 416b is aligned with the emitter and receiver of sensor 424 so that the beam from the emitter is received by the receiver. The resulting signal generated by the sensor 424 will then indicate that frame 414 and holder 412 are in the extended position. Similarly, sensor 422 may be a holder retraction sensor for which a beam from the sensor emitter is blocked by rail 416a until frame 414 and holder 412 are in the retracted position and a notch formed in the rail 416a is aligned with the emitter and receiver of sensor 422 so that the beam from the emitter is received by the receiver. The resulting signal generated by the sensor 422 will then indicate that frame 414 and holder 412 are in the retracted position.

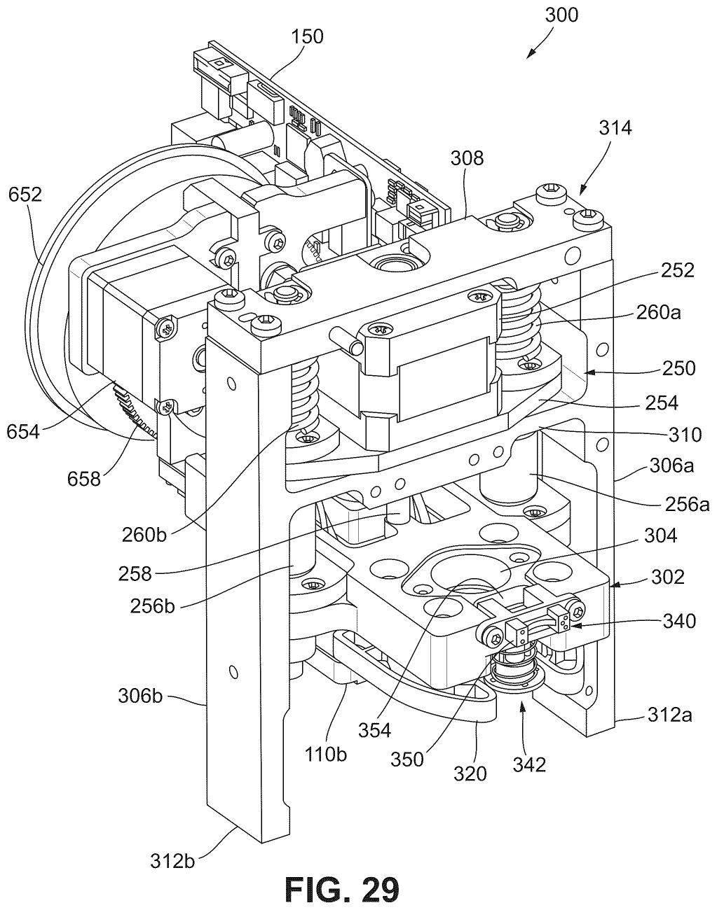

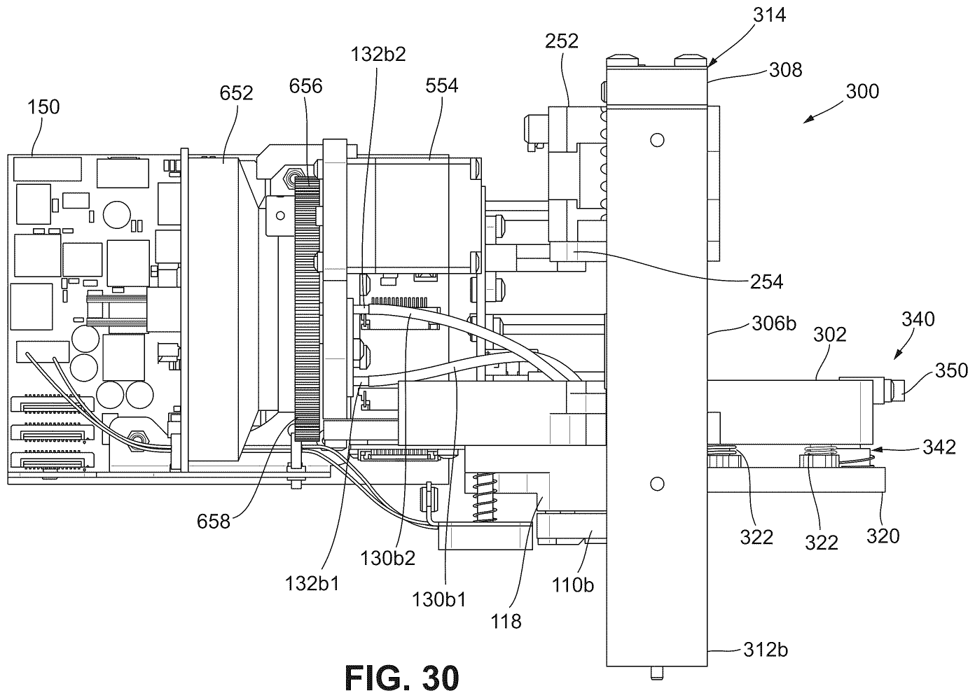

Referring to FIGS. 29-31, upper chassis 300 includes an upper block 302 and a motor mount 314 comprising side supports 306a, 306b, a top crossbar 308 extending between side supports 306a, 306b (but not necessarily between the top ends of the side supports 306a, 306b), and an intermediate crossbar 310 extending between side supports 306a, 306b at a spaced-apart position below the top crossbar 308. Lower ends 312a, 312b of side supports 306a, 306b, respectively, are attached to base plate 408 of the lower chassis 400 at location 410 (see FIGS. 25 and 26). A pressure plate 320 made from, e.g., a molded plastic or similar material (e.g., Delrin), is attached to a bottom side of upper block 302 by means of spring mounts 322 (see FIG. 30). In one non-limiting example, there are four spring mounts 322 between the pressure plate 320 and the upper block 302; two spring mounts 322 are visible in FIG. 30. A spring mount is a connection—e.g., a bolt or a rod—between pressure plate 320 and upper block 302 that creates a gap between pressure plate 320 and upper block 302, and a spring (e.g., a coil compression spring) is disposed within the gap so that the pressure plate 320 and upper block 302 are held apart. Upper block 302 is configured for automated (e.g., motorized) movement with respect to base plate 408 of lower chassis 400, as will be described below, until pressure plate 320 bears against a top portion of the fluidic cartridge 500 supported on the cartridge support cradle 404, e.g., the top portion of the sample preparation section 504 of the fluidic cartridge 500 placed within the instrument 10, and the pressure plate 320 is able to deflect with respect to upper block 302 upon application of sufficient force to overcome the force of the springs of spring mounts 322.

### Syringe Driver

Referring to FIGS. 1, 2, and 20, syringe driver 360 comprises a motor 368, which is preferably a servo motor, operatively coupled to a syringe plunger 362 for effecting axial, up-and-down movement of the syringe plunger 362. In this context, a servo motor is an electromechanical device that produces torque and velocity based on the supplied current and voltage and operated under feedback control and may be a brushless DC motor or any other motor capable of operation under feedback control.

Plunger 362 includes the plunger head 364 defined by a groove 365 circumscribing the syringe plunger 362 above an end of the syringe plunger and configured to engage the plunger recess 546 formed in the stopper 540, and the plunger head 364 seats in the plunger pocket 548 (see FIGS. 18, 19, 21, 22). Syringe plunger 362 further includes laterally-extending plunger ribs, or posts, 366. Referring to FIG. 20, motor 368 is supported on a drive block 380, which may be attached to, or is otherwise fixed with respect to, side supports 306a, 306b and/or intermediate crossbar 310 of the motor mount 314. An encoder 370 (e.g., a rotary encoder) may be a coupled to the motor 368. Motor 368 turns a lead screw 372 coupled to a drive follower 374. Drive follower 374 is mounted to a drive bracket 376 in such a manner as to resist movement or rotation of the follower 374 with respect to the drive bracket 376. An end of the syringe plunger 362 is fixed to the drive bracket 376 (also so as to resist movement and or rotation of the syringe plunger 362 with respect to the drive bracket 376) at an end of the bracket 376 opposite the end at which the drive follower 374 is attached to the bracket 376. Plunger 362 extends through a bushing 382 disposed within the drive block 380. Rotation of the drive screw 372 by the motor 368 causes corresponding up or down movement of the drive follower 374, and the motion of the drive follower 374 is transmitted to the syringe plunger 362 by the drive bracket 376. The bushing 382 prevents binding of the syringe plunger 362 caused by the off-axis application of force to the syringe plunger 362 by the lead screw 372, follower 374, and drive bracket 376.

The syringe driver 360 may further include a sensor for detecting when, or confirming that, the driver 360 has moved the syringe plunger 362 to a specified position (e.g., a “home” position). In the illustrated embodiment, drive bracket 376 includes a home tab 378 extending therefrom, and a home sensor 384 (e.g., a slotted optical detector) is positioned to detect the presence of the home tab 378 when the drive bracket 376 and the syringe plunger 362 are at a home position, which, in the illustrated example, is the top-most position of the syringe plunger 362.

To engage the stopper 540 of a fluidic cartridge 500 positioned below the syringe driver 360, the syringe plunger 362 is lowered by the motor 368 of the syringe driver 360 and passes through a syringe a drive hole 304 formed in the upper block 302. FIG. 21 is a partial longitudinal cross-section of the fluidic cartridge 500 through sample chamber W1 and syringe barrel SB of the cartridge body 502 and through a portion of the instrument 10 and showing syringe plunger 362, pressure plate 320, and upper block 302 in raised positions with respect to the cartridge 500. As the syringe plunger 362 descends, the lower end of the plunger enters into the blocker 570. The outer diameter of the syringe plunger 362 is smaller than the inner diameter of the center tube 586 of the blocker 570, thereby enabling the syringe plunger 362 to descend into the center tube 586. The width of the syringe plunger 362 at the plunger ribs 366 is greater than the inner diameter of the center tube 586 of the blocker 570. Radial clearances 577a, 577b of blocker 570 allow the plunger ribs 366 to pass into the stopper as the syringe plunger 362 continues to descend into the center tube 586, and the plunger ribs 366 engage the cam edges 590a, 590b of the cam walls 588a, 588b, respectively. Due to the helical curvature of the cam edges 590a, 590b, the descending plunger ribs 366 engaging the cam edges 590a, 590b causes the blocker 570 to rotate with respect to the blocker ring 550. Rotation of the blocker 570 moves the flanges 584a, 584b, 584c of the blocker 570 out of overlapping engagement with the flanges 558a, 558b, 558c of the blocker ring 550, thereby releasing the blocker 570 from the blocker ring 550.

FIG. 22 is the same partial longitudinal cross-section as FIG. 21 showing syringe plunger 362 lowered into the stopper 540. The plunger head 364 is received within the plunger pocket 548 of the stopper 540, and, with the blocker 570 released from the blocker ring 550, the syringe driver 360 is able to move the stopper 540 up and down within the syringe barrel SB via the syringe plunger 362. To ensure that the plunger head 364 is received within the plunger pocket 548 of the stopper 540, motor 368 may be operated to lower the syringe plunger 362 until motor stall. With the blocker 570 released from the blocker ring 550, and the stopper 540 attached to the end of the syringe plunger 362, the blocker 570 is held onto the end of the syringe plunger 362 by the stopper 540 and moves up and down with the syringe plunger 362 and stopper 540. When the stopper 540 is first raised from the bottom of the syringe barrel SB after connecting stopper 540 to the syringe plunger 362, one of the valves V1 to V10 between one of the through holes H1c to H10c and an empty one of the chambers W1 to W10 may be opened to vent the system and avoid generating a vacuum within the syringe barrel SB as the stopper 540 is raised.

Syringe driver 360, via plunger 362 engaged with the elastomeric stopper 540, moves the stopper 540 up within the syringe barrel SB to create a vacuum to draw fluids from other chambers of the cartridge into the syringe barrel SB or moves the stopper 540 down within the syringe barrel SB to create pressure to move fluids from the syringe barrel SB to other chambers or reaction chambers of the cartridge. The volume of fluid that is drawn into the syringe barrel SB when the stopper is raised corresponds to the volume of space between the bottom of the syringe barrel SB and the bottom of the stopper, which in turn corresponds to the distance the stopper is raised above the bottom of the barrel. When the syringe plunger and syringe stopper are moved down to the bottom of the syringe barrel, the elastomeric stopper will compress to some extent, which is desired to ensure that most or all fluid is expelled from the syringe barrel SB. Accordingly, when the plunger 362 is reversed to raise the stopper 540, some amount of that upward movement results in the uncompressing (rebound) of the stopper 540 without actually raising the stopper above the bottom of the syringe barrel SB. It is unknown how much compression the stopper has been subjected to when it is pressed against the bottom of the barrel. Some amount of rebound in the stopper is expected when the plunger is retracted, but the exact amount may not be precisely known, and may vary from instrument to instrument and cartridge to cartridge (e.g., from stopper to stopper). Accordingly, precise control of the amount the stopper is raised above the bottom of the syringe barrel SB is a challenge. In addition, variations in the thicknesses of the cartridge and stopper, possible bowing in the cartridge, and other manufacturing and mechanical tolerances can affect the precision of the movement of the stopper, and thus the precision of the volume drawn into the syringe barrel SB by the syringe.

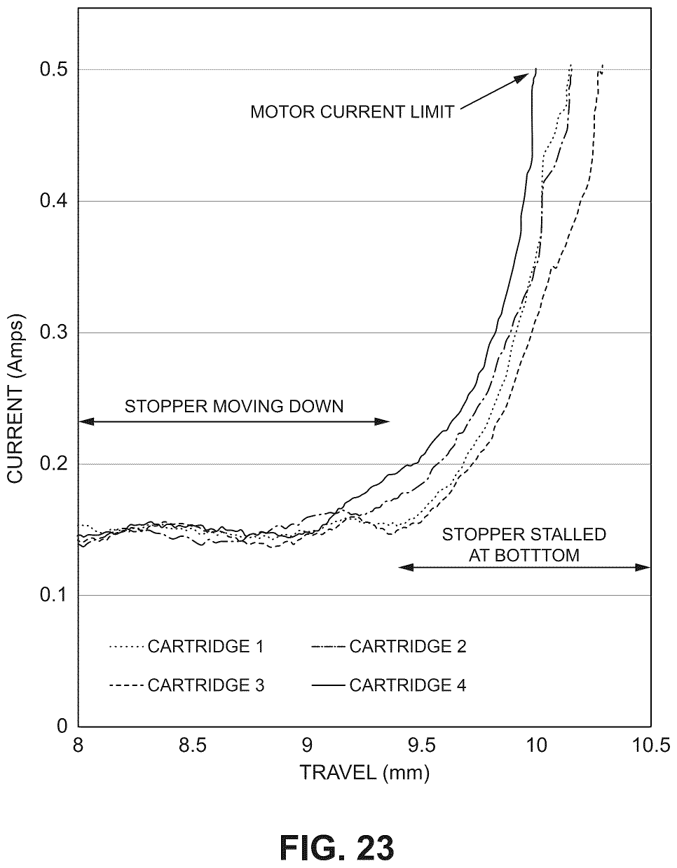

To address these challenges, motor 368 is a motor, such as a servo motor, for which electrical current (amps) drawn by the motor is proportional to resistance encountered (or force/torque generated) by the motor. FIG. 23 is a plot of motor current demand versus stopper travel for four different fluidic cartridges. Motor voltage (volts) and/or motor power demand (watts) and/or any motor operational parameter that is directly or indirectly proportional to motor output, such as resistance or torque, can be monitored instead of or in addition to motor current demand. As the stoppers move from 8.0 to 9.1 mm, current drawn by the motor is a relatively constant level between 0.14 and 0.16 amps. But after about 9.1 mm to 9.4 mm of stopper travel (depending on the cartridge), the motor current demand curve for each cartridge experiences a steep increase. The initiation of the steep rise (or inflection) of each curve represents the point of travel at which the stopper 540 contacts the bottom of the syringe barrel SB. Current to the motor will increase along this steep portion of the curve until the motor current demand limit (or motor current limit) is reached (0.5 amps in the illustration), indicating that the motor has stalled at a travel of 10.1 mm to 10.3 mm and there is no further downward movement or compression of the stopper. Motor stall can also be detected by the encoder 570 detecting no further movement (rotation) by the motor 368. The encoder 370 counts a number of steps before motor stall to track the amount of movement (e.g., rotation) of the motor 368 between the inflection point (i.e., initiation of the steep portion of the motor current curve) and motor stall (motor current limit reached). When the syringe plunger is withdrawn, the syringe plunger 362 is moved by motor 368 of syringe driver 360 by the same number of steps to uncompress the stopper and position that plunger at the position at which the motor current inflection occurred—i.e., the point at which the stopper first contacts the bottom of the syringe barrel SB. That is, the reverse operation of the motor by the number of steps between the inflection point and motor stall is assumed to not raise the stopper 540 above the floor of the syringe barrel SB Next, the motor 368 can be operated for a specified number of encoder steps to move stopper to a specified position above the bottom of the syringe barrel.

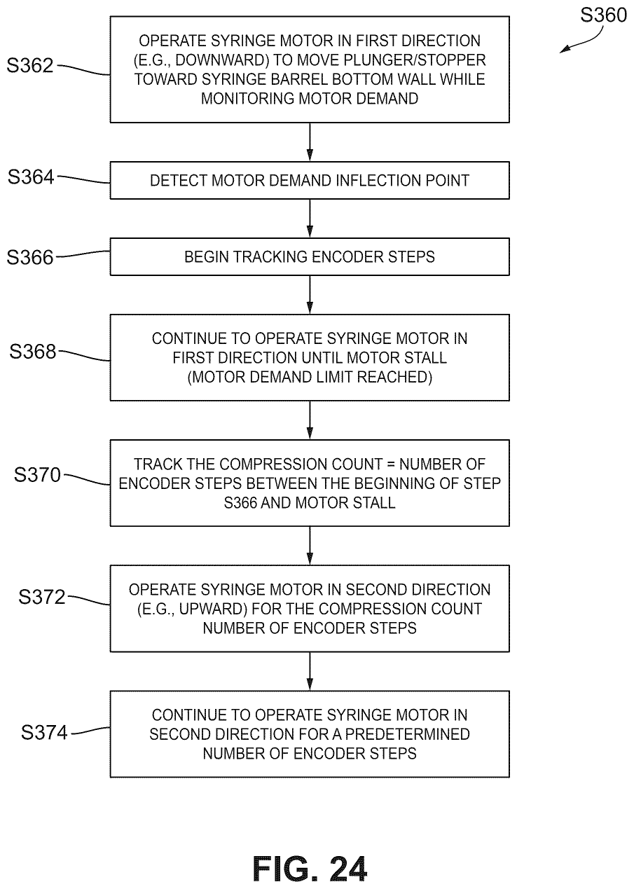

FIG. 24 shows a flow diagram illustrating a method S360 for using the demand (e.g., current drawn) of the motor 368 and the output of the encoder 370 to control the position of the stopper 540 and thus the volume of fluid drawn into the syringe barrel SB. Method S360 may be performed with or used in conjunction with a controller comprising any of the computer systems, devices, mechanisms, elements, or components disclosed herein, among other devices. Method S360 may be coded and stored as a computer-executable control algorithm for controlling the operation(s) of one or more of the computer systems, devices, mechanisms, elements, or components disclosed herein, among other devices. In various embodiments, some of the method steps shown may be performed concurrently, in a different order than shown, or may be omitted. Additional method steps may also be performed as desired. Flow begins at step S362.

To lower the stopper 540 to the bottom of the syringe barrel SB, in step S362, the controller operates syringe motor 368 in a first direction (e.g., downward) to move the syringe plunger 362 and the stopper 540 toward the bottom of the syringe barrel SB while monitoring motor demand (e.g., current drawn) by the motor 368.

In step S364, the controller detects an inflection point in the motor demand signal by any known means, such as, by detecting a change in signal magnitude that exceeds a predefined magnitude or by detecting a signal slope (first derivative of signal magnitude) or change in signal slope (second derivative of signal magnitude) that exceeds a predefined threshold. The stopper 540 has now contacted the bottom of the syringe barrel SB. The amount of change in the demand signal that is indicative of an inflection may vary, for example, with the hardness (durometer) of the stopper 540. In some instances, a change of about 10% may indicate an inflection. The amount of change that is defined as a threshold indicating an inflection point may be system-dependent. In addition, the manner of detecting a change in signal may be system dependent. For example, if inflection is detected by a change in magnitude of the motor demand signal by subtracting one motor demand value from an earlier value, the time span between comparisons—e.g., between consecutive demand signals, every other demand signal, every fifth demand signal, etc.—can be system dependent. If inflection is detected by a change in slope of the motor demand calculated by subtracting one motor demand value from an earlier value and dividing the difference by the time span between the first and second values, the time span between the first and second values—e.g., consecutive demand signals, every other demand signal, every fifth demand signal, etc.—can be system dependent.

In step S366, upon detecting a motor demand inflection point in step S364, the controller begins tracking steps of the encoder 370.

In step S368, the controller continues to operate motor 368 in the first direction until controller detects the motor demand limit reached indicating the motor is stalled.

In step S370, the controller records the number of encoder steps between the beginning of step S366 and motor stall. Since operation of the motor during step S368 primarily results in compression of the stopper 540, the number of encoder steps to motor stall will be referred to as the compression count.

To raise the stopper 540 from the bottom of the syringe barrel SB, in step S372, the controller operates motor 368 in a second direction (e.g., upward) for the compression count number of steps of the encoder 370. This raises the syringe plunger 362 back to the position at which the inflection point was detected in step S364 (i.e., the position at which the stopper 540 first contacted the bottom of the syringe barrel SB) to thereby decompress the stopper 540 without actually lifting the stopper 540 above the bottom of the syringe barrel SB.

In step S374, the controller operates motor 368 in the second direction for a predetermined number of steps of the encoder 370. Operating the motor 368 for the predetermined number of steps of the encoder 370 moves the syringe plunger 362 and the stopper 540 to a desired position above the bottom of the syringe barrel SB.

To remove the stopper 540 and the blocker 570 from the end of the syringe plunger 362, the syringe plunger is raised within the syringe barrel SB until the stopper 540 contacts the blocker ring 550. As the diameter of the stopper 540 is larger than the inner diameter of the annular rim 552 of the blocker ring 550, the stopper 540 cannot move past the blocker ring 550 and continued upward movement of the syringe plunger 362 will withdraw the plunger head 364 of the syringe plunger 362 from the plunger pocket 548 of the stopper 540. To facilitate removal of the stopper 540 from the syringe plunger 362, valves V1 to V10 connected to center through holes H1c to H10c within the syringe barrel SB may be closed, thus creating a vacuum within the syringe barrel SB below the stopper 540 as the syringe plunger 362 and stopper 540 are raised within the syringe barrel SB, which may assist in pulling the stopper 540 off the end of the syringe plunger 362. With plunger head 364 withdrawn from the plunger pocket 548, the syringe plunger 362 is raised so that the end of the syringe plunger 362 is withdrawn from the plunger recess 546 of the stopper 540, but preferably without completely raising the syringe plunger 362 above the syringe barrel SB or the stopper ring 550. The syringe plunger 362 is then lowered into the syringe barrel SB where the end of the syringe plunger 362 contacts the stopper 540, and the syringe plunger 362 is further lowered to push the stopper 540 to the bottom of the syringe barrel SB, but without applying enough force to insert the plunger head 364 into the plunger pocket 548 of the stopper 540. The syringe plunger 362 is then withdrawn from the syringe barrel SB, and, with the stopper 540 no longer attached to the end of the syringe plunger 362, the blocker 570 will not be retained on the syringe plunger 362. The blocker 570 will slip off the end of the syringe plunger 362 with the cap portion 572 of the blocker 570 resting on the blocker ring 550 and the center tube 586 of the blocker 570 extending into the syringe barrel SB.

### Thermal Modules

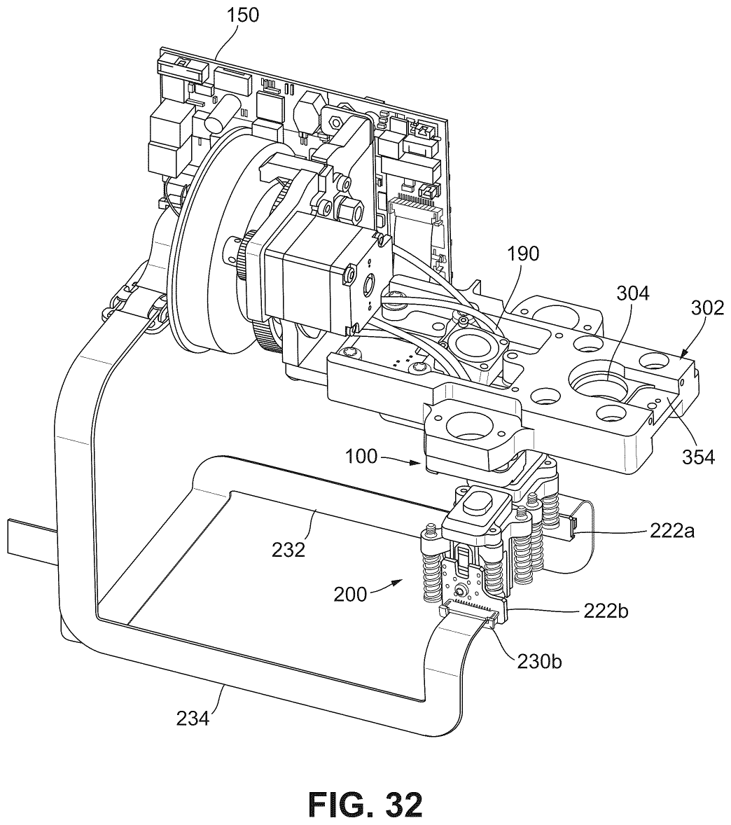

Referring to FIG. 32, which is a partial view of the instrument 10, instrument 10 includes a first thermal module (or first heater) 100 attached to the upper block 302 of the upper chassis and a second thermal module (or second heater) 200 that is part of the lower chassis for applying heat to the reaction/detection chambers 510a1, 510a2, 510b1, 510b2 of the fluidic cartridge 500 that is received between the first and second thermal modules/heaters. In the illustrated embodiment, when the fluidic cartridge 500 is placed in the instrument 10 (i.e., fluidic cartridge 500 is placed on holder 412, and holder 412 is moved to the retracted position), the second thermal module 200 engages the bottom side of the fluidic cartridge 500 at the reaction/detection chambers 510a1, 510a2, 510b1, 510b2, and the first thermal module 100 engages a top side of the fluidic cartridge 500 at the reaction/detection chambers 510a1, 510a2, 510b1, 510b2 when the upper block 302 is lowered with respect to the cartridge holder 412. In the illustrated embodiment, first thermal module 100 is disposed vertically above the second thermal module 200, so thermal modules 100, 200 may be referred to herein as the upper thermal module 100 and lower thermal module 200. Relative positions of the first and second thermal modules 100, 200 are not critical; second thermal module 200 may be located vertically above first thermal module 100, or first and second thermal modules 100, 200 may be located laterally side-by-side.

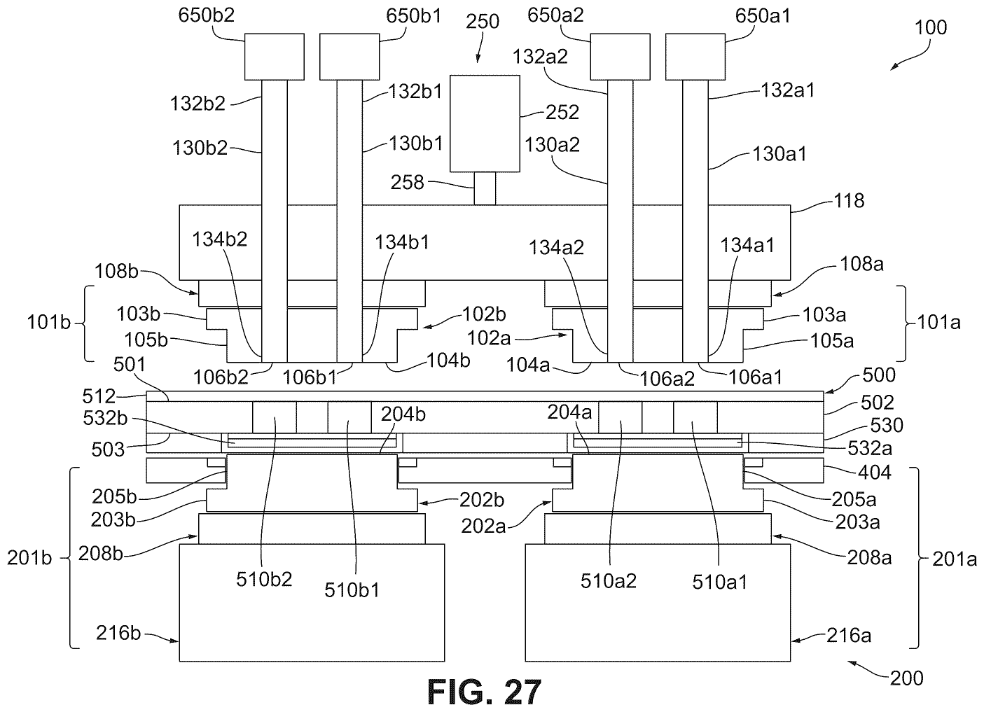

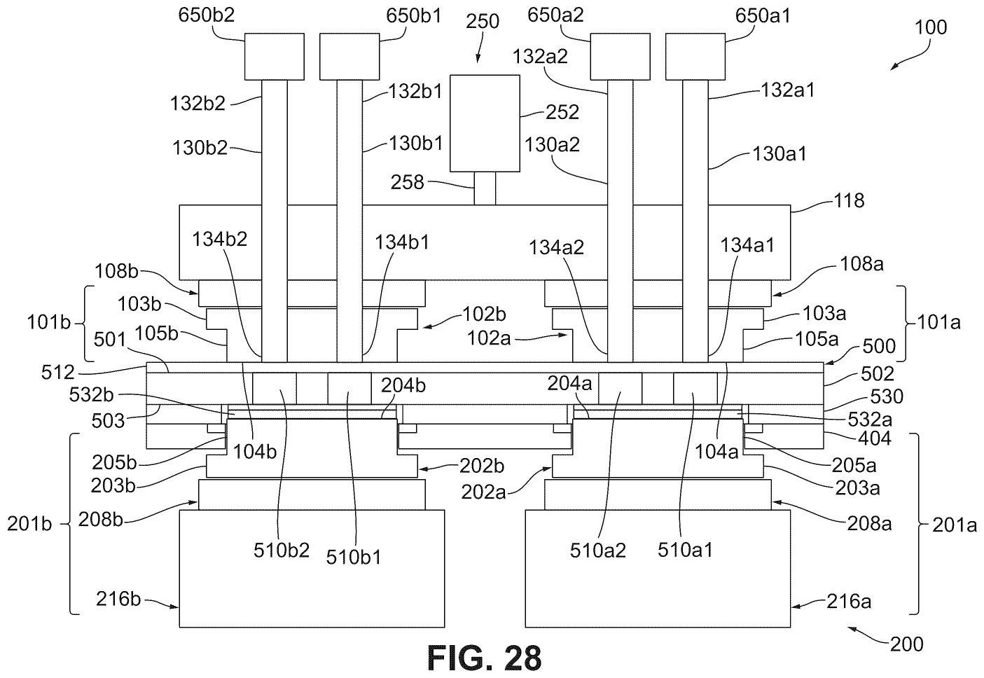

FIGS. 27 and 28 are schematic cross-sections through the first and second thermal modules 100, 200 and through the reaction/detection chambers 510a1, 510a2, 510b1, 510b2 of cartridge 500. To avoid over-cluttering the drawings, cross-sectional lines are omitted from FIGS. 27 and 28. In the illustrated embodiment, fluidic cartridge 500 comprises cartridge body 502 having grooves and/or cavities formed therein as described above with top film 512 affixed to the top face 501 and bottom film 530 affixed to the bottom face 503 of the cartridge body to form channels and reaction chambers of the cartridge 500. In FIGS. 27 and 28, top film 512 and bottom film 530 enclose cavities to the form reaction/detection chambers 510a1, 510a2, 510b1, 510b2. In FIG. 27 the first thermal module 100 is not in contact with the reaction/detection chambers 510a1, 510a2, 510b1, 510b2, and in FIG. 28 the first thermal module 100 is in contact with the reaction/detection chambers 510a1, 510a2, 510b1, 510b2. As will be described below, one or both of the first thermal module 100 and the second thermal module 200 is movable with respect to other so that the first and second thermal modules can be moved into and out of mutual engagement (contact) with the reaction/detection chambers 510a1, 510a2, 510b1, 510b2 of the cartridge 500.

In the illustrated embodiment, first thermal module 100 includes a first thermal assembly 101a and a second thermal assembly 101b that may be independent of the first thermal assembly. Similarly, second thermal module 200 includes a first thermal assembly 201a and a second thermal assembly 201b that may be independent of the first thermal assembly. First thermal assembly 101a of first thermal module 100 is associated with first thermal assembly 201a of second thermal module 200, and together the first thermal assemblies 101a and 201a are associated with reaction/detection chambers 510a1, 510a2 of the cartridge 500. Similarly, second thermal assembly 101b of first thermal module 100 is associated with second thermal assembly 201b of second thermal module 200, and together the second thermal assemblies 101b and 201b are associated with reaction/detection chambers 510b1, 510b2 of the cartridge 500. In the illustrated embodiment, first thermal module 100 includes two thermal assemblies 101a, 101b, and second thermal module 200 includes two thermal assemblies 201a, 201b. First and second thermal modules 100, 200 may include a number of thermal assemblies corresponding to the number of reaction/detection chambers of the cartridge 500, or each thermal assembly may be configured (i.e., sized and shaped) to engage more than one reaction/detection chamber, and thus, the first and second thermal modules 100, 200 may each have more or less than two thermal assemblies, depending on the number of reaction/detection chambers of the cartridge or the configuration of each thermal assembly.

### First Thermal Module

Referring to FIGS. 27 and 28, first thermal assembly 101a of first (upper) thermal module 100 includes a thermal element 108a (which may comprise a thermoelectric module, such as a Peltier device, or any other device, mechanism, or system, other than a light source, that heats, cools, or selectively heats or cools) and an associated thermal block 102a disposed in thermal contact with the thermal element 108a. Thermal block 102a may include a base portion 103a, which is in contact with thermal element 108a, and a projection 105a, which defines an exposed contact surface 104a that contacts the fluidic cartridge 500 at the reaction/detection chambers 510a1, 510a2.

Second thermal assembly 101b of first thermal module 100 includes a thermal element 108b (which may comprise a thermoelectric module, such as a Peltier device, or any other device, mechanism, or system, other than a light source, that heats, cools, or selectively heats or cools) and an associated thermal block 102b disposed in thermal contact with the thermal element 108b. Thermal block 102b may include a base portion 103b, which may be in contact with thermal element 108b, and a projection 105b which defines an exposed contact surface 104b that contacts the fluidic cartridge 500 at the reaction/detection chambers 510b1, 510b2. Thus, in the illustrated example, contact surface 104a contacts a group of chambers including chambers 510a1, 510a2, and contact surface 104b contacts a group of chambers including chambers 510b1, 510b2.

Thermal blocks 102a, 102b are preferably made (e.g., molded and/or machined) from a thermally conductive material, such as a thermally-conductive ceramic or a metal, such as aluminum.

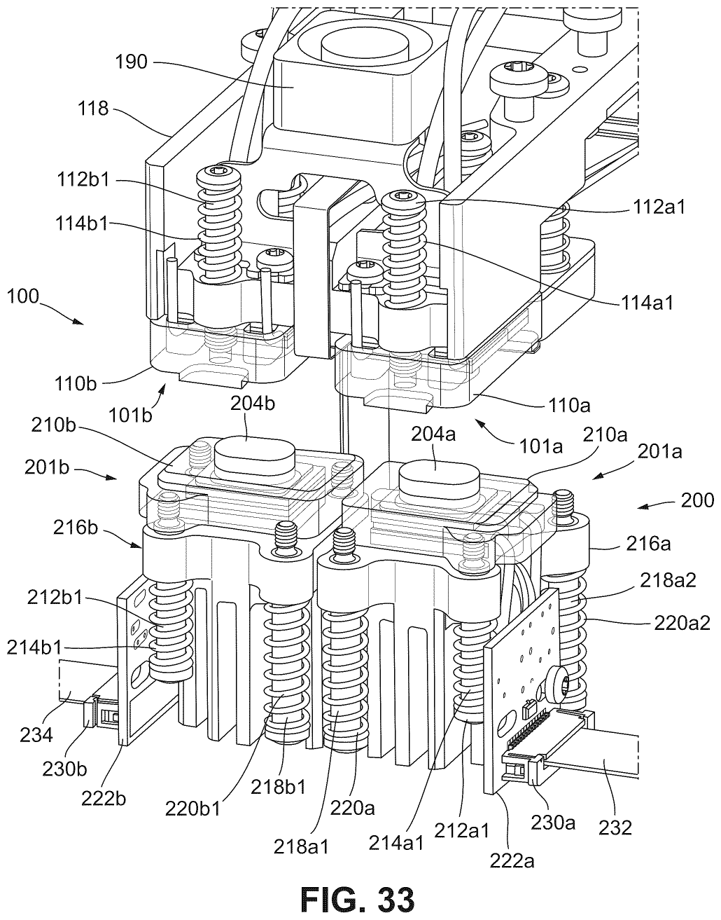

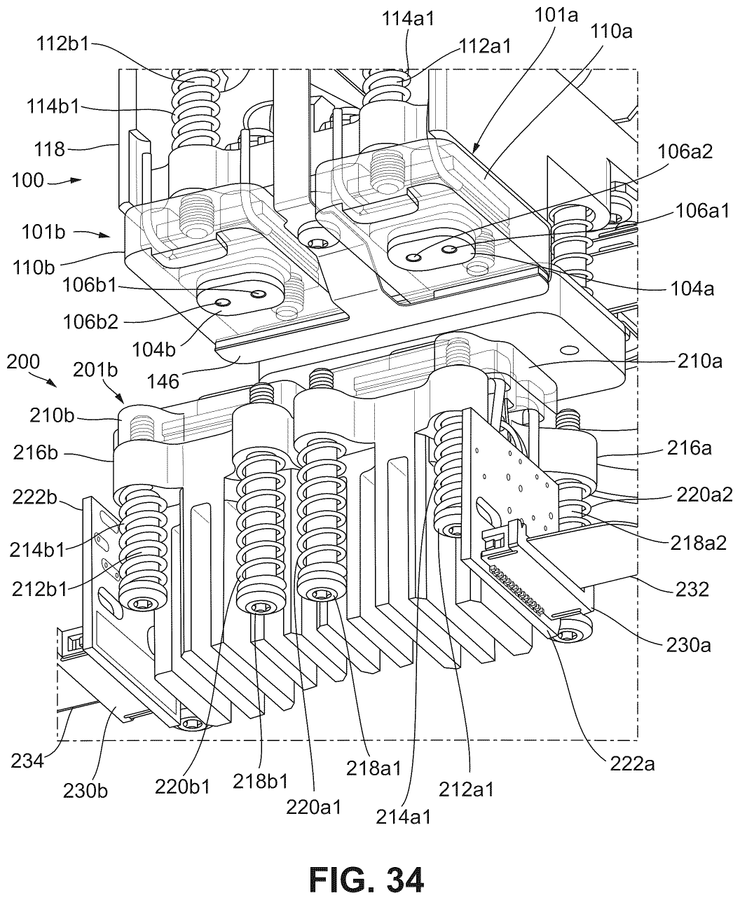

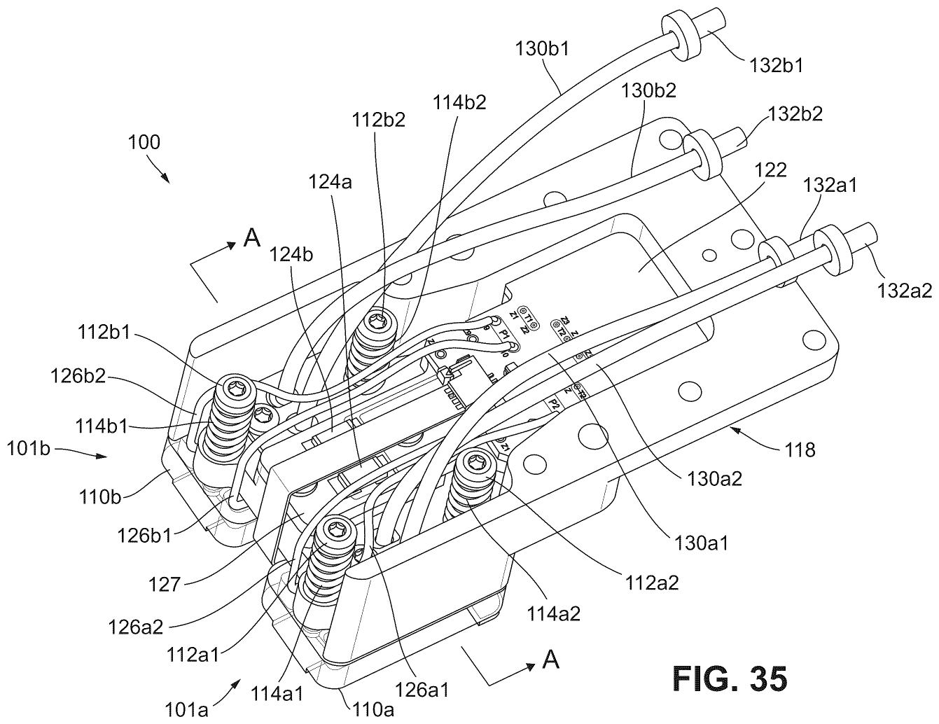

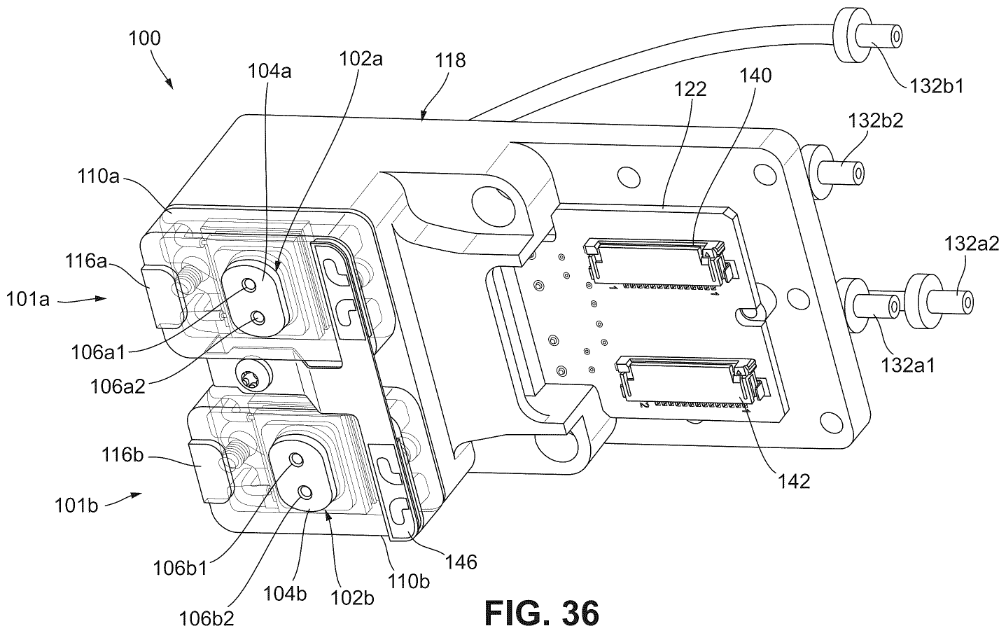

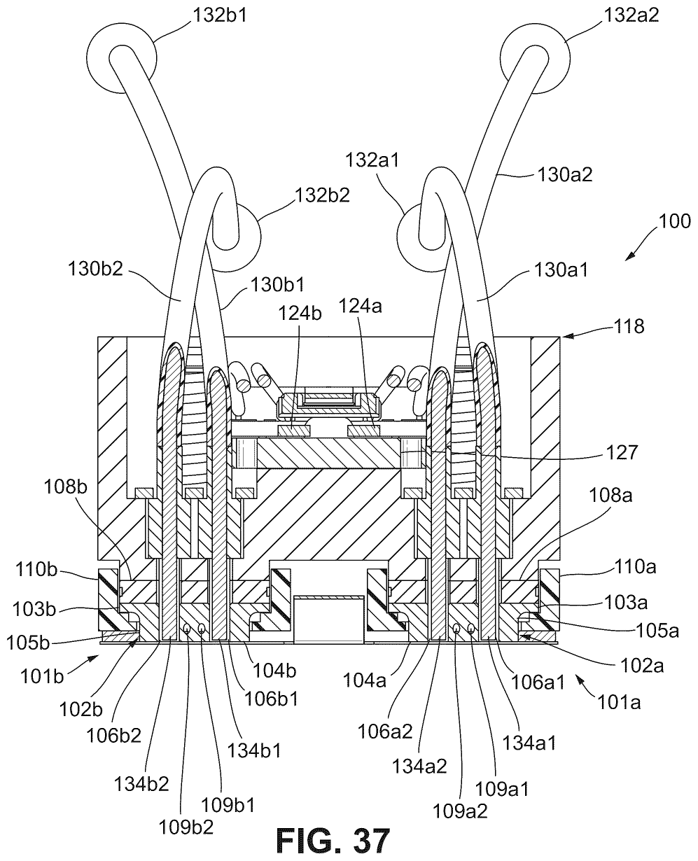

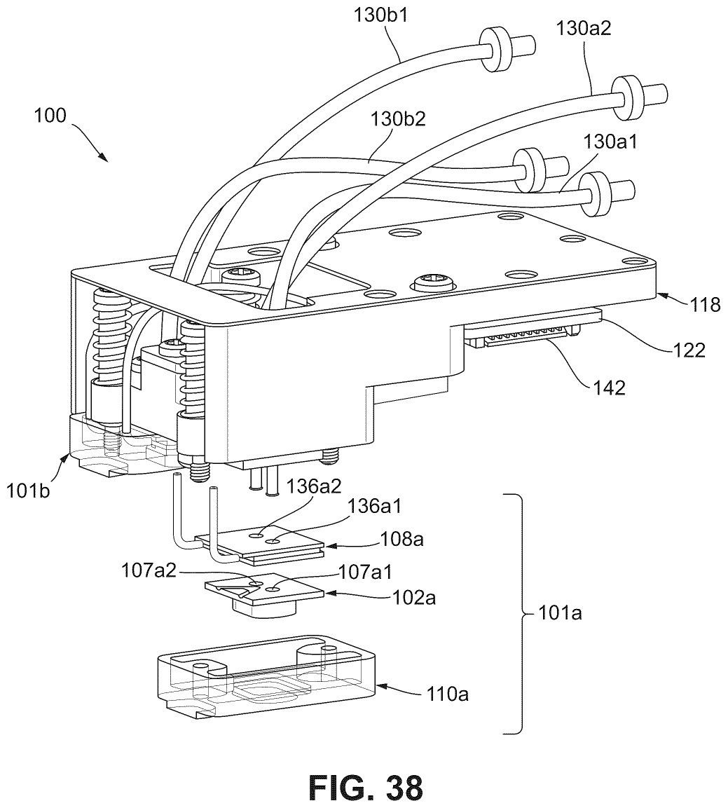

FIG. 33 is a top, partial perspective view of the first thermal module 100 and second thermal module 200, and FIG. 34 is a bottom, partial perspective view of the first thermal module 100 and the second thermal module 200. FIG. 35 is a top perspective view of the first thermal module 100, FIG. 36 is a bottom perspective view of the first thermal module 100, and FIG. 37 is a cross-sectional view of the first thermal module 100 through the line A-A in FIG. 35. FIG. 38 is a perspective view of the first thermal module 100 with first thermal assembly 101a shown in an exploded view.

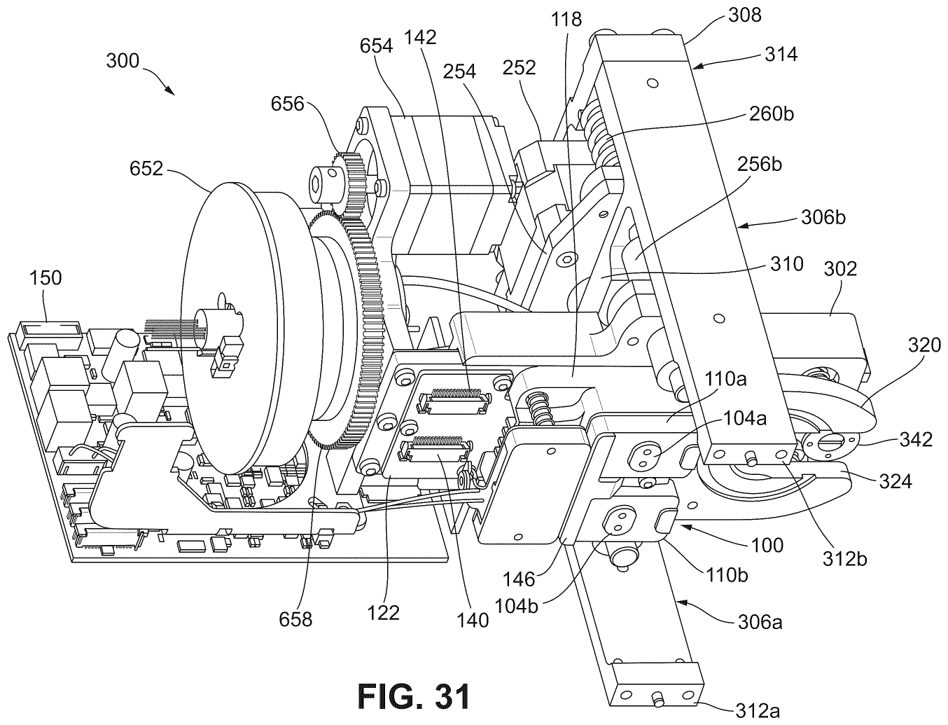

As shown in FIGS. 33, 34, 37, 38, a cover 110a may be positioned over thermal element 108a and associated thermal block 102a. Cover 110a is not shown in FIGS. 27 and 28. Projection 105a of thermal block 102a extends into or through an opening formed in the cover 110a to expose contact surface 104a. Thermal element 108a and associated thermal block 102a may be held in place with respect to mounting block 118 of the first thermal module 100, e.g., by means of fasteners such as cover bolts 112a1, 112a2, extending through holes in the mounting block 118 and threaded into the cover 110a to secure the cover 110a to the mounting block 118. Mounting block 118 is attached to or part of upper block 302 (see, e.g., FIGS. 30 and 31) and is preferably made (e.g., molded and/or machined) from a thermally conductive material, such as a thermally-conductive ceramic or a metal, such as aluminum. As shown in FIGS. 33 and 35, a cover bolt spring 114a1 may be disposed coaxially over cover bolt 112a1 between a head of the bolt 112a1 and the mounting block 118. Similarly, a cover bolt spring 114a2 may be disposed coaxially over cover bolt 112a2 between a head of the bolt 112a2 and the mounting block 118. The purpose of the cover bolt springs 114a1 and 114a2 is to control the force that will be applied to the cover 110a when the cover bolts 112a1, 112a2 are tightened into the mating threads of cover 110a because the cover bolts 112a1, 112a2 are not tightened against the mounting block 118 but are tightened against the cover bolt springs 114a1, 114a2, respectively.

As also shown in FIGS. 33, 35, 37, a cover 110b may be positioned over thermal element 108b and associated thermal block 102b. Cover 110b is not shown in FIGS. 27 and 28. Projection 105b of thermal block 102b extends through an opening formed in the cover 110b to expose contact surface 104b. Thermal element 108b and associated thermal block 102b are held in place with respect to mounting block 118, e.g., by means of fasteners, such as cover bolts 112b1, 112b2 extending through-holes in mounting block 118 and threaded into the cover 110b to secure the cover 110b to mounting block 118. As shown in FIG. 35, cover bolt spring 114b1 may be disposed coaxially over cover bolt 112b1 between a head of the bolt 112b1 and mounting block 118. Similarly, a cover bolt spring 114b2 may be disposed coaxially over cover bolt 112b2 between a head of the bolt 112b2 and mounting block 118. The purpose of the cover bolt springs 114b1 and 114b2 is to control the force that will be applied to the cover 110b when the cover bolts 112b1, 112b2 are tightened into the mating threads of cover 110b because the cover bolts 112b1, 112b2 are not tightened against the mounting block 118 but are tightened against the cover bolt springs 114b1, 114b2, respectively.

As shown in FIG. 35, power lines 126a1, 126a2 connect a connector board 122 to thermal element 108a, and power lines 126b1, 126b2 connect connector board 122 to thermal element 108b. Power lines 126a1, 126a2, 126b1, 126b2 are not shown in FIGS. 27 and 28. Connector board 122 may include one or more connectors (see, e.g., connectors 140, 142 in FIGS. 31 and 36) for connecting connector board 122 to a control board (e.g., printed circuit board or “PCB”) 150 (see FIGS. 1 and 2), e.g., via one or more ribbon cables (not shown in FIGS. 11 and 16) or the like.

At least one of the first thermal module 100 and the second thermal module 200 is configured to permit detection of optical signals emitted by the contents of the reaction/detection chambers 510a1, 510a2, 510b1, 510b2 while the first thermal module 100 and second thermal module 200 are in contact with and applying heat to the reaction/detection chambers 510a1, 510a2, 510b1, 510b2. In one embodiment, as shown in FIGS. 27, 28, 34, 36, 37, two through-holes are formed through the thermal block 102a forming openings 106a1, 106a2 in contact surface 104a of the first thermal assembly 101a of first thermal module 100, and two aligned holes are formed through the thermal element 108a of the first thermal assembly 101a. Optical fibers 130a1, 130a2 are aligned with or extend fully or partially into the through-holes and may terminate at the openings 106a1, 106a2 formed in the contact surface 104a. Optical fiber 130a1 has a proximal end 132a1 and a distal end 134a1, and optical fiber 130a2 has a proximal end 132a2 and a distal end 134a2 (see FIGS. 27 and 28). Distal ends 134a1 and 134a2 are positioned at or proximate to contact surface 104a at openings 106a1, 106a2, respectively (see FIGS. 27, 28, and 37). For example, distal ends 134a1 and 134a2 may be flush with contact surface 104a, may be recessed into the through-holes with respect to the contact surface 104a, or may extend beyond the contact surface 104a.

Similarly, as shown in FIGS. 27, 28, 34, 36, 37, two through-holes are formed through the thermal block 102b forming two openings 106b1, 106b2 in contact surface 104b of the second thermal assembly 101b of first thermal module 100, and two aligned holes are formed through the thermal element 108b of the second thermal assembly 101b. Optical fibers 130b1, 130b2 are aligned with or extend fully or partially into the through-holes and may terminate at the openings 106b1, 106b2 formed in the contact surface 104b. Optical fiber 130b1 has a proximal end 132b1 and a distal end 134b1, and optical fiber 130b2 has a proximal end 132b2 and a distal end 134b2 (see FIGS. 27 and 28). Distal ends 134b1 and 134b2 are positioned at or proximate to contact surface 104b at openings 106b1, 106b2, respectively (see FIGS. 27 and 28). For example, distal ends 134b1 and 134b2 may be flush with contact surface 104b, may be recessed into the through-holes with respect to the contact surface 104b, or may extend beyond the contact surface 104b.

In some instances, where the distal end of an optical fiber is recessed into a contact surface of a thermal assembly, during thermal cycling in which the heated contact surface is in contact with a wall of a reaction chamber, the material forming the wall of the reaction chamber may, due to the pressure applied by the contact surface, deform outwardly into the recess formed between the end of optical fiber and the contact surface. This may create a region at which bubbles within the reaction chamber can accumulate, and this accumulation of bubbles can degrade the ability to transmit optical signals from the optical fiber to the reaction chamber and/or from the reaction chamber to the optical fiber, thereby degrading signal detection via the fiber. On the other hand, if the end of the optical fiber protrudes from the contact surface, by even a small amount, the protruding fiber will deform the wall of the reaction chamber inwardly and create an indentation that will press bubbles away from the end of the optical fiber. Thus, in some embodiments, it is preferable that the distal ends 134a1 and 134a2 extend beyond the contact surface 104a, and that the distal ends 134b1 and 134b2 extend beyond the contact surface 104b. The amount by which the optical fibers protrude past the contact surfaces may be from 0.05 mm to 0.35 mm, with a nominal protrusion of 0.15 mm.

Through-holes are formed in the thermal elements 108a, 108b and in the thermal blocks 102a, 102b. (See FIG. 38 showing through-holes 136a1, 136a2 formed in thermal element 108a and through-holes 107a1, 107a2 formed in thermal block 102a). Optical fibers 130a1, 130a2, 130b1, 130b2 extend into or through or are aligned with the through-holes formed in the thermal elements 108a, 108b and in the thermal blocks 102a, 102b. In this embodiment, because holes are formed in the thermal elements 108a, 108b of the first thermal module, but are not formed in the thermal elements 208a, 208b of the second thermal module 200, thermal elements 108a, 108b of the first thermal module 100 may be larger than thermal elements 208a, 208b of the second thermal module 200.

In an alternate embodiment, a single through-hole and associated optical fiber or more than two through-holes and associated optical fibers are formed through the thermal elements 108a/b and through the thermal blocks 102a/b of first thermal module 100.

As shown in FIGS. 27 and 28, each of the proximal ends 132a1, 132a2, 132b1, 132b2 of optical fibers 130a1, 130a2, 130b1, 130b2, respectively, is or may be coupled to an optical device 650a1, 650a2, 650b1, 650b2 for emitting an optical signal to be transmitted by the corresponding optical fiber 130a1, 130a2, 130b1, 130b2 to a corresponding one of the reaction/detection chambers 510a1, 510a2, 510b1, 510b2 aligned with the corresponding fiber, for receiving and detecting an optical signal transmitted by the corresponding optical fiber 130a1, 130a2, 130b1, 130b2 from the corresponding reaction/detection chamber 510a1, 510a2, 510b1, 510b2, or for both emitting an optical signal to be transmitted by the corresponding optical fiber 130a1, 130a2, 130b1, 130b2 to the corresponding reaction/detection chamber 510a1, 510a2, 510b1, 510b2 and for receiving and detecting an optical signal transmitted by the corresponding optical fiber 130a1, 130a2, 130b1, 130b2 from the corresponding reaction/detection chambers 510a1, 510a2, 510b1, 510b2. FIGS. 27 and 28 show each optical device 650a1, 650a2, 650b1, 650b2 associated with a single corresponding optical fiber 130a1, 130a2, 130b1, 130b2. In other embodiments, two or more fibers may be associated with the same optical device.

An optical device 650a1, 650a2, 650b1, 650b2 may comprise a photodetector for detecting light (e.g., chemiluminescence) transmitted by the corresponding optical fiber that is spontaneously emitted by the contents of the reaction/detection chambers 510a1, 510a2, 510b1, 510b2 during or after a reaction within the reaction/detection chamber in which an analyte of interest (e.g., target molecule) is present, where the detected light—or absence thereof—is indicative of the presence or absence of the analyte of interest.

Alternatively, one or more optical devices 650a1, 650a2, 650b1, 650b2 may comprise a fluorometer, including both an excitation light source (e.g., an optical emitter, such as an LED) and an emission detector (e.g., an optical detector, such as a photodiode). Excitation light of a prescribed excitation wavelength from the excitation light source is transmitted by the corresponding fiber optical fiber 130a1, 130a2, 130b1 or 130b2 to the reaction/detection chambers 510a1, 510a2, 510b1, 510b2. Light (e.g., fluorescence) of a prescribed emission wavelength emitted by a fluorescent dye (or fluorophore molecule) during or after a reaction within the reaction/detection chamber in which an analyte of interest (e.g., target molecule) is present is transmitted by the corresponding fiber 130a1, 130a2, 130b1, or 130b2 from the reaction/detection chamber to the emission light detector.

A fluorometer may include additional optical components, such as one or more lenses, optical filters, collimators, reflectors, dichroic devices, etc., to focus and condition light emitted by the excitation light source so that excitation light transmitted by the fiber to the reaction/detection chamber substantially corresponds to the prescribed excitation wavelength and to focus and condition light transmitted by the fiber from the reaction/detection chamber so that light received by the emission detector substantially corresponds to the prescribed emission wavelength.

In applications involving both an excitation light signal transmitted from the excitation source to the contents of the reaction/detection chamber and a resulting emission light signal transmitted from the contents of the reaction/detection chamber to the emission light detector, one optical fiber may be employed for transmitting the excitation light signal to the reaction/detection chamber and another optical fiber may be employed for transmitting the resulting emission light signal from the reaction/detection chamber or one fiber may be used for both transmitting an excitation light signal and transmitting a resulting emission light signal. In applications involving excitation light signals of different prescribed excitation wavelengths and light signals of different prescribed emission wavelengths, fluorometers configured to emit excitation signals and detect emission signals of different prescribed wavelengths may be coupled to the different optical fibers 130a1, 130a2, 130b1, 130b2. Alternatively, fluorometers configured detect signals of different prescribed wavelengths may be supported on a moveable platform so that different fluorometers may be selectively coupled to each of the different optical fibers 130a, 130a2, 130b1, 130b2 to interrogate each of the reaction/detection chambers for each of the prescribed wavelengths corresponding to different dyes of different probes for detecting different analytes of interest.

Examples of optical devices and systems employing such optical devices are described in International Publication No. WO 2023/248185A1, “Compact detection system,” and U.S. Pat. No. 9,465,161, “Indexing signal detection module.”

As shown in FIGS. 27 and 28, each of the proximal ends 132a1, 132a2, 132b1, 132b2 of optical fibers 130a1, 130a2, 130b1, 130b2, respectively, is coupled to an associated optical device 650a1, 650a2, 650b1, 650b2, each of which may comprise an optical emitter and an associated optical detector. Each optical emitter is associated with one of the optical detectors. Each optical emitter may include a light emitting diode (LED), and each optical detector may include a photodiode.

Referring to FIGS. 1, 2, and 29-31, optical devices 650a1, 650a2, 650b1, 650b2 may be housed within a rotating detector housing 652. A detector housing motor 654 (e.g., a stepper motor) has a drive gear 656 engaged with a driven gear 658 that is connected to the housing 652. As motor 654 rotates the housing 652 via drive gear 656 and driven gear 658, different ones of the optical devices 650a1, 650a2, 650b1, 650b2 are rotated into alignment with different ones of the proximal ends 132a1, 132a2, 132b1, 132b2 of optical fibers 130a1, 130a2, 130b1, 130b2, respectively.

Where thermal elements 108a, 108b are thermoelectric modules, they may be mounted in contact with mounting block 118 (see, e.g., FIGS. 27, 28, and 37), which functions as a heat sink to draw heat away from the thermal elements 108a, 108b. In one non-limiting example, a heat dissipation device, such as fan 190 (see FIGS. 32 and 33), may be provided to facilitate heat dissipation away from the mounting block 118 (mounting block 118 is not shown in FIG. 32).

As shown in FIGS. 35 and 37, heating elements 124a, 124b connected to a thermally conductive heater board 127 may be attached to mounting block 118 to maintain mounting block 118 at a desired temperature to facilitate efficient operation of thermoelectric modules 108a, 108b by minimizing temperature differentials between the thermoelectric modules 108a, 108b and the mounting block 118. Heating elements 124a, 124b, which may comprise resistors, may be connected for power and control to connector board 122. Thermistors (not shown) mounted to or within the heater board 127 may be provided for controlling power to the heating elements 124a, 124b to control the temperature of the heater board 127, and thus control temperature of the mounting block 118, and for which purpose an EPROM (erasable programmable read-only memory) (not shown) may be provided on connector board 122 for storing thermal parameters for the thermistors.

As shown in FIGS. 31, 34, and 36, instrument 10 may include a capacitive flow sensor 146 that is movable with the first thermal module 100. Capacitive flow sensor 146 is configured to detect fluid flow in the fluidic cartridge 500 within flow channels located downstream of the reaction/detection chambers 510a1, 510a2, 510b1, 510b2.

### Second Thermal Module

Referring to FIGS. 27 and 28, first thermal assembly 201a of second (lower) thermal module 200 includes a thermal element 208a (which may comprise a thermoelectric module, such as a Peltier device, or any other device, mechanism, or system, other than a light source, that heats, cools, or selectively heats or cools) and an associated thermal block 202a disposed in thermal contact with thermal element 208a. Thermal block 202a includes a base portion 203a, which may be in contact with thermal element 208a, and a projection 205a which defines an exposed contact surface 204a which projects through gasket 403 of cartridge support cradle 404 (see FIGS. 25, 27, and 28) and contacts a bottom side of the fluidic cartridge 500 at the reaction/detection chambers 510a1 and 510a2.

Referring to FIGS. 27 and 28, second thermal assembly 201b of second thermal module 200 includes a thermal element 208b (which may comprise a thermoelectric module, such as a Peltier device, or any other device, mechanism, or system, other than a light source, that heats, cools, or selectively heats or cools) and an associated thermal block 202b disposed in thermal contact with thermal element 208b. Thermal block 202b includes a base portion 203b, which may be in contact with thermal element 208b, and a projection 205b which defines an exposed contact surface 204b which projects through gasket 403 of cartridge support cradle 404 (see FIGS. 25, 27, and 28) and contacts a bottom side of the fluidic cartridge 500 at the reaction/detection chambers 510b1 and 510b2. Thus, in the illustrated example, contact surface 204a contacts a group of chambers including chambers 510a1, 510a2, and contact surface 204b contacts a group of chambers including chambers 510b1, 510b2.

Thermal blocks 202a, 202b are preferably made (e.g., molded and/or machined) from a thermally conductive material, such as a thermally-conductive ceramic or a metal, such as aluminum.

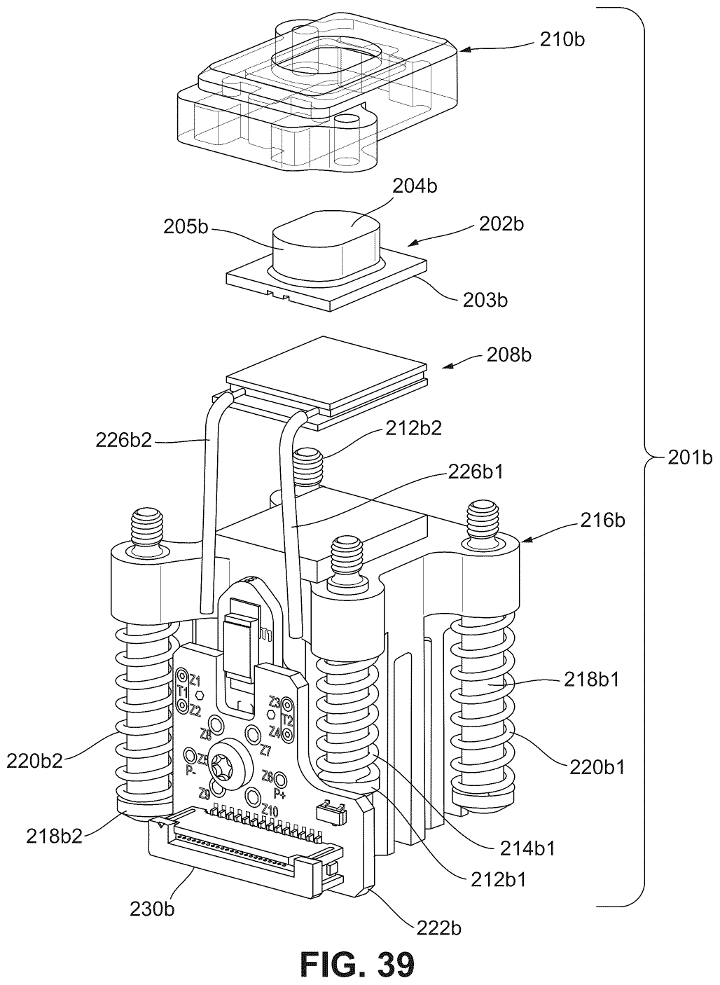

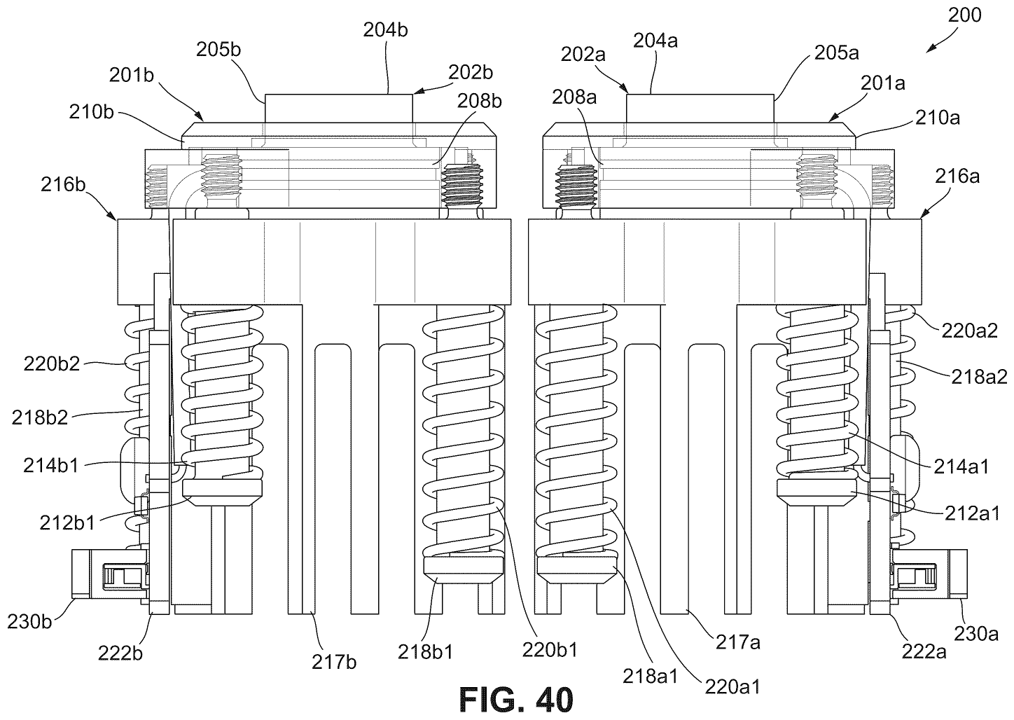

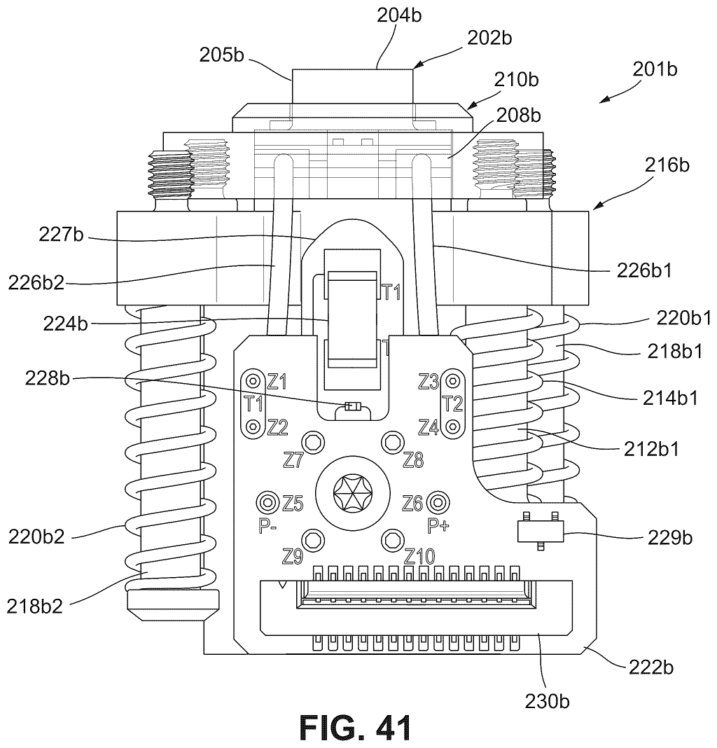

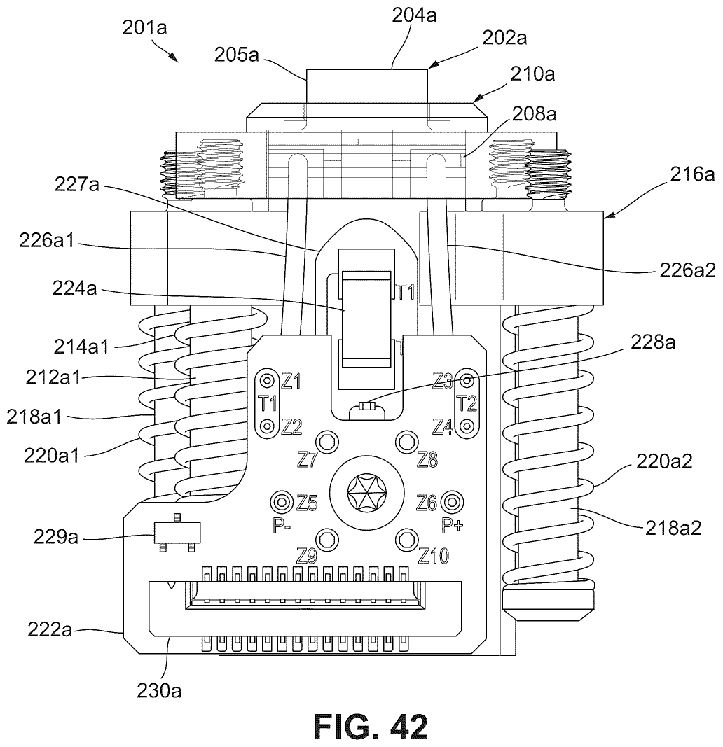

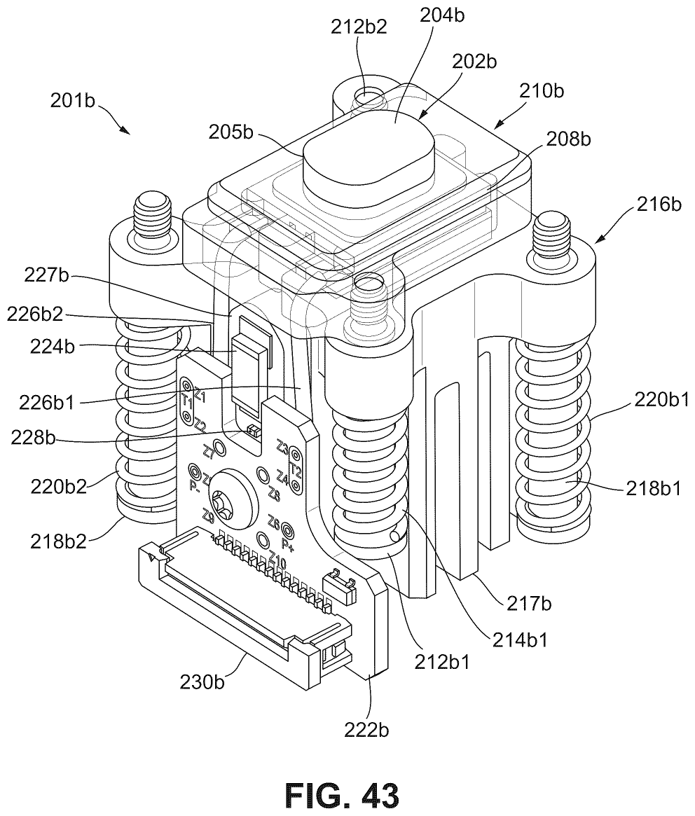

FIG. 39 is an exploded, perspective view of second thermal assembly 201b of second thermal module 200. FIG. 40 is a front view of the second thermal module 200, FIG. 41 is a left-side view of the second thermal assembly 201b of the second thermal module 200, and FIG. 42 is a right-side view of the first thermal assembly 201a of the second thermal module 200. FIG. 43 is a top perspective view of second thermal assembly 201b of second thermal module 200.

As shown in FIG. 39, a cover 210b may be positioned over thermal element 208b and associated thermal block 202b of second thermal assembly 201b. Cover 210b is not shown in FIGS. 27 and 28. As shown in FIGS. 40, 41 and 43, projection 205b of thermal block 202b projects through an opening formed in the cover 210b. Thermal element 208b and associated thermal block 202b of thermal assembly 201b may be held in place with respect to a heat sink 216b, e.g., by means of fasteners, such as cover bolts 212b1, 212b2 extending through-holes in the heat sink 216a and threaded into the cover 210b to secure the cover 210b to the heat sink 216b. As noted, thermal element 208b may be a thermoelectric module, e.g., a Peltier device, and heat sink 216b functions to draw heat away from the thermal element and dissipate the heat. Heat sink 216b is attached to or part of base plate 408 (see FIG. 25), and, in one non-limiting example, includes a plurality of heat dissipation fins 217b. A cover bolt spring 214b1 may be disposed coaxially over cover bolt 212b1 between a head of the bolt 212b1 and the heat sink 216b. Similarly, and although not visible in the drawings, a cover bolt spring may be disposed coaxially over cover bolt 212b2 between a head of the bolt 212b2 and the heat sink 216b. The purpose of the cover bolt springs is to control the force that will be applied to the cover 210b when the cover bolts 212b1, 212b2 are tightened into the mating threads of cover 210b, because the cover bolts 212b1, 212b2 are not tightened against the heat sink 216b but are tightened against the cover bolt springs.

As shown in FIGS. 40 and 42, a cover 210a may be positioned over thermal element 208a and associated thermal block 202a. Cover 210a is not shown in FIGS. 27 and 28. Projection 205a of thermal block 202a projects through an opening formed in the cover 210a The thermal element 208a and associated thermal block 202a of thermal assembly 201a may be held in place with respect to a heat sink 216a, e.g., by means of fasteners, such as a cover bolt 212a1 extending through a hole in the heat sink 216a and threaded into the cover 210a. A second cover bolt—not shown in the drawings—extends through a hole in the heat sink 216a and into the cover 210a at a corner of the cover 210a diagonally across from cover bolt 212a1. As noted, thermal element 208a may be a thermoelectric module, e.g., a Peltier device, and heat sink 216a functions to draw heat away from the thermal element 208a and dissipate the heat. Heat sink 216a is attached to or part of base plate 408 (see FIG. 25), and, in one non-limiting example, includes a plurality of heat dissipation fins 217a. A cover bolt spring 214b1 is disposed coaxially over cover bolt 212b1 between a head of the bolt 212b1 and the heat sink 216b. Similarly, a cover bolt spring is disposed coaxially over the second cover bolt between a head of the bolt and the heat sink. The purpose of the cover bolt springs is to control the force that will be applied to the cover 210a when the cover bolts 212a1 are tightened into the mating threads of cover 210a, because the cover bolts 212a1 are not tightened against the heat sink 216a but are tightened against the cover bolt springs 214a1.

Heat sinks 216a, 216b are preferably made (e.g., molded and/or machined) from a thermally conductive material, such as a thermally-conductive ceramic or a metal, such as aluminum.

Thermal assemblies 201a and 201b are mirror images of each other, and thus illustrations of thermal assembly 201a corresponding to the illustrations of thermal assembly 201b in FIGS. 39 and 43 are not provided.

In an embodiment, covers 110a, 110b, 210a, 210b are made from a plastic material, such as Ultem® (polyetherimide), which may be at least semi-transparent, or an acetal resin, such as Delrin® (polyoxymethylene (POM)). Desirable material properties of the cover material include machinability or moldability, good mechanical strength, and low thermal conductivity (e.g., 0.17 W/(m K) to 0.5 W/(m K)).

As shown in FIGS. 33 and 42, in one embodiment, first thermal assembly 201a of second thermal module 200 includes two heat sink bolts 218a1, 218a2 for securing heat sink 216a to an attaching structure within the lower chassis 400, for example, to the cartridge support frame 402 and/or the base plate 408. Similarly, as shown in FIGS. 33, 39-41, and 43, second thermal assembly 201b of second thermal module 200 includes two heat sink bolts 218b1, 218b2 for securing heat sink 216b to an attaching structure within the lower chassis 400, for example, to the cartridge support frame 402 and/or the base plate 408. A heat sink bolt spring 220a1 is disposed coaxially over heat sink bolt 218a1 between a head of the bolt 218a1 and the heat sink 216a, and a heat sink bolt spring 220a2 is disposed coaxially over heat sink bolt 218a2 between a head of the bolt 218a2 and the heat sink 216a. Similarly, a heat sink bolt spring 220b1 is disposed coaxially over heat sink bolt 218b1 between a head of the bolt 218b1 and the heat sink 216b, and a heat sink bolt spring 220b2 is disposed coaxially over heat sink bolt 218b2 between a head of the bolt 218b2 and the heat sink 216b. Each of the heat sink bolts 218a1, 218a2 extends through an associated opening formed through the heat sink 216a and is threaded into cartridge support frame 402 and/or the base plate 408, and each of the heat sink bolts 218b1, 218b2 extends through an associated opening formed through the heat sink 216b and is threaded into cartridge support frame 402 and/or the base plate 408. The purpose of the heat sink bolt springs 220a1, 220a2 is to allow the heat sink 216a, thermal module 208a, and thermal block 202a to deflect, or “float,” with respect to the structure to which heat sink 216a is attached when a downward force of sufficient magnitude is applied to the contact surface 204a of the thermal block 202a. Similarly, the purpose of the heat sink bolt springs 220b1, 220b2 is to allow the heat sink 216b, thermal module 208b, and thermal block 202b to deflect, or “float,” with respect to the structure to which heat sink 216b is attached when a downward force of sufficient magnitude is applied to the contact surface 204b of the thermal block 202b.

As shown in FIG. 42, power lines 226a1, 226a2 connect a connector board 222a to the thermal element 208a (not shown in FIG. 42) of thermal assembly 201a, and a connector 230a is provided for connecting the connector board 222a to control board 150 by a connector ribbon cable 232 (see FIG. 32). As shown in FIGS. 39, 41, and 43, power lines 226b1, 226b2 connect a connector board 222b to the thermal element 208b, and a connector 230b is provided for connecting the connector board 222b to control board 150 by a connector ribbon cable 234 (see FIG. 32).

In an alternate embodiment, rather than employing separate heat sinks 216a, 216b, the thermal elements 208a, 208b, associated thermal blocks 202a, 202b, and covers 210a, 210b of thermal assemblies 201a, 201b may be secured to a single heat sink that is large enough to accommodate more than one thermal element and associated thermal block and cover. On the other hand, having a separate heat sink for each thermal assembly may help the assembly and the thermal block contact surface take up differences in the positions of the mating surfaces due to system tolerances and cartridge warpage.

As shown in FIG. 42, at least one heating element 224a connected to a thermally conductive heater board 227a may be provided to maintain heat sink 216a at a desired temperature to facilitate efficient operation of thermoelectric module 208a of the thermal assembly 201a by minimizing temperature differentials between the thermoelectric module and the heat sink 216a. Heating element 224a, which may comprise a resistor, may be connected for power to connector board 222a. A thermistor 228a mounted to or embedded within the heater board 227a may be provided for controlling power to the heating element 224a to control the temperature of the heater board 227a, and thus control temperature of the heat sink 216a, and for which purpose an EPROM (erasable programmable read-only memory) 229a may be provided on connector board 222a for storing thermal parameters for the thermistor 228a.

Similarly, as shown in FIGS. 41 and 43, at least one heating element 224b connected to a thermally conductive heater board 227b may be provided to maintain heat sink 216b at a desired temperature to facilitate efficient operation of thermoelectric module 208b by minimizing temperature differentials between the thermoelectric module 208b and the heat sink 216b. Heating element 224b, which may comprise a resistor, may be connected for power to connector board 222b. A thermistor 228b mounted to or embedded within the heater board 227b may be provided for controlling power to the heating element 224b to control the temperature of the heater board 227b, and thus control temperature of the heat sink 216b, and for which purpose an EPROM (erasable programmable read-only memory) 229b may be provided on connector board 222b for storing thermal parameters for the thermistor 228b.

As shown in FIGS. 27 and 28 the contact surfaces 204a, 204b of the second thermal module 200 are situated in facing, or aligned, opposition with respect to associated contact surfaces 104a, 104b, respectively, of the first thermal module 100. When a test platform (e.g., cartridge 500) is placed on the cartridge support cradle 404 between the first thermal module 100 and the second thermal module 200, the contact surfaces 104a, 204a are aligned with each other and with opposed sides of the reaction/detection chambers 510a1, 510a2 disposed between them, and the contact surfaces 104b, 204b are aligned with each other and with opposed sides of the reaction/detection chambers 510b1, 510b2 disposed between them.

In an alternate embodiment, one or more through-holes are formed through one or more of the thermal elements 208a, 208b and one or more of the thermal blocks 202a, 208b of the second thermal module 200 forming one or more corresponding openings (not shown) in contact surface(s) 204a, 204b of the second thermal module 200, and an optical fiber (not shown) is associated with each through-hole of the second thermal module to transmit an optical signal through the thermal element and the thermal block. Optical fibers extending through the second thermal module 200 may be coupled to optical devices(s) for transmitting excitation optical signals to and/or receiving emission optical signals from the reaction/detection chambers through the second thermal module 200 in much the same way such optical devices are described above with respect to first thermal module 100.

The first and second thermal modules 100, 200 are constructed and arranged for relative movement toward and away from each other. Relative movement of the first thermal module 100 and the second thermal module 200 toward each other places the contact surfaces 104a, 204a in contact with opposite sides of the reaction/detection chambers 510a1, 510a2 to facilitate conductive thermal transfer between the contact surfaces 104a, 204a and the reaction/detection chambers 510a1, 510a2 and places the contact surfaces 104b, 204b in contact with opposite sides of the reaction/detection chambers 510b1, 510b2 to facilitate conductive thermal transfer between the contact surfaces 104b, 204b and the reaction/detection chambers 510b1, 510b2.

### Thermal Module Actuator

To effect relative movement between the first thermal module 100 and the second thermal module 200, either or both of the first thermal module 100 and the second thermal module 200 is configured to be movable toward and away from the other. The relative movement may be vertical when the first and second thermal modules 100, 200 are arranged one above the other. In another example, the relative movement may be lateral (horizontal, or non-vertical) when the first and second thermal modules 100, 200 are arranged side-by-side. In one non-limiting example, second thermal module 200 is fixed within the instrument 10, and the first thermal module 100 is movable (e.g., vertically) with respect to the second thermal module 200. As illustrated schematically in FIGS. 27 and 28, a thermal module actuator 250 is configured to effect automated relative movement between the first thermal module (first heater) 100 and the second thermal module (second heater) 200. Thermal module actuator 250 may comprise an actuator motor 252 that is fixed within the upper chassis 300, e.g., to motor mount 314 (see FIGS. 1, 2, 29-31), and a lead screw 258 attached at one end directly or indirectly to mounting block 118. In the illustrated example, thermal module actuator 250 is configured to effect automated movement of the movable first thermal module 100 toward or away from the fixed second thermal module 200. In FIG. 27, first thermal module 100 is shown in a first, or raised, position above a top surface of the fluidic cartridge 500 so as to form gaps between the contact surfaces 104a, 104b and the cartridge 500. Fluidic cartridge 500 is supported on the contact surfaces 204a, 204b of the second thermal module 200 and on the cartridge support cradle 404. In FIG. 28, first thermal module 100 has been lowered by the thermal module actuator 250 to a second, or lowered or engaged, position at which detection regions of the test platform/cartridge are sandwiched between the first thermal module/heater 100 and the second thermal module/heater 200. In this context, the detection regions are “sandwiched” between the first thermal module/heater 100 and the second thermal module/heater 200 if the detection regions are disposed between the first thermal module/heater 100 and the second thermal module/heater 200 and in contact with or in sufficiently close proximity to the first thermal module/heater 100 and the second thermal module/heater 200 to enable effective thermal transfer between the first thermal module/heater 100 and the second thermal module/heater 200 and the detection regions (e.g., contact surfaces 104a, 104b are in thermal contact—which may include direct physical contact—with a top surface of reaction/detection chambers 510a1, 510a2, 510b1, 510b2, and contact surfaces 204a, 204b are in thermal contact—which may include direct physical contact—with a bottom surface of the reaction/detection chambers).

With reference to FIG. 29, thermal module actuator 250 comprises motor 252 (e.g., a stepper motor) mounted on a motor mounting plate 254 that is supported on, but not connected to, the intermediate crossbar 310 of the motor mount 314 at a position that is generally at a midpoint between the side supports 306a, 306b. Linear bearings/guide rods 256a, 256b are attached at one end to upper block 302 and at an opposite end to top crossbar 308 and extend through intermediate crossbar 310 and motor mounting plate 254 on opposite sides of motor 252. The lead screw (linear drive) 258 extends from motor 252, through the motor mounting plate 254 and intermediate crossbar 310, and to the upper block 302 to which the mounting block 118 of the thermal assemblies 101a, 101b of the first thermal module 100 are attached. Rotation of the lead screw 258 by the motor 252 raises or lowers the upper block 302, and the first thermal module 100 and mounting block 118 attached to the upper block 302, by moving the upper block 302 toward or away from the motor 252. During movement by motor 252 and lead screw 258, the upper block 302 is guided by the linear bearings 256a, 256b to avoid tilting and binding of the upper block 302.

FIG. 21 is a partial cross-section of the cartridge body 502 and the upper block 302 and the pressure plate 320 with the upper block 302 and the pressure plate 320 in a raised position with respect to the cartridge body 502, and FIG. 22 is the same cross-section with the upper block 302 and the pressure plate 320 in a lowered position with respect to the cartridge body 502. When the pressure plate 320 contacts the top of the cartridge 500, further downward movement of the upper block 302 and mounting block 118 is arrested, and continued rotation of the lead screw 258 will then separate the motor 252 and motor mounting plate 254 from the intermediate crossbar 310. Springs 260a, 260b coaxially surrounding portions of linear bearings/guide rods 256a, 256b, respectively, between the motor mounting plate 254 and the top crossbar 308 on opposite sides of the motor 252 will compress as the motor mounting plate 254 separates from the intermediate crossbar 310, thereby increasing the spring force in each of the springs 260a, 260b, and thereby controlling the amount of downward force exerted by the lead screw 258 onto the upper block 302, depending on the spring constants of the springs 260a, 260b. In some embodiments, an optical sensor (not shown) comprising an emitter/receiver pair will detect a beam of light from the emitter to the receiver through a gap between the motor mounting plate 254 and the intermediate crossbar 310 to generate a signal to deactivate the motor 252 when the motor mounting plate 254 is lifted off the intermediate crossbar 310.

As shown, for example, in FIG. 36, cover 110a of first thermal assembly 101a may include a raised portion 116a, and cover 110b of second thermal assembly 101b may include a raised portion 116b. When first thermal module 100 is lowered by the thermal module actuator 250 so that the contact surfaces 104a, 104b contact reaction/detection chambers 510a1, 510a2, 510b1, 510b2, respectively, raised portions 116a, 116b bear against a portion of the reaction/detection section 506 of fluidic cartridge 500 at which valves are located, and the raised portions 116a, 116b provide a backing when valve actuator heads 406 push up against a side of the cartridge opposite raised portions 116a, 116b to actuate the corresponding valves V1 to V18 in the cartridge 500.

### Contact Detector

Instrument 10 may include a mechanism for holding a cap closed on a sample chamber W1 of a fluidic cartridge 500 within the instrument 10 and for generating a signal to indicate that a cartridge 500 is positioned on the cartridge support cradle 404 and that a cap 516 (or lysis vessel 700) is situated over the sample chamber of the cartridge. Referring to FIGS. 2 and 29, such a mechanism may comprise a contact detector 340 comprising, as shown in FIG. 29, a plunger 342 and an optical detector 350 attached to the upper block 302. In general, the contact detector includes a component, e.g., plunger 342, that contacts the cap of the cartridge as the first thermal module 100 and the second thermal module 200 are brought into contact with the cartridge, and a portion of the component moves into a position to interrupt an optical beam, thereby generating a signal confirming that the cartridge/cap are present. If the cartridge and/or cap are not present, the component does not move as the first thermal module 100 and the second thermal module 200 are brought into contact with the cartridge and the optical beam is not interrupted.

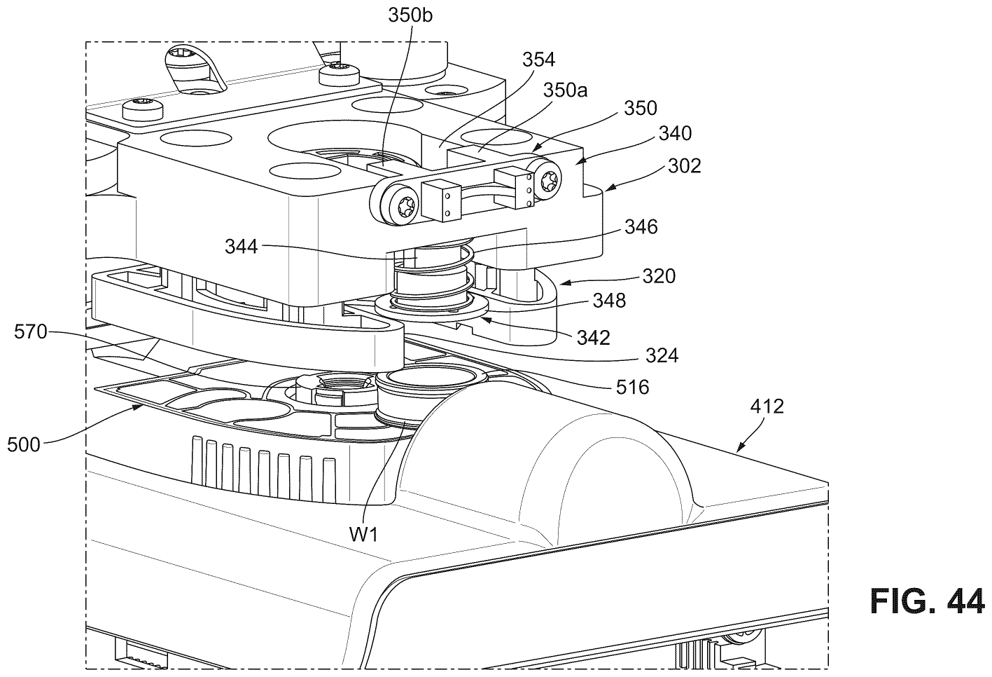

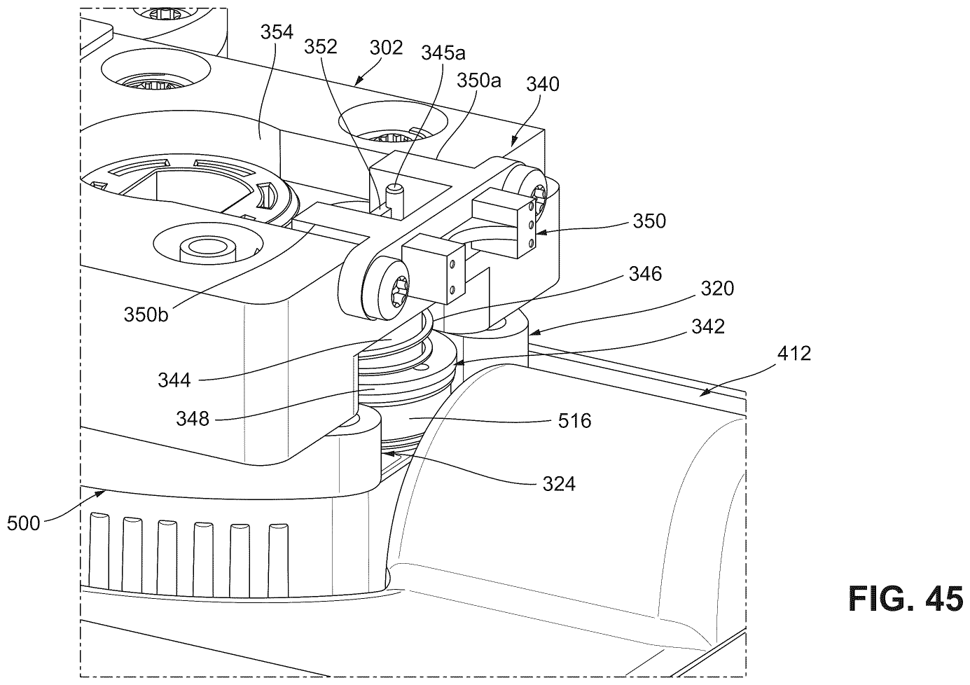

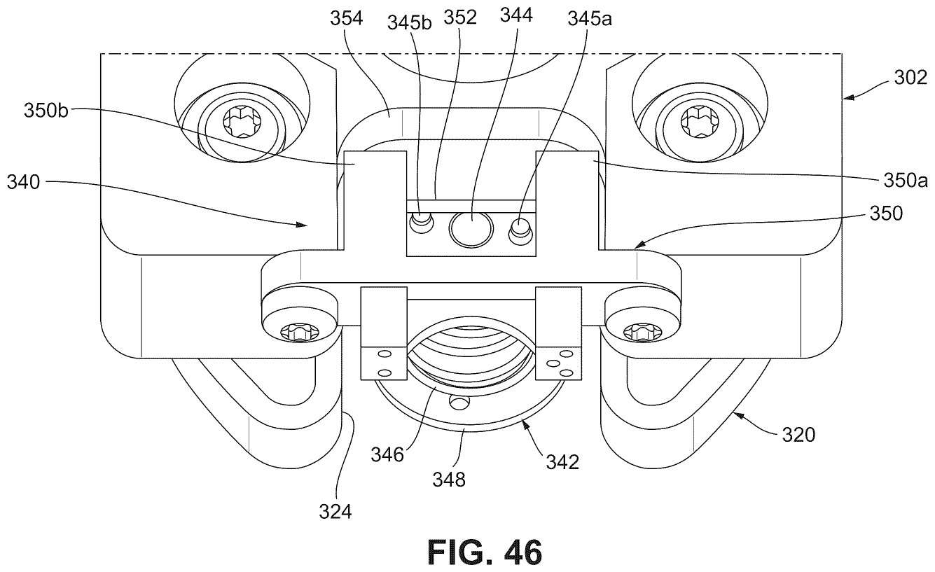

FIG. 44 is a partial perspective view of the instrument showing upper block 302 in a raised position above fluidic cartridge 500 held in holder 412 so that pressure plate 320 and plunger 342 are not in contact with cartridge 500. FIG. 45 is a partial perspective view of the instrument showing block 302 in a lowered position with respect to fluidic cartridge 500 held in holder 412 so that pressure plate 320 and plunger 342 are in contact with cartridge 500. FIG. 46 is a partial, top perspective view showing the contact detector 340 without the fluidic cartridge 500 or holder 412.

As shown in FIGS. 44-46, an example of an optical sensor 350 included in the contact detector 340 includes an optical transmitter 350a and an optical receiver 350b disposed within a recess 354 (see also, FIG. 32 showing recess 354) formed in the top of the upper block 302. Plunger 342 includes a plunger rod 344 extending through the upper block 302, and a plunger pad 348 on a lower end of the plunger rod 344 and disposed within a cutout 324 formed in the pressure plate 320 (see also FIG. 31 showing cutout 324). A spring 346 is disposed around the plunger rod 344 between the upper block 302 and the plunger pad 348.

FIG. 21 is a partial cross-section of the cartridge body 502 and the upper block 302, the pressure plate 320, and the plunger 342 of the contact detector 340 with the upper block 302, the pressure plate 320, and the plunger 342 in a raised position with respect to the cartridge body 502, and FIG. 22 is the same cross-section with the upper block 302, the pressure plate 320, and the plunger 342 in a lowered position with respect to the cartridge body 502. When the upper block 302 is in the first (raised) position (FIGS. 21 and 44), no portion of the plunger 342 is disposed between the optical transmitter 350a and the optical receiver 350b, and an optical beam 352 from the transmitter 350a is received by the receiver 350b. As shown in FIGS. 22 and 45, when a fluidic cartridge 500 is positioned on the cartridge support cradle 404 below the upper block 302, and the upper block 302 is lowered by the thermal module actuator 250 to its second position onto the cartridge 500, the pressure plate 320 contacts the top of the fluidic cartridge 500 and the plunger pad 348 of the plunger 342 contacts a top edge of the peripheral wall 520 of the cap 516 (see FIGS. 15-17)—or a top edge of the peripheral wall 706 of the lysis vessel 700 (see FIG. 54)—inserted into the sample chamber W1 of the cartridge 500. Vent hole 523 formed in the radial wall 522 of the cap 516 and side vent holes 521a, 521b (see FIG. 17) allow pressure equalization within the sample chamber W1 when the cap 516 is covered by the plunger pad 348 to permit sample fluid to be drawn from the sample chamber W1 by the syringe. As the upper block 302 is lowered, the plunger rod 344 of the plunger 342, which is biased in a downward position by spring 346, is pushed up through the upper block 302. An upper end of the rod 344 passes between the optical transmitter 350a and receiver 350b to alter (e.g., interrupt or block) an optical beam 352 between them (see FIG. 46), thereby causing a signal or changing a signal (from unblocked to blocked) to indicate that a fluidic cartridge 500 is positioned on the cartridge support cradle 404. If no cartridge is positioned on the cradle 404 when the upper block 302 is lowered, the plunger 342 will not be pushed up and the plunger rod 344 will not break the optical beam 352, thereby indicating that a cartridge is absent.

Alternatively or in addition, contact detector 340 may include rods 345a, 345b on either side of plunger rod 344 and which are coupled to the plunger pad 348 so that as plunger pad 348 is moved up due to contact with the cap 516 or lysis vessel 700, rods 345a, 345b move up to alter the optical beam 352. Rods 345a, 345b may be narrower than plunger rod 344 and may be slightly offset to ensure that they reliably interrupt the beam 352 when in the raised position.

In another embodiment, the rod 344 of plunger 342 is disposed between the optical transmitter 350a and the optical receiver 350b to block the beam 352 when the upper block 302 is in the first position. A hole is formed through the rod 344, and when the plunger 342 is moved upon contacting the cartridge when the upper block 302 is moved to the second position, the hole is aligned with the optical transmitter 350a and the optical receiver 350b, thereby allowing the optical beam 352 to pass from the optical transmitter 350a to the optical receiver 350b. Again, it is the change in signal caused by the beam 352 becoming unblocked as the upper block 302 moves from the first position to the second position and the plunger 342 contacts a cartridge disposed between the first thermal module 100 and the second thermal module 200 that indicates the presence of the cartridge.

Plunger 342, pushing down on the cap over the sample chamber W1 with the force of the spring 346, will help hold a cap in a closed position over the sample chamber W1 while the fluidic cartridge 500 is being operated on by the instrument 10.

If fluidic cartridge 500 includes a chamber expander 830 (see FIGS. 65-72), plunger 342 will contact top wall 878 of cap 870 to hold the cap 870 in a closed position. When a fluidic cartridge 500 with a chamber expander 830 is positioned on the cartridge support cradle 404 below the upper block 302, and the upper block 302 is lowered by the thermal module actuator 250 to its second position onto the cartridge 500, the pressure plate 320 contacts the top of the fluidic cartridge 500 and the plunger pad 348 of the plunger 342 contacts a top wall 878 of the cap 870. Expansion chamber 830 extending above fluidic cartridge 500 fits within cutout 324 formed in the pressure plate 320 to permit pressure plate 320 to contact cartridge 500. Venting grooves 886a, 886b and vent holes 885 and 884 formed in top wall 878 of cap 870 allow pressure equalization within the sample chamber W1 and interior space 848 when the cap 870 is covered by the plunger pad 348 to permit sample fluid to be drawn from the sample chamber W1 by the syringe. As described above, the plunger rod 344 of the plunger 342 contacting the cap 870 is pushed up through the upper block 302 where upper end of rod 344 passes between the optical transmitter 350a and receiver 350b to alter (e.g., block) the optical beam 352 between them (see FIG. 46), thereby causing a signal or changing a signal (from unblocked to blocked) to indicate that a fluidic cartridge 500 is positioned on the cartridge support cradle 404.

In a system and process that employs a fluidic cartridge with a bead delivery cap 900, means are required for deforming the deformable wall 930 of the cap 900 to release the lysis beads 926, 924 into the sample chamber W1. In one non-limiting example, after sample is dispensed into the sample chamber W1, and the bead delivery cap 900 is inserted into the chamber W1, deformable wall 930 may be manually pressed, e.g., with a user's finger, to collapse the deformable wall 930 and rupture the frangible membrane 934 to release the magnetic element 926 and the plurality of non-magnetic beads 924 into the sample chamber W1. Alternatively, instrument 10 may include a device that automatically applies a collapsing force to the deformable wall 930. In one non-limiting example, the contact detector 340 shown in FIGS. 44-46 may be modified to apply a collapsing force to the deformable wall 930 when the upper block 302 and pressure plate 320 are lowered by the thermal module actuator 250 into contact with a fluidic cartridge 500.

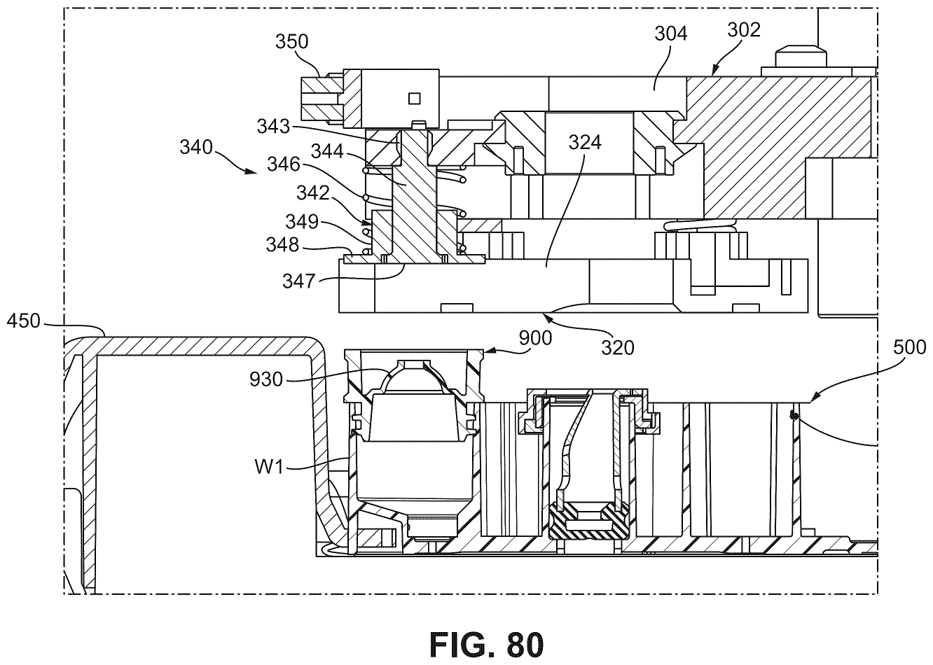

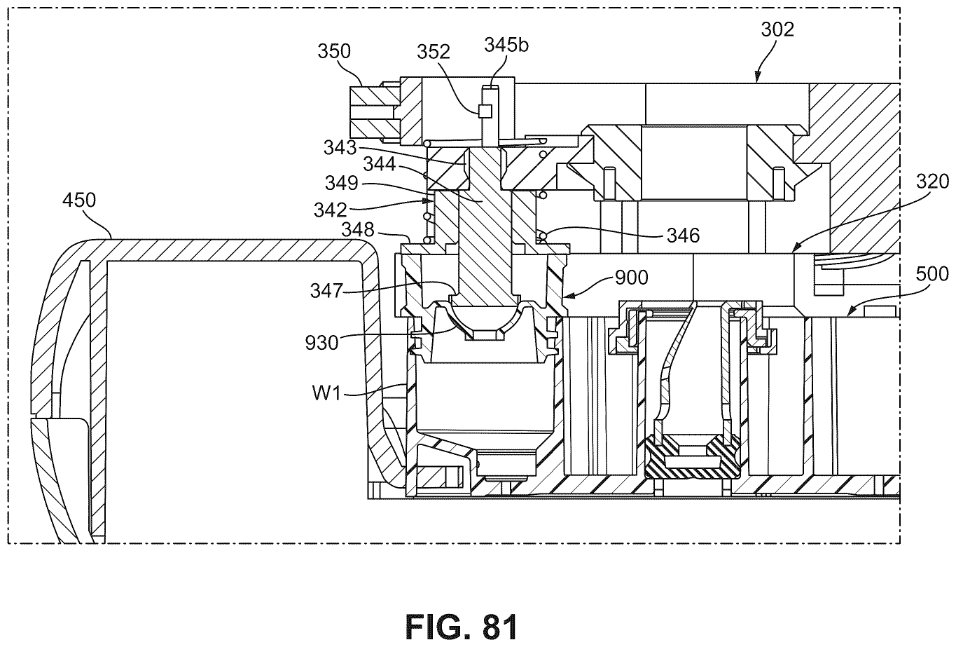

FIG. 80 is a partial cross-section of the fluidic cartridge 500, the upper block 302 and pressure plate 320 of the upper chassis of the instrument, and contact detector 340. The upper block 302, the pressure plate 320, and the contact detector 340 are in a raised position with respect to the fluidic cartridge 500 in FIG. 80. FIG. 81 is a partial cross-section of the fluidic cartridge 500, the upper block 302 and pressure plate 320 of the upper chassis of the instrument, and the contact detector 340. The upper block 302, the pressure plate 320, and the contact detector 340 are in a lowered position with respect to the fluidic cartridge in FIG. 81. For clarity, certain features of the bead delivery cap 900 are omitted from FIGS. 80 and 81, such as, the frangible membrane 934, the magnetic element 926, and the non-magnetic beads 924.

In the illustrated embodiment, a bead delivery cap actuator comprises a center post 344 decoupled from the plunger 342 and anchored at a top end 343 to the upper block 302 (e.g., by mating threads) and having a contact pad 347 at a lower end that is wider than the center post 344. Plunger 342 includes plunger pad 348 and a collar 349 through which the center post 344 extends and with respect to which the center post 344 can slide. Side posts 345a, 345b (see FIG. 46) are attached to and move with the plunger 342. In one non-limiting example, side posts 345a, 345b extend from a top surface of collar 349.

As shown in FIG. 80, with the upper block 302 in the raised position, plunger 342, urged by spring 346, bears against the contact pad 347 of center post 344. Center post 344 extends through an opening form through the plunger 342, where the portion of the opening extending through the collar 349 has a width slightly larger than the width of the center post 344, and the portion of the opening extending through the plunger pad 348 has a width slightly larger than the width of the contact pad 347. Accordingly, the center post 342 is able slide within the opening through the plunger 342 and the contact pad 347 is flush with the plunger pad 348.

As shown FIG. 81, with the upper block 302 in the lowered position, and the pressure plate 320 contacting the top of the fluidic cartridge 500, plunger pad 348 contacts the top of cap 900 and pushes the plunger 342 up against the spring 346 into the upper block 302. Side posts 345a, 345b (only side post 345b is visible in FIG. 81) project up into the recess 354 to interrupt the optical beam 352 of the optical sensor 350, thereby causing a signal confirming the presence of the cap 900. The center post 344, being anchored to the upper block 302 and decoupled from the plunger 342, moves down with the upper block 302, below the plunger pad 348, so that the contact pad 347 contacts the deformable wall 930. The center post 344 is configured to have a range of motion with respect to the plunger 342 to extend sufficiently below the top of the cap 900 to collapse the deformable wall 930 and cause the frangible membrane 934 to rupture thereby releasing the magnetic element 926 and the plurality of non-magnetic beads 924 into the sample chamber W1.

### Operation

### Lysis Method

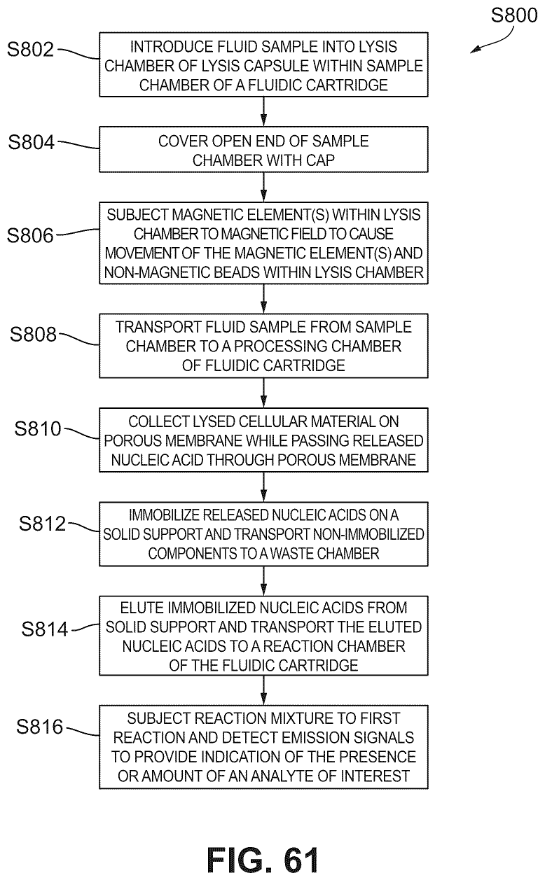

The following description presents an example of an operation for performing on-board lysis within a cartridge 500. FIG. 61 shows a flow diagram illustrating an embodiment of a method S800 for performing lysis and a molecular assay using instrument 10 and cartridge 500. Method S800 may be performed with or used in conjunction with any of the computer systems, devices, mechanisms, elements, or components disclosed herein, among other devices. Method S800 may be coded and stored as a computer-executable control algorithm for controlling the operation(s) of one or more of the computer systems, devices, mechanisms, elements, or components disclosed herein, among other devices. In various embodiments, some of the method steps shown may be performed concurrently, in a different order than shown, or may be omitted. Additional method steps may also be performed as desired. Flow begins at step S802.

Method S800 is described with reference to lysis capsule 600, but the process described could be performed without significant modification, except as noted herein, with lysis capsules 600′, 600″, 818, with bead delivery cap 900, or with mechanical lysis sample chamber 1002, 1002′, 1002″.

In step S802 a fluid sample is introduced, manually or robotically, into the lysis chamber 622 of the lysis capsule 600 previously placed in the sample chamber W1 of cartridge 500. Alternatively, if the fluidic cartridge includes a mechanical lysis sample chamber 1002, 1002′, 1002″, fluid sample is dispensed directly into the open sample chamber W1 within which the lytic agents (with non-magnetic beads 1024 and magnetic element 1026) have been pre-positioned. In one non-limiting example, the sample is dispensed, e.g., with a pipettor, through the first porous membrane 618 of the capsule 600 into the lysis capsule. If an expandible cartridge 800 with a chamber expander 830 is employed, sample is dispensed into the interior space 848 of expansion chamber 846 through opening 856, and sample will pass through first porous membrane 821 and into the lysis capsule 818. An amount of sample may be added that exceeds the volumetric capacity of lysis capsule 818, and sample may partially or fully fill the interior space 848 of the chamber expander 830.

In step S804, the sample chamber is closed with a cap 516 by inserting peripheral wall 525 of cap 516 into the sample chamber W1. If the cartridge includes a chamber expander 830, the chamber expander 830 is closed with cap 870 by inserting the insert sleeve 872 into the opening 856.

FIG. 63 shows a flow diagram illustrating an embodiment of a method S830 for introducing a fluid sample into a lysis chamber if lysis vessel 700, comprising an integrated cap and lysis capsule, is employed, whereby method S830 would replace steps S802 and S804 of method S800. In step S832, fluid sample is dispensed, e.g., with a manual or robotic pipettor, directly into the sample chamber W1. In step S834, sleeve 725 of lysis vessel 700 is inserted into the sample chamber W1 to close the sample chamber W1, forcing the fluid sample within chamber W1 through the porous membrane 720 and into lysis chamber 722.

In another workflow for introducing lysis beads into a fluid sample if bead delivery cap 900 is employed, fluid sample is dispensed into the sample chamber W1, and lower sleeve 910 of bead delivery cap 900 is inserted into the sample chamber W1 to close the sample chamber W1. Cartridge 500 is placed in the instrument 10, and when the upper block 302 is lowered, plunger pad 348 contacts the top of cap 900 and pushes the plunger 342 up against the spring 346 into the upper block 302 (see FIGS. 80, 81). The center post 344 moves down with the upper block 302, below the plunger pad 348, and the contact pad 347 collapses the deformable wall 930 and ruptures the frangible membrane 934 to release the magnetic element 926 and the plurality of non-magnetic beads 924 into the sample chamber W1. This workflow replaces steps S802 and S804 of method S800.

Returning to FIG. 61 and method S800, the fluidic cartridge 500 is then placed into the cartridge holder 412, thereby positioning the lysis capsule 600 or lysis vessel 700 within the sample chamber W1 in close proximity to the electromagnet 452 within the electromagnet housing 450. The fluidic cartridge 500 is then moved into the instrument by retracting the cartridge holder 412 into the instrument 10.

Within the instrument, in step S806, the electromagnet 452 is activated by the oscillating circuit 454 (FIG. 57) to apply a magnetic field to the magnetic element 626, 626′, 626″ within the lysis chamber 622, 622′, 622″. Or, as shown in FIG. 63, in step S836, the electromagnet 452 is activated by the oscillating circuit 454 to apply a magnetic field to the magnet 726 within the lysis chamber 722. For other embodiments, such as bead delivery cap 900 and mechanical lysis sample chamber 1002, 1002′, 1002″, the magnetic field is applied to the magnetic element 926 and magnetic element 1026, respectively, within the sample chamber W1. In step S806 of FIG. 61 or step S836 of FIG. 63, the magnetic field may be a variable or oscillating magnetic field, meaning that the north and south poles of the electromagnetic magnet are flipped at a high frequency, thereby causing corresponding movement (agitation) of magnetic element 626, 626′, 626″, magnetic element 726, magnetic element 926, or magnetic element 1026 as the magnet constantly seeks to align its north and south poles with the oscillated magnetic field of the electromagnet 452. As noted above, the magnetic field may be oscillated at a frequency of 20 to 200 Hz. In one non-limiting example, the frequency itself may be variable. For example, the frequency may sweep from 60 Hz to 100 Hz and back to 60 Hz, or the frequency may be pulsed between two or more different, discrete frequencies, e.g., rapidly switching the frequency from 60 Hz to 100 Hz back to 60 Hz, or the frequency may be hopped, stepping between different discrete frequencies, and so-on. As different magnetic field oscillation frequencies may be more effective for effecting lysis of different sample materials—depending on material properties (e.g., viscosity)—varying the frequency of the magnetic field oscillations helps ensure an effective magnetic field oscillation frequency is applied, regardless of the sample material properties. Varying the frequency of the magnetic field oscillations also helps ensure a random, chaotic movement of the magnetic element and non-magnetic beads and helps to prevent the magnetic element from reaching a resonance with a particular magnetic field oscillation frequency, whereby the magnet merely spins without imparting sufficient motion to the non-magnetic beads.

If the cartridge includes a chamber expander 830, it is possible that a portion of the sample dispensed into the sample chamber W1 will be contained within the interior space 848 of the expansion chamber 846 above the lysis capsule 818 (see FIGS. 66, 67). Oscillation of the magnet 823 within the lysis chamber defined between membranes 821, 825 will cause a fluid flow (such as a vortex) that will draw sample from above the first porous membrane 821 through the membrane 821 and into the lysis capsule 818. Accordingly, all fluid sample dispensed into the sample chamber W1 will be exposed to the mechanical lysing process.

In one non-limiting example, the magnetic field is applied for 3 to 5 minutes during step S806 or step S836.

At the conclusion of step S806 of FIG. 61 or step S836 of FIG. 63, in step S808 of FIG. 61, the lysed sample fluid is moved from the lysis chamber within sample chamber W1 to a processing chamber, such as a purification column within the purification chamber W4, to purify the released nucleic acid. In one non-limiting example, the lysed sample fluid is moved from the sample chamber to the processing chamber at a rate of 5.0 to 30.0 μl/sec., for example, by control the speed at which motor 368 of syringe driver 360 moves the syringe plunger 362 (see FIG. 20) to draw sample fluid from the sample chamber to the syringe barrel SB.

While the lysed sample fluid is moved from the lysis chamber within sample chamber W1, the electromagnet 452 may be activated by the oscillating circuit 454 (FIG. 57) to apply the oscillating or variable magnetic field to the magnetic element within the lysis chamber to continue agitating the magnetic element and the non-magnetic beads. In one non-limiting example, the oscillating or variable magnetic field may be applied to the magnetic element at the same frequency during step S808 as during step S806 of FIG. 61 or step S836 of FIG. 63. This process, known as a sweeping process, is effective to keep particles within the lysis chamber in suspension until all or most of the fluid is removed from the lysis chamber, thereby reducing or avoiding clogging of the filter(s) and/or membrane(s) at the bottom of the lysis chamber.

In step S810, as the released nucleic acid material is transported from the sample chamber W1 to a processing chamber, lysed cellular material is collected on a porous membrane and/or filter element at the bottom of the sample chamber, such as the second porous membrane 620 of lysis capsule 600 (and/or on filter 630 if such a filter is included) or porous membrane 720 of lysis vessel 700 (and/or on filter 730 if such a filter is included).

In step S812, the released nucleic acid is immobilized on a solid support within the purification column of the purification chamber W4, and non-immobilized components of the fluid sample are transported to a waste chamber, such as chamber W11 or chamber W12.

In step S814, immobilized nucleic acid is eluted from the solid support by transferring an amount of elution buffer from chamber W10 to the purification chamber W4. The eluted nucleic acid is combined with one or more reagents (e.g., PCR mix 1 from chamber W5 and/or PCR mix 2 from chamber W7), and the resulting reaction mixture is transported from the purification chamber W4 to one of the reaction/detection chambers 510a1, 510a2, 510b1, 510b2.

In step S816, the nucleic acid reaction mixture is subjected to first reaction conditions by, for example, applying first prescribed thermal conditions—e.g., thermal cycling for a PCR reaction—to the reaction/detection chambers 510a1, 510a2, 510b1, 510b2. Emission signals (e.g., fluorescent signals) from within the reaction/detection chambers 510a1, 510a2, 510b1, 510b2 are detected to indicate the presence or amount of an analyte of interest. The emission signals may be detected while or after the nucleic acid reaction mixture is subjected to the first reaction conditions.

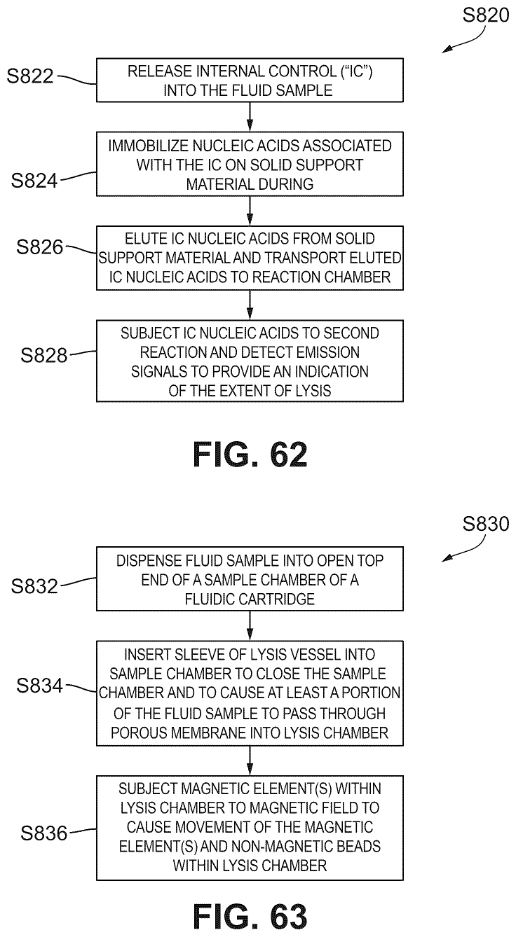

FIG. 62 shows a flow diagram illustrating an embodiment of a method S820 having steps which may be combined with method S800 of FIG. 61 for an operation where an internal control is employed.

In step S822, an internal control is released into the fluid sample. The internal control reagent may be added directly to the fluid sample prior to introducing the fluid sample into the sample chamber W1, or, if method S820 is incorporated into method S800 (FIG. 61), the internal control reagent may be added directly to the fluid sample before step S802 or step S832 (if incorporated into method S830 (FIG. 63)). Alternatively, the internal control reagent may be added to the fluid sample after the fluid sample has been dispensed into the sample chamber W1, or the internal control reagent may be added to the fluid sample within lysis capsule 600 after step S802 and before step S804 or within lysis vessel 700 after step S832 and before step S834. Alternatively, if method S820 is incorporated into method S800 (FIG. 61), the internal control reagent may be provided in a non-liquid form dried to a portion of the lysis capsule 600, 600′, 600″, lysis vessel 700 (for method S830, FIG. 63), or mechanical lysis sample chamber 1002, 1002′, 1002″ and then contacting the non-liquid internal control reagent with fluid sample, thereby dissolving the internal control reagent.

In step S824, after the internal control reagent, now combined with the fluid sample, is transported from the sample chamber W1 to a processing chamber, such as a purification column within the purification chamber W4, the internal control nucleic acids are immobilized on a solid support within the purification column of the purification chamber W4. Step S824 may be performed in combination with step S812 of method S800.

In step S826, immobilized internal control nucleic acids are eluted from the solid support by transferring an amount of elution buffer from chamber W10 to the purification chamber W4, and the eluted nucleic acid is combined with one or more reagents—e.g., PCR mix 1 from chamber W5 and/or PCR mix 2 from chamber W7—and the resulting reaction mixture is transported from the purification chamber W4 to one of the reaction/detection chambers 510a1, 510a2, 510b1, 510b2 of cartridge 500. Step S826 may be performed in combination with step S814 of method S800.

In step S828, the internal control nucleic acid reaction mixture is subjected to second reaction conditions by, for example, applying second prescribed thermal conditions—e.g., thermal cycling for a PCR reaction—to the reaction/detection chambers 510a1, 510a2, 510b1, 510b2. The second reaction conditions may be the same as the first reaction conditions. Emission signals (e.g., fluorescent signals) from within the reaction/detection chambers 510a1, 510a2, 510b1, 510b2 are detected to indicate the presence or amount of the internal control. Step S828 may be performed in combination with step S816 of method S800. The emission signals may be detected while or after the nucleic acid reaction mixture is subjected to the second reaction conditions.

### Molecular Assay

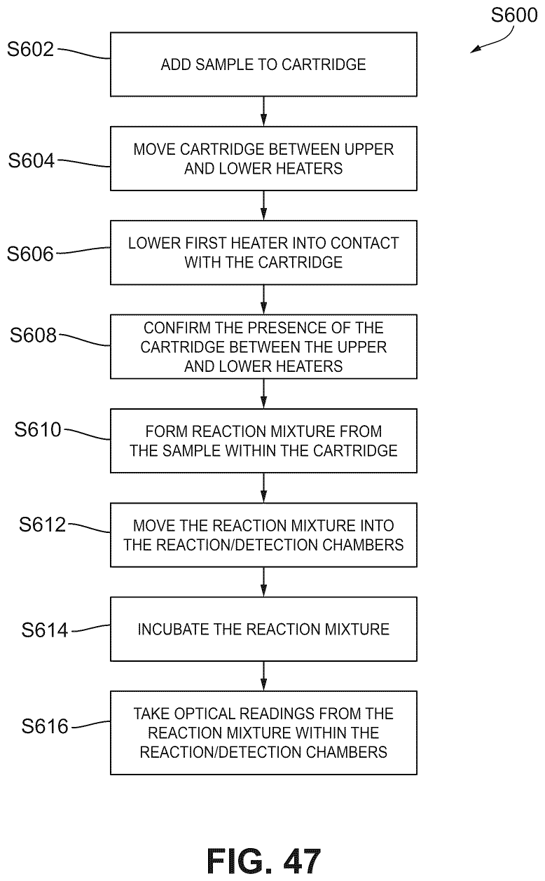

The following description presents an example of an operation for performing an assay using instrument 10 and a cartridge 500. FIG. 47 shows a flow diagram illustrating an embodiment of a method S600 for performing a molecular assay using instrument 10 and cartridge 500. Method S600 may be performed with or used in conjunction with any of the computer systems, devices, mechanisms, elements, or components disclosed herein, among other devices. Method S600 may be coded and stored as a computer-executable control algorithm for controlling the operation(s) of one or more of the computer systems, devices, mechanisms, elements, or components disclosed herein, among other devices. In various embodiments, some of the method steps shown may be performed concurrently, in a different order than shown, or may be omitted. Additional method steps may also be performed as desired. Flow begins at step S602.

In step S602, sample is added to the fluidic cartridge 500 by dispensing a fluid sample into the sample chamber W1 of the fluidic cartridge 500 and placing cap 516 over the sample chamber W1. If an expandable fluidic cartridge 800 with a chamber expander 830 is used, sample is dispensed into interior space 848 of the expansion chamber 846 of chamber expander 830, and cap 870 is inserted into opening 856 to close chamber 846. Reagents and other materials necessary for performing the intended procedure—e.g., a molecular assay—are contained within one or more chambers W2-W5, W7-W10 of the sample preparation section 504 of the cartridge 500. Protective cover 566 is peeled off the venting membrane 562 of the protective venting cover 560.

Fluidic cartridge 500 (although the remaining steps could be performed with expandible cartridge 800, the remaining description of the operation will refer only to fluidic cartridge 500) is then placed on the cartridge holder 412, and, in step S604 the cartridge is placed between upper and lower heaters (e.g., between thermal assemblies 101a, 101b of first thermal module 100 and thermal assemblies 201a, 201b of the second thermal module 200) by retracting the cartridge holder 412 into the instrument 10 between the first and second thermal modules 100, 200. Due to springs 417 disposed between holder 412 and rails 416a, 416b, within recesses 415a, 415b, respectively, (see FIG. 25) which position the holder 412 above the frame 414, the fluidic cartridge 500 is supported slightly above the cartridge support cradle 404 with the reaction/detection chambers 510a1, 510a2 positioned above the contact surface 204a of the first thermal assembly 201a of the second thermal module 200 and the reaction/detection chambers 510b1, 510b2 positioned above the contact surface 204b of the second thermal assembly 201b of the second thermal module 200.

In step S606, the first heater is lowered into contact with the cartridge by lowering the first thermal module 100 by the thermal module actuator 250 to place pressure plate 320 in contact with the top of fluidic cartridge 500 and to place contact surface 104a of first thermal assembly 101a in contact with an outer surface of a portion of fluidic cartridge 500 forming an upper wall of reaction/detection chambers 510a1, 510a2 and to place contact surface 104b of second thermal assembly 101b in contact with an outer surface of a portion of fluidic cartridge 500 forming an upper wall of reaction/detection chambers 510b1, 510b2. Contact by the pressure plate 320 with a top surface of fluidic cartridge 500 (e.g., contact with the venting membrane 562 of cartridge 500) also compresses springs 417 between holder 412 and rails 416a, 416b and pushes fluidic cartridge 500 down into contact with the cartridge support cradle 404 to place contact surface 204a of first thermal assembly 201a in contact with an outer surface of a portion of fluidic cartridge 500 forming a lower wall of reaction/detection chambers 510a1, 510a2 and to place contact surface 204b of second thermal assembly 201b in contact with an outer surface of a portion of fluidic cartridge 500 forming a lower wall of reaction/detection chambers 510b1, 510b2.

In step S608, the presence of the fluidic cartridge 500 between the upper heater (first thermal module 100) and the lower heater (second thermal module 200) will be confirmed by the contact detector 340 as described above.

In step S610, a reaction mixture is formed with the sample in the cartridge 500. At least a portion of the sample contained in chamber W1 and one or more other materials contained within chambers of the sample preparation section 504 are combined by selectively actuating the syringe plunger 362 and stopper 540 with syringe driver 360 within the syringe barrel SB while opening or closing selected ones of the valves V1 to V18 with associated valve actuator heads 406 actuated by a valve actuator of the instrument 10 to move materials from one chamber to another. In one non-limiting example, a fluid sample added to the sample chamber W1 is lysed—either within the sample chamber W1 or prior to addition to the sample chamber W1—to release nucleic acids within the sample. In one non-limiting example, fluid sample may be electromagnetically lysed within a lysis capsule placed in the sample chamber W1 (e.g., lysis capsules 600, 600′, 600″ shown in FIGS. 49-52 or lysis vessel 700 shown in FIGS. 53-55) by a method S800 shown in FIG. 61 and described herein, and which may be combined with method S820 shown in FIG. 62 and/or method S830 shown in FIG. 63 described herein. In another example, fluid sample may be electromagnetically lysed with lytic agents (magnetic and non-magnetic beads) added to the sample within the sample chamber W1 via a bead delivery cap 900 (FIGS. 75-79). In another example, fluid sample may be electromagnetically lysed with lytic agents (magnetic and non-magnetic beads) prepositioned within mechanical lysis sample chamber 1002, 1002′, 1002″ (FIGS. 82-84). Lysed sample material is drawn by the syringe from the sample chamber W1 by closing all valves except valve V1 and raising the syringe plunger 362 and stopper 540 to draw sample into the syringe barrel SB. Lysed sample material drawn from the sample chamber W1 passes through sample filter 538, if provided, to remove debris and amplification inhibitors.

Sample is then moved from the syringe barrel to the purification column within insert 536 situated within a purification chamber W4 by closing all valves except valve V4 and lowering the syringe plunger 362 and stopper 540 to push sample from the syringe barrel SB to the purification chamber W4. Within the purification column of the purification chamber W4, target nucleic acid from the lysed sample material binds to and is immobilized on a solid support of the purification column within insert 536, which may be a silica-based purification column. Non-immobilized material (e.g., cellular material not bound to the solid support that could interfere with amplification and/or detection of a targeted nucleic acid) is moved by the syringe from the purification chamber W4 to one of the waste chambers W11 or W12. The purification column within the purification chamber W4 may be washed one or more times with wash buffer from one or both of chambers W2 and W3, after which the used wash buffer is sent to waste chamber W11 or W12. Finally, the nucleic acid bound to the purification column in the purification chamber W4 is eluted from the purification column using an elution buffer from chamber W10. The eluted nucleic acids are then transferred from the purification chamber W4 to one or more of the reaction/detection chambers 510a1, 510a2, 510b1, 510b2, where, in one non-limiting example, the nucleic acids are subjected to a reaction providing an indication of the presence or amount of an analyte of interest.

If the reaction to be performed on the sample is a PCR-based assay, a master-mix (i.e., a solution including all the components for a PCR reaction that are not analyte-specific) is formed and combined with a portion of the sample and an analyte-specific probe to form the reaction mixture. In step S612, the reaction mixture is drawn into the syringe barrel SB by the syringe plunger 362 and stopper 540 driven by the syringe driver 360—e.g., from chamber W4 by closing sample preparation valves V1 to V3 and V5 to V12 and opening sample preparation valve V4 with a valve actuator—and then pushed by the syringe plunger 362 and stopper 540 into one or more of the reaction/detection chambers 510a1, 510a2, 510b1, 510b2. In some examples, a reaction mixture having a different analyte-specific probe is produced for each of the reaction/detection chambers 510a1, 510a2, 510b1, 510b2 for detecting a different analyte of interest in each of the reaction/detection chambers. One or more reagents, e.g., PCR master-mix and/or an analyte-specific probe, may be pre-placed in the reaction/detection chambers 510a1, 510a2, 510b1, 510b2 so that the reaction mixtures are formed in the reaction/detection chambers when processed sample is added to the reaction/detection chambers.

In one non-limiting example, flow of the reaction mixture from the syringe barrel SB to the chambers 510a1, 510a2, 510b1, 510b2 is controlled as follows. To move reaction mixture from the syringe barrel SB to the reaction chamber 510a1, a valve actuator is operated to actuate (retract) associated valve actuator rods to open valves V14 and V18, and the syringe plunger 362 and stopper 540 are lowered by the syringe driver 360 to expel an amount of reaction mixture from the syringe barrel SB into the reaction chamber 510a1. To move reaction mixture from the syringe barrel SB to the reaction chamber 510a2, a valve actuator is operated to actuate (retract) associated valve actuator rods to open valves V14 and V17, and the syringe plunger 362 and stopper 540 are lowered by the syringe driver 360 to expel an amount of reaction mixture from the syringe barrel SB into the reaction chamber 510a2. To move reaction mixture from the syringe barrel SB to the reaction chamber 510b1, a valve actuator is operated to actuate (retract) associated valve actuator rods to open valves V13 and V16, and the syringe plunger 362 and stopper 540 are lowered to expel an amount of reaction mixture from the syringe barrel SB into the reaction chamber 510b1. To move reaction mixture from the syringe barrel SB to the reaction chamber 510b2, a valve actuator is operated to actuate (retract) associated valve actuator rods to open valves V13 and V15, and the syringe plunger 362 and stopper 540 are lowered to expel an amount of reaction mixture from the syringe barrel SB into the reaction chamber 510b2.

Capacitive flow sensor 146 may be used to detect fluid flow within flow channels located downstream of the reaction/detection chambers 510a1, 510a2, 510b1, 510b2. Detection of fluid flow within the downstream channels may be employed as a feedback control signal to ensure proper filling of the reaction/detection chambers 510a1, 510a2, 510b1, 510b2—e.g., by causing reaction mixture to be pushed into the reaction/detection chambers 510a1, 510a2, 510b1, 510b2 until fluid flow is detected at the flow sensor 146. Alternatively, detection of fluid flow within the downstream channels may be employed as a process control signal to ensure proper filling of the reaction/detection chambers 510a1, 510a2, 510b1, 510b2—e.g., by causing a specified volume of reaction mixture to be pushed into the reaction/detection chambers 510a1, 510a2, 510b1, 510b2, whereby fluid flow detected at the flow sensor 146 will confirm that the reaction/detection chambers 510a1, 510a2, 510b1, 510b2 have been filled.

In step S614, the reaction mixture within each of the reaction/detection chambers 510a1, 510a2, 510b1, 510b2 is incubated.

To heat the reaction/detection chambers, power is applied to one or more of the thermal elements 108a, 108b, 208a, 208b to generate thermal energy that is applied, e.g., by thermal conduction via the corresponding thermal blocks 102a, 102b, 202a, 202b, to the associated reaction/detection chambers 510a1, 510a2, 510b1, 510b2, respectively, to heat, cool, or alternately heat and cool the contents of the reaction/detection chambers. The thermal assemblies 101a, 101b of first thermal module 100 and the thermal assemblies 201a, 201b of the second thermal module 200 can be configured to apply a desired thermal profile to the contents of the chambers 510a1, 510a2, 510b1, 510b2. In some examples, the thermal profile may be an isothermal profile, an ascending or descending temperature ramp profile, or a thermal cycling profile. As previously noted, the contents of the chambers 510a1, 510a2, 510b1, 510b2 may include reaction mixtures that include a sample solution, amplification reagents for amplifying any analyte of interest (e.g., nucleic acid) that may be present in the sample solution when exposed to appropriate amplification conditions (including prescribed thermal conditions), and a detectable probe configured to emit a detectable optical signal when bound to any analyte of interest that may be present in the sample solution or an amplification product thereof. The detectable probe may emit a detectable optical signal spontaneously (e.g., a chemiluminescent signal) or when excited by an optical excitation signal of a prescribed wavelength (e.g., fluorescence emitted by a fluorescent dye or a fluorophore).

In one non-limiting example, where the test to be performed is a real-time PCR nucleic acid amplification assay, a first step may be to heat the reaction mixture contained in the reaction/detection chambers at temperature within the range of 40° C. to 60° C. (e.g. 46° C.) for period of 1 to 20 minutes (e.g. 5 minutes) to activate a reverse transcriptase (RT) within the reaction mixture when the target is RNA. When the target nucleic acid is a DNA, RT is not used, and this step may be omitted. A next step is to heat the reaction mixture at temperature of about 95° C. for a period of 30 seconds to 2 minutes to activate a hot start Taq polymerase enzyme within the reaction mixture. After activating the RT (in the case of an RNA target) and Taq polymerase, thermal cycling may begin. The thermal cycle may comprise two temperatures per cycle—e.g., 60° C. (the annealing temperature) for a period of about 5 to 30 seconds (e.g., 22 seconds) and then 90° C. to 95° C. (the melt temperature) for a period of about 1 to 5 seconds. In one non-limiting example, 40 to 50 thermal cycles may be performed, and fluorescence from the contents of the reaction/detection chambers may be measured once each cycle (e.g., at 60° C.) to obtain 40 to 50 data points and from which an emergence of a fluorescent signal is detected or no fluorescent signal is detected due to the absence of the signal.

Although each chamber 510a1, 510a2, 510b1, 510b2 is exposed to the same temperature profile by the first thermal module 100 and the second thermal module 200, the thermal elements 108a, 108b of the first and second thermal assemblies 101a, 101b, respectively, of the first thermal module 100, and the thermal elements 208a, 208b of the first and second thermal assemblies 201a, 201b, respectively, of the second thermal module 200 are independently controlled. The first thermal assemblies 101a, 201a of the first and second thermal modules 100, 200, respectively, apply the same temperature profile to chambers 510a1, 510a2, and the second thermal assemblies 101b, 201b of the first and second thermal modules 100, 200, respectively, apply the same temperature profile to chambers 510b1, 510b2. The temperature profile applied to chambers 510a1, 510a2 may be the same as or different from the temperature profile applied to chambers 510b1, 510b2.

As shown in FIGS. 31 and 36, first thermal assembly 101a of first thermal module 100 has a separate and independent connector 140 connecting connector board 122 to control board 150 (e.g., via a ribbon cable (not shown)), and second thermal assembly 101b of first thermal module 100 has a separate and independent connector 142 connecting connector board 122 to control board 150 (e.g., via a ribbon cable (not shown)). As shown in FIGS. 32-34 and 39-42, first thermal assembly 201a of second thermal module 200 has a separate and independent connector 230a connecting connector board 222a to control board 150 via connector ribbon cable 232, and second thermal assembly 201b of second thermal module 200 has a separate and independent connector 230b connecting connector board 222b to control board 150 via connector ribbon cable 234. One or more controllers are provided for controlling the temperature of each thermal element 108a, 108b, 208a, 208b, and the controller(s) may be incorporated on the control board 150 or may be remote from the control board 150.

As noted above and explained below, in one non-limiting example, power to and thermal energy generated by each of thermal elements 108a, 108b, 208a, 208b are independently controlled. To facilitate independent control of the thermal elements 108a, 108b, 208a, 208b, the controller(s) controlling the thermal elements may receive independent control feedbacks. For example, as shown in FIG. 37, first thermal assembly 101a of the first thermal module 100 may include thermistors or other thermal/temperature sensors 109a1, 109a2 embedded in the thermal block 102a, and second thermal assembly 101b of the first thermal module 100 may include thermistors or other thermal/temperature sensors 109b1, 109b2 embedded in the thermal block 102b that are independent of the thermistors 109a1, 109a2. Although each thermal assembly is shown having two thermistors, each thermal assembly may include fewer than, or more than, two thermistors. Thermistors 109a1, 109a2 provide temperature feedback signals to the controller(s) controlling power to the thermal element 108a to control the temperature of thermal element 108a and the temperature of thermal block 102a, and, for this purpose, thermistors 109a1, 109a2 may be connected to the controller(s) via the control board 150. Similarly, thermistors 109b1, 109b2 provide temperature feedback signals to the controller(s) controlling power to the thermal element 108b to control the temperature of thermal element 108b and the temperature of thermal block 102b, and, for this purpose, thermistors 109b1, 109b2 may be connected to the controller(s) via the control board 150. Control signals provided by thermistors 109a1, 109a2 are independent of control signals provided by thermistors 109b1, 109b2, and vice versa.

Similarly, first thermal assembly 201a of the second thermal module 200 may include one or more thermistors or other thermal/temperature sensors (not shown) embedded in the thermal block 202a, and second thermal assembly 201b of the second thermal module 200 may include one or more thermistors or other thermal/temperature sensors (not shown) embedded in the thermal block 202b. The thermistor(s) of the first thermal assembly 201a of the second thermal module 200 provide temperature feedback signals to the controller(s) controlling power to the thermal element 208a to control the temperature of thermal element 208a and the temperature of thermal block 202a, and, for this purpose, the thermistor(s) of thermal block 202a may be connected to the controller(s) via the control board 150. Similarly, the thermistor(s) of the second thermal assembly 201b of the second thermal module 200 provide temperature feedback signals to the controller(s) controlling power to the thermal element 208b to control the temperature of thermal element 208b and the temperature of thermal block 202b, and, for this purpose, the thermistor(s) of thermal block 202b may be connected to the controller(s) via the control board 150. Control signals provided by thermistor(s) of the first thermal assembly 201a are independent of control signals provided by thermistor(s) of the second thermal assembly 201b, and vice versa.

While each thermal assembly 101a, 101b, 201a, 201b is independently controlled, in an embodiment, all thermal assemblies may be controlled to the same temperature profile, as explained below.

One control input option for controlling the temperature of a thermal cycler is to hold the heating element (e.g., thermal elements 108a, 108b, 208a, 208b) at a first, lower temperature (e.g., 60° C.) for the required time and then apply a nearly instantaneous pulse of maximum power to increase the temperature of the heating element to a second, higher temperature (e.g., 90° C.) as quickly as possible and then allow the system (i.e., the thermal assembly) to stabilize at the second temperature. But, due to differences in the thermal characteristics (thermal inertia) of the different systems with which each heating element is associated, as well as differences in the performance of different heating elements, the time required for the various system components to stabilize at the second temperature can vary so that the contact surfaces 104a, 104b of thermal assemblies 101a, 101b, respectively, of the first thermal module 100 and the contact surfaces 204a, 204b of the thermal assemblies 201a, 201b, respectively, of the second thermal module 200 may reach the desired second temperature at different times. Thus, the different thermal assemblies heating opposite sides of the reaction/detection chambers 510a1, 510a2, 510b1, 510b2 may not be thermally synchronized. Factors that can affect how fast the system reaches a temperature set point include the size of the thermal element, the age of the thermal element, ambient temperature, thickness of the films 512, 530 on the fluidic cartridge 500 and whether a thermally-conductive laminate seal 532a, 532b is placed over the reaction/detection chambers (see FIG. 8), the size and material (thermal mass) of thermal blocks 102a, 102b, 202a, 202b, the size and material (thermal mass) of the mounting block 118 and the heat sinks 216a, 216b, etc.

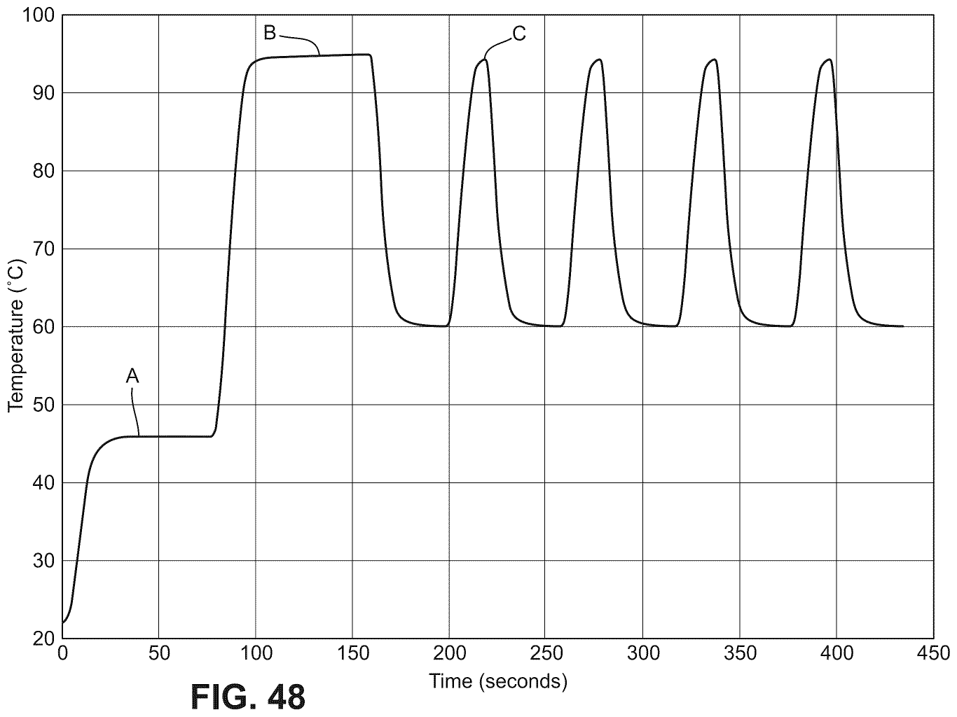

It has been discovered that, instead of applying a nearly instantaneous pulse of maximum power to increase the temperature of the heating element from the first temperature to the second temperature, applying a power input to the different thermal assemblies in the form of a power versus time profile (referred to as a power profile or power curve) in a smooth continuous fashion and controlled via thermal feedback allows each thermal assembly to “keep up” thermally, and thus, all thermal assemblies will follow the same temperature profile (i.e., temperature vs. time performance) and reach the desired temperature set points at the same time to remain thermally synchronized. An example of a temperature profile (or thermal waveform) for controlling the thermal assemblies 101a, 101b, 201a, 201b is shown in FIG. 48. The temperature profile includes a part “A” representing RT enzyme incubation at about 46° C. for a period of about 50 seconds, a part “B” representing enzyme hot start at about 95° C. for a period of about 67 seconds, and part “C” representing thermal cycles, wherein each cycle comprises incubation at about 60° C. for a period of about 22 seconds and incubation at about 95° C. for a period of about 5 seconds. Note also that within each cycle within part “C,” the transition from 60° C. to 95° C. is smooth and continuous over a period of about 22 seconds.

In one embodiment, the thermal elements 108a, 108b of the first and second thermal assemblies 101a, 101b, respectively, of the first thermal module 100, and thermal elements 208a, 208b of the first and second thermal assemblies 201a, 201b, respectively, of the second thermal module 200 are controlled independently to achieve a common temperature, or thermal, response profile, such as that shown in FIG. 48, for each of the thermal assemblies 101a, 101b, 201a, 201b. In one non-limiting example, to achieve the same temperature profile of FIG. 48 in the thermal assemblies 101a, 101b, 201a, 201b, the power profiles (power vs. time) applied to each of the thermal elements 108a, 108b, 208a, 208b of the thermal assemblies may vary depending on the thermal inertia of the first and second thermal assemblies 101a, 101b, 201a, 201b of the first and second thermal modules 100, 200. Power is applied to each of the thermal elements 108a, 108b, 208a, 208b independently of the power applied to other thermal elements and the applied power to each thermal element may be in response to measurements of a thermal sensor (e.g., output of a thermistor) coupled to the thermal element (which is independent of the temperature sensor of the other thermal elements) as compared to the desired thermal profile. That is, each thermal assembly is driven to the same temperature profile (e.g., FIG. 48) by independently applying power to the thermal element of the thermal assembly in response to comparisons of measurements of the temperature sensor of the thermal assembly to the desired temperature profile.

In step S616, optical readings are taken from the reaction mixture within the reaction/detection chambers. As thermal energy is being applied to the reaction mixtures within the detection/reaction chambers 510a1, 510a2, 510b1, 510b2, each detection/reaction chamber can be interrogated for the emission of one or more detectable optical signals via optical fibers 130a1, 130a2, 130b1, 130b2 and signal detectors (optical devices 650a1, 650a2, 650b1, 650b2) constructed and arranged to detect optical signals transmitted by the fibers. As noted above, the signal detector(s) may comprise a photodetector for detecting light spontaneously emitted (e.g., chemiluminescence) from the reaction/detection chambers 510a1, 510a2, 510b1, 510b2 and which is indicative of the presence or absence of an analyte of interest (e.g., target molecule). In another example, the signal detector(s) may comprise a fluorometer including an excitation light source for emitting excitation of light of a prescribed excitation wavelength that is transmitted by the fiber to the reaction/detection chambers 510a1, 510a2, 510b1, 510b2 and an emission detector for detecting light of a prescribed emission wavelength that is emitted by the contents of the chamber (i.e., excitation light is absorbed by a fluorescent dye or a fluorophore, which then emits fluorescent light of a different wavelength) and transmitted by the fiber from the reaction/detection chamber to the emission detector.

For detecting the amount of an analyte present in a sample, an emission time signal may be analyzed by known processes to determine an emergence cycle of a signal (e.g., fluorescent signal) above a background signal from a real-time detector (e.g., fluorometer) during a polymerase chain reaction (PCR) amplification. Real-Time PCR monitors the amplification of a targeted analyte (i.e., DNA or RNA) in real-time. A targeted analyte of the sample will be amplified during PCR and generate a fluorescent signal, which may be recorded in relative fluorescence unit (RFU) readings. This recorded data is processed in a series of steps (sometimes referred to as the TCycle (or Ct) Algorithm) in order to determine the targeted analyte status in the original sample (e.g., valid, invalid, positive, negative and/or concentration). A cycle refers to one round of a thermal processing reaction in a thermal cycler. Typically a PCR reaction goes through multiple cycles (e.g., 35-50 cycles, 35-45 cycles, 40-50 cycles, etc.). Multiple fluorescence measurements per reaction/detection chambers 510a1, 510a2, 510b1, 510b2 may be taken within each cycle. Ct is the number of cycles before which the analyte specific signal has reached a preset threshold limit during the amplification (also called emergence cycle).

### Hardware and Software

Aspects of the subject matter disclosed herein may be implemented via control and computing hardware components, software (which may include firmware), data input components, and data output components. Hardware components include computing and control modules (e.g., system controller(s)), such as processing circuitry, configured to effect computational and/or control steps by receiving one or more input values, executing one or more algorithms stored on non-transitory machine-readable media (e.g., software) that provide instruction for manipulating or otherwise acting on or in response to the input values, and output one or more output values. Such processing circuitry may include one or more processors (e.g., one or more general purpose microprocessors and/or one or more other processors, such as one or more computer(s), an application specific integrated circuit (ASIC), field-programmable gate arrays (FPGAs), and the like), which processors may be co-located in a single housing or in a single data center or may be geographically distributed (i.e., the processing circuitry may be encompassed by a distributed computing apparatus). Such outputs may be displayed or otherwise indicated to a user for providing information to the user, for example information as to the status of the instrument or of a process being performed thereby, or such outputs may comprise inputs to other processes and/or control algorithms. Data input components comprise elements by which data is input for use by the control and computing hardware components. Such data inputs may comprise signals generated by sensors or scanners, such as, position sensors, speed sensors, accelerometers, environmental (e.g., temperature) sensors, motor encoders, barcode scanners, or RFID scanners, as well as manual input elements, such as keyboards, stylus-based input devices, touch screens, microphones, switches, manually-operated scanners, etc. Data inputs may further include data retrieved from memory. Data output components may comprise hard drives or other storage media, monitors, printers, indicator lights, or audible signal elements (e.g., chime, buzzer, horn, bell, etc.).

The above-described techniques can be implemented in digital and/or analog electronic circuitry, or in computer hardware, firmware, software, or in combinations of them. The implementation can be as a computer program product, i.e., a computer program tangibly embodied in a machine-readable storage device, for execution by, or to control the operation of, a data processing apparatus, e.g., a programmable processor, a computer, and/or multiple computers. A computer program can be written in any form of computer or programming language, including source code, compiled code, interpreted code, and/or machine code, and the computer program can be deployed in any form, including as a stand-alone program or as a subroutine, element, or other unit suitable for use in a computing environment. A computer program can be deployed to be executed on one computer or on multiple computers at one or more sites.

Method steps can be performed by one or more processors executing a computer program to perform functions of the invention by operating on input data and/or generating output data. Method steps can also be performed by, and an apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit). Subroutines can refer to portions of the computer program and/or the processor/special circuitry that implement one or more functions.

Processors suitable for the execution of a computer program include, by way of example, both general and special purpose microprocessors, and any one or more processors of any kind of digital or analog computer. Generally, a processor receives instructions and data from a read-only memory or a random-access memory or both. The essential elements of a computer are a processor for executing instructions and one or more memory devices for storing instructions and/or data. Memory devices, such as a cache, can be used to temporarily store data. Memory devices can also be used for long-term data storage. Generally, a computer also includes, or is operatively coupled to receive data from or transfer data to, or both, one or more mass storage devices for storing data, e.g., magnetic, magneto-optical disks, or optical disks. A computer can also be operatively coupled to a communications network in order to receive instructions and/or data from the network and/or to transfer instructions and/or data to the network. Computer-readable storage devices suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. The processor and the memory can be supplemented by and/or incorporated in special purpose logic circuitry.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the scope of the following appended claims.

1. A lysis vessel for performing cell lysis, comprising:
a laterally extending member having a vent extending therethrough;
a sleeve depending from the laterally extending member, wherein a bottom end of the sleeve defines an open bottom end of the vessel;
a first porous membrane affixed to a top or bottom surface of the laterally extending member, the first porous membrane covering the vent, a second porous membrane affixed to the bottom end of the sleeve, the second porous membrane covering the open bottom end, wherein the bottom surface of the laterally extending member, an inner surface of the sleeve and the first and second porous membranes define a lysis chamber;
a plurality of non-magnetic beads contained within the lysis chamber; and
at least one magnetic element contained within the lysis chamber, wherein the first and the second porous membranes are sized to retain the plurality of non-magnetic beads and the at least one magnetic element within the lysis chamber.

2. The lysis vessel of claim 1, further comprising a peripheral wall extending upward from the periphery of the laterally extending member.

3. The lysis vessel of claim 2, wherein the peripheral wall is adapted for manual gripping.

4. The lysis vessel of claim 1, wherein the second porous membrane comprises a mesh, the mesh being liquid permeable.

5. The lysis vessel of claim 4, wherein the second porous membrane is hydrophilic.

6. The lysis vessel of claim 4, wherein the first porous membrane is gas permeable but not liquid permeable.

7. The lysis vessel of claim 6, wherein the first porous membrane has a porosity of 0.2 μm to 0.4 μm, and the second porous membrane has a porosity of 30 μm to 100 μm.

8. The lysis vessel of claim 1, wherein each of the plurality of non-magnetic beads is comprised of a ceramic or a glass.

9. The lysis vessel of claim 1, wherein each of the plurality of non-magnetic beads has a spherical shape, and wherein each of the plurality of non-magnetic beads has diameter of 100 μm to 2000 μm.

10. The lysis vessel of claim 1, wherein the at least one magnetic element is plated or encapsulated with a non-magnetic material.

11. The lysis vessel of claim 1, wherein the at least one magnetic element occupies a greater volume than any of the plurality of non-magnetic beads.

12. The lysis vessel of claim 10, wherein the at least one magnetic element has the shape of a cube, and wherein the width of each face of the cube is 2.0 millimeters to 4.3 millimeters.

13. The lysis vessel of claim 1, wherein the plurality of non-magnetic beads occupies a volume of 50% to 75% of the volume of the lysis chamber, and wherein the at least one magnetic element occupies a volume of 4.5% to 11% of the volume of the lysis chamber.

14. The lysis vessel of claim 1, further comprising an internal control contained within the lysis chamber, wherein the internal control is provided to validate an assay result and/or to validate the effectiveness of the cell lysis procedure.

15. The lysis vessel of claim 14, wherein at least a portion of the internal control is contained in an internal control reagent disposed on the second porous membrane, and wherein the internal control reagent is adapted to dissolve when contacted by a fluid sample.

16. The lysis vessel of claim 14, wherein at least a portion of the internal control is contained in an internal control reagent disposed on at least a portion of the plurality of non-magnetic beads, and wherein the internal control reagent is adapted to dissolve when contacted by a fluid sample.

17. The lysis vessel of claim 14, wherein at least a portion of the internal control is contained in an internal control reagent disposed on at least one of the bottom surface of the laterally extending member and the inner surface of the sleeve, and wherein the internal control reagent is adapted to dissolve when contacted by a fluid sample.

18. The lysis vessel of claim 14, wherein at least a portion of the internal control is embedded in or contained within an internal control pellet adapted to dissolve when contacted by a fluid sample and/or to disintegrate when the plurality of non-magnetic beads is agitated, the internal control pellet being contained within the lysis chamber.

19. A fluidic cartridge, comprising:
a cartridge body comprising a sample chamber, the sample chamber having an open top end; and
the lysis vessel of claim 1 disposed within the sample chamber.

20. A method for processing cells contained in a fluid sample, comprising:
(A) dispensing the fluid sample into a sample chamber of a fluidic cartridge, the sample chamber having an open top end;
(B) after (A), inserting the sleeve of the lysis vessel of claim 1 into the open top end of the sample chamber;
(C) after (B), subjecting the at least one magnetic element to a magnetic field, thereby causing movement of the at least one magnetic element within the lysis chamber of the lysis vessel, the movement of the at least one magnetic element within the lysis chamber of the lysis vessel causing movement of the plurality of non-magnetic beads within the lysis chamber of the lysis vessel, and the movement of the plurality of non-magnetic beads within the lysis chamber of the lysis vessel causing cells contained within the fluid sample within the lysis chamber of the lysis vessel to lyse and release nucleic acids.
